(19) United States
(12) Patent Application Publication
(10) Pub. No.: US 2024/0084304 A1
(43) Pub. Date: Mar. 14, 2024

(54) COMPOSITION AND METHOD FOR INHIBITING ANGIOTENSINOGEN (AGT) PROTEIN EXPRESSION

(71) Applicant: Shanghai Argo Biopharmaceutical Co., Ltd., Shanghai (CN)

(72) Inventors: Dongxu Shu, Ningbo (CN); Pengcheng Patrick Shao, Warrington Township, PA (US)

(73) Assignee: Shanghai Argo Biopharmaceutical Co., Ltd., Shanghai (CN)

(21) Appl. No.: 18/473,829

(22) Filed: Sep. 25, 2023

Related U.S. Application Data
(63) Continuation of application No. PCT/CN2022/131861, filed on Nov. 15, 2022.

(30) Foreign Application Priority Data
Nov. 16, 2021 (WO) PCT/CN2021/130832
Mar. 18, 2022 (WO) PCT/CN2022/081578

(51) Int. Cl. C12N 15/113; A61K 9/00; A61K 45/06; A61K 47/54; A61P 9/12

(52) U.S. Cl. CPC C12N 15/113; A61K 9/0019; A61K 45/06; A61K 47/549; A61P 9/12

(57) ABSTRACT

Provided are a composition and method for inhibiting angiotensinogen (AGT) protein expression. Specifically, provided are a composition and method capable of being used for reducing AGT gene expression and treating AGT-related diseases and disorders. Provided are an AGT dsRNA reagent capable of being used for reducing AGT expression in a cell and an object, an AGT antisense polynucleotide reagent, a composition containing the AGT dsRNA reagent, and a composition containing the AGT antisense polynucleotide reagent.

### FIELD OF THE INVENTION

The invention relates, in part, to compositions and methods that can be used to inhibit the expression of Angiotensinogen (AGT) protein.

### BACKGROUND

The renin-angiotensin-aldosterone system (RAAS) plays a key role in blood pressure regulation. The RAAS cascade begins with the secretion of renin into the circulation by glomerular cells of the kidney. Renin secretion is stimulated by several factors, including Na⁺ loading the distal tubule, β-sympathetic stimulation, and/or decreased renal perfusion. Active renin in plasma splits angiotensinogen (produced by the liver) into angiotensin I, which is subsequently converted to angiotensin II by circulating and locally expressed angiotensin-converting enzyme (ACE). Most of the effects of angiotensin II on the RAAS are exerted through its binding to the angiotensin II type 1 receptor (AT1R), resulting in arterial vasoconstriction, tubular and glomerular effects such as enhanced Na⁺ Regulation of reabsorption or glomerular filtration rate. Furthermore, AT1R stimulation, together with other stimuli (e.g., corticotropin, anti-diuretic hormone, catecholamines, endothelin, serotonin) and Mg²⁺ and K⁺ levels, leads to aldosterone release, which subsequently promotes distal renal tubule Na⁺ and K⁺ in excretion.

Dysregulation of the RAAS caused by, for example, excessive angiotensin II production and/or AT1R stimulation causes hypertension, which can lead to, increased oxidative stress, promotion of inflammation, hypertrophy, and fibrosis in the heart, kidneys, and arteries, and lead to, for example left ventricular Fibrosis, arterial remodeling, and glomerulosclerosis.

Hypertension is the most prevalent, manageable disease in developed countries, affecting 20-50% of the adult population. Hypertension is a major risk factor for various diseases, disorders, and conditions such as shortened life expectancy, chronic kidney disease, stroke, myocardial infarction, heart failure, aneurysm (e.g., aortic aneurysm), peripheral arterial disease, cardiac injury (For example, cardiac dilation or hypertrophy) and other cardiovascular-related diseases, disorders and/or conditions. Furthermore, hypertension has been shown to be an important risk factor for cardiovascular morbidity and mortality, accounting for or constituting 62% of all strokes and 49% of all heart disease cases. In 2017, a change in the guidelines for the diagnosis, prevention, and treatment of hypertension occurred, providing targets for even lower blood pressure to further reduce the risk of developing hypertension-related diseases and conditions (e.g., Reboussin et al; Systematic Review for the 2017ACC/AHA/AAPA/ABC/ACPM/AGS/APhA/ASH/ASPC/NMA/PCNA Guideline for the Prevention, Detection, Evaluation, and Management of High Blood Pressure in Adults: A Report of the American College of Cardiology/American Heart Association Task Force on Clinical Practice Guidelines. J Am Coll Cardiol. 2017 Nov. 7. pii: S0735-1097(17)41517-8. doi: 10.1016/j.jacc 0.2017.11.004; and Whelton et al. (2017ACC/AHA/AAPA/ABC/ACPM/AGS/APhA/ASH/ASPC/NMA/PCNA Guideline for the Prevention, Detection, Evaluation, and Management of High Blood Pressure in Adults: A Report of the American College of Cardiology/American Heart Association Task Force on Clinical Practice Guidelines. J Am Coll Cardiol. 2017 Nov. 7. pii: 50735-1097(17)41519-1.doi: 10.1016/j.jacc.2017.11.006).

Despite the large number of antihypertensive drugs available for the treatment of hypertension, more than two-thirds of subjects are not controlled on one antihypertensive drug and require two or more antihypertensive drugs from different drug classes. This further reduces the number of subjects with controlled blood pressure due to decreased compliance and increased side effects with increased dosing.

Accordingly, there is a need in the art for alternative therapies and combination therapies for the treatment of hypertension and other angiotensinogen-related diseases.

### SUMMARY OF THE INVENTION

According to one aspect of the present invention, there is provided a double-stranded ribonucleic acid (dsRNA) agent for inhibiting the expression of Angiotensinogen (AGT), the dsRNA agent comprising a sense strand and an antisense strand, nucleotide positions 2 to 18 in the antisense strand including a region of complementarity to an AGT RNA transcript, wherein the region of complementarity comprises at least 15 contiguous nucleotides that differ by 0, 1, 2, or 3 nucleotides from one of the antisense sequences listed in Tables 1-4, and optionally includes a targeting ligand. In some embodiments, the region complementary to the AGT RNA transcript comprises at least 15, 16, 17, 18, or 19 contiguous nucleotides that differ by no more than 3 nucleotides from one of the antisense sequences listed in Tables 1-4. In some embodiments, the antisense strand of the dsRNA is at least substantially complementary to any one of a target region of SEQ ID NO: 519 and is provided in any one of Tables 1-4. In some embodiments, the antisense strand of the dsRNA is fully complementary to any one of target region of SEQ ID NO: 519 and is provided in any one of Tables 1-4. In some embodiments, the dsRNA agent comprises any one of the sense strand sequences listed in Tables 1-4, wherein the sense strand sequence is at least substantially complementary to the antisense strand sequence in the dsRNA agent. In some embodiments, the dsRNA agent comprises any one of the sense strand sequences listed in Tables 1-4, wherein the sense strand sequence is fully complementary to the antisense strand sequence in the dsRNA agent. In some embodiments, the dsRNA agent comprises any one of the antisense strand sequences listed in Tables 1-4. In some embodiments, the dsRNA agent includes the sequences set forth as a duplex sequence in any of Tables 1-4.

In some embodiments, the dsRNA agent comprises a sense strand that differs by 0, 1, 2, or 3 nucleotides from formula (A): 5′-Z₁AGCUUGUUUGUGAAACZ₂-3′ (SEQ ID NO:656) formula (A), wherein the nucleotide sequence Z₁ is 0-15 nucleotides in length, Z₂ is selected from one of A, U, C, G or absent. In some embodiments, Z₁ is a nucleotide sequence comprising 1-4 nucleotide motifs. In certain embodiments, the nucleotide sequence Z₁ comprises 1, 2, 3, or 4 nucleotides in length. In certain embodiments, Z₂ is A. In certain embodiments, Z₁ is a nucleotide sequence comprising CACC or GACC. In certain embodiments, the nucleotide sequence Z₁ is selected from one of C, AC, UC, GC, CC, ACC, UCC, GCC, CCC, GACC, AACC, UACC, CACC, CGACC, CCGACC, ACCGACC, AACCGACC, CAACCGACC, CCAACCGACC (SEQ ID NO:660), UCCAACCGACC (SEQ ID NO:661), UUCCAACCGACC (SEQ ID NO:662), AUUCCAACCGACC (SEQ ID NO:663), AAUUCCAACCGACC (SEQ ID NO:664) or GAAUUCCAACCGACC (SEQ ID NO:665). In some embodiments, the dsRNA agent comprises an antisense strand that differs by 0, 1, 2, or 3 nucleotides from formula (B): 5′-Z₃GUUUCACAAACAAGCUZ₄-3′ (SEQ ID NO:657) formula (B), wherein Z 3 is selected from A, U, C, G or absent, the nucleotide sequence Z₄ is 0-15 nucleotides in length. In certain embodiments, Z₄ is a nucleotide sequence comprising 1-4 nucleotides in length. In certain embodiments, Z₄ is a nucleotide sequence comprising 1, 2, 3, or 4 nucleotides. In certain embodiments, Z₃ is U. In certain embodiments, the nucleotide sequence Z₄ is selected from nucleotide sequences comprising GGUC or GGUG. In certain embodiments, the nucleotide sequence Z 4 is selected from G, GU, GC, GA, GG, GGU, GGA, GGC, GGG, GGUG, GGUC, GGUU, GGUA, GGUCG, GGUCGG, GGUCGGU, GGUCGGUU, GGUCGGUUG, GGUCGGUUGG (SEQ ID NO:666), GGUCGGUUGGA (SEQ ID NO:667), GGUCGGUUGGAA (SEQ ID NO:668), GGUCGGUUGGAAU (SEQ ID NO:669), GGUCGGUUGGAAUU (SEQ ID NO:670) or GGUCGGUUGGAAUUC (SEQ ID NO:671). In some embodiments, the dsRNA agent comprises a sense strand and an antisense strand, the sense strand and antisense strand respectively comprise a nucleotide sequence that differ by 0, 1, 2, or 3 nucleotides from formula (A) and formula (B) as described herein, and optionally comprising a targeting ligand. In certain embodiments, the sense strand (A) and antisense strand (B) of the dsRNA agent are each no more than 35 nucleotides in length. In certain embodiments, the nucleotide sequence Z₁ and Z₄ are fully or partially complementary. In some embodiments, the dsRNA agent comprises a sense strand that differs by 0, 1, 2, or 3 nucleotides from formula (A′): 5′-Z₁′CAGCUUGUUUGUGAAACA-3′ (SEQ ID NO: 658) Formula (A′), the dsRNA agent comprises an antisense strand that differs by 0, 1, 2 or 3 nucleotides from formula (B′): 5′-UGUUUCACAAACAAGCUGZ₄′-3′ (SEQ ID NO:659) Formula (B′), the nucleotide sequence Z₁′ and Z₄′ independently include 0-13 nucleotides. In some embodiments, the nucleotide sequences Z₁′ and Z₄′ independently comprise 1, 2, or 3 nucleotides. In certain embodiments, the nucleotide sequence Z₁′ is selected from one of A, U, G, C, AC, UC, GC, CC, GAC, AAC, UAC, CAC, CGAC, CCGAC, ACCGAC, AACCGAC, CAACCGAC, or GAAUUCCAACCGAC (SEQ ID NO:672). The nucleotide sequence Z₄′ is selected from one of U, C, A, G, GU, GA, GC, GG, GUG, GUC, GUU, GUA, GUCG, GUCGG, GUCGGU, GUCGGUU, GUCGGUUG or GUCGGUUGGAAUUC (SEQ ID NO:673).

In some embodiments, the dsRNA agent comprises a sense strand and an antisense strand, the antisense strand comprises at least 15 contiguous nucleotides that differ by 0, 1, 2, or 3 nucleotides from any one of the antisense sequences selected from the group consisting of

(SEQ ID NO: 162)
5′-UACUCAUUAGAAGAAAAGGUG-3′;
(SEQ ID NO: 163)
5′-UCUUAGACCAAGGAGAAACGG-3′;
(SEQ ID NO: 167)
5′-UGUUUCACAAACAAGCUGGUC-3′;
(SEQ ID NO: 523)
5′-UGUUUCACAAACAAGCUGGUG-3′;
(SEQ ID NO: 184)
5′-UUCGGUUGGAAUUCUUUUUGC-3′;
(SEQ ID NO: 653)
5′-GUUUCACAAACAAGCUGG-3′.

In some embodiments, the dsRNA agent comprises a sense strand and an antisense strand, the sense and antisense strand comprises at least 15 contiguous nucleotides that differ by 1, 2, or 3 nucleotides from any one of the nucleotide sequences listed below, respectively:

Sense strand:
(SEQ ID NO: 65)
5′-CACCUUUUCUUCUAAUGAGUA-3′,
Antisense strand:
(SEQ ID NO: 162)
5′-UACUCAUUAGAAGAAAAGGUG-3′.

In some embodiments, the dsRNA agent comprises a sense strand and an antisense strand, the sense and antisense strand comprises at least 15 contiguous nucleotides that differ by 1, 2, or 3 nucleotides from any one of the nucleotide sequences listed below, respectively:

Sense strand:
(SEQ ID NO: 66)
5′-CCGUUUCUCCUUGGUCUAAGA-3′,
Antisense strand:
(SEQ ID NO: 163)
5′-UCUUAGACCAAGGAGAAACGG-3′.

In some embodiments, the dsRNA agent comprises a sense strand and an antisense strand, the sense and antisense strand comprises at least 15 contiguous nucleotides that differ by 1, 2, or 3 nucleotides from any one of the nucleotide sequences listed below, respectively:

Sense strand:
(SEQ ID NO: 70)
5′-GACCAGCUUGUUUGUGAAACA-3′,
Antisense strand:
(SEQ ID NO: 167)
5′-UGUUUCACAAACAAGCUGGUC-3′.

In some embodiments, the dsRNA agent comprises a sense strand and an antisense strand, the sense and antisense strand comprises at least 15 contiguous nucleotides that differ by 1, 2, or 3 nucleotides from any one of the nucleotide sequences listed below, respectively:

Sense strand:
(SEQ ID NO: 522)
5′-CACCAGCUUGUUUGUGAAACA-3′
Antisense strand:
(SEQ ID NO: 523)
5′-UGUUUCACAAAACAAGCUGGUG-3′;

In some embodiments, the dsRNA agent comprises a sense strand and an antisense strand, the sense and antisense strand comprises at least 15 contiguous nucleotides that differ by 1, 2, or 3 nucleotides from any one of the nucleotide sequences listed below, respectively:

Sense strand:
(SEQ ID NO: 87)
5′-GCAAAAAGAAUUCCAACCGAA-3′,
Antisense strand:
(SEQ ID NO: 184)
5′-UUCGGUUGGAAUUCUUUUUGC-3′.

In some embodiments, the dsRNA agent comprises a sense strand and an antisense strand, the sense and antisense strand comprises at least 15 contiguous nucleotides that differ by 1, 2, or 3 nucleotides from any one of the nucleotide sequences listed below, respectively:

Sense strand:
(SEQ ID NO: 652)
5′-CCAGCUUGUUUGUGAAAC-3′,
Antisense strand:
(SEQ ID NO: 653)
5′-GUUUCACAAACAAGCUGG-3′.

In some embodiments, the dsRNA agent duplex is selected from any one duplex of AD00158-19-2, AD00158-19-1, AD00158-3, AD00158-1, AD00158-2, AD00158, AD00159, AD00159-1, AD00159-2, AD00159-19-1, AD00159-19-2, AD00163, AD00163-1, AD00163-2, AD00163-19-1, AD00163-19-2, AD00163-3, AD00300-1, AD00300-19-1, AD00300-19-2 in Table 1.

In some embodiments, the dsRNA agent duplex is selected from any one duplex of AV01227, AV01228, AV01229, AV01230, AV01231, AV01232, AV01233, AV01234, AV01235, AV01236, AV01237, AV01238, AV01239, AV01240, AV01241, AV01242, AV01243, AV01244, AV01245, AV01246, AV01247, AV01248, AV01249, AV01250, AV01251, AV01252, AV01253, AV01254, AV01255, AV01256, AV01257 or AV01711 in Table 1.

In some embodiments, the dsRNA agent comprises at least one modified nucleotide. In certain embodiments, all or substantially all of the nucleotides of the antisense strand are modified nucleotides. In some embodiments, at least one modified nucleotide includes: 2′-O-methyl nucleotide, 2′-Fluoro nucleotide, 2′-deoxy nucleotide, 2′3′-seco nucleotide mimic, locked nucleotide, unlocked nucleic acid nucleotide (UNA), glycol nucleic acid nucleotide (GNA), 2′-F-Arabino nucleotide, 2′-methoyxyethyl nucleotide, abasic nucleotide, ribitol, inverted nucleotide, inverted abasic nucleotide, inverted 2′-Ome nucleotide, inverted 2′-deoxy nucleotide, 2′-amino-modified nucleotide, 2′-alkyl-modified nucleotide, mopholino nucleotide, and 3′-OMe nucleotide, a nucleotide including a 5′-phosphorothioate group, or a terminal nucleotide linked to a cholesteryl derivative or dodecanoic acid bisdecylamide group, a 2′-amino-modified nucleotide, a phosphoramidite, or a non-natural base including nucleotide.

In some embodiments, these double-stranded ribonucleic acids (dsRNA) agents comprise a sense strand and an antisense strand that is complementary to at least a portion of an mRNA corresponding to a target gene, wherein the antisense strand of dsRNA comprises a nucleotide sequence shown as formula (C) and the sense strand of dsRNA comprises a nucleotide sequence shown as formula (D),

The antisense strand comprises shown as formula (C) listed in the direction of 3′ to 5′:

3′-(N_{L})ₙ N_{M1} N_{L} N_{M2} N_{L} N_{F} N_{L} N_{M3} N_{L} N_{M4} N_{L} N_{M5} N_{M6} N_{L} N_{M7} N_{M8} N_{L} N_{F} N_{L}-5′ formula (C),

The sense strand comprises shown as formula (D) listed in the direction of 5′ to 3′:

5′-(N′_{L})_{n′}N′_{L}N′_{L}N′_{L}N′_{N1}N′_{N2}N′_{N3}N′_{N4}N′_{F}N′_{L}N′_{N5}N′_{N6}N′_{L}N′_{L}N′_{L}N′_{L}N′_{L}N′_{L}N′L-3′ formula (D),

Wherein, each N_{F} represents a 2′-fluoro-modified nucleotide; N_{M1}, N_{M2}, N_{M3}, N_{M4}, N_{M5}, N_{M6}, N_{M7}, and N_{M8} independently represent modified or unmodified nucleotides, There are only three 2′-fluoro-modified nucleotides or only one 2′-fluoro-modified in N_{M1}, N_{M2}, N_{M3}, N_{M4}, N_{M5}, N_{M6}, N_{M7}, and N_{M8}; each N_{L} independently represents a modified or unmodified nucleotide, and the modification is not a 2′-fluoro-modified nucleotide; each N′_{F} represents a 2′-fluoro-modified nucleotide; N′_{N1}, N′_{N2}, N′_{N3}, N′_{N4}, N′_{N5}, and N′_{N6} independently represent modified or unmodified nucleotides, There are only two 2′-fluoro-modified nucleotides in N′_{N1}, N′_{N2}, N′_{N3}, N′_{N4}, N′_{N5}, and N′_{N6}, each N′_{L} independently represents a modified or unmodified nucleotide, and the modification is not a 2′-fluoro-modified nucleotide; and n and n′ may be an integer of 0 to 7.

In certain embodiments, the nucleotide at positions 2, 7, 12, 14, and 16 (counting from the first paired nucleotide from the 5′ end) of the antisense strand represented by formula (C) are 2′-fluorine-modified nucleotides; and The nucleotide at positions 9, 11, and 13 (counting from the first paired nucleotide from the 3′ end) of the sense strand represented by formula (D) are 2′-fluoro-modified nucleotides. In certain embodiments, the nucleotides at positions N_{M2}, N_{M3}, and N_{M6} of the antisense strand represented by formula (C) are 2′-fluoro-modified nucleotides; The nucleotides at positions N′_{N3}, and N′_{N5} of the sense strand shown in formula (D) are 2′-fluoro-modified nucleotides.

In some embodiments, the dsRNA agent includes an E-vinylphosphonate nucleotide at the 5′ end of the guide strand. In certain embodiments, the dsRNA agent includes at least one phosphorothioate internucleoside linkage. In certain embodiments, the sense strand includes at least one phosphorothioate internucleoside linkage. In some embodiments, the antisense strand includes at least one phosphorothioate internucleoside linkage. In some embodiments, the sense strand includes 1, 2, 3, 4, 5, or 6 phosphorothioate internucleoside linkages. In some embodiments, the antisense strand includes 1, 2, 3, 4, 5, or 6 phosphorothioate internucleoside linkages.

In certain embodiments, all or substantially all nucleotides of the sense and antisense strands are modified nucleotides. In some embodiments, the modified sense strand is a modified sense strand sequence listed in Tables 2-4. In some embodiments, the modified antisense strand is a modified antisense strand sequence listed in Tables 2-4.

In certain embodiments, the sense strand is complementary or substantially complementary to the antisense strand, and the region of complementarity is between 16 and 23 nucleotides in length. In some embodiments, the complementary region is 19-21 nucleotides in length. In certain embodiments, the complementary region is 14, 15, 16, 17, 18, 19, 20, 21, 22, 23, 24, 25, 26, 27, 28, 29 or 30 nucleotides in length.

In some embodiments, each strand is no more than 40 nucleotides in length. In some embodiments, each strand is no more than 30 nucleotides in length. In some embodiments, each strand is no more than 25 nucleotides in length. In some embodiments, each strand is no more than 23 nucleotides in length. In some embodiments, each strand is 4, 15, 16, 17, 18, 19, 20, 21, 22, 23, 24, 25, 26, 27, 28, 29 or 30 nucleotides in length.

In certain embodiments, a dsRNA agent comprises at least one modified nucleotide and further comprises one or more targeting or linking groups. In some embodiments, one or more targeting groups or linking groups are conjugated to the sense strand. In some embodiments, the targeting group or linking group includes N-acetyl-galactosamine (GalNAc). In some embodiments, the targeting group has the following structure:

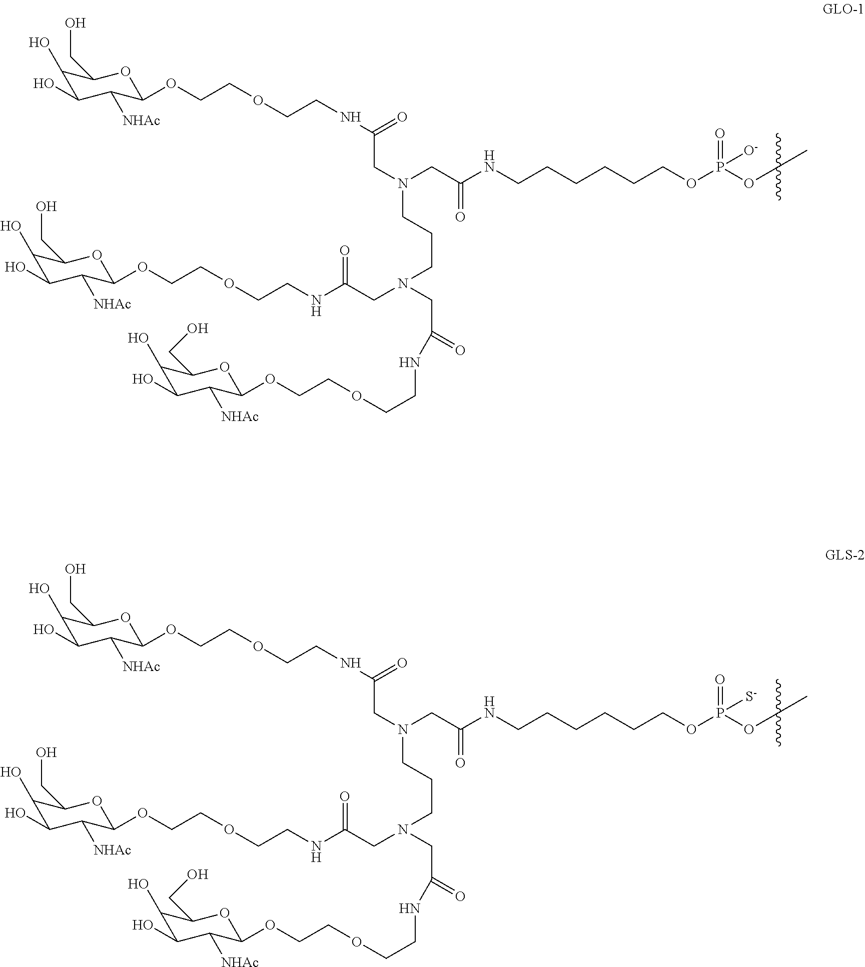 [SMILES: *OP(=O)([O-])OCCCCCCNC(=O)CN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCNC(=O)CN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

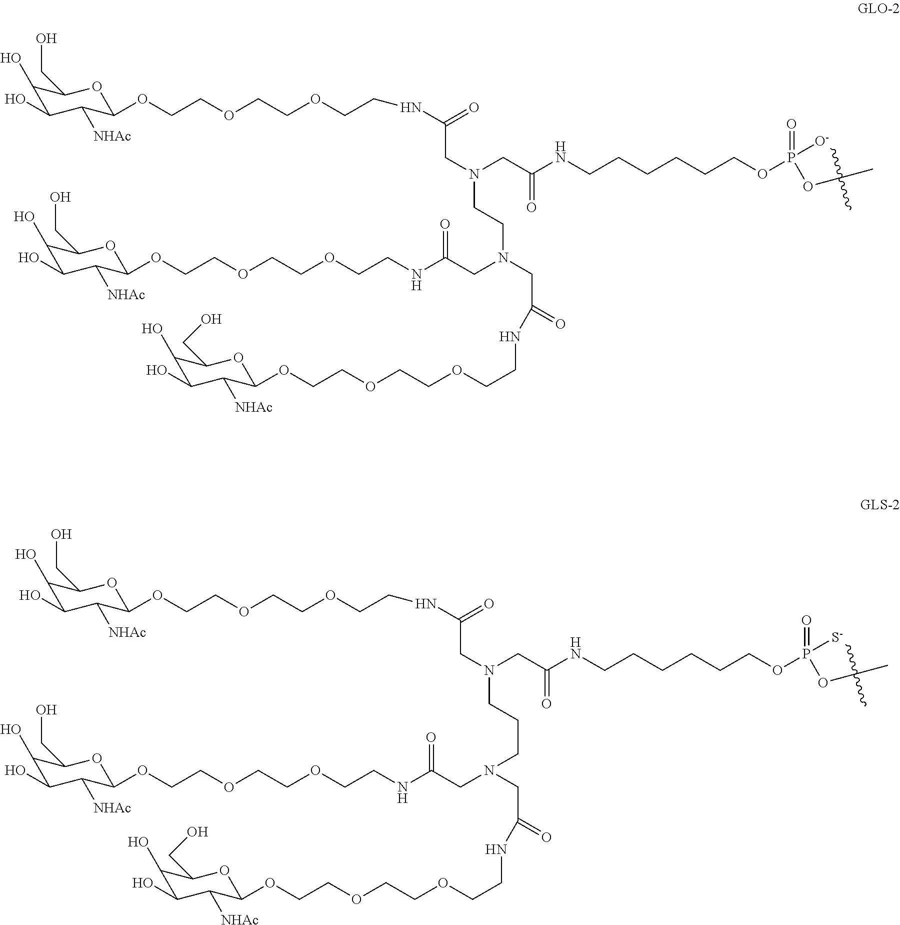 [SMILES: *OP(=O)([O-])OCCCCCCNC(=O)CN(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCCCCCNC(=O)CN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

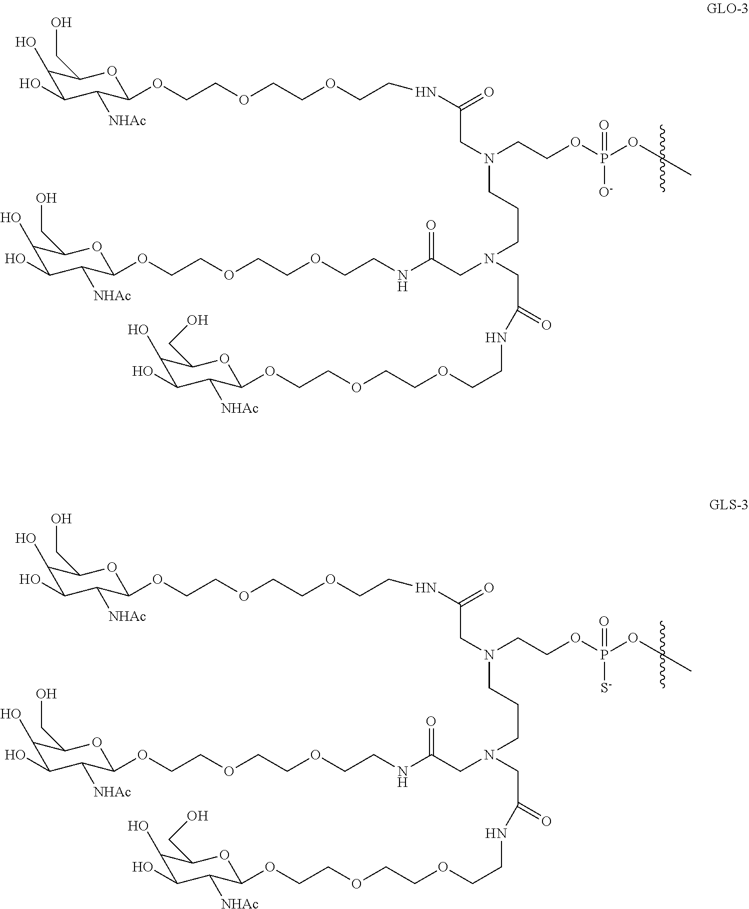 [SMILES: *OP(=O)([O-])OCCN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

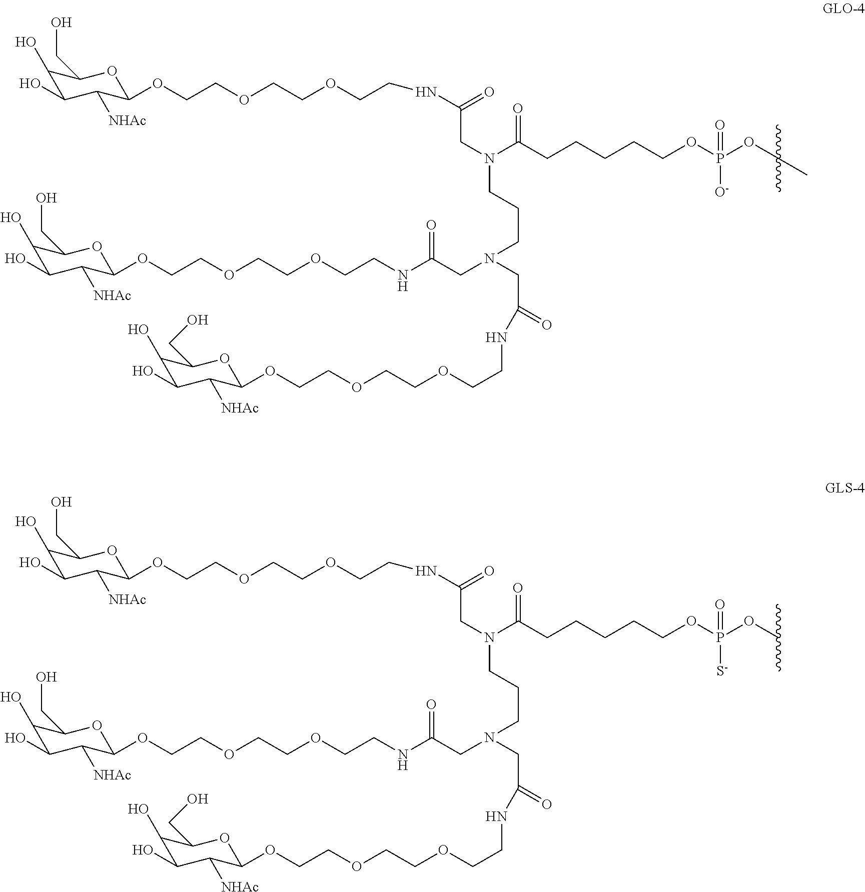 [SMILES: *OP(=O)([O-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

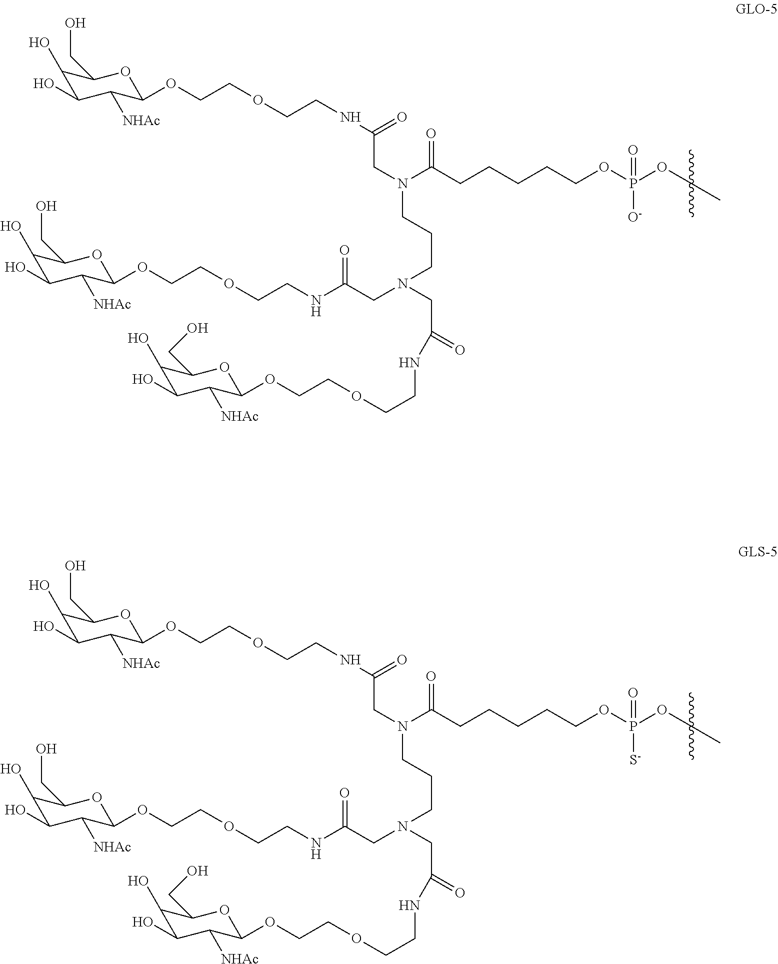 [SMILES: *OP(=O)([O-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

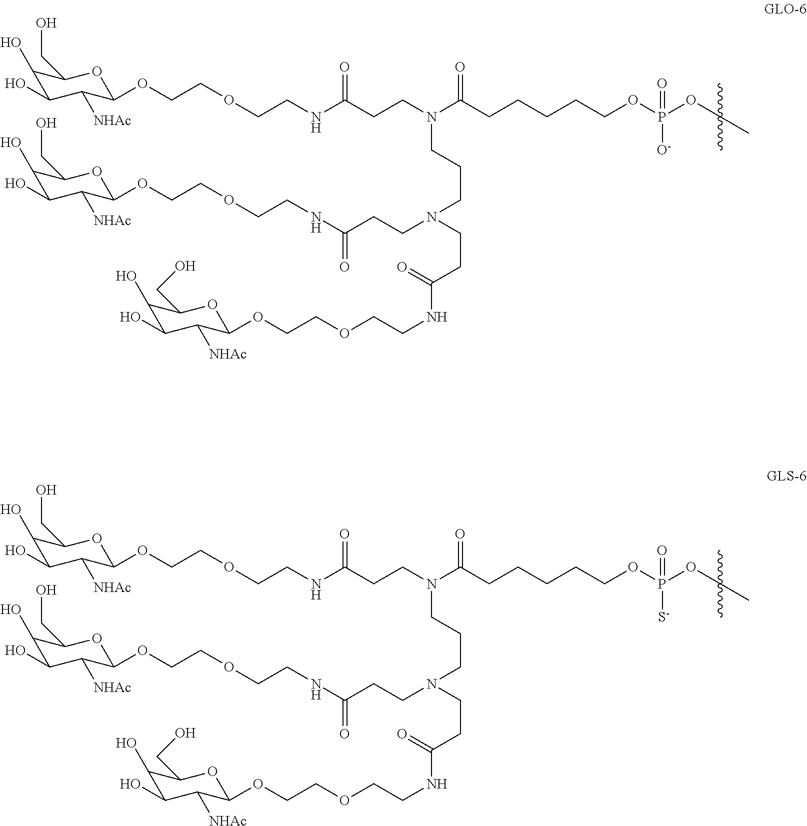 [SMILES: *OP(=O)([O-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O ; *OP(=O)([S-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O]

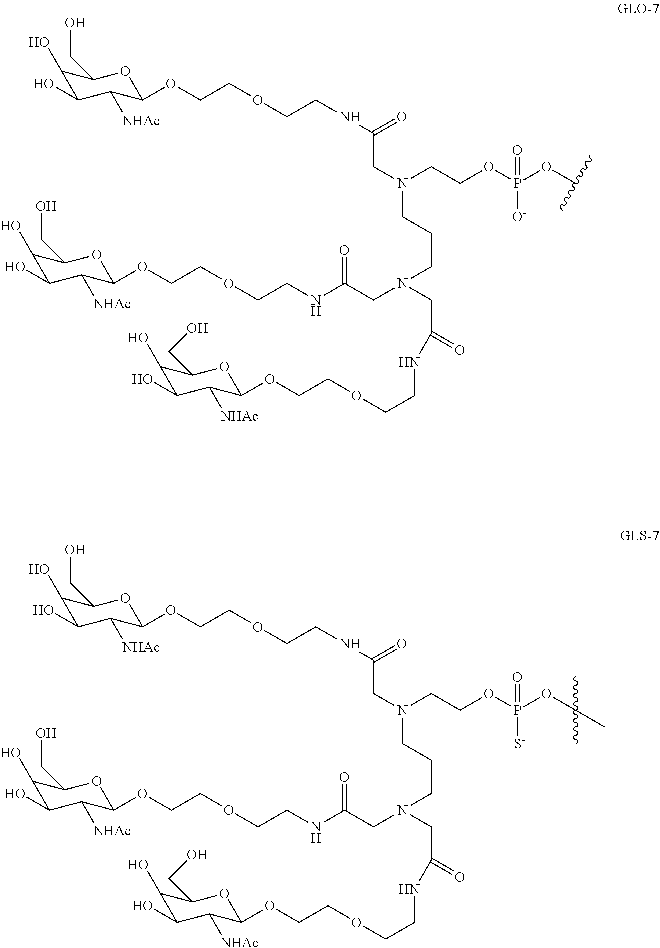 [SMILES: *OP(=O)([O-])OCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

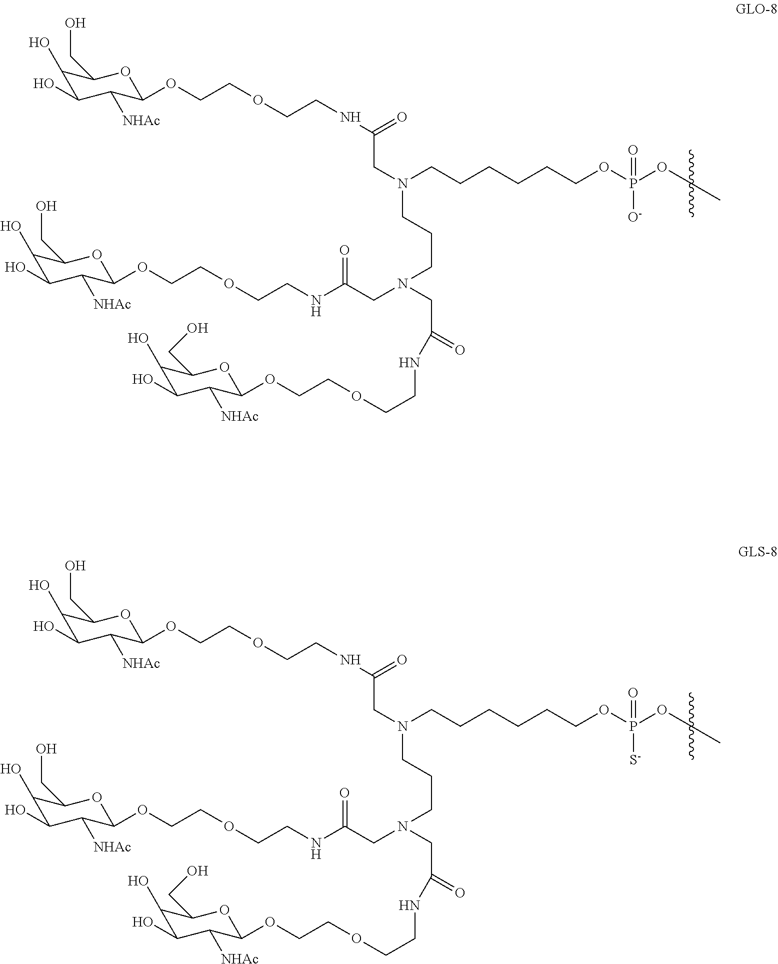 [SMILES: *OP(=O)([O-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

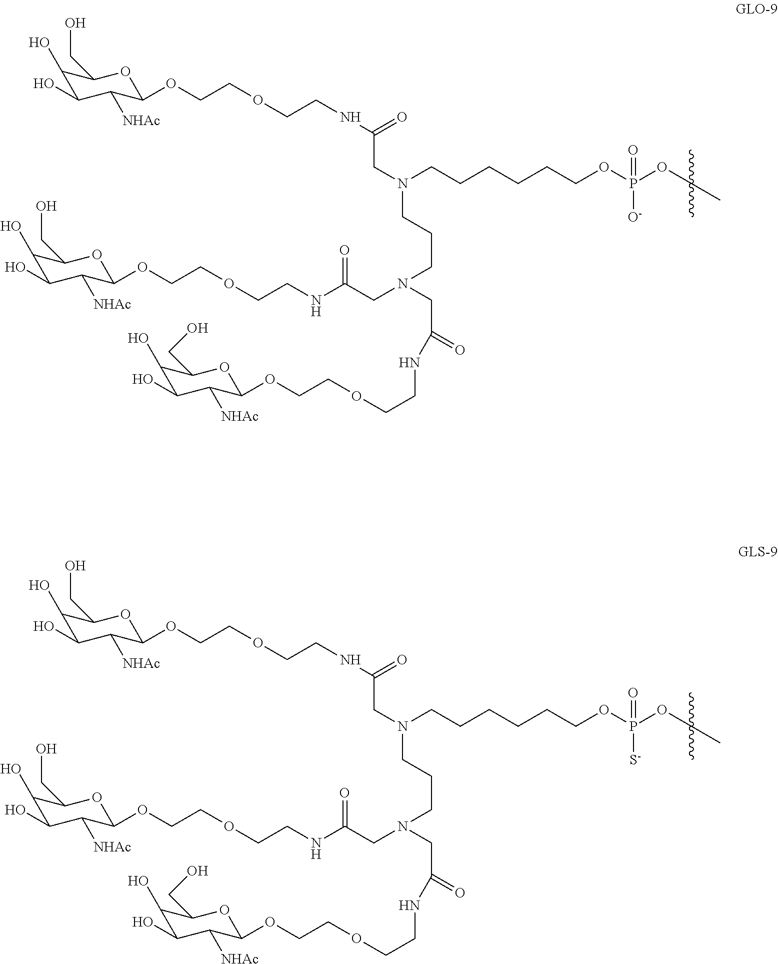 [SMILES: *OP(=O)([O-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

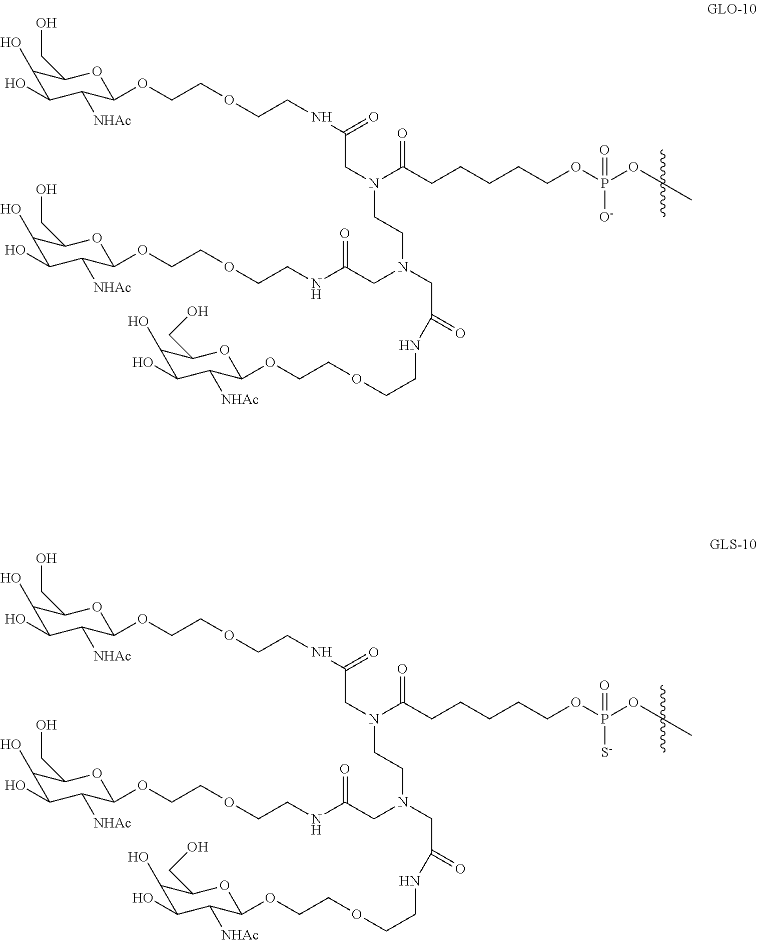 [SMILES: *OP(=O)([O-])OCCCCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

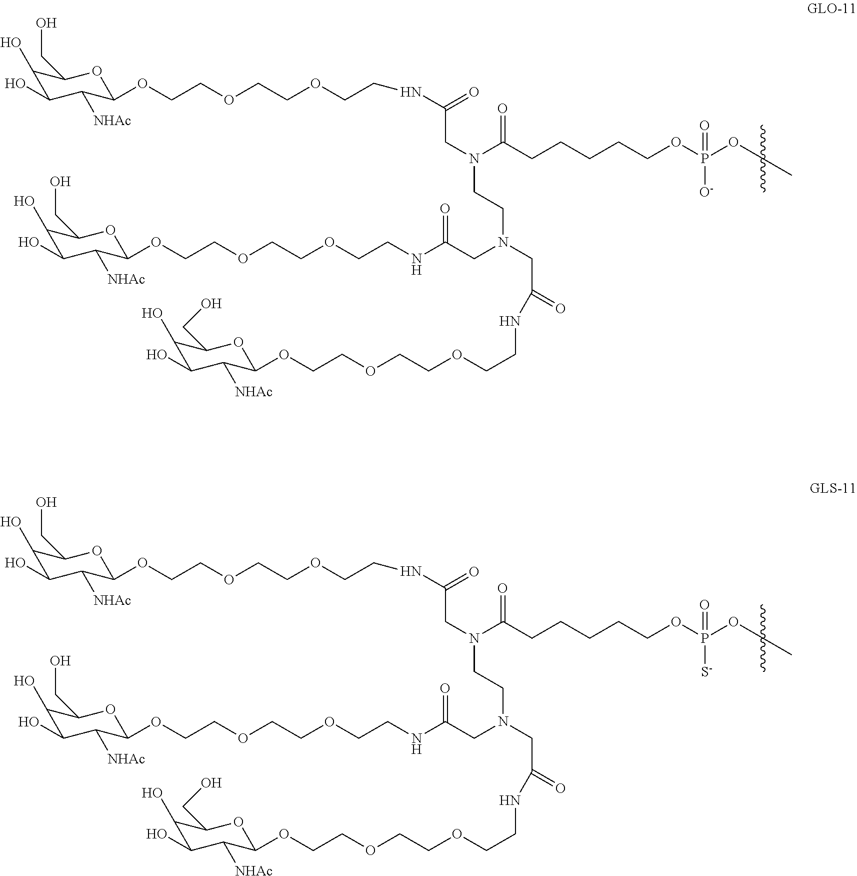 [SMILES: *OP(=O)([O-])OCCCCCC(=O)N(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCCCCC(=O)N(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

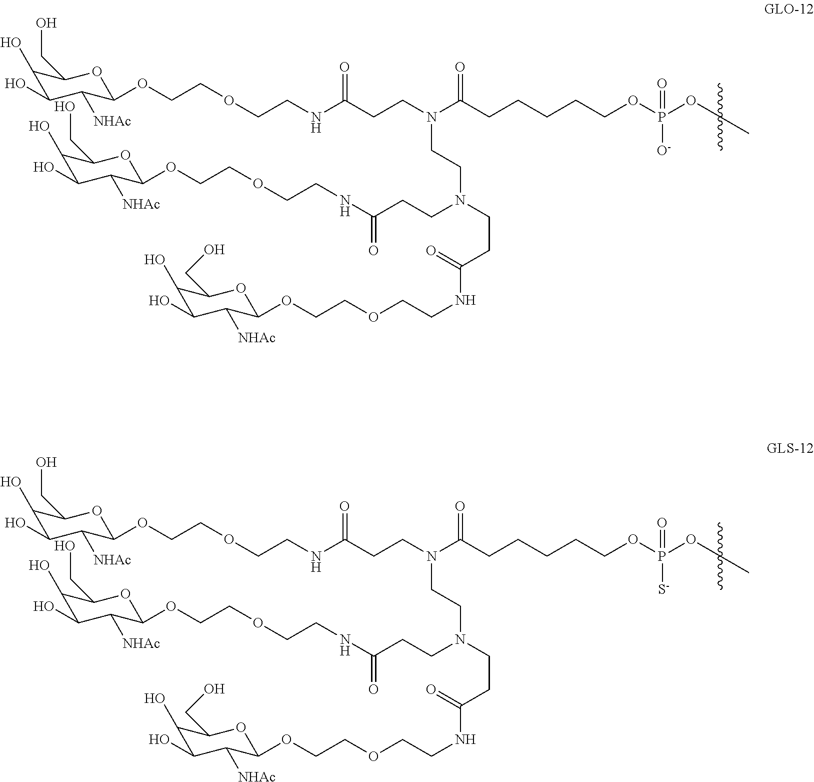 [SMILES: *OP(=O)([O-])OCCCCCC(=O)N(CCC(=O)NCCOCCOC1OC(CO)C(O)C(O)C1NC(C)=O)CCN(CCC(=O)NCCOCCOC1OC(CO)C(O)C(O)C1NC(C)=O)CCC(=O)NCCOCCOC1OC(CO)C(O)C(O)C1NC(C)=O ; *OP(=O)([S-])OCCCCCC(=O)N(CCC(=O)NCCOCCOC1OC(CO)C(O)C(O)C1NC(C)=O)CCN(CCC(=O)NCCOCCOC1OC(CO)C(O)C(O)C1NC(C)=O)CCC(=O)NCCOCCOC1OC(CO)C(O)C(O)C1NC(C)=O]

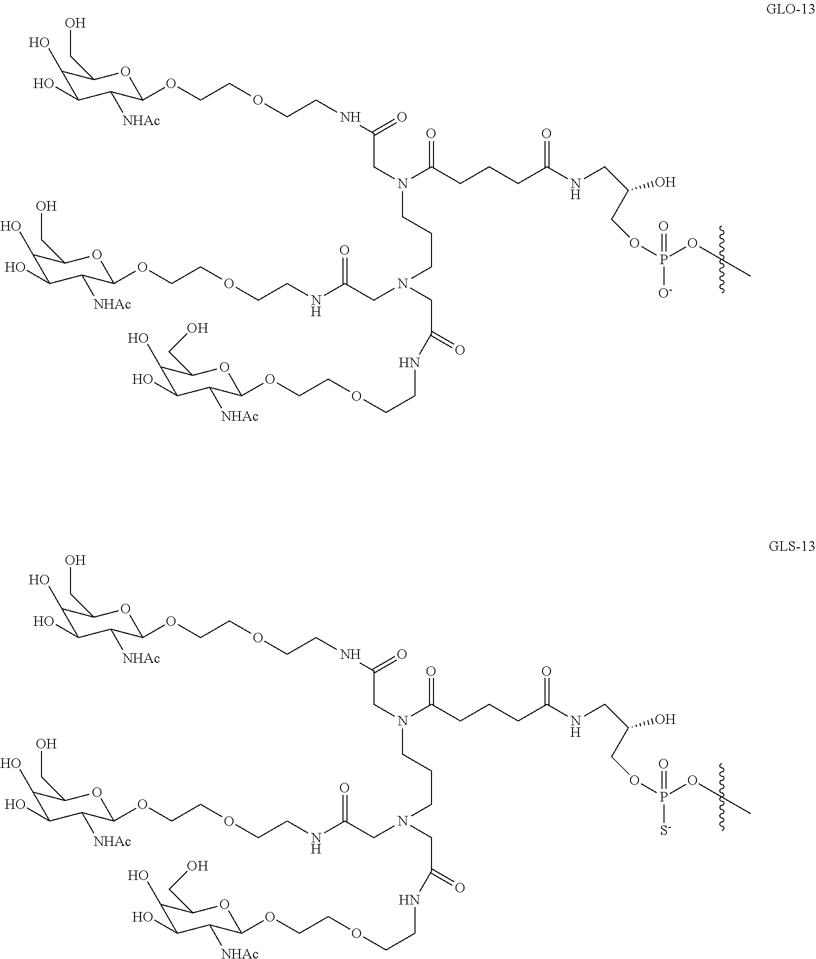 [SMILES: *OP(=O)([O-])OC[C@@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OC[C@@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

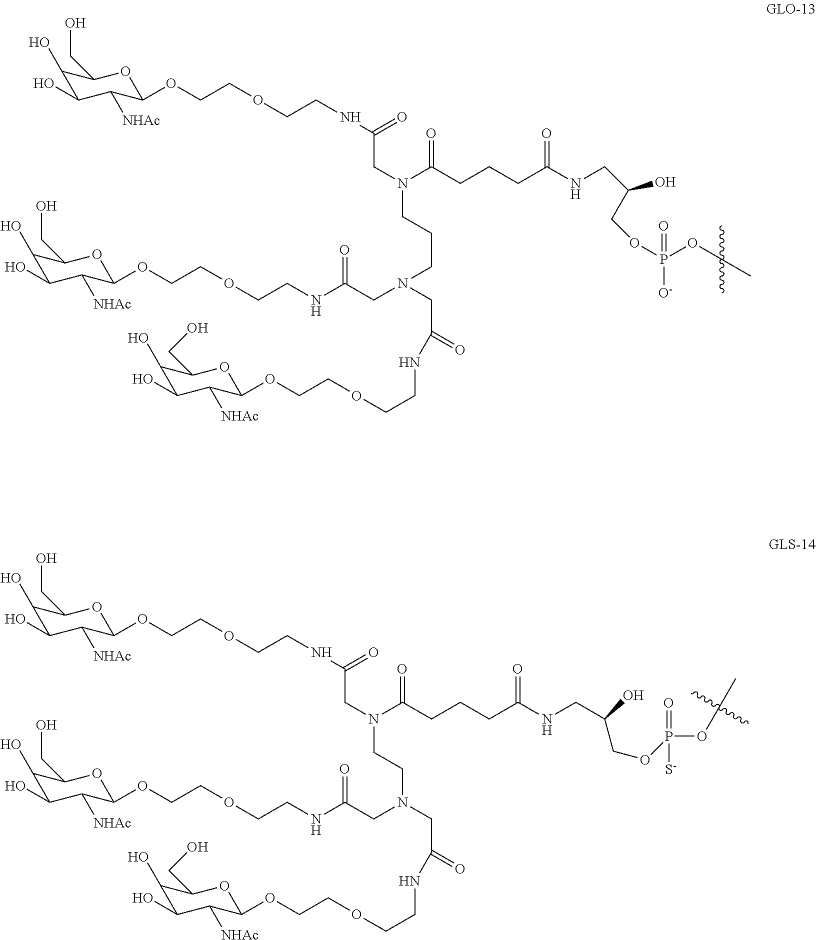 [SMILES: *OP(=O)([O-])OC[C@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OC[C@H](O)CNC(=O)CCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

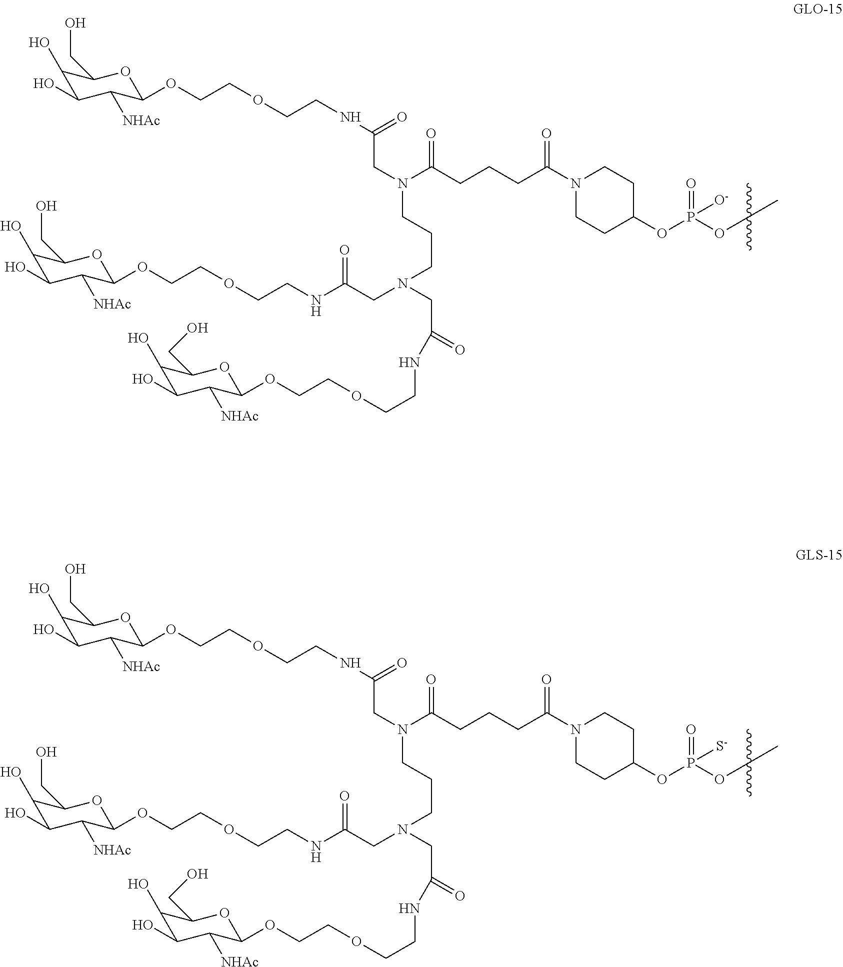 [SMILES: *OP(=O)([O-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1 ; *OP(=O)([S-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1]

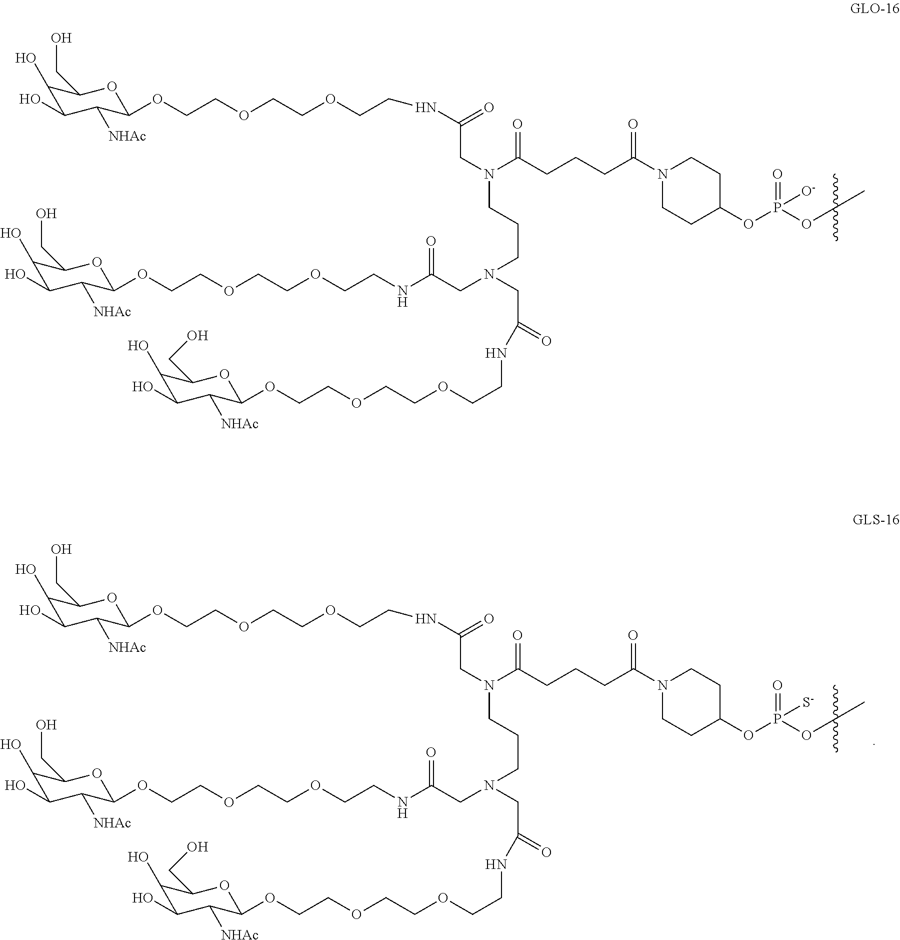 [SMILES: *OP(=O)([O-])OC1CCN(C(=O)CCCC(=O)N(CCCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOCCOC2OC(CO)C(O)C(O)C2NC(C)=O)CC1 ; *OP(=O)([S-])OC1CCN(C(=O)CCCC(=O)N(CCCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOCCOC2OC(CO)C(O)C(O)C2NC(C)=O)CC1]

In certain embodiments, the dsRNA agent comprises a targeting group conjugated to the 5′-terminal end of the sense strand. In some embodiments, the dsRNA agent comprises a targeting group conjugated to the 3′-terminal end of the sense strand. In some embodiments, the antisense strand comprises an inverted abasic residue at the 3′-terminal end. In certain embodiments, the sense strand comprises one or two inverted abasic residues at the 3′ and/or 5′-terminal ends. In some embodiments, the dsRNA agent has two blunt ends. In some embodiments, at least one strand includes a 3′ overhang of at least 1 nucleotide. In some embodiments, at least one strand includes a 3′ overhang that is at least 2 nucleotides.

In certain embodiments, the present invention relates to unlocked nucleic acid (UNA) oligomers for use in therapy. Unlocked nucleic acid (UNA) is an acyclic analog of RNA in which the bond between the C2′ and C3′ atoms of the ribose ring has been severed. Incorporation of UNA has been shown to be well tolerated, and in some cases, even enhance the activity of siRNA gene silencing (Meghan A. et al. “Locked vs. unlocked nucleic acids (LNA vs. UNA): contrasting structures work towards common therapeutic goals”. Chem. Soc. Rev., 2011, 40, 5680-5689).

UNA is a thermolabile modification, and replacing ribonucleotides with UNA reduces base-pairing strength and duplex stability. Strategically placing UNA at the seed region of the antisense strand of siRNA can reduce off-target activity in the mechanism of gene silencing mediated by microRNA (miRNA). miRNA mainly recognizes target genes through base pairing between the antisense seed region (2-8 from the 5′ end) and target mRNA for gene suppression. Each miRNA potentially regulates a large number of genes. The siRNA antisense strand loaded by the RNA-induced silencing complex (RISC) can also potentially regulate a large number of unintended genes through miRNA-mediated mechanisms. Therefore, adding thermolabile nucleotides, such as UNA, to the seed region of siRNA can reduce off-target activity (Lam J K, Chow M Y, Zhang Y, Leung S W. siRNA Versus miRNA as Therapeutics for Gene Silencing. Mol Ther Nucleic Acids. 2015 Sep. 15; 4(9):e252. doi: 10.1038/mtna.2015.23. PMID: 26372022; PMCID: PMC4877448.). In particular, such RNA oligonucleotides or complexes of RNA oligonucleotides contain at least one UNA nucleotide monomer in the seed region (Narendra Vaish et al. “Improved specificity of gene silencing by siRNAs containing unlocked nucleobase analog”. Nucleic Acids Research, 2011, Vol. 39, No. 5 1823-1832).

According to the present technology, potential advantages of incorporating UNA in RNA oligonucleotides or complexes of RNA oligonucleotides include, but are not limited to:

1. Reduce off-target activity. The addition of UNA in the siRNA seed region will reduce the base-pairing strength of the seed region, thereby reducing the potential off-target activity caused by the micro-RNA mechanism.

2. UNA is well tolerated in terms of siRNA activity. In some cases, UNA can lead to enhanced activity.

Exemplary UNA monomers that can be used in this technical solution include, but are not limited to:

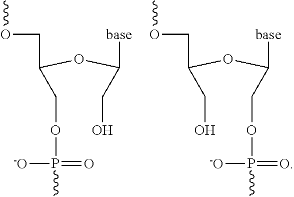

In some embodiments, the dsRNA agent is a modified duplex selected from any one of duplex: AD00158-19-2, AD00158-19-1, AD00158-3, AD00158-1, AD00158-2, AD00158, AD00159, AD00159-1, AD00159-2, AD00159-19-1, AD00159-19-2, AD00163, AD00163-1, AD00163-2, AD00163-19-1, AD00163-19-2, AD00163-3, AD00300-1, AD00300-19-1, AD00300-19-2 in Tables 2-4.

In some embodiments, the dsRNA agent is a modified duplex selected from any one of duplex: AV01227, AV01228, AV01229, AV01230, AV01231, AV01232, AV01233, AV01234, AV01235, AV01236, AV01237, AV01238, AV01239, AV01240, AV01241, AV01242, AV01243, AV01244, AV01245, AV01246, AV01247, AV01248, AV01249, AV01250, AV01251, AV01252, AV01253, AV01254, AV01255, AV01256, AV01257 in Tables 2-4.

According to another aspect of the present invention, a composition including any embodiment of the aforementioned dsRNA agent aspect of the invention is provided. In certain embodiments, the composition also includes a pharmaceutically acceptable carrier. In some embodiments, the composition also includes one or more additional therapeutic agents. In certain embodiments, the compositions are packaged in kits, containers, packs, dispensers, pre-filled syringes, or vials. In some embodiments, the composition is formulated for subcutaneous or intravenous(IV) administration.

According to another aspect of the present invention, there is provided a cell includes any embodiment of the aforementioned dsRNA agent aspect of the invention. In some embodiments, the cells are mammalian cells, optionally human cells.

According to another aspect of the present invention, there is provided a method for inhibiting the expression of AGT gene in a cell, the method including: (i) preparing a cell including an effective amount of any embodiment of the aforementioned dsRNA agent aspect of the invention or any embodiment of an aforementioned composition of the invention. In certain embodiments, the method also includes: (ii) maintaining the prepared cell for a time sufficient to obtain degradation of the mRNA transcript of the AGT gene, thereby inhibiting the expression of the AGT gene in the cells. In some embodiments, the cells are in a subject and the dsRNA agent is administered to the subject subcutaneously. In some embodiments, the cells are in a subject and the dsRNA agent is administered to the subject by IV administration. In certain embodiments, the method further comprises assessing inhibition of the AGT gene after administering the dsRNA agent to the subject, wherein the means for assessing comprises: (i) determining one or more physiological characteristics of an AGT-associated disease or condition in the subject, and (ii) comparing the determined physiological characteristic to a baseline pre-treatment physiological characteristic of the AGT-associated disease or condition and/or a control physiological characteristic of the AGT-associated disease or condition, wherein the comparison indicates a presence or absence of inhibition of expression of the AGT gene in the subject. In some embodiments, the determined physiological characteristic is the AGT level in the blood. In some embodiments, the determined physiological characteristic is blood pressure, which includes systolic blood pressure (SBP), diastolic blood pressure (DBP) and mean arterial pressure (MAPR). A reduction of the AGT levels in the blood and/or of blood pressure indicates a reduction of AGT gene expression in the subject.

According to another aspect of the present invention, there is provided a method of inhibiting the expression of the AGT gene in a subject, which comprises administering to the subject an effective amount of an embodiment of the aforementioned dsRNA agent aspect or an embodiment of the aforementioned composition. In some embodiments, the dsRNA agent is administered to the subject subcutaneously. In certain embodiments, the dsRNA agent is administered to the subject by IV administration. In some embodiments, the method further comprises: assessing inhibition of the AGT gene following administration of the dsRNA agent, wherein the means for assessing comprises: (i) determining one or more physiological characteristics of an AGT-associated disease or condition in the subject; (ii) comparing the determined physiological characteristic to a baseline pre-treatment physiological characteristic of an AGT-associated disease or condition and/or a control physiological characteristic of an AGT-associated disease or condition; wherein the comparison indicates the presence or absence of inhibition of expression of the AGT gene in the subject. In some embodiments, the determined physiological characteristic is the AGT level in the blood; in some embodiments, the determined physiological characteristic is blood pressure, which includes systolic blood pressure (SBP), diastolic blood pressure (DBP) and mean arterial pressure (MAPR). A reduction of the AGT levels in the blood and/or of blood pressure indicates reduction a of AGT gene expression in the subject.

According to another aspect of the present invention, there is provided a method for treating a disease or condition related to the AGT protein, which comprises: administering to a subject an effective amount of any embodiment of the aforementioned dsRNA agents of the present invention or any embodiment of the aforementioned combination of the present invention, to inhibit AGT gene expression. In certain embodiments, the AGT-associated disease or condition is selected from the group consisting of: hypertension, borderline hypertension, essential hypertension, secondary hypertension, isolated systolic or diastolic hypertension, pregnancy-associated hypertension Blood pressure, diabetic hypertension, Intractable hypertension, refractory hypertension, paroxysmal hypertension, renovascular hypertension, Goldblatt's hypertension, ocular hypertension, glaucoma, pulmonary hypertension Blood pressure, portal hypertension, systemic venous hypertension, systolic hypertension, unstable hypertension; hypertensive heart disease, hypertensive nephropathy, atherosclerosis, arteriosclerosis, vascular disease, diabetic nephropathy Diabetic retinopathy, chronic heart failure, cardiomyopathy, diabetic cardiomyopathy, glomerulosclerosis, aortic stenosis, aortic aneurysm, ventricular fibrosis, heart failure, myocardial infarction, angina, stroke, renal disease, Kidney failure, systemic sclerosis, intrauterine growth retardation (IUGR), fetal growth restriction. In some embodiments, the method further comprises: administering to the subject an additional treatment regimen. In some embodiments, the additional treatment regimen includes treatment of an AGT-associated disease or condition. In certain embodiments, the additional treatment regimen comprises: administering to the subject one or more AGT antisense polynucleotides of the invention; administering to the subject a non-AGT dsRNA therapeutic agent; and effecting behavioral modification in the subject. In some embodiments, the non-AGT dsRNA therapeutic agent is one of the following: additional therapeutic agents such as diuretics, angiotensin converting enzyme (ACE) inhibitors, angiotensin II receptor antagonists, beta-blockers, vasodilators, calcium channel blockers, aldosterone antagonists, α2-agonists, renin inhibitors, α-blockers, peripherally acting adrenergic agents, selective D1 receptor partial agonists, Non-selective alpha-adrenergic antagonists, synthetic, steroidal antimineralocorticoids, or combinations of any of the foregoing, and therapeutic agents for hypertension formulated as pharmaceutical combinations.

In some embodiments, the dsRNA agent is administered subcutaneously to the subject. In certain embodiments, the dsRNA agent is administered to the subject by IV administration. In some embodiments, the method further comprises determining the efficacy of the administered double-stranded ribonucleic acid (dsRNA) agent in the subject. In some embodiments, the means for determining the efficacy of a treatment in a subject comprises: (i) determining one or more physiological characteristics of an AGT-associated disease or condition in the subject; (ii) comparing the determined physiological characteristic(s) to a baseline pre-treatment physiological characteristic of the AGT-associated disease or condition, wherein the comparison indicates one or more of a presence, absence, and level of efficacy of the administration of the double-stranded ribonucleic acid (dsRNA) agent to the subject. In some embodiments, the determined physiological characteristic is AGT level in the blood; in some embodiments, the determined physiological characteristic is blood pressure, which includes systolic blood pressure (SBP), diastolic blood pressure (DBP) and mean arterial pressure (MAPR). A reduction of AGT levels in blood and/or reduction of blood pressure indicates the presence of efficacy of administering a double-stranded ribonucleic acid (dsRNA) agent to a subject.

According to another aspect of the present invention, a method of decreasing a level of AGT protein in a subject compared to a baseline pre-treatment level of AGT protein in the subject is provided, the method including administering to the subject an effective amount of any embodiment of an aforementioned dsRNA agent aspect of the invention, or any embodiment of an aforementioned composition of the invention, to decrease the level of AGT gene expression. In some embodiments, the dsRNA agent is administered to the subject subcutaneously or by IV.

According to another aspect of the present invention, a method of altering a physiological characteristic of AGT-associated disease or condition in a subject compared to a baseline pre-treatment physiological characteristic of the AGT-associated disease or condition in the subject is provided, the method including administering to the subject an effective amount of any embodiment of an aforementioned dsRNA agent aspect of the invention, or any embodiment of an aforementioned composition of the invention, to alter the physiological characteristic of the AGT-associated disease or condition in the subject. In some embodiments, the dsRNA agent is administered to the subject subcutaneously or by IV. In certain embodiments, the physiological characteristic is AGT levels in the blood; in some embodiments, the determined physiological characteristic is blood pressure, which includes systolic blood pressure (SBP), diastolic blood pressure (DBP), and mean arterial pressure (MAPR).

### BRIEF DESCRIPTION OF THE SEQUENCES

Duplexes AD00051 to AD00122-19-2, AD00163-3, AV01227 to AV01257, AV01711 are shown in Table 1 and their sense strand sequences are shown.

Duplexes AD00051 to AD00122-19-2, AD00163-3, AV01227 to AV01257, AV01711 are shown in Table 1 and their antisense strand sequences are shown.

SEQ ID NO: 519 is Homo sapiens Angiotensinogen (AGT) mRNA [NCBI Reference
Sequence: NM_001384479.1]:
GAAGAAGCTGCCGTTGTTCTGGGTACTACAGCAGAAGGGTATGCGGAAGCGAGCACC
CCAGTCTGAGATGGCTCCTGCCGGTGTGAGCCTGAGGGCCACCATCCTCTGCCTCCTG
GCCTGGGCTGGCCTGGCTGCAGGTGACCGGGTGTACATACACCCCTTCCACCTCGTC
ATCCACAATGAGAGTACCTGTGAGCAGCTGGCAAAGGCCAATGCCGGGAAGCCCAA
AGACCCCACCTTCATACCTGCTCCAATTCAGGCCAAGACATCCCCTGTGGATGAAAAG
GCCCTACAGGACCAGCTGGTGCTAGTCGCTGCAAAACTTGACACCGAAGACAAGTTG
AGGGCCGCAATGGTCGGGATGCTGGCCAACTTCTTGGGCTTCCGTATATATGGCATGC
ACAGTGAGCTATGGGGCGTGGTCCATGGGGCCACCGTCCTCTCCCCAACGGCTGTCTT
TGGCACCCTGGCCTCTCTCTATCTGGGAGCCTTGGACCACACAGCTGACAGGCTACA
GGCAATCCTGGGTGTTCCTTGGAAGGACAAGAACTGCACCTCCCGGCTGGATGCGCA
CAAGGTCCTGTCTGCCCTGCAGGCTGTACAGGGCCTGCTAGTGGCCCAGGGCAGGGC
TGATAGCCAGGCCCAGCTGCTGCTGTCCACGGTGGTGGGCGTGTTCACAGCCCCAGG
CCTGCACCTGAAGCAGCCGTTTGTGCAGGGCCTGGCTCTCTATACCCCTGTGGTCCTC
CCACGCTCTCTGGACTTCACAGAACTGGATGTTGCTGCTGAGAAGATTGACAGGTTC
ATGCAGGCTGTGACAGGATGGAAGACTGGCTGCTCCCTGATGGGAGCCAGTGTGGAC
AGCACCCTGGCTTTCAACACCTACGTCCACTTCCAAGGGAAGATGAAGGGCTTCTCC
CTGCTGGCCGAGCCCCAGGAGTTCTGGGTGGACAACAGCACCTCAGTGTCTGTTCCC
ATGCTCTCTGGCATGGGCACCTTCCAGCACTGGAGTGACATCCAGGACAACTTCTCG
GTGACTCAAGTGCCCTTCACTGAGAGCGCCTGCCTGCTGCTGATCCAGCCTCACTATG
CCTCTGACCTGGACAAGGTGGAGGGTCTCACTTTCCAGCAAAACTCCCTCAACTGGA
TGAAGAAACTATCTCCCCGGACCATCCACCTGACCATGCCCCAACTGGTGCTGCAAG
GATCTTATGACCTGCAGGACCTGCTCGCCCAGGCTGAGCTGCCCGCCATTCTGCACAC
CGAGCTGAACCTGCAAAAATTGAGCAATGACCGCATCAGGGTGGGGGAGGTGCTGA
ACAGCATTTTTTTTGAGCTTGAAGCGGATGAGAGAGAGCCCACAGAGTCTACCCAAC
AGCTTAACAAGCCTGAGGTCTTGGAGGTGACCCTGAACCGCCCATTCCTGTTTGCTGT
GTATGATCAAAGCGCCACTGCCCTGCACTTCCTGGGCCGCGTGGCCAACCCGCTGAG
CACAGCATGAGGCCAGGGCCCCAGAACACAGTGCCTGGCAAGGCCTCTGCCCCTGG
CCTTTGAGGCAAAGGCCAGCAGCAGATAACAACCCCGGACAAATCAGCGATGTGTCA
CCCCCAGTCTCCCACCTTTTCTTCTAATGAGTCGACTTTGAGCTGGAAAGCAGCCGTT
TCTCCTTGGTCTAAGTGTGCTGCATGGAGTGAGCAGTAGAAGCCTGCAGCGGCACAA
ATGCACCTCCCAGTTTGCTGGGTTTATTTTAGAGAATGGGGGTGGGGAGGCAAGAAC
CAGTGTTTAGCGCGGGACTACTGTTCCAAAAAGAATTCCAACCGACCAGCTTGTTTGT
GAAACAAAAAAGTGTTCCCTTTTCAAGTTGAGAACAAAAATTGGGTTTTAAAATTAA
AGTATACATTTTTGCATTGCCTTCGGTTTGTATTTAGTGTCTTGAATGTAAGAACATGAC
CTCCGTGTAGTGTCTGTAATACCTTAGTTTTTTCCACAGATGCTTGTGATTTTTGAACA
ATACGTGAAAGATGCAAGCACCTGAATTTCTGTTTGAATGCGGAACCATAGCTGGTTA
TTTCTCCCTTGTGTTAGTAATAAACGTCTTGCCACAATAAGCCTCCAAAAA.
SEQ ID NO: 520 is Mus musculus Angiotensinogen (AGT) mRNA [NCBI Reference
Sequence: NM_007428.4]
ATGACTCCCACGGGGGCAGGCCTGAAGGCCACCATCTTCTGCATCTTGACCTGGGTC
AGCCTGACGGCTGGGGACCGCGTATACATCCACCCCTTCCATCTCCTTTACCACAACA
AGAGCACCTGCGCCCAGCTGGAGAACCCCAGTGTGGAGACACTCCCAGAGTCAACG
TTCGAGCCTGTGCCCATTCAGGCCAAGACCTCCCCTGTGAATGAGAAGACCCTGCAT
GATCAGCTCGTGCTGGCCGCCGAGAAGCTAGAGGATGAGGACCGGAAGCGGGCTGC
CCAGGTCGCAATGATCGCCAACTTCGTGGGCTTCCGCATGTACAAGATGCTGAATGAG
GCAGGAAGTGGGGCCAGTGGGGCCATCCTCTCACCACCAGCTCTCTTTGGCACCCTG
GTCTCTTTCTACCTTGGATCCTTAGATCCCACGGCCAGCCAGCTGCAGACGCTGCTGG
ATGTCCCTGTGAAGGAGGGAGACTGCACCTCCCGACTAGATGGACACAAGGTCCTCG
CTGCCCTGCGGGCCATTCAGGGCTTGCTGGTCACCCAGGGTGGGAGCAGCAGCCAGA
CACCCCTGCTACAGTCCATTGTGGTGGGGCTCTTCACTGCTCCAGGCTTTCGTCTAAA
GCACTCATTTGTTCAGAGCCTGGCTCTCTTTACCCCTGCCCTCTTCCCACGCTCTCTGG
ATTTATCCACTGACCCAGTTCTTGCCACTGAGAAAATCAACAGGTTCATAAAGGCTGT
GACAGGGTGGAAGATGAACTTGCCACTGGAGGGGGTCAGTACAGACAGCACCCTAC
TTTTCAACACCTACGTTCACTTCCAAGGAACGATGAGAGGTTTCTCTCAGCTGCCTGG
AGTCCATGAATTCTGGGTGGACAACAGCATCTCGGTGTCTGTGCCCATGATCTCCGGC
ACTGGCAACTTCCAGCACTGGAGTGACACCCAGAACAACTTCTCCGTGACGTGCGTG
CCCCTAGGTGAGAGAGCCACCCTGCTGCTCATCCAGCCCCACTGCACCTCAGATCTCG
ACAGGGTGGAGGCCCTCATCTTCCGGAACGACCTCCTGACTTGGATAGAGAACCCGC
CTCCTCGGGCCATCCGCCTGACTCTGCCCCAGCTGGAAATCCGAGGATCCTACAATCT
GCAGGACCTGCTGGCTGAGGACAAGCTGCCCACCCTTTTGGGTGCGGAGGCAAATCT
GAACAACATTGGTGACACCAACCCCCGAGTGGGAGAGGTTCTCAATAGCATCCTCCT
CGAACTCAAAGCAGGAGAGGAGGAACAGCCGACCACGTCTGTCCAGCAGCCTGGCT
CACCGGAGGCACTGGATGTGACCCTGAGCAGCCCCTTCCTGTTCGCCATCTACGAGC
AGGACTCAGGCACGCTGCACTTTCTGGGCAGAGTGAATAACCCCCAGAGTGTGGTGT
GA
SEQ ID NO: 521 is cynomolgus monkey Angiotensinogen (AGT) mRNA [NCBI Reference
Sequence: NM_001283634.1]
ATGCAGAAGCGAGCACCCCAGTCCGAGATGGCTCCTGCCAGCGTGAGCCTGAGGGC
CACCATCCTCTGCCTCCTGGCCTGGGCTGGCCTGGCCACAGGTGACCGGGTGTACATA
CACCCCTTCCACCTCGTCATCCACAATGAGAGTACCTGTGAGCAGCTGGCAAAGGCC
GATGCTGGGAAGCCCAAAGATCCCACCTTCACACCTGTTCCGATACAGGCCAAGACG
TCTCCTGTGGATGAAAAGGCCCTGCAGGACCAGCTAGTGCTGGTTGCCGCAAAACTC
GACACCGAGGACAAGTTGAGAGCCGCGATGGTCGGGATGCTGGCCAACTTCTTGGGC
TTCCGTATATATGGCATGCACAGTGAGCTATGGGGCGTGGTCCATGGGGCCACCATCCT
CTCCCCAACGGCTGTCTTTGGCACCCTGGCCTCTCTCTACCTGGGAGCGTTGGACCAC
ACAGCCGACAGGCTACAGGCAATCCTGGGCGTCCCTTGGAAGGACAAGAACTGCAC
CTCCCGGCTGGATGCGCACAAGGTCCTCTCTGCCCTGCAGGCTGTACAGGGCCTGCT
GGTGGCCCAGGGCAGGGCTGACGGCCAGTCCCAGCTGCTGTTGTCCACAGTGGTGG
GTCTCTTCACAGCCCCAGATCTGCACCTGAAGCAGCCGTTTGTGCAGGGCCTGGCTC
TCTATGCCCCTGTGGTCCTCCCACGCTCTCTGGACTTCACAGACCTGGAAGTCGCTGC
TGAGAAGATTGACAGGTTCATGCAGGCTGTGACAGGATGGAAGATTAGCAGCCCCCT
GACGGGAGCCAGTGCGGACAGCACCCTGGTTTTCAACACCTACGTCCATTTCCAAGG
GAAGATGAGGGACTTCTTCCTGCTGGCTGAGCCCCAGGAGTTCTGGGTGGACAACAG
CACCTCAGTGTCTGTCCCCATGCTGTCTGGCGTGGGCACCTTCCAGCACTGGAGCGA
CGCCCAGGACAACTTCTCAGTGACTCAAGTGCCCTTTACTGAGAGCGCCTGCTTGCT
GCTGATTCAGCCTCACTACGCCTCTGACCTGGACAAGGTGGAGGGTCTCACTTTCCA
GCAAAACTCCCTCAACTGGATGAAGAAACTGTCTCCCCGGGCCATCCACCTGACCAT
GCCCCGACTGGTGCTGCGAGGATCTTATGACCTGCAGGACCTGCTTGCCCAGGCTGA
GCTGCCCGCCATTCTGGGCACCGAGCTGAACCTGCAAAAATTGAGCAATGACAACCT
CAGGGTGGGGAAGGTGCTGAACAGCATTCTTTTTGAACTCGAAGCGGATGAGAGAG
AGCCCACAGAGTCTACCCGACAGCTGAACAGGCCTGAGTTCTTGGAGGTGACCCTGG
ACCGCCCATTCCTGTTTGCTGTGTATGATCAAAGTGCCACTGCCCTGCACTTCCTGGG
CCGTGTGGCCAACCCGCTGAGCCCAGCATGA

In the sequences shown in Table 2, chemical modifications are indicated as: upper case: 2′-fluoro; lower case: 2′-OMe; thiophosphate: *.

In the sequences shown in Table 3, the delivery molecules used in the in vivo studies are indicated as “GLO-0” at the 3′ end of each sense strand. Chemical modifications are expressed as: upper case: 2′-fluoro; lower case: 2′-OMe; thiophosphate: *; unlocked nucleic acid: UNA (Note: AD00052, AD00113-AD00260: no UNA; AD00282-AD00301: UNA version).

In the sequences shown in Table 4, chemical modifications are indicated as: upper case: 2′-fluoro; lower case: 2′-OMe; thiophosphate: *; Invab=inverted abasic.

### BRIEF DESCRIPTION OF THE DRAWINGS

FIG. 1 is a graph showing the serum AGT protein levels in cynomolgus monkeys after administration of 2 mg/kg of AD00158-1, AD00158-2, AD00163-1, AD00159-1, and AD00300-1, respectively;

FIG. 2 is a graph showing the serum AGT protein level in cynomolgus monkeys after administration of 10 mg/kg of AD00163-3;

FIG. 3 is a graph showing the changes in serum SBP in cynomolgus monkeys after administration of 10 mg/kg of AD00163-3;

FIG. 4 is a graph showing the mean blood pressure (MBP) of cynomolgus monkeys after administration of 10 mg/kg of AD00163-3;

FIG. 5 is a graph showing the diastolic blood pressure (DBP) in cynomolgus monkeys after administration of 10 mg/kg of AD00163-3.

### DETAILED DESCRIPTION

The invention in part, includes RNAi agents, for example, though not limited to double stranded (ds) RNAi agents, which are capable of inhibiting the expression of Angiotensinogen (AGT) gene. The invention, in part also includes compositions comprising AGT RNAi agents and methods of use of the compositions. The AGT RNAi agents disclosed herein can be attached to delivery compounds for delivery to cells, including delivery to hepatocytes. Pharmaceutical compositions of the invention may comprise at least one dsAGT agent and a delivery compound.

In some embodiments of the invention, the delivery compound is a GalNAc-containing delivery compound. AGT RNAi agents delivered to cells are capable of inhibiting AGT gene expression, thereby reducing the activity of the gene's AGT protein product in the cell. The dsRNAi agents of the invention are useful in the treatment of AGT-associated diseases and conditions. Such dsRNAi agents include, for example, the duplexes AD00051 to AD00122-19-2 shown in Table 1. In some embodiments, preferred dsRNAi agents include, for example, duplexes AD00158, AD00163, AD00159, AD00290, AD00300 or AD00122. In other embodiments, preferred dsRNAi agents include, for example, AD00158-1, AD00158-2, AD00163-1, AD00163-3, AD00159-1 or AD00300-1. In other embodiments, such dsRNAi agents include duplex variants, eg, variants of duplex AD00158, AD00163, AD00163-3, AD00159, AD00290, AD00300 or AD00122.

In some embodiments of the invention, reducing AGT expression in a cell or a subject treats a disease or condition associated with AGT expression in the cell or subject, respectively. Non-limiting examples of diseases and conditions treatable by reducing AGT activity are: hypertension, hypertension, borderline hypertension, essential hypertension, secondary hypertension, isolated systolic or diastolic hypertension, pregnancy-related hypertension, diabetic hypertension, Refractory hypertension, refractory hypertension, paroxysmal hypertension, renovascular hypertension, Goldblatt hypertension, ocular hypertension, Glaucoma, pulmonary hypertension, portal hypertension, systemic venous hypertension, systolic hypertension, unstable hypertension; hypertensive heart disease, hypertensive nephropathy, atherosclerosis, arteriosclerosis, vascular disease, diabetic nephropathy, diabetic retinopathy, chronic heart failure, cardiomyopathy, diabetic cardiomyopathy, glomerulosclerosis, aortic stenosis, aortic aneurysm, ventricular fibrosis, heart failure, myocardial infarction, angina pectoris, stroke, kidney disease, kidney failure, systemic sclerosis, intrauterine growth retardation (IUGR), fetal growth restriction.

The following describes ways to make and use compositions comprising AGT single-stranded (ssRNA) and double-stranded (dsRNA) agents to inhibit AGT gene expression, and compositions and methods for treating diseases and conditions caused or regulated by AGT gene expression. The term “RNAi” is also known in the art and may be referred to as “siRNA”.

As used herein, the term “RNAi” refers to an agent that comprises RNA and mediates targeted cleavage of an RNA transcript via an RNA-induced silencing complex (RISC) pathway. As is known in the art, an RNAi a target region refers to a contiguous portion of the nucleotide sequence of an mRNA molecule formed during the transcription of a gene, including messenger RNA (mRNA) that is a product of RNA processing of a primary transcription product. The target portion of the sequence will be at least long enough to serve as a substrate for RNAi-directed cleavage at or near that portion. A target sequence may be from 8-30 nucleotides long (inclusive), from 10-30 nucleotides long (inclusive), from 12-25 nucleotides long (inclusive), from 15-23 nucleotides long (inclusive), from 16-23 nucleotides long (inclusive), or from 18-23 nucleotides long (inclusive), including all shorter lengths within each stated range. In some embodiments of the invention, a target sequence is 9, 10, 11, 12, 13, 14, 15, 16, 17, 18, 19, 20, 21, 22, 23, 24, 25, or 26 nucleotides long. In certain embodiment, a target sequence is between 9 and 26 nucleotides long (inclusive), including all sub-ranges and integers there between. For example, though not intended to be limiting, in certain embodiments of the invention a target sequence is 8, 9, 10, 11, 12, 13, 14, 15, 16, 17, 18, 19, 20, 21, 22, 23, 24, 25, 26, 27, 28, 29, or 30 nucleotides long, with the sequence fully or at least substantially complementary to at least part of an RNA transcript of an AGT gene. Some aspects of the invention include pharmaceutical compositions comprising one or more AGT dsRNA agents and a pharmaceutically acceptable carrier. In certain embodiments of the invention, an AGT RNAi as described herein inhibits expression of AGT protein.

As used herein, a “dsRNA agent” means a composition that contains an RNA or RNA-like (e.g., chemically modified RNA) oligonucleotide molecule that is capable of degrading or inhibiting translation of messenger RNA (mRNA) transcripts of a target mRNA in a sequence specific manner. Although not wishing to be limited to a particular theory, dsRNA agents of the invention may operate through the RNA interference mechanism (i.e., inducing RNA interference through interaction with the RNA interference pathway machinery (RNA-induced silencing complex or RISC) of mammalian cells), or by any alternative mechanism(s) or pathway(s). Methods for silencing genes in plant, invertebrate, and vertebrate cells are well known in the art [see, for example, (Sharp et al., Genes Dev. 2001, 15:485; Bernstein, et al., (2001) Nature 409:363; Nykanen, et al., (2001) Cell 107:309; and Elbashir, et al., (2001) Genes Dev. 15:188)], the disclosure of each of which is incorporated herein by reference in its entirety.]. Art-known gene silencing procedures can be used in conjunction with the disclosure provided herein to inhibit expression of AGT.

dsRNA agents disclosed herein are comprised of a sense strand and an antisense strand, and include, but are not limited to: short interfering RNAs (siRNAs), RNAi agents, micro RNAs (miRNAs), short hairpin RNAs (shRNA), and dicer substrates. The antisense strand of the dsRNA agents described herein is at least partially complementary to the mRNA being targeted. It is understood in the art that different lengths of dsRNA duplex structure can be used to inhibit target gene expression. For example, dsRNAs having a duplex structure of 19, 20, 21, 22, and 23 base pairs are known to be effective to induce RNA interference (Elbashir et al., EMBO 2001, 20:6877-6888). It is also known in the art that shorter or longer RNA duplex structures are also effective to induce RNA interference. AGT dsRNAs in certain embodiments of the invention can include at least one strand of a length of minimally 21 nt or may have shorter duplexes based on one of the sequences set forth in any one of Tables 1-5 minus 1, 2, 3, or 4 nucleotides on one or both ends may also be effective as compared to the dsRNAs set forth in Tables 1-5, respectively. In some embodiments of the invention, AGT dsRNA agents may have a partial sequence of at least 15, 16, 17, 18, 19, 20, or more contiguous nucleotides from one or more sequences of Tables 1-5, and differ in their ability to inhibit the expression of an AGT gene by not more than 5, 10, 15, 20, 25, or 30% from the level of inhibition resulting from a dsRNA comprising the full sequence, which is also referred to herein as the “parent” sequence.

Certain embodiments of the compositions and methods of the invention include single-stranded RNA in the composition and/or administer single-stranded RNA to a subject. For example, the antisense strands listed in any one of Tables 1-4 may be a composition or in a composition administered to a subject to reduce AGT polypeptide activity and/or expression of the AGT gene in the subject. Tables 1-4 show certain AGT dsRNA agent antisense strand and sense strand core stretch base sequences. Single-stranded antisense molecules that may be included in certain compositions of the invention and/or administered in certain methods of the invention are referred to herein as “single-stranded antisense agents” or “antisense polynucleotide agents”. Single-stranded sense molecules that may be included in certain compositions and/or administered in certain methods of the invention are referred to herein as “single-stranded sense agents” or “sense polynucleotide agents.” The term “base sequence” herein refers to a polynucleotide sequence without chemical modifications or delivery compounds. For example, the sense strand shown in Table 1 corresponds to the corresponding base sequence in Table 3; however, the respective chemical modification and delivery compounds are shown in the corresponding sequences in Table 3. Sequences disclosed herein may be assigned identifiers. For example, a single-stranded sense sequence can be identified by “sense strand SS #”; a single-stranded antisense sequence can be identified by “antisense strand AS #”, and a duplex that includes a sense strand and an antisense strand may be identified with a “Duplex AD #”.

Table 1 includes the sense and antisense strands and provides the identification numbers of duplexes formed by the sense and antisense strands on the same row in Table 1. In certain embodiments of the invention, the antisense sequence comprises nucleobase u or nucleobase a in its first position. In certain embodiments of the invention, the antisense sequence comprises the nucleobase u in its first position. As used herein, the term “matching position” refers in a sense to the position in each strand that “pairs” with each other when the two strands act as a duplex. For example, in a 21 nucleobase sense strand and a 21 nucleobase antisense strand, nucleobase in position 1 of the sense strand and position 21 in the antisense strand are in “matching positions”. In yet another non-limiting example in a 23 nucleobase sense strand and a 23 nucleobase antisense strand, nucleobase 2 of the sense strand and position 22 of the antisense strand are in matching positions. In another non-limiting example, in an 18 nucleobase sense strand and an 18 nucleobase antisense strand, nucleobase in position 1 of the sense strand and nucleobase 18 in the antisense strand are in matching positions, and nucleobase 4 in the sense strand and nucleobase 15 in the antisense strand are in matching positions. A skilled artisan will understand how to identify matching positions in sense and antisense strands that are or will be duplexed strands and paired strands.

The final column in Table 1 indicates a Duplex AD #/AV # for a duplex that includes the sense and antisense sequences in the same table row. For example, Table 1 discloses the duplex assigned “Duplex AD #AD00051”, which includes sense strand and antisense strand. Thus, each row in Table 1 identifies a duplex of the invention, each comprising sense and antisense sequences shown in the same row, with the assigned identifier for each duplex shown at the end of the row in a column.

In some embodiments of the methods of the invention, an RNAi agent comprising the polynucleotide sequence shown in Table 1 is administered to the subject. In some embodiments of the present invention, the RNAi agent administered to the subject comprises a duplex comprising at least one of the base sequences listed in Table 1 and comprising 0, 1, 2, 3, 4, 5, 6, 7, 8, 9, 10, 11, 12, 13, 14, 15, 16, 17, 18, 19, 20, 21, 22, 23 or 24 sequence modifications. In some embodiments of the methods of the invention, further comprising linking the RNAi agent of the polynucleotide sequence shown in Table 1 to a delivery molecule, a non-limiting example of which is a delivery compound comprising GalNAc.

TABLE 1
Unmodified AGT RNAi agents antisense and sense strand sequences. All sequences
are shown in 5′ to 3′ direction. Duplex AD#/AV# is the number assigned to the
duplex of both strands in the same row in the table.

| Sense | SEQ | Antisense | SEQ | Duplex AD#/ |
|---|---|---|---|---|
| strand sequence 5′→3′ | ID NO | strand sequence 5′→3′ | ID NO | AV# |
| GCGUCAUCCACAAUGAGAGUA | 1 | UACUCUCAUUGUGGAUGACGC | 98 | AD00051 |
| GUCAUCCACAAUGAGAGUACA | 2 | UGUACUCUCAUUGUGGAUGAC | 99 | AD00052 |
| GAUCCACAAUGAGAGUACCUA | 3 | UAGGUACUCUCAUUGUGGAUC | 100 | AD00053 |
| GUCCACAAUGAGAGUACCUGA | 4 | UCAGGUACUCUCAUUGUGGAC | 101 | AD00054 |
| GUUCUUGGGCUUCCGUAUAUA | 5 | UAUAUACGGAAGCCCAAGAAC | 102 | AD00055 |
| GUUGGGCUUCCGUAUAUAUGA | 6 | UCAUAUAUACGGAAGCCCAAC | 103 | AD00056 |
| GUGGGCUUCCGUAUAUAUGGA | 7 | UCCAUAUAUACGGAAGCCCAC | 104 | AD00057 |
| GGCUUCCGUAUAUAUGGCAUA | 8 | UAUGCCAUAUAUACGGAAGCC | 105 | AD00058 |
| GCUUCCGUAUAUAUGGCAUGA | 9 | UCAUGCCAUAUAUACGGAAGC | 106 | AD00059 |
| GCGUAUAUAUGGCAUGCACAA | 10 | UUGUGCAUGCCAUAUAUACGC | 107 | AD00060 |
| GGUUCCUUGGAAGGACAAGAA | 11 | UUCUUGUCCUUCCAAGGAACC | 108 | AD00061 |
| GAGAAGAUUGACAGGUUCAUA | 12 | UAUGAACCUGUCAAUCUUCUC | 109 | AD00062 |
| GAUGCAGGCUGUGACAGGAUA | 13 | UAUCCUGUCACAGCCUGCAUC | 110 | AD00063 |
| GGAGUUCUGGGUGGACAACAA | 14 | UUGUUGUCCACCCAGAACUCC | 111 | AD00064 |
| GCAACAGCACCUCAGUGUCUA | 15 | UAGACACUGAGGUGCUGUUGC | 112 | AD00065 |
| GGGGUCUCACUUUCCAGCAAA | 16 | UUUGCUGGAAAGUGAGACCCC | 113 | AD00066 |
| GUCACUUUCCAGCAAAACUCA | 17 | UGAGUUUUGCUGGAAAGUGAC | 114 | AD00067 |
| GCCAGCAAAACUCCCUCAACA | 18 | UGUUGAGGGAGUUUUGCUGGC | 115 | AD00068 |
| GAGCAAAACUCCCUCAACUGA | 19 | UCAGUUGAGGGAGUUUUGCUC | 116 | AD00069 |
| GGAGCUGAACCUGCAAAAAUA | 20 | UAUUUUUGCAGGUUCAGCUCC | 117 | AD00070 |
| GCUGAACCUGCAAAAAUUGAA | 21 | UUCAAUUUUUGCAGGUUCAGC | 118 | AD00071 |
| GGAACCGCCCAUUCCUGUUUA | 22 | UAAACAGGAAUGGGCGGUUCC | 119 | AD00072 |
| GAACCGCCCAUUCCUGUUUGA | 23 | UCAAACAGGAAUGGGCGGUUC | 120 | AD00073 |
| GUUCCUGUUUGCUGUGUAUGA | 24 | UCAUACACAGCAAACAGGAAC | 121 | AD00074 |
| GCUGUUUGCUGUGUAUGAUCA | 25 | UGAUCAUACACAGCAAACAGC | 122 | AD00075 |
| GUGUUUGCUGUGUAUGAUCAA | 26 | UUGAUCAUACACAGCAAACAC | 123 | AD00076 |
| GUUGCUGUGUAUGAUCAAAGA | 27 | UCUUUGAUCAUACACAGCAAC | 124 | AD00077 |
| GUCCCACCUUUUCUUCUAAUA | 28 | UAUUAGAAGAAAAGGUGGGAC | 125 | AD00078 |
| GACCUUUUCUUCUAAUGAGUA | 29 | UACUCAUUAGAAGAAAAGGUC | 126 | AD00079 |
| GCCUUUUCUUCUAAUGAGUCA | 30 | UGACUCAUUAGAAGAAAAGGC | 127 | AD00080 |
| GCGUUUCUCCUUGGUCUAAGA | 31 | UCUUAGACCAAGGAGAAACGC | 128 | AD00081 |
| GUUUCUCCUUGGUCUAAGUGA | 32 | UCACUUAGACCAAGGAGAAAC | 129 | AD00082 |
| GGUUUGCUGGGUUUAUUUUAA | 33 | UUAAAAUAAACCCAGCAAACC | 130 | AD00083 |
| GUUUGCUGGGUUUAUUUUAGA | 34 | UCUAAAAUAAACCCAGCAAAC | 131 | AD00084 |
| GUUGCUGGGUUUAUUUUAGAA | 35 | UUCUAAAAUAAACCCAGCAAC | 132 | AD00085 |
| GGGGUUUAUUUUAGAGAAUGA | 36 | UCAUUCUCUAAAAUAAACCCC | 133 | AD00086 |
| GGGUUUAUUUUAGAGAAUGGA | 37 | UCCAUUCUCUAAAAUAAACCC | 134 | AD00087 |
| GGGCAAGAACCAGUGUUUAGA | 38 | UCUAAACACUGGUUCUUGCCC | 135 | AD00088 |
| GGCAAGAACCAGUGUUUAGCA | 39 | UGCUAAACACUGGUUCUUGCC | 136 | AD00089 |
| GCAAGAACCAGUGUUUAGCGA | 40 | UCGCUAAACACUGGUUCUUGC | 137 | AD00090 |
| GCUGUUCCAAAAAGAAUUCCA | 41 | UGGAAUUCUUUUUGGAACAGC | 138 | AD00091 |
| GGUUCCAAAAAGAAUUCCAAA | 42 | UUUGGAAUUCUUUUUGGAACC | 139 | AD00092 |
| GUUCCAAAAAGAAUUCCAACA | 43 | UGUUGGAAUUCUUUUUGGAAC | 140 | AD00093 |
| GCAAAAAGAAUUCCAACCGAA | 44 | UUCGGUUGGAAUUCUUUUUGC | 141 | AD00094 |
| GAAAAAGAAUUCCAACCGACA | 45 | UGUCGGUUGGAAUUCUUUUUC | 142 | AD00095 |
| GCAACCGACCAGCUUGUUUGA | 46 | UCAAACAAGCUGGUCGGUUGC | 143 | AD00096 |
| GAACCGACCAGCUUGUUUGUA | 47 | UACAAACAAGCUGGUCGGUUC | 144 | AD00097 |
| GGACCAGCUUGUUUGUGAAAA | 48 | UUUUCACAAACAAGCUGGUCC | 145 | AD00098 |
| GACCAGCUUGUUUGUGAAACA | 49 | UGUUUCACAAACAAGCUGGUC | 146 | AD00099 |
| GCCAGCUUGUUUGUGAAACAA | 50 | UUGUUUCACAAACAAGCUGGC | 147 | AD00100 |
| GUCAUCCACAAUGAGAGUACA | 51 | UGUACUCUCAUUGUGGAUGAC | 148 | AD00101 |
| GUGGGCUUCCGUAUAUAUGGA | 52 | UCCAUAUAUACGGAAGCCCAC | 149 | AD00113 |
| GCUGUUUGCUGUGUAUGAUCA | 53 | UGAUCAUACACAGCAAACAGC | 150 | AD00114 |
| GGUUUGCUGGGUUUAUUUUAA | 54 | UUAAAAUAAACCCAGCAAACC | 151 | AD00115 |
| GCCAGCUUGUUUGUGAAACAA | 55 | UUGUUUCACAAACAAGCUGGC | 152 | AD00116 |
| GUCCCACCUUUUCUUCUAAUA | 56 | UAUUAGAAGAAAAGGUGGGAC | 153 | AD00122 |
| GCCUUUUCUUCUAAUGAGUCA | 57 | UGACUCAUUAGAAGAAAAGGC | 154 | AD00123 |
| GGGGUUUAUUUUAGAGAAUGA | 58 | UCAUUCUCUAAAAUAAACCCC | 155 | AD00124 |
| GGUUCCAAAAAGAAUUCCAAA | 59 | UUUGGAAUUCUUUUUGGAACC | 156 | AD00125 |
| GUUCCAAAAAGAAUUCCAACA | 60 | UGUUGGAAUUCUUUUUGGAAC | 157 | AD00126 |
| CAUCCACAAUGAGAGUACCUA | 61 | UAGGUACUCUCAUUGUGGAUG | 158 | AD00154 |
| CUUCUUGGGCUUCCGUAUAUA | 62 | UAUAUACGGAAGCCCAAGAAG | 159 | AD00155 |
| CAUGCAGGCUGUGACAGGAUA | 63 | UAUCCUGUCACAGCCUGCAUG | 160 | AD00156 |
| GCUGAACCUGCAAAAAUUGAA | 64 | UUCAAUUUUUGCAGGUUCAGC | 161 | AD00157 |
| CACCUUUUCUUCUAAUGAGUA | 65 | UACUCAUUAGAAGAAAAGGUG | 162 | AD00158 |
| CCGUUUCUCCUUGGUCUAAGA | 66 | UCUUAGACCAAGGAGAAACGG | 163 | AD00159 |
| ACUGUUCCAAAAAGAAUUCCA | 67 | UGGAAUUCUUUUUGGAACAGU | 164 | AD00160 |
| CAAAAAGAAUUCCAACCGACA | 68 | UGUCGGUUGGAAUUCUUUUUG | 165 | AD00161 |
| CGACCAGCUUGUUUGUGAAAA | 69 | UUUUCACAAACAAGCUGGUCG | 166 | AD00162 |
| GACCAGCUUGUUUGUGAAACA | 70 | UGUUUCACAAACAAGCUGGUC | 167 | AD00163 |
| UCGUCAUCCACAAUGAGAGUA | 71 | UACUCUCAUUGUGGAUGACGA | 168 | AD00252 |
| GUCCACAAUGAGAGUACCUGA | 72 | UCAGGUACUCUCAUUGUGGAC | 169 | AD00253 |
| AGGGUCUCACUUUCCAGCAAA | 73 | UUUGCUGGAAAGUGAGACCCU | 170 | AD00254 |
| CUGUUUGCUGUGUAUGAUCAA | 74 | UUGAUCAUACACAGCAAACAG | 171 | AD00255 |
| UUUGCUGUGUAUGAUCAAAGA | 75 | UCUUUGAUCAUACACAGCAAA | 172 | AD00256 |
| GUUUCUCCUUGGUCUAAGUGA | 76 | UCACUUAGACCAAGGAGAAAC | 173 | AD00257 |
| GCAAGAACCAGUGUUUAGCGA | 77 | UCGCUAAACACUGGUUCUUGC | 174 | AD00258 |
| CCAAAAAGAAUUCCAACCGAA | 78 | UUCGGUUGGAAUUCUUUUUGG | 175 | AD00259 |
| CAACCGACCAGCUUGUUUGUA | 79 | UACAAACAAGCUGGUCGGUUG | 176 | AD00260 |
| GACCUUUUCUUCUAAUGAGUA | 80 | UACUCAUUAGAAGAAAAGGUC | 177 | AD00158-1 |
| GACCUUUCUUUCUAGCGAGUA | 81 | UACUCAUUAGAAGAAAAGGUC | 178 | AD00158-2 |
| GACCUUUUCUUCUAAUGAGUA | 82 | UACUCAUUAGAAGAAAAGGUC | 179 | AD00158-3 |
| CACCAGCUUGUUUGUGAAACA | 83 | UGUUUCACAAACAAGCUGGUG | 180 | AD00163-1 |
| CACCAGCUUGUUUGUGAAACA | 84 | UGUUUCACAAACAAGCUGGUG | 181 | AD00163-2 |
| GCGUUUCUCCUUGGUCUAAGA | 85 | UCUUAGACCAAGGAGAAACGC | 182 | AD00159-1 |
| GCGUUUCUCCUUGGUCUAAGA | 86 | UCUUAGACCAAGGAGAAACGC | 183 | AD00159-2 |
| GCAAAAAGAAUUCCAACCGAA | 87 | UUCGGUUGGAAUUCUUUUUGC | 184 | AD00300-1 |
| CCUUUUCUUCUAAUGAGUA | 88 | UACUCAUUAGAAGAAAAGG | 185 | AD00158-19-1 |
| CCAGCUUGUUUGUGAAACA | 89 | UGUUUCACAAACAAGCUGG | 186 | AD00163-19-1 |
| GUUUCUCCUUGGUCUAAGA | 90 | UCUUAGACCAAGGAGAAAC | 187 | AD00159-19-1 |
| AAAAAGAAUUCCAACCGAA | 91 | UUCGGUUGGAAUUCUUUUU | 188 | AD00300-19-1 |
| CCCACCUUUUCUUCUAAUA | 92 | UAUUAGAAGAAAAGGUGGG | 189 | AD00122-19-1 |
| CCUUUUCUUCUAAUGAGUU | 93 | AACUCAUUAGAAGAAAAGG | 190 | AD00158-19-2 |
| CCAGCUUGUUUGUGAAACU | 94 | AGUUUCACAAACAAGCUGG | 191 | AD00163-19-2 |
| GUUUCUCCUUGGUCUAAGU | 95 | ACUUAGACCAAGGAGAAAC | 192 | AD00159-19-2 |
| AAAAAGAAUUCCAACCGAU | 96 | AUCGGUUGGAAUUCUUUUU | 193 | AD00300-19-2 |
| CCCACCUUUUCUUCUAAUU | 97 | AAUUAGAAGAAAAGGUGGG | 194 | AD00122-19-2 |
| CACCAGCUUGUUUGUGAAACA | 522 | UGUUUCACAAACAAGCUGGUG | 523 | AD00163-3 |
| CACCAGCUUGUUUGUGAAACA | 528 | UGUUUCACAAACAAGCUGGUG | 559 | AV01227 |
| CACCAGCUUGUUUGUGAAACU | 529 | AGUUUCACAAACAAGCUGGUG | 560 | AV01228 |
| CACCAGCUUGUUUGUGAAACC | 530 | GGUUUCACAAACAAGCUGGUG | 561 | AV01229 |
| CACCAGCUUGUUUGUGAAACG | 531 | CGUUUCACAAACAAGCUGGUG | 562 | AV01230 |
| AGCUUGUUUGUGAAACA | 532 | UGUUUCACAAACAAGCU | 563 | AV01231 |
| CAGCUUGUUUGUGAAACA | 533 | UGUUUCACAAACAAGCUG | 564 | AV01232 |
| ACAGCUUGUUUGUGAAACA | 534 | UGUUUCACAAACAAGCUGU | 565 | AV01233 |
| UCAGCUUGUUUGUGAAACA | 535 | UGUUUCACAAACAAGCUGA | 566 | AV01234 |
| GCAGCUUGUUUGUGAAACA | 536 | UGUUUCACAAACAAGCUGC | 567 | AV01235 |
| CCAGCUUGUUUGUGAAACA | 537 | UGUUUCACAAACAAGCUGG | 568 | AV01236 |
| ACCAGCUUGUUUGUGAAACA | 538 | UGUUUCACAAACAAGCUGGU | 569 | AV01237 |
| UCCAGCUUGUUUGUGAAACA | 539 | UGUUUCACAAACAAGCUGGA | 570 | AV01238 |
| GCCAGCUUGUUUGUGAAACA | 540 | UGUUUCACAAACAAGCUGGC | 571 | AV01239 |
| CCCAGCUUGUUUGUGAAACA | 541 | UGUUUCACAAACAAGCUGGG | 572 | AV01240 |
| GACCAGCUUGUUUGUGAAACA | 542 | UGUUUCACAAACAAGCUGGUC | 573 | AV01241 |
| AACCAGCUUGUUUGUGAAACA | 543 | UGUUUCACAAACAAGCUGGUU | 574 | AV01242 |
| UACCAGCUUGUUUGUGAAACA | 544 | UGUUUCACAAACAAGCUGGUA | 575 | AV01243 |
| CGACCAGCUUGUUUGUGAAACA | 545 | UGUUUCACAAACAAGCUGGUCG | 576 | AV01244 |
| CCGACCAGCUUGUUUGUGAAACA | 546 | UGUUUCACAAACAAGCUGGUCGG | 577 | AV01245 |
| ACCGACCAGCUUGUUUGUGAAACA | 547 | UGUUUCACAAACAAGCUGGUCGGU | 578 | AV01246 |
| AACCGACCAGCUUGUUUGUGAAACA | 548 | UGUUUCACAAACAAGCUGGUCGGUU | 579 | AV01247 |
| CAACCGACCAGCUUGUUUGUGAAACA | 549 | UGUUUCACAAACAAGCUGGUCGGUUG | 580 | AV01248 |
| GAAUUCCAACCGACCAGCUUGUUUGUGAAACA | 550 | UGUUUCACAAACAAGCUGGUCGGUUGGAAUUC | 581 | AV01249 |
| CACCAGCUUGUUUGUGAAACA | 551 | UGUUUCACAAACAAGCUGGUGUU | 582 | AV01250 |
| CACCAGCUUGUUUGUGAAACA | 552 | UGUUUCACAAACAAGCUGGUGGA | 583 | AV01251 |
| CACCAGCUUGUUUGUGAAACA | 553 | UGUUUCACAAACAAGCUGGUG | 584 | AV01252 |
| CACCAGCUUGUUUGUGAAACA | 554 | UGUUUCACAAACAAGCUGGUG | 585 | AV01253 |
| CACCAGCUUGUUUGUGAAACA | 555 | UGUUUCACAAACAAGCUGGUG | 586 | AV01254 |
| CACCAGCUUGUUUGUAAAACA | 556 | UGUUUCACAAACAAGCUGGUG | 587 | AV01255 |
| CACCAGCUUGUUUGUGAAAUA | 557 | UAUUUCACAAACAAGCUGGUG | 588 | AV01256 |
| CACCAGCUUGUUUGUGAAACA | 558 | UGUUUCACAAACAAGCUGGUG | 589 | AV01257 |
| CCAGCUUGUUUGUGAAAC | 652 | GUUUCACAAACAAGCUGG | 653 | AV01711 |

Table 2 shows certain chemically modified AGT RNAi agent antisense strand and sense strand sequences of the invention. In some embodiments of methods of the invention, an RNAi agent having the polynucleotide sequence shown in Table 2 is administered to the cell and/or subject. In some embodiments of the methods of the invention, an RNAi agent having the polynucleotide sequence shown in Table 2 is administered to the subject. In some embodiments of the invention, the RNAi agent administered to the subject comprises the duplexes noted in the first column of Table 2, and comprises the sequence modifications of sense and antisense strand sequence shown in the third and sixth columns of the same row in Table 2, respectively. In some embodiments of the methods of the invention, the sequences shown in Table 2 may be linked to (also referred to herein as “conjugated to”) a compound capable of delivering an RNAi agent to cells and/or tissues of a subject. Non-limiting examples of delivery compounds that may be used in certain embodiments of the present invention are GalNAc-containing compounds. In Table 2, the first column indicates the duplex AD # or AV # of the base sequence, corresponding to Table 1. For the base sequence identified by the duplex AD #, not only the base sequence contained in the sense and antisense strands is shown, but also the designated chemical modification shown in the same row of Table 2 is shown. For example, the first row of Table 1 shows the bases single-stranded sequence of sense and antisense, which together form a duplex, identified as: duplex AD #AD00051; and in the duplex AD #AD00051 listed in Table 2, As a duplex, it contains the base sequences of AD00051-SS and AD00051-AS, and contains chemical modifications in the sense and antisense sequences shown in the third and sixth columns, respectively. “Sense Strand SS #” in column 2 of Table 2 is the assigned identifier for the sense sequence (including modifications) shown in column 3 in the same row. The “antisense strand AS #” in the fifth column of Table 2 is the assigned identifier for the antisense sequence (including modifications) shown in the sixth column.

TABLE 2
Antisense and sense strand sequences of chemically modified AGT RNAi agents are provided. All
sequences are shown 5′ to 3′. These sequences were used in some of the in vitro testing studies
described herein. Chemical modifications are expressed as: upper case: 2′-fluoro; lower case:
2′-OMe; thiophosphate: *

| | Sense | | SEQ | | | SEQ |
|---|---|---|---|---|---|---|
| Duplex | strand | | ID | Antisense | | ID |
| AD# | SS# | Sense strand sequence | NO | strand AS# | Antisense strand sequence | NO |
| AD00051 | AD00051-SS | g*c*gucaucCaCaAugagagu*a | 195 | AD00051-AS | u*A*cucuCauugUgGaUgac*g*c | 245 |
| AD00053 | AD00053-SS | g*a*uccacaAuGaGaguaccu*a | 196 | AD00053-AS | u*A*gguaCucucAuUgUgga*u*c | 246 |
| AD00054 | AD00054-SS | g*u*ccacaaUgAgAguaccug*a | 197 | AD00054-AS | u*C*agguAcucuCaUuGugg*a*c | 247 |
| AD00055 | AD00055-SS | g*u*ucuuggGcUuCcguauau*a | 198 | AD00055-AS | u*A*uauaCggaaGcCcAaga*a*c | 248 |
| AD00056 | AD00056-SS | g*u*ugggcuUcCgUauauaug*a | 199 | AD00056-AS | u*C*auauAuacgGaAgCcca*a*c | 249 |
| AD00057 | AD00057-SS | g*u*gggcuuCcGuAuauaugg*a | 200 | AD00057-AS | u*C*cauaUauacGgAaGccc*a*c | 250 |
| AD00058 | AD00058-SS | g*g*cuuccgUaUaUauggcau*a | 201 | AD00058-AS | u*A*ugccAuauaUaCgGaag*c*c | 251 |
| AD00059 | AD00059-SS | g*c*uuccguAuAuAuggcaug*a | 202 | AD00059-AS | u*C*augcCauauAuAcGgaa*g*c | 252 |
| AD00060 | AD00060-SS | g*c*guauauAuGgCaugcaca*a | 203 | AD00060-AS | u*U*gugcAugccAuAuAuac*g*c | 253 |
| AD00061 | AD00061-SS | g*g*uuccuuGgAaGgacaaga*a | 204 | AD00061-AS | u*U*cuugUccuuCcAaGgaa*c*c | 254 |
| AD00062 | AD00062-SS | g*a*gaagauUgAcAgguucau*a | 205 | AD00062-AS | u*A*ugaaCcuguCaAuCuuc*u*c | 255 |
| AD00063 | AD00063-SS | g*a*ugcaggCuGuGacaggau*a | 206 | AD00063-AS | u*A*uccuGucacAgCcUgca*u*c | 256 |
| AD00064 | AD00064-SS | g*g*aguucuGgGuGgacaaca*a | 207 | AD00064-AS | u*U*guugUccacCcAgAacu*c*c | 257 |
| AD00065 | AD00065-SS | g*c*aacagcAcCuCagugucu*a | 208 | AD00065-AS | u*A*gacaCugagGuGcUguu*g*c | 258 |
| AD00066 | AD00066-SS | g*g*ggucucAcUuUccagcaa*a | 209 | AD00066-AS | u*U*ugcuGgaaaGuGaGacc*c*c | 259 |
| AD00067 | AD00067-SS | g*u*cacuuuCcAgCaaaacuc*a | 210 | AD00067-AS | u*G*aguuUugcuGgAaAgug*a*c | 260 |
| AD00068 | AD00068-SS | g*c*cagcaaAaCuCccucaac*a | 211 | AD00068-AS | u*G*uugaGggagUuUuGcug*g*c | 261 |
| AD00069 | AD00069-SS | g*a*gcaaaaCuCcCucaacug*a | 212 | AD00069-AS | u*C*aguuGagggAgUuUugc*u*c | 262 |
| AD00070 | AD00070-SS | g*g*agcugaAcCuGcaaaaau*a | 213 | AD00070-AS | u*A*uuuuUgcagGuUcAgcu*c*c | 263 |
| AD00071 | AD00071-SS | g*c*ugaaccUgCaAaaauuga*a | 214 | AD00071-AS | u*U*caauUuuugCaGgUuca*g*c | 264 |
| AD00072 | AD00072-SS | g*g*aaccgcCcAuUccuguuu*a | 215 | AD00072-AS | u*A*aacaGgaauGgGcGguu*c*c | 265 |
| AD00073 | AD00073-SS | g*a*accgccCaUuCcuguuug*a | 216 | AD00073-AS | u*C*aaacAggaaUgGgCggu*u*c | 266 |
| AD00074 | AD00074-SS | g*u*uccuguUuGcUguguaug*a | 217 | AD00074-AS | u*C*auacAcagcAaAcAgga*a*c | 267 |
| AD00075 | AD00075-SS | g*c*uguuugCuGuGuaugauc*a | 218 | AD00075-AS | u*G*aucaUacacAgCaAaca*g*c | 268 |
| AD00076 | AD00076-SS | g*u*guuugcUgUgUaugauca*a | 219 | AD00076-AS | u*U*gaucAuacaCaGcAaac*a*c | 269 |
| AD00077 | AD00077-SS | g*u*ugcuguGuAuGaucaaag*a | 220 | AD00077-AS | u*C*uuugAucauAcAcAgca*a*c | 270 |
| AD00078 | AD00078-SS | g*u*cccaccUuUuCuucuaau*a | 221 | AD00078-AS | u*A*uuagAagaaAaGgUggg*a*c | 271 |
| AD00079 | AD00079-SS | g*a*ccuuuuCuUcUaaugagu*a | 222 | AD00079-AS | u*A*cucaUuagaAgAaAagg*u*c | 272 |
| AD00080 | AD00080-SS | g*c*cuuuucUuCuAaugaguc*a | 223 | AD00080-AS | u*G*acucAuuagAaGaAaag*g*c | 273 |
| AD00081 | AD00081-SS | g*c*guuucuCcUuGgucuaag*a | 224 | AD00081-AS | u*C*uuagAccaaGgAgAaac*g*c | 274 |
| AD00082 | AD00082-SS | g*u*uucuccUuGgUcuaagug*a | 225 | AD00082-AS | u*C*acuuAgaccAaGgAgaa*a*c | 275 |
| AD00083 | AD00083-SS | g*g*uuugcuGgGuUuauuuua*a | 226 | AD00083-AS | u*U*aaaaUaaacCcAgCaaa*c*c | 276 |
| AD00084 | AD00084-SS | g*u*uugcugGgUuUauuuuag*a | 227 | AD00084-AS | u*C*uaaaAuaaaCcCaGcaa*a*c | 277 |
| AD00085 | AD00085-SS | g*u*ugcuggGuUuAuuuuaga*a | 228 | AD00085-AS | u*U*cuaaAauaaAcCcAgca*a*c | 278 |
| AD00086 | AD00086-SS | g*g*gguuuaUuUuAgagaaug*a | 229 | AD00086-AS | u*C*auucUcuaaAaUaAacc*c*c | 279 |
| AD00087 | AD00087-SS | g*g*guuuauUuUaGagaaugg*a | 230 | AD00087-AS | u*C*cauuCucuaAaAuAaac*c*c | 280 |
| AD00088 | AD00088-SS | g*g*gcaagaAcCaGuguuuag*a | 231 | AD00088-AS | u*C*uaaaCacugGuUcUugc*c*c | 281 |
| AD00089 | AD00089-SS | g*g*caagaaCcAgUguuuagc*a | 232 | AD00089-AS | u*G*cuaaAcacuGgUuCuug*c*c | 282 |
| AD00090 | AD00090-SS | g*c*aagaacCaGuGuuuagcg*a | 233 | AD00090-AS | u*C*gcuaAacacUgGuUcuu*g*c | 283 |
| AD00091 | AD00091-SS | g*c*uguuccAaAaAgaauucc*a | 234 | AD00091-AS | u*G*gaauUcuuuUuGgAaca*g*c | 284 |
| AD00092 | AD00092-SS | g*g*uuccaaAaAgAauuccaa*a | 235 | AD00092-AS | u*U*uggaAuucuUuUuGgaa*c*c | 285 |
| AD00093 | AD00093-SS | g*u*uccaaaAaGaAuuccaac*a | 236 | AD00093-AS | u*G*uuggAauucUuUuUgga*a*c | 286 |
| AD00094 | AD00094-SS | g*c*aaaaagAaUuCcaaccga*a | 237 | AD00094-AS | u*U*cgguUggaaUuCuUuuu*g*c | 287 |
| AD00095 | AD00095-SS | g*a*aaaagaAuUcCaaccgac*a | 238 | AD00095-AS | u*G*ucggUuggaAuUcUuuu*u*c | 288 |
| AD00096 | AD00096-SS | g*c*aaccgaCcAgCuuguuug*a | 239 | AD00096-AS | u*C*aaacAagcuGgUcGguu*g*c | 289 |
| AD00097 | AD00097-SS | g*a*accgacCaGcUuguuugu*a | 240 | AD00097-AS | u*A*caaaCaagcUgGuCggu*u*c | 290 |
| AD00098 | AD00098-SS | g*g*accagcUuGuUugugaaa*a | 241 | AD00098-AS | u*U*uucaCaaacAaGcUggu*c*c | 291 |
| AD00099 | AD00099-SS | g*a*ccagcuUgUuUgugaaac*a | 242 | AD00099-AS | u*G*uuucAcaaaCaAgCugg*u*c | 292 |
| AD00100 | AD00100-SS | g*c*cagcuuGuUuGugaaaca*a | 243 | AD00100-AS | u*U*guuuCacaaAcAaGcug*g*c | 293 |
| AD00101 | AD00101-SS | g*u*cauccaCaAuGagaguac*a | 244 | AD00101-AS | u*G*uacuCucauUgUgGaug*a*c | 294 |
| AV01227 | AV01227-SS | c*a*ccagcuUgUuUgugaaac*a | 590 | AV01227-AS | u*G*uuucAcaaaCaAgCugg*u*g | 621 |
| AV01228 | AV01228-SS | c*a*ccagcuUgUuUgugaaac*u | 591 | AV01228-AS | a*G*uuucAcaaaCaAgCugg*u*g | 622 |
| AV01229 | AV01229-SS | c*a*ccagcuUgUuUgugaaac*c | 592 | AV01229-AS | g*G*uuucAcaaaCaAgCugg*u*g | 623 |
| AV01230 | AV01230-SS | c*a*ccagcuUgUuUgugaaac*g | 593 | AV01230-AS | c*G*uuucAcaaaCaAgCugg*u*g | 624 |
| AV01231 | AV01231-SS | a*g*cuUgUuUgugaaac*a | 594 | AV01231-AS | u*G*uuucAcaaaCaAg*C*u | 625 |
| AV01232 | AV01232-SS | c*a*gcuUgUuUgugaaac*a | 595 | AV01232-AS | u*G*uuucAcaaaCaAgC*u*g | 626 |
| AV01233 | AV01233-SS | a*c*agcuUgUuUgugaaac*a | 596 | AV01233-AS | u*G*uuucAcaaaCaAgCu*g*u | 627 |
| AV01234 | AV01234-SS | u*c*agcuUgUuUgugaaac*a | 597 | AV01234-AS | u*G*uuucAcaaaCaAgCu*g*a | 628 |
| AV01235 | AV01235-SS | g*c*agcuUgUuUgugaaac*a | 598 | AV01235-AS | u*G*uuucAcaaaCaAgCu*g*c | 629 |
| AV01236 | AV01236-SS | c*c*agcuUgUuUgugaaac*a | 599 | AV01236-AS | u*G*uuucAcaaaCaAgCu*g*g | 630 |
| AV01237 | AV01237-SS | a*c*cagcuUgUuUgugaaac*a | 600 | AV01237-AS | u*G*uuucAcaaaCaAgCug*g*u | 631 |
| AV01238 | AV01238-SS | u*c*cagcuUgUuUgugaaac*a | 601 | AV01238-AS | u*G*uuucAcaaaCaAgCug*g*a | 632 |
| AV01239 | AV01239-SS | g*c*cagcuUgUuUgugaaac*a | 602 | AV01239-AS | u*G*uuucAcaaaCaAgCug*g*c | 633 |
| AV01240 | AV01240-SS | c*c*cagcuUgUuUgugaaac*a | 603 | AV01240-AS | u*G*uuucAcaaaCaAgCug*g*g | 634 |
| AV01241 | AV01241-SS | g*a*ccagcuUgUuUgugaaac*a | 604 | AV01241-AS | u*G*uuucAcaaaCaAgCugg*u*c | 635 |
| AV01242 | AV01242-SS | a*a*ccagcuUgUuUgugaaac*a | 605 | AV01242-AS | u*G*uuucAcaaaCaAgCugg*u*u | 636 |
| AV01243 | AV01243-SS | u*a*ccagcuUgUuUgugaaac*a | 606 | AV01243-AS | u*G*uuucAcaaaCaAgCugg*u*a | 637 |
| AV01244 | AV01244-SS | c*g*accagcuUgUuUgugaaac*a | 607 | AV01244-AS | u*G*uuucAcaaaCaAgCuggu*c*g | 638 |
| AV01245 | AV01245-SS | c*c*gaccagcuUgUuUgugaaa | 608 | AV01245-AS | u*G*uuucAcaaaCaAgCugguc* | 639 |
| | | c*a | | | g*g | |
| AV01246 | AV01246-SS | a*c*cgaccagcuUgUuUgugaaa | 609 | AV01246-AS | u*G*uuucAcaaaCaAgCuggucg* | 640 |
| | | c*a | | | g*u | |
| AV01247 | AV01247-SS | a*a*ccgaccagcuUgUuUgugaaa | 610 | AV01247-AS | u*G*uuucAcaaaCaAgCuggucgg* | 641 |
| | | c*a | | | u*u | |
| AV01248 | AV01248-SS | c*a*accgaccagcuUgUuUgugaa | 611 | AV01248-AS | u*G*uuucAcaaaCaAgCuggucgg | 642 |
| | | ac*a | | | u*u*g | |
| AV01249 | AV01249-SS | g*a*auuccaaccgaccagcuUgUu | 612 | AV01249-AS | u*G*uuucAcaaaCaAgCuggucggu | 643 |
| | | Ugugaaac*a | | | uggaau*u*c | |
| AV01250 | AV01250-SS | c*a*ccagcuUgUuUgugaaac*a | 613 | AV01250-AS | u*G*uuucAcaaaCaAgCuggug* | 644 |
| | | | | | u*u | |
| AV01251 | AV01251-SS | c*a*ccagcuUgUuUgugaaac*a | 614 | AV01251-AS | u*G*uuucAcaaaCaAgCuggug* | 645 |
| | | | | | g*a | |
| AV01252 | AV01252-SS | c*a*ccagcuUgUuUgugaaac*a | 615 | AV01252-AS | u*G*uuu(cUNA)AcaaaCaAgCug | 646 |
| | | | | | g*u*g | |
| AV01253 | AV01253-SS | c*a*ccagcuUgUuUgugaaac*a | 616 | AV01253-AS | u*G*uuuc(aUNA)caaaCaAgCug | 647 |
| | | | | | g*u*g | |
| AV01254 | AV01254-SS | c*a*ccagcuUGUuugugaaac*a | 617 | AV01254-AS | u*G*uuuCacaaacaAgCugg*u*g | 648 |
| AV01255 | AV01255-SS | c*a*ccagcuUgUuUguaaaac*a | 618 | AV01255-AS | u*G*uuucAcaaaCaAgCugg*u*g | 649 |
| AV01256 | AV01256-SS | c*a*ccagcuUgUuUgugaaau*a | 619 | AV01256-AS | u*A*uuucAcaaaCaAgCugg*u*g | 650 |
| AV01257 | AV01257-SS | c*a*ccagcuUgUuUgugaaa*c*a | 620 | AV01257-AS | u*G*uuucAcaaaCaAgCugg*u*g | 651 |

Table 3 shows certain chemically modified AGT RNAi agent antisense strand and sense strand sequences of the invention. In some embodiments of methods of the invention, RNAi agents shown in Table 3 are administered to a cell and/or subject. In some embodiments of methods of the invention, an RNAi agent with a polynucleotide sequence shown in Table 3 is administered to a subject. In some embodiments of the invention an RNAi agent administered to a subject comprises a duplex identified in a row in Table 3, column one and includes the sequence modifications and/or delivery compound show in the sense and antisense strand sequences in the same row in Table 3, columns three and six, respectively. The sequences were used in certain in vivo testing studies described elsewhere herein. In some embodiments of methods of the invention, a sequence shown in Table 3 may be attached to (also referred to herein as “conjugated to”) a compound for delivery, a non-limiting example of which is a GalNAc-containing compound, with a delivery compound identified in Table 3 as “GLX-n” on sense strands in column three. As used herein, “GLX” is used to represent “GLS” or “GLO” delivery compound (“X” can be “S” or “0”), and GLX-n can be any GLS and GLO that can be linked during synthesis Delivery of compounds to the 3′ or 5′-terminus of oligonucleotides. For example, but not limited to: GLX-13 and GLX-14 can be connected to the 3′-terminus of the oligonucleotide of the present invention during the synthesis process, GLX-5 and GLX-15 can be connected to the 5′-terminus of oligonucleotide of the present invention during the synthesis. In some embodiments, As used herein and shown in Table 3, “GLX-n” is used to indicate the attached GalNAc-containing compound is any one of compounds GLS-1, GLS-2, GLS-3, GLS-4, GLS-5, GLS-6, GLS-7, GLS-8, GLS-9, GLS-10, GLS-11, GLS-12, GLS-13, GLS-14, GLS-15, GLS-16, GLO-1, GLO-2, GLO-3, GLO-4, GLO-5, GLO-6, GLO-7, GLO-8, GLO-9, GLO-10, GLO-11, GLO-12, GLO-13, GLO-14, GLO-15, and GLO-16. In some implementations, GLO-0 is expressed as a GalNAc-containing compound that has been disclosed in the prior art for connection, for example but not limited to, such as the compounds for attaching to GalNAc-containing disclosed in Jayaprakash, et al., (2014) J. Am. Chem. Soc., 136, 16958-16961 are fully incorporated herein. In some implementations, one skilled in the art will be able to make and use dsRNA compounds of the invention with attached delivery compounds including, but not limited to: GLS-1, GLS-2, GLS-3, GLS-4, GLS-5, GLS-6, GLS-7, GLS-8, GLS-9, GLS-10, GLS-11, GLS-12, GLS-13, GLS-14, GLS-15, GLS-16, GLO-1, GLO-2, GLO-3, GLO-4, GLO-5, GLO-6, GLO-7, GLO-8, GLO-9, GLO-10, GLO-11, GLO-12, GLO-13, GLO-14, GLO-15, and GLO-16. The structure of each of these is provided elsewhere herein. The first column of Table 3 provides the duplex AD # of the duplex assigned to the sense and antisense sequences in that row of the table. For example, duplex AD #AD00052 is a duplex composed of sense strand AD00052-SS and antisense strand AD00052-AS. Each row in Table 3 provides a sense strand and an antisense strand and discloses the duplexes formed by the indicated sense and antisense strands. “Sense Strand SS #” in the second column of Table 3 is the assigned identifier for the sense sequence (including modifications) shown in column 3 of the same row. The “antisense strand AS #” in the fifth column of Table 3 is the assigned identifier for the antisense sequence (including modifications) shown in the sixth column. The identifier for certain attached GalNAc-containing GLO compounds is shown as GLOM, and it is understood that the compound shown as GLOM may be substituted by the other of the GLO-n or GLS-n compounds, and the compound also includes in an embodiment of the methods and/or compositions of the invention.

TABLE 3
provides the antisense and sense strand sequences of the chemically modified AGT RNAi agents used for in vivo testing. All sequences are
shown 5′ to 3′. These sequences were used in some of the in vivo testing studies described elsewhere herein. The delivery molecules used
in the in vivo studies are indicated as ″GLO-0″ at the 3′ end of each sense strand. Chemical modifications are expressed as: upper case:
2′-fluoro; lower case: 2′-OMe; thiophosphate: *; unlocked nucleic acid: UNA (Note: AD00052, AD00113-AD00260: no UNA; AD00282-AD00301:
UNA version)

| Duplex | sense strand | | SEQ ID | Antisense | | SEQ ID |
|---|---|---|---|---|---|---|
| AD# | SS# | sense strand sequence | NO | strand AS# | antisense strand sequence | NO |
| AD00052 | AD00052-SS | g*u*cauccaCaAuGagaguac*a(GLO-0) | 295 | AD00052-AS | u*G*uacuCucauUgUgGaug*a*c | 344 |
| AD00113 | AD00113-SS | g*u*gggcuuCcGuAuauaugg*a(GLO-0) | 296 | AD00113-AS | u*C*cauaUauacGgAaGccc*a*c | 345 |
| AD00114 | AD00114-SS | g*c*uguuugCuGuGuaugauc*a(GLO-0) | 297 | AD00114-AS | u*G*aucaUacacAgCaAaca*g*c | 346 |
| AD00115 | AD00115-SS | g*g*uuugcuGgGuUuauuuua*a(GLO-0) | 298 | AD00115-AS | u*U*aaaaUaaacCcAgCaaa*c*c | 347 |
| AD00116 | AD00116-SS | g*c*cagcuuGuUuGugaaaca*a(GLO-0) | 299 | AD00116-AS | u*U*guuuCacaaAcAaGcug*g*c | 348 |
| AD00122 | AD00122-SS | g*u*cccaccUuUuCuucuaau*a(GLO-0) | 300 | AD00122-AS | u*A*uuagAagaaAaGgUggg*a*c | 349 |
| AD00123 | AD00123-SS | g*c*cuuuucUuCuAaugaguc*a(GLO-0) | 301 | AD00123-AS | u*G*acucAuuagAaGaAaag*g*c | 350 |
| AD00124 | AD00124-SS | g*g*gguuuaUuUuAgagaaug*a(GLO-0) | 302 | AD00124-AS | u*C*auucUcuaaAaUaAacc*c*c | 351 |
| AD00125 | AD00125-SS | g*g*uuccaaAaAgAauuccaa*a(GLO-0) | 303 | AD00125-AS | u*U*uggaAuucuUuUuGgaa*c*c | 352 |
| AD00126 | AD00126-SS | g*u*uccaaaAaGaAuuccaac*a(GLO-0) | 304 | AD00126-AS | u*G*uuggAauucUuUuUgga*a*c | 353 |
| AD00154 | AD00154-SS | c*a*uccacaAuGaGaguaccu*a(GLO-0) | 305 | AD00154-AS | u*A*gguaCucucAuUgUgga*u*g | 354 |
| AD00155 | AD00155-SS | c*u*ucuuggGcUuCcguauau*a(GLO-0) | 306 | AD00155-AS | u*A*uauaCggaaGcCcAaga*a*g | 355 |
| AD00156 | AD00156-SS | c*a*ugcaggCuGuGacaggau*a(GLO-0) | 307 | AD00156-AS | u*A*uccuGucacAgCcUgca*u*g | 356 |
| AD00157 | AD00157-SS | g*c*ugaaccUgCaAaaauuga*a(GLO-0) | 308 | AD00157-AS | u*U*caauUuuugCaGgUuca*g*c | 357 |
| AD00158 | AD00158-SS | c*a*ccuuuuCuUcUaaugagu*a(GLO-0) | 309 | AD00158-AS | u*A*cucaUuagaAgAaAagg*u*g | 358 |
| AD00159 | AD00159-SS | c*c*guuucuCcUuGgucuaag*a(GLO-0) | 310 | AD00159-AS | u*C*uuagAccaaGgAgAaac*g*g | 359 |
| AD00160 | AD00160-SS | a*c*uguuccAaAaAgaauucc*a(GLO-0) | 311 | AD00160-AS | u*G*gaauUcuuuUuGgAaca*g*u | 360 |
| AD00161 | AD00161-SS | c*a*aaaagaAuUcCaaccgac*a(GLO-0) | 312 | AD00161-AS | u*G*ucggUuggaAuUcUuuu*u*g | 361 |
| AD00162 | AD00162-SS | c*g*accagcUuGuUugugaaa*a(GLO-0) | 313 | AD00162-AS | u*U*uucaCaaacAaGcUggu*c*g | 362 |
| AD00163 | AD00163-SS | g*a*ccagcuUgUuUgugaaac*a(GLO-0) | 314 | AD00163-AS | u*G*uuucAcaaaCaAgCugg*u*c | 363 |
| AD00252 | AD00252-SS | u*c*gucaucCaCaAugagagu*a(GLO-0) | 315 | AD00252-AS | u*A*cucuCauugUgGaUgac*g*a | 364 |
| AD00253 | AD00253-SS | g*u*ccacaaUgAgAguaccug*a(GLO-0) | 316 | AD00253-AS | u*C*agguAcucuCaUuGugg*a*c | 365 |
| AD00254 | AD00254-SS | a*g*ggucucAcUuUccagcaa*a(GLO-0) | 317 | AD00254-AS | u*U*ugcuGgaaaGuGaGacc*c*u | 366 |
| AD00255 | AD00255-SS | c*u*guuugcUgUgUaugauca*a(GLO-0) | 318 | AD00255-AS | u*U*gaucAuacaCaGcAaac*a*g | 367 |
| AD00256 | AD00256-SS | u*u*ugcuguGuAuGaucaaag*a(GLO-0) | 319 | AD00256-AS | u*C*uuugAucauAcAcAgca*a*a | 368 |
| AD00257 | AD00257-SS | g*u*uucuccUuGgUcuaagug*a(GLO-0) | 320 | AD00257-AS | u*C*acuuAgaccAaGgAgaa*a*c | 369 |
| AD00258 | AD00258-SS | g*c*aagaacCaGuGuuuagcg*a(GLO-0) | 321 | AD00258-AS | u*C*gcuaAacacUgGuUcuu*g*c | 370 |
| AD00259 | AD00259-SS | c*c*aaaaagAaUuCcaaccga*a(GLO-0) | 322 | AD00259-AS | u*U*cgguUggaaUuCuUuuu*g*g | 371 |
| AD00260 | AD00260-SS | c*a*accgacCaGcUuguuugu*a(GLO-0) | 323 | AD00260-AS | u*A*caaaCaagcUgGuCggu*u*g | 372 |
| AD00282 | AD00282-SS | g*u*cccaccUuUuCuucuaau*a(GLO-0) | 324 | AD00282-AS | u*A*uuAg(aUNA)agaaAaGgUggg*a*c | 373 |
| AD00283 | AD00283-SS | g*u*uccaaaAaGaAuuccaac*a(GLO-0) | 325 | AD00283-AS | u*G*uuGg(aUNA)auucUuUuUgga*a*c | 374 |
| AD00284 | AD00284-SS | g*c*cuuuucUuCuAaugaguc*a(GLO-0) | 326 | AD00284-AS | u*G*acUc(aUNA)uuagAaGaAaag*g*c | 375 |
| AD00285 | AD00285-SS | g*c*cagcuuGuUuGugaaaca*a(GLO-0) | 327 | AD00285-AS | u*U*guUu(cUNA)acaaAcAaGcug*g*c | 376 |
| AD00286 | AD00286-SS | g*g*uuccaaAaAgAauuccaa*a(GLO-0) | 328 | AD00286-AS | u*U*ugGa(aUNA)uucuUuUuGgaa*c*c | 377 |
| AD00287 | AD00287-SS | g*g*uuugcuGgGuUuauuuua*a(GLO-0) | 329 | AD00287-AS | u*U*aaAa(uUNA)aaacCcAgCaaa*c*c | 378 |
| AD00288 | AD00288-SS | c*a*ccuuuuCuUcUaaugagu*a(GLO-0) | 330 | AD00288-AS | u*A*cuCa(uUNA)uagaAgAaAagg*u*g | 379 |
| AD00289 | AD00289-SS | g*a*ccagcuUgUuUgugaaac*a(GLO-0) | 331 | AD00289-AS | u*G*uuUc(aUNA)caaaCaAgCugg*u*c | 380 |
| AD00290 | AD00290-SS | c*c*guuucuCcUuGgucuaag*a(GLO-0) | 332 | AD00290-AS | u*C*uuAg(aUNA)ccaaGgAgAaac*g*g | 381 |
| AD00291 | AD00291-SS | c*g*accagcUuGuUugugaaa*a(GLO-0) | 333 | AD00291-AS | u*U*uuCa(cUNA)aaacAaGcUggu*c*g | 382 |
| AD00292 | AD00292-SS | c*a*aaaagaAuUcCaaccgac*a(GLO-0) | 334 | AD00292-AS | u*G*ucGg(uUNA)uggaAuUcUuuu*u*g | 383 |
| AD00293 | AD00293-SS | u*c*gucaucCaCaAugagagu*a(GLO-0) | 335 | AD00293-AS | u*A*cuCu(cUNA)auugUgGaUgac*g*a | 384 |
| AD00294 | AD00294-SS | g*u*ccacaaUgAgAguaccug*a(GLO-0) | 336 | AD00294-AS | u*C*agGu(aUNA)cucuCaUuGugg*a*c | 385 |
| AD00295 | AD00295-SS | a*g*ggucucAcUuUccagcaa*a(GLO-0) | 337 | AD00295-AS | u*U*ugCu(gUNA)gaaaGuGaGacc*c*u | 386 |
| AD00296 | AD00296-SS | c*u*guuugcUgUgUaugauca*a(GLO-0) | 338 | AD00296-AS | u*U*gaUc(aUNA)uacaCaGcAaac*a*g | 387 |
| AD00297 | AD00297-SS | u*u*ugcuguGuAuGaucaaag*a(GLO-0) | 339 | AD00297-AS | u*C*uuUg(aUNA)ucauAcAcAgca*a*a | 388 |
| AD00298 | AD00298-SS | g*u*uucuccUuGgUcuaagug*a(GLO-0) | 340 | AD00298-AS | u*C*acUu(aUNA)gaccAaGgAgaa*a*c | 389 |
| AD00299 | AD00299-SS | g*c*aagaacCaGuGuuuagcg*a(GLO-0) | 341 | AD00299-AS | u*C*gcUa(aUNA)acacUgGuUcuu*g*c | 390 |
| AD00300 | AD00300-SS | c*c*aaaaagAaUuCcaaccga*a(GLO-0) | 342 | AD00300-AS | u*U*cgGu(uUNA)ggaaUuCuUuuu*g*g | 391 |
| AD00301 | AD00301-SS | c*a*accgacCaGcUuguuugu*a(GLO-0) | 343 | AD00301-AS | u*A*caAa(cUNA)aagcUgGuCggu*u*g | 392 |
| AD00302 | AD00302-SS | c*c*aaccgaCcAgCuuguuug*a(GLO-0) | 524 | AD00302-AS | u*C*aaAc(aUNA)agcuggUcGguu*g*g | 525 |

Table 4 shows the antisense and sense strand sequences of certain chemically modified AGT RNAi agents of the invention. In some embodiments of the methods of the invention, an RNAi agent having the polynucleotide sequence shown in Table 4 is administered to the subject. In some embodiments of the invention an RNAi agent administered to a subject comprises a duplex identified in a row in Table 4, column one and includes the sequence modifications and/or delivery compound show in the sense and antisense strand sequences in the same row in Table 4, columns three and six, respectively. In some embodiments of the methods of the invention, the sequences shown in Table 4 may be linked to compounds capable of delivering the RNAi agent to cells and/or tissues of a subject. Non-limiting examples of delivery compounds that may be used in certain embodiments of the present invention are GalNAc-containing compounds. In Table 4, the term “GLX-n” denotes a compound containing GalNAc in the indicated sense strand. For example, the terms “GLO-0” and “GLS-5” each denote a different GalNAc-containing compound attached to the sense strand. It is to be understood that a compound shown as GLO-0 may be replaced by another of the GLO-n or GLS-n compounds and the resulting compounds are also included in the method and/or composition embodiments of the present invention. Similarly, a compound shown as GLS-5 may also be replaced by another of the GLS-n or GLO-n compounds, and the resulting compounds are included in the method and/or composition embodiments of the present invention. In Table 4, the compound GLX-n used to indicate the attached GalNAC-containing compound is compound GLS-1, GLS-2, GLS-3, GLS-4, GLS-5, GLS-6, GLS-7, GLS-8, GLS-9, GLS-10, GLS-11, GLS-12, GLS-13, GLS-14, GLS-15, GLS-16, GLO-1, GLO-2, GLO-3, GLO-4, GLO-5, GLO-6, GLO-7, GLO-8, GLO-9, GLO-10, GLO-11, GLO-12, GLO-13, GLO-14, GLO-15 and GLO-16, the structure of each of which is provided elsewhere herein. The first column of Table 4 indicates the duplex AD # corresponding to the duplex shown in Table 3. The duplex AD # has identified the duplex sequence corresponding to Table 3, indicating that the sense, antisense and duplex sequences in Table 4 are identical to the base sequence with the same duplex AD # in Table 3, but the sequences and duplexes in Table 4 have different chemical modifications and/or delivery compounds compared to the corresponding sequences and duplexes shown in Table 3. For example, as shown in Table 4, the sequences of AD00113-1-SS, AD00113-1-AS and their duplexes AD #AD00113-1 and AD00113-SS (sense), AD00113-AS (antisense) shown in Table 3, respectively and double-stranded AD #AD00113 have the same base sequence, while chemical modification and/or delivery compounds are as indicated in each table. The first column of Table 4 identifies the duplex AD # number; the duplexes identified by the numbers in each row contain the sense and antisense strands shown in the third and sixth columns, respectively, in the same row, and contain modified and have a GLO- or GLS-delivery compound attached on 3′ or 5′-end of the sense strand.

TABLE 4
provides the antisense and sense strand sequences of chemically modified AGT RNAi agents. These sequences were used in certain in vivo
studies described elsewhere herein. All sequences are shown 5′ to 3′. Chemical modifications are indicated as: upper case: 2′-fluoro;
lower case: 2′-OMe; thiophosphate: *; Invab = inverted abasic.

| Duplex | sense | | SEQ | Antisense | Antisense | SEQ |
|---|---|---|---|---|---|---|
| AD# | strand SS# | sense strand sequence 5′→3′ | ID NO | strand AS# | strand sequence 5′→3′ | ID NO |
| AD00113-1 | AD00113-1-SS | (GLS-5)*(Invab)*gugggcuuCcGuAuauaugga*(Invab) | 393 | AD00113-1-AS | u*C*cauaUauacGgAaGccc*a*c | 456 |
| AD00114-1 | AD00114-1-SS | (GLS-5)*(Invab)*gcuguuugCuGuGuaugauca*(Invab) | 394 | AD00114-1-AS | u*G*aucaUacacAgCaAaca*g*c | 457 |
| AD00115-1 | AD00115-1-SS | (GLS-5)*(Invab)*gguuugcuGgGuUuauuuuaa*(Invab) | 395 | AD00115-1-AS | u*U*aaaaUaaacCcAgCaaa*c*c | 458 |
| AD00116-1 | AD00116-1-SS | (GLS-5)*(Invab)*gccagcuuGuUuGugaaacaa*(Invab) | 396 | AD00116-1-AS | u*U*guuuCacaaAcAaGcug*g*c | 459 |
| AD00122-1 | AD00122-1-SS | (GLS-5)*(Invab)*gucccaccUuUuCuucuaaua*(Invab) | 397 | AD00122-1-AS | u*A*uuagAagaaAaGgUggg*a*c | 460 |
| AD00123-1 | AD00123-1-SS | (GLS-5)*(Invab)*gccuuuucUuCuAaugaguca*(Invab) | 398 | AD00123-1-AS | u*G*acucAuuagAaGaAaag*g*c | 461 |
| AD00124-1 | AD00124-1-SS | (GLS-5)*(Invab)*gggguuuaUuUuAgagaauga*(Invab) | 399 | AD00124-1-AS | u*C*auucUcuaaAaUaAacc*c*c | 462 |
| AD00125-1 | AD00125-1-SS | (GLS-5)*(Invab)*gguuccaaAaAgAauuccaaa*(Invab) | 400 | AD00125-1-AS | u*U*uggaAuucuUuUuGgaa*c*c | 463 |
| AD00126-1 | AD00126-1-SS | (GLS-5)*(Invab)*guuccaaaAaGaAuuccaaca*(Invab) | 401 | AD00126-1-AS | u*G*uuggAauucUuUuUgga*a*c | 464 |
| AD00154-1 | AD00154-1-SS | (GLS-5)*(Invab)*cauccacaAuGaGaguaccua*(Invab) | 402 | AD00154-1-AS | u*A*gguaCucucAuUgUgga*u*g | 465 |
| AD00155-1 | AD00155-1-SS | (GLS-5)*(Invab)*cuucuuggGcUuCcguauaua*(Invab) | 403 | AD00155-1-AS | u*A*uauaCggaaGcCcAaga*a*g | 466 |
| AD00156-1 | AD00156-1-SS | (GLS-5)*(Invab)*caugcaggCuGuGacaggaua*(Invab) | 404 | AD00156-1-AS | u*A*uccuGucacAgCcUgca*u*g | 467 |
| AD00157-1 | AD00157-1-SS | (GLS-5)*(Invab)*gcugaaccUgCaAaaauugaa*(Invab) | 405 | AD00157-1-AS | u*U*caauUuuugCaGgUuca*g*c | 468 |
| AD00160-1 | AD00160-1-SS | (GLS-5)*(Invab)*acuguuccAaAaAgaauucca*(Invab) | 406 | AD00160-1-AS | u*G*gaauUcuuuUuGgAaca*g*u | 469 |
| AD00161-1 | AD00161-1-SS | (GLS-5)*(Invab)*caaaaagaAuUcCaaccgaca*(Invab) | 407 | AD00161-1-AS | u*G*ucggUuggaAuUcUuuu*u*g | 470 |
| AD00162-1 | AD00162-1-SS | (GLS-5)*(Invab)*cgaccagcUuGuUugugaaaa*(Invab) | 408 | AD00162-1-AS | u*U*uucaCaaacAaGcUggu*c*g | 471 |
| AD00252-1 | AD00252-1-SS | (GLS-5)*(Invab)*ucgucaucCaCaAugagagua*(Invab) | 409 | AD00252-1-AS | u*A*cucuCauugUgGaUgac*g*a | 472 |
| AD00253-1 | AD00253-1-SS | (GLS-5)*(Invab)*guccacaaUgAgAguaccuga*(Invab) | 410 | AD00253-1-AS | u*C*agguAcucuCaUuGugg*a*c | 473 |
| AD00254-1 | AD00254-1-SS | (GLS-5)*(Invab)*agggucucAcUuUccagcaaa*(Invab) | 411 | AD00254-1-AS | u*U*ugcuGgaaaGuGaGacc*c*u | 474 |
| AD00255-1 | AD00255-1-SS | (GLS-5)*(Invab)*cuguuugcUgUgUaugaucaa*(Invab) | 412 | AD00255-1-AS | u*U*gaucAuacaCaGcAaac*a*g | 475 |
| AD00256-1 | AD00256-1-SS | (GLS-5)*(Invab)*uuugcuguGuAuGaucaaaga*(Invab) | 413 | AD00256-1-AS | u*C*uuugAucauAcAcAgca*a*a | 476 |
| AD00257-1 | AD00257-1-SS | (GLS-5)*(Invab)*guuucuccUuGgUcuaaguga*(Invab) | 414 | AD00257-1-AS | u*C*acuuAgaccAaGgAgaa*a*c | 477 |
| AD00258-1 | AD00258-1-SS | (GLS-5)*(Invab)*gcaagaacCaGuGuuuagcga*(Invab) | 415 | AD00258-1-AS | u*C*gcuaAacacUgGuUcuu*g*c | 478 |
| AD00259-1 | AD00259-1-SS | (GLS-5)*(Invab)*ccaaaaagAaUuCcaaccgaa*(Invab) | 416 | AD00259-1-AS | u*U*cgguUggaaUuCuUuuu*g*g | 479 |
| AD00260-1 | AD00260-1-SS | (GLS-5)*(Invab)*caaccgacCaGcUuguuugua*(Invab) | 417 | AD00260-1-AS | u*A*caaaCaagcUgGuCggu*u*g | 480 |
| AD00282-1 | AD00282-1-SS | (GLS-5)*(Invab)*gucccaccUuUuCuucuaaua*(Invab) | 418 | AD00282-1-AS | u*A*uuAg(aUNA)agaaAaGgUggg*a*c | 481 |
| AD00283-1 | AD00283-1-SS | (GLS-5)*(Invab)*guuccaaaAaGaAuuccaaca*(Invab) | 419 | AD00283-1-AS | u*G*uuGg(aUNA)auucUuUuUgga*a*c | 482 |
| AD00284-1 | AD00284-1-SS | (GLS-5)*(Invab)*gccuuuucUuCuAaugaguca*(Invab) | 420 | AD00284-1-AS | u*G*acUc(aUNA)uuagAaGaAaag*g*c | 483 |
| AD00285-1 | AD00285-1-SS | (GLS-5)*(Invab)*gccagcuuGuUuGugaaacaa*(Invab) | 421 | AD00285-1-AS | u*U*guUu(cUNA)acaaAcAaGcug*g*c | 484 |
| AD00286-1 | AD00286-1-SS | (GLS-5)*(Invab)*gguuccaaAaAgAauuccaaa*(Invab) | 422 | AD00286-1-AS | u*U*ugGa(aUNA)uucuUuUuGgaa*c*c | 485 |
| AD00287-1 | AD00287-1-SS | (GLS-5)*(Invab)*gguuugcuGgGuUuauuuuaa*(Invab) | 423 | AD00287-1-AS | u*U*aaAa(uUNA)aaacCcAgCaaa*c*c | 486 |
| AD00288-1 | AD00288-1-SS | (GLS-5)*(Invab)*caccuuuuCuUcUaaugagua*(Invab) | 424 | AD00288-1-AS | u*A*cuCa(uUNA)uagaAgAaAagg*u*g | 487 |
| AD00289-1 | AD00289-1-SS | (GLS-5)*(Invab)*gaccagcuUgUuUgugaaaca*(Invab) | 425 | AD00289-1-AS | u*G*uuUc(aUNA)caaaCaAgCugg*u*c | 488 |
| AD00290-1 | AD00290-1-SS | (GLS-5)*(Invab)*ccguuucuCcUuGgucuaaga*(Invab) | 426 | AD00290-1-AS | u*C*uuAg(aUNA)ccaaGgAgAaac*g*g | 489 |
| AD00291-1 | AD00291-1-SS | (GLS-5)*(Invab)*cgaccagcUuGuUugugaaaa*(Invab) | 427 | AD00291-1-AS | u*U*uuCa(cUNA)aaacAaGcUggu*c*g | 490 |
| AD00292-1 | AD00292-1-SS | (GLS-5)*(Invab)*caaaaagaAuUcCaaccgaca*(Invab) | 428 | AD00292-1-AS | u*G*ucGg(uUNA)uggaAuUcUuuu*u*g | 491 |
| AD00293-1 | AD00293-1-SS | (GLS-5)*(Invab)*ucgucaucCaCaAugagagua*(Invab) | 429 | AD00293-1-AS | u*A*cuCu(cUNA)auugUgGaUgac*g*a | 492 |
| AD00294-1 | AD00294-1-SS | (GLS-5)*(Invab)*guccacaaUgAgAguaccuga*(Invab) | 430 | AD00294-1-AS | u*C*agGu(aUNA)cucuCaUuGugg*a*c | 493 |
| AD00295-1 | AD00295-1-SS | (GLS-5)*(Invab)*agggucucAcUuUccagcaaa*(Invab) | 431 | AD00295-1-AS | u*U*ugCu(gUNA)gaaaGuGaGacc*c*u | 494 |
| AD00296-1 | AD00296-1-SS | (GLS-5)*(Invab)*cuguuugcUgUgUaugaucaa*(Invab) | 432 | AD00296-1-AS | u*U*gaUc(aUNA)uacaCaGcAaac*a*g | 495 |
| AD00297-1 | AD00297-1-SS | (GLS-5)*(Invab)*uuugcuguGuAuGaucaaaga*(Invab) | 433 | AD00297-1-AS | u*C*uuUg(aUNA)ucauAcAcAgca*a*a | 496 |
| AD00298-1 | AD00298-1-SS | (GLS-5)*(Invab)*guuucuccUuGgUcuaaguga*(Invab) | 434 | AD00298-1-AS | u*C*acUu(aUNA)gaccAaGgAgaa*a*c | 497 |
| AD00299-1 | AD00299-1-SS | (GLS-5)*(Invab)*gcaagaacCaGuGuuuagcga*(Invab) | 435 | AD00299-1-AS | u*C*gcUa(aUNA)acacUgGuUcuu*g*c | 498 |
| AD00301-1 | AD00301-1-SS | (GLS-5)*(Invab)*caaccgacCaGcUuguuugua*(Invab) | 436 | AD00301-1-AS | u*A*caAa(cUNA)aagcUgGuCggu*u*g | 499 |
| AD00302-1 | AD00302-1-SS | (GLS-5)*(Invab)*ccaaccgaCcAgCuuguuuga*(Invab) | 437 | AD00302-1-AS | u*C*aaAc(aUNA)agcuggUcGguu*g*g | 500 |
| AD00158-1 | AD00158-1-SS | (GLS-5)*(Invab)*gaccuuuuCuUcUaaugagua*(Invab) | 438 | AD00158-1-AS | u*A*cucaUuagaAgAaAagg*u*c | 501 |
| AD00158-2 | AD00158-2-SS | (GLS-5)*(Invab)*gaccuuucUuUcUagcgagua*(Invab) | 439 | AD00158-2-AS | u*A*cucaUuagaAgAaAagg*u*c | 502 |
| AD00158-3 | AD00158-3-SS | g*a*ccuuuuCuUcUaaugagu*a(GLO-0) | 440 | AD00158-3-AS | u*A*cucaUuagaAgAaAagg*u*c | 503 |
| AD00163-1 | AD00163-1-SS | (GLS-5)*(Invab)*caccagcuUgUuUgugaaaca*(Invab) | 441 | AD00163-1-AS | u*G*uuucAcaaaCaAgCugg*u*g | 504 |
| AD00163-2 | AD00163-2-SS | c*a*ccagcuUgUuUgugaaac*a(GLO-0) | 442 | AD00163-2-AS | u*G*uuucAcaaaCaAgCugg*u*g | 505 |
| AD00159-1 | AD00159-1-SS | (GLS-5)*(Invab)*gcguuucuCcUuGgucuaaga*(Invab) | 443 | AD00159-1-AS | u*C*uuagAccaaGgAgAaac*g*c | 506 |
| AD00159-2 | AD00159-2-SS | g*c*guuucuCcUuGgucuaag*a(GLO-0) | 444 | AD00159-2-AS | u*C*uuagAccaaGgAgAaac*g*c | 507 |
| AD00300-1 | AD00300-1-SS | (GLS-5)*(Invab)*gcaaaaagAaUuCcaaccgaa*(Invab) | 445 | AD00300-1-AS | u*U*cgGu(uUNA)ggaaUuCuUuuu*g*c | 508 |
| AD00158- | AD00158-19-1- | (GLS-5)*(Invab)*ccuuuuCuUcUaaugagua*(Invab) | 446 | AD00158-19-1- | u*A*cucaUuagaAgAaAa*g*g | 509 |
| 19-1 | SS | | | AS | | |
| AD00163- | AD00163-19-1- | (GLS-5)*(Invab)*ccagcuUgUuUgugaaaca*(Invab) | 447 | AD00163-19-1- | u*G*uuucAcaaaCaAgCu*g*g | 510 |
| 19-1 | SS | | | AS | | |
| AD00159- | AD00159-19-1- | (GLS-5)*(Invab)*guuucuCcUuGgucuaaga*(Invab) | 448 | AD00159-19-1- | u*C*uuagAccaaGgAgAa*a*c | 511 |
| 19-1 | SS | | | AS | | |
| AD00300- | AD00300-19-1- | (GLS-5)*(Invab)*aaaaagAaUuCcaaccgaa*(Invab) | 449 | AD00300-19-1- | u*U*cgGu(uUNA)ggaaUuCuUu*u*u | 512 |
| 19-1 | SS | | | AS | | |
| AD00122- | AD00122-19-1- | (GLS-5)*(Invab)*cccaccUuUuCuucuaaua*(Invab) | 450 | AD00122-19-1- | u*A*uuagAagaaAaGgUg*g*g | 513 |
| 19-1 | SS | | | AS | | |
| AD00158- | AD00158-19-2- | (GLS-5)*(Invab)*ccuuuuCuUcUaaugaguu*(Invab) | 451 | AD00158-19-2- | a*A*cucaUuagaAgAaAa*g*g | 514 |
| 19-2 | SS | | | AS | | |
| AD00163- | AD00163-19-2- | (GLS-5)*(Invab)*ccagcuUgUuUgugaaacu*(Invab) | 452 | AD00163-19-2- | a*G*uuucAcaaaCaAgCu*g*g | 515 |
| 19-2 | SS | | | AS | | |
| AD00159- | AD00159-19-2- | (GLS-5)*(Invab)*guuucuCcUuGgucuaagu*(Invab) | 453 | AD00159-19-2- | a*C*uuagAccaaGgAgAa*a*c | 516 |
| 19-2 | SS | | | AS | | |
| AD00300- | AD00300-19-2- | (GLS-5)*(Invab)*aaaaagAaUuCcaaccgau*(Invab) | 454 | AD00300-19-2- | a*U*cgGu(uUNA)ggaaUuCuUu*u*u | 517 |
| 19-2 | SS | | | AS | | |
| AD00122- | AD00122-19-2- | (GLS-5)*(Invab)*cccaccUuUuCuucuaauu*(Invab) | 455 | AD00122-19-2- | a*A*uuagAagaaAaGgUg*g*g | 518 |
| 19-2 | SS | | | AS | | |
| AD00163-3 | AD00163-3-SS | (GLS-15)*(Invab)*caccagcuUgUuUgugaaaca*(Invab) | 526 | AD00163-3-AS | u*G*uuucAcaaaCaAgCugg*u*g | 527 |
| AD00159-3 | AD00159-3-SS | (GLS-15)*(Invab)*gcguuucuCcUuGgucuaaga*(Invab) | 654 | AD00159-3-AS | u*C*uuagAccaaGgAgAaac*g*c | 655 |

### Mismatches

It is known to skilled in art, mismatches are tolerated for efficacy in dsRNA, especially if the mismatches are within terminal region of dsRNA. Certain mismatches are better tolerated in a dsRNA, for example, mismatches with wobble base pairs G:U and A:C are tolerated better for efficacy (Du et el., A systematic analysis of the silencing effects of an active siRNA at all single-nucleotide mismatched target sites. Nucleic Acids Res. 2005 Mar. 21; 33(5):1671-7. Doi: 10.1093/nar/gki312. Nucleic Acids Res. 2005; 33(11):3698). In some embodiments of methods and compounds of the invention an AGT dsRNA agent may contain one or more mismatches to the AGT target sequence. In some embodiments, AGT dsRNA agent of the invention includes no mismatches. In certain embodiments, AGT dsRNA agent of the invention includes no more than 1, no more than 2, or no more than 3 mismatches to the AGT target sequence. In some embodiments of the invention, an antisense strand of an AGT dsRNA agent contains mismatches to an AGT target sequence that are not located in the center of the region of complementarity. In some embodiments, the antisense strand of the AGT dsRNA agent includes 1, 2, 3, 4, or more mismatches that are within the last 5, 4, 3, 2, or 1 nucleotides from one or both of the 5′ end and the 3′ end of the region of complementarity. Methods described herein and/or methods known in the art can be used to determine whether an AGT dsRNA agent containing a mismatch to an AGT target sequence is effective in inhibiting the expression of the AGT gene.

### Complementarity

As used herein, unless otherwise indicated, the term “complementary,” when used to describe a first nucleotide sequence (e.g., AGT dsRNA agent sense strand or targeted AGT mRNA) in relation to a second nucleotide sequence (e.g., AGT dsRNA agent antisense strand or a single-stranded antisense polynucleotide), means the ability of an oligonucleotide or polynucleotide including the first nucleotide sequence to hybridize [form base pair hydrogen bonds under mammalian physiological conditions (or similar conditions in vitro)] and form a duplex or double helical structure under certain conditions with an oligonucleotide or polynucleotide including the second nucleotide sequence. Other conditions, such as physiologically relevant conditions as can be encountered inside an organism, can apply. A skilled artisan will be able to determine the set of conditions most appropriate for a test of complementarity of two sequences in accordance with the ultimate application of the hybridized nucleotides. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification.

Complementary sequences, for example, within an AGT dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. It will be understood that in embodiments when two oligonucleotides are designed to form, upon hybridization, one or more single stranded overhangs, such overhangs are not regarded herein as mismatches with regard to the determination of complementarity. For example, an AGT dsRNA agent comprising one oligonucleotide 19 nucleotides in length and another oligonucleotide 20 nucleotides in length, wherein the longer oligonucleotide comprises a sequence of 19 nucleotides that is fully complementary to the shorter oligonucleotide, can yet be referred to as “fully complementary” for the purposes described herein. Thus, as used herein, “fully complementary” means that all (100%) of the bases in a contiguous sequence of a first polynucleotide will hybridize with the same number of bases in a contiguous sequence of a second polynucleotide. The contiguous sequence may comprise all or a part of a first or second nucleotide sequence.

The term “substantially complementary” as used herein means that in a hybridized pair of nucleobase sequences, at least about 85%, but not all, of the bases in a contiguous sequence of a first polynucleotide will hybridize with the same number of bases in a contiguous sequence of a second polynucleotide. The term “substantially complementary” can be used in reference to a first sequence with respect to a second sequence if the two sequences include one or more, for example at least 1, 2, 3, 4, or 5 mismatched base pairs upon hybridization for a duplex up to 15, 16, 17, 18, 19, 20, 21, 22, 23, 24, 25, 26, 27, 28, 29 or 30 base pairs (bp), while retaining the ability to hybridize under the conditions most relevant to their ultimate application, e.g., inhibition of AGT gene expression via a RISC pathway. The term, “partially complementary” may be used herein in reference to a hybridized pair of nucleobase sequences, in which at least 75%, but not all, of the bases in a contiguous sequence of a first polynucleotide will hybridize with the same number of bases in a contiguous sequence of a second polynucleotide. In some embodiments, “partially complementary” means at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the bases in a contiguous sequence of a first polynucleotide will hybridize with the same number of bases in a contiguous sequence of a second polynucleotide

The terms “complementary,” “fully complementary,” “substantially complementary,” and “partially complimentary” are used herein in reference to the base matching between the sense strand and the antisense strand of an AGT dsRNA agent, between the antisense strand of an AGT dsRNA agent and a sequence of a target AGT mRNA, or between a single-stranded antisense oligonucleotide and a sequence of a target AGT mRNA. It will be understood that the term “antisense strand of an AGT dsRNA agent” may refer to the same sequence of an “AGT antisense polynucleotide agent”.

As used herein, the term “substantially identical” or “substantial identity” used in reference to a nucleic acid sequence means a nucleic acid sequence comprising a sequence with at least about 85% sequence identity or more, preferably at least 90%, at least 91%, at least 92%, at least 93%, at least 94%, at least 95%, at least 96%, at least 97%, at least 98%, or at least 99%, compared to a reference sequence. Percentage of sequence identity is determined by comparing two optimally aligned sequences over a comparison window. The percentage is calculated by determining the number of positions at which the identical nucleic acid base occurs in both sequences to yield the number of matched positions, dividing the number of matched positions by the total number of positions in the window of comparison and multiplying the result by 100 to yield the percentage of sequence identity. The inventions disclosed herein encompasses nucleotide sequences substantially identical to those disclosed herein. e.g., in Tables 1-4. In some embodiments, the sequences disclosed herein are exactly identical, or at least about 85%, 86%, 87%, 88%, 89%, 90%, 91%, 92%, 93%, 94%, 95%, 96%, 97%, 98%, or 99% percent identical to those disclosed herein, e.g., in Tables 1-4.

As used herein, the term “strand comprising a sequence” means an oligonucleotide comprising a chain of nucleotides that is described by the sequence referred to using the standard nucleotide nomenclature. The term “double-stranded RNA” or “dsRNA,” as used herein, refers to an RNAi that includes an RNA molecule or complex of molecules having a hybridized duplex region comprising two anti-parallel and substantially or fully complementary nucleic acid strands, which are referred to as having “sense” and “antisense” orientations with respect to a target AGT RNA. The duplex region can be of any length that permits specific degradation of a desired target AGT RNA through a RISC pathway, but will typically range from 9 to 30 base pairs in length, e.g., 15-30 base pairs in length. Considering a duplex between 9 and 30 base pairs, the duplex can be any length in this range, for example, 9, 10, 11, 12, 13, 14, 15, 16, 17, 18, 19, 20, 21, 22, 23, 24, 25, 26, 27, 28, 29, or 30, and any sub-range therein between, including, but not limited to 15-30 base pairs, 15-26 base pairs, 15-23 base pairs, 15-22 base pairs, 15-21 base pairs, 15-20 base pairs, 15-19 base pairs, 15-18 base pairs, 15-17 base pairs, 18-30 base pairs, 18-26 base pairs, 18-23 base pairs, 18-22 base pairs, 18-21 base pairs, 18-20 base pairs, 19-30 base pairs, 19-26 base pairs, 19-23 base pairs, 19-22 base pairs, 19-21 base pairs, 19-20 base pairs, 20-30 base pairs, 20-26 base pairs, 20-25 base pairs, 20-24 base pairs, 20-23 base pairs, 20-22 base pairs, 20-21 base pairs, 21-30 base pairs, 21-26 base pairs, 21-25 base pairs, 21-24 base pairs, 21-23 base pairs, or 21-22 base pairs. AGT dsRNA agents generated in the cell by processing with Dicer and similar enzymes are generally in the range of 19-22 base pairs in length. One strand of the duplex region of an AGT dsDNA agent comprises a sequence that is substantially complementary to a region of a target AGT RNA. The two strands forming the duplex structure can be from a single RNA molecule having at least one self-complementary region, or can be formed from two or more separate RNA molecules. Where the duplex region is formed from two strands of a single molecule, the molecule can have a duplex region separated by a single stranded chain of nucleotides (herein referred to as a “hairpin loop”) between the 3′-end of one strand and the 5′-end of the respective other strand forming the duplex structure. In some embodiments of the invention, a hairpin look comprises at least 1, 2, 3, 4, 5, 6, 7, 8, 9, 10, 11, 12, 13, 14, 15, 16, 17, 18, 19, 20, or more unpaired nucleotides. Where the two substantially complementary strands of an AGT dsRNA agent are comprised by separate RNA molecules, those molecules need not, but can be covalently connected. Where the two strands are connected covalently by means other than a hairpin loop, the connecting structure is referred to as a “linker.” The term “siRNA” is also used herein to refer to a dsRNA agent as described herein.

In some embodiments of the invention an AGT dsRNA agent may include a sense and antisense sequence that have no-unpaired nucleotides or nucleotide analogs at one or both terminal ends of the dsRNA agent. An end with no unpaired nucleotides is referred to as a “blunt end” and as having no nucleotide overhang. If both ends of a dsRNA agent are blunt, the dsRNA is referred to as “blunt ended.” In some embodiments of the invention, a first end of a dsRNA agent is blunt, in some embodiments a second end of a dsRNA agent is blunt, and in certain embodiments of the invention, both ends of an AGT dsRNA agent are blunt.

In some embodiments of dsRNA agents of the invention, the dsRNA does not have one or two blunt ends. In such instances there is at least one unpaired nucleotide at the end of a strand of a dsRNA agent. For example, when a 3′-end of one strand of a dsRNA extends beyond the 5′-end of the other strand, or vice versa, there is a nucleotide overhang. A dsRNA can comprise an overhang of at least el, 2, 3, 4, 5, 6, or more nucleotides. A nucleotide overhang can comprise or consist of a nucleotide/nucleoside analog, including a deoxynucleotide/nucleoside. It will be understood that in some embodiments a nucleotide overhang is on a sense strand of a dsRNA agent, on an antisense strand of a dsRNA agent, or on both ends of a dsRNA agent and nucleotide(s) of an overhang can be present on the 5′ end, 3′ end or both ends of either an antisense or sense strand of a dsRNA. In certain embodiments of the invention, one or more of the nucleotides in an overhang is replaced with a nucleoside thiophosphate.

As used herein, the term “antisense strand” or “guide strand” refers to the strand of an AGT dsRNA agent that includes a region that is substantially complementary to an AGT target sequence. As used herein the term “sense strand,” or “passenger strand” refers to the strand of an AGT dsRNA agent that includes a region that is substantially complementary to a region of the antisense strand of the AGT dsRNA agent.

### Modifications

In some embodiments of the invention the RNA of an AGT RNAi agent is chemically modified to enhance stability and/or one or more other beneficial characteristics. Nucleic acids in certain embodiments of the invention may be synthesized and/or modified by methods well established in the art, for example, those described in Current protocols in Nucleic Acid Chemistry,” Beaucage, S. L. et al. (Eds.), John Wiley & Sons, Inc., New York, N.Y., USA, which is incorporated herein by reference. Modifications that can be present in certain embodiments of AGT dsRNA agents of the invention include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in certain embodiments of AGT dsRNA agents, AGT antisense polynucleotides, and AGT sense polynucleotides of the invention include, but are not limited to RNAs comprising modified backbones or no natural internucleoside linkages. As a non-limiting example, an RNA having a modified backbone may not have a phosphorus atom in the backbone. RNAs that do not have a phosphorus atom in their internucleoside backbone may be referred to as oligonucleosides. In certain embodiments of the invention, a modified RNA has a phosphorus atom in its internucleoside backbone.

It will be understood that the term “RNA molecule” or “RNA” or “ribonucleic acid molecule” encompasses not only RNA molecules as expressed or found in nature, but also analogs and derivatives of RNA comprising one or more ribonucleotide/ribonucleoside analogs or derivatives as described herein or as known in the art. The terms “ribonucleoside” and “ribonucleotide” may be used interchangeably herein. An RNA molecule can be modified in the nucleobase structure or in the ribose-phosphate backbone structure, e.g., as described herein below, and molecules comprising ribonucleoside analogs or derivatives must retain the ability to form a duplex. As non-limiting examples, an RNA molecule can also include at least one modified ribonucleoside including but not limited to a 2′-O-methyl modified nucleoside, a nucleoside comprising a 5′ phosphorothioate group, a terminal nucleoside linked to a cholesteryl derivative or dodecanoic acid bisdecylamide group, a locked nucleoside, an abasic nucleoside, a 2′-deoxy-2′-fluoro modified nucleoside, a 2′-amino-modified nucleoside, 2′-alkyl-modified nucleoside, morpholino nucleoside, a phosphoramidate or a non-natural base comprising nucleoside, or any combination thereof. In some embodiments of the invention, an RNA molecule comprises at least 1, 2, 3, 4, 5, 6, 7, 8, 9, 10, 11, 12, 13, 14, 15, 16, 17, 18, 19, 20, or up to the full length of the AGT dsRNA agent molecule's ribonucleosides that are modified ribonucleosides. The modifications need not be the same for each of such a plurality of modified ribonucleosides in an RNA molecule.

dsRNA agents, AGT antisense polynucleotides, and/or AGT sense polynucleotides of the invention may, in some embodiments comprise one or more independently selected modified nucleotide and/or one or more independently selected non-phosphodiester linkage. As used herein the term “independently selected” used in reference to a selected element, such as a modified nucleotide, non-phosphodiester linkage, etc., means that two or more selected elements can but need not be the same as each other.

As used herein, a “nucleotide base,” “nucleotide,” or “nucleobase” is a heterocyclic pyrimidine or purine compound, which is a standard constituent of all nucleic acids, and includes the bases that form the nucleotides adenine (a), guanine (g), cytosine (c), thymine (t), and uracil (u). A nucleobase may further be modified to include, though not intended to be limiting: universal bases, hydrophobic bases, promiscuous bases, size-expanded bases, and fluorinated bases. The term “ribonucleotide” or “nucleotide” may be used herein to refer to an unmodified nucleotide, a modified nucleotide, or a surrogate replacement moiety. Those in the art will recognize that guanine, cytosine, adenine, and uracil can be replaced by other moieties without substantially altering the base pairing properties of an oligonucleotide comprising a nucleotide bearing such replacement moiety.

In one embodiment, modified RNAs contemplated for use in methods and compositions described herein are peptide nucleic acids (PNAs) that have the ability to form the required duplex structure and that permit or mediate the specific degradation of a target RNA via a RISC pathway. In certain embodiments of the invention, an AGT RNA interference agent includes a single stranded RNA that interacts with a target AGT RNA sequence to direct the cleavage of the target AGT RNA.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Means of preparing phosphorus-containing linkages are routinely practiced in the art and such methods can be used to prepare certain modified AGT dsRNA agents, certain modified AGT antisense polynucleotides, and/or certain modified AGT sense polynucleotides of the invention.

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Means of preparing modified RNA backbones that do not include a phosphorus atom are routinely practiced in the art and such methods can be used to prepare certain modified AGT dsRNA agents, certain modified AGT antisense polynucleotides, and/or certain modified AGT sense polynucleotides of the invention.

In certain embodiments of the invention, RNA mimetics are included in AGT dsRNAs, AGT antisense polynucleotides, and/or AGT sense polynucleotides, such as, but not limited to: replacement of the sugar and the internucleoside linkage, i.e., the backbone, of the nucleotide units with novel groups. In such embodiments, base units are maintained for hybridization with an appropriate AGT nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Means of preparing RNA mimetics are routinely practiced in the art and such methods can be used to prepare certain modified AGT dsRNA agents of the invention.

Some embodiments of the invention include RNAs with phosphorothioate backbones and oligonucleosides with heteroatom backbones, and in particular —CH₂—NH—CH₂—, —CH₂—N(CH₃)—O—CH₂-[known as a methylene (methylimino) or MMI backbone], —CH₂—O—N(CH₃)—CH₂—, —CH₂—N(CH₃)—N(CH₃)—CH₂— and —N(CH₃)—CH₂—-[wherein the native phosphodiester backbone is represented as —O—P—O—CH₂—]. Means of preparing RNAs with phosphorothioate backbones and oligonucleosides with heteroatom backbones are routinely practiced in the art and such methods can be used to prepare certain modified AGT dsRNA agents, certain AGT antisense polynucleotides, and/or certain AGT sense polynucleotides of the invention.

Modified RNAs can also contain one or more substituted sugar moieties. AGT dsRNAs, AGT antisense polynucleotides, and/or AGT sense polynucleotides of the invention may comprise one of the following at the 2′ position: OH; F; O—, S—, or N-alkyl; O—, S—, or N-alkenyl; S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Exemplary suitable modifications include O[(CH₂)ₙO]ₘCH₃, O(CH₂)_{b}OCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an AGT dsRNA agent, or a group for improving the pharmacodynamic properties of an AGT dsRNA agent, AGT antisense polynucleotide, and/or AGT sense polynucleotide, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂. Means of preparing modified RNAs such as those described are routinely practiced in the art and such methods can be used to prepare certain modified AGT dsRNA agents of the invention.

Other modifications include 2′-methoxy (2′—OCH₃), 2′-aminopropoxy (2′-OCH₂CH₂CH₂NH₂) and 2′-fluoro (2′-F). Similar modifications can also be made at other positions on the RNA of an AGT dsRNA agent, AGT antisense polynucleotide, and/or AGT sense polynucleotide of the invention, particularly the 3′ position of the sugar on the 3′ terminal nucleotide or in 2′-5′ linked AGT dsRNAs, AGT antisense polynucleotides, or AGT sense polynucleotides, and the 5′ position of 5′ terminal nucleotide. AGT dsRNA agents, AGT antisense polynucleotides, and/or AGT sense polynucleotides may also have sugar mimetics such as cyclobutyl moieties in place of the pentofuranosyl sugar. Means of preparing modified RNAs such as those described are routinely practiced in the art and such methods can be used to prepare certain modified AGT dsRNA agents, AGT antisense polynucleotides, and/or AGT sense polynucleotides of the invention.

An AGT dsRNA agent, AGT antisense polynucleotide, and/or AGT sense polynucleotide may, in some embodiments, include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Additional nucleobases that may be included in certain embodiments of AGT dsRNA agents of the invention are known in the art, see for example: Modified Nucleosides in Biochemistry, Biotechnology and Medicine, Herdewijn, P. Ed. Wiley-VCH, 2008; The Concise Encyclopedia Of Polymer Science And Engineering, pages 858-859, Kroschwitz, J. L, Ed. John Wiley & Sons, 1990, English et al., Angewandte Chemie, International Edition, 1991, 30, 613, Sanghvi, Y S., Chapter 15, dsRNA Research and Applications, pages 289-302, Crooke, S. T. and Lebleu, B., Ed., CRC Press, 1993. Means of preparing dsRNAs, AGT antisense strand polynucleotides and/or AGT sense strand polynucleotides that comprise nucleobase modifications and/or substitutions such as those described herein are routinely practiced in the art and such methods can be used to prepare certain modified AGT dsRNA agents, AGT sense polynucleotides, and/or AGT antisense polynucleotides of the invention.

Certain embodiments of AGT dsRNA agents, AGT antisense polynucleotides, and/or AGT sense polynucleotides of the invention include RNA modified to include one or more locked nucleic acids (LNA). A locked nucleic acid is a nucleotide with a modified ribose moiety comprising an extra bridge connecting the 2′ and 4′ carbons. This structure effectively “locks” the ribose in the 3′-endo structural conformation. The addition of locked nucleic acids in an AGT dsRNA agent, AGT antisense polynucleotides, and/or AGT sense polynucleotides of the invention may increase stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193). Means of preparing dsRNA agents, AGT antisense polynucleotides, and/or AGT sense polynucleotides that comprise locked nucleic acid(s) are routinely practiced in the art and such methods can be used to prepare certain modified AGT dsRNA agents of the invention.

Certain embodiments of AGT dsRNA compounds, sense polynucleotides, and/or antisense polynucleotides of the invention, include at least one modified nucleotide, wherein the at least one modified nucleotide comprises: a 2′-O-methyl nucleotide, 2′-Fluoro nucleotide, 2′-deoxy nucleotide, 2′3′-seco nucleotide mimic, locked nucleotide, 2′-F-Arabino nucleotide, 2′-methoyxyethyl nucleotide, 2′-amino-modified nucleotide, 2′-alkyl-modified nucleotide, mopholino nucleotide, and 3′-Ome nucleotide, a nucleotide comprising a 5′-phosphorothioate group, or a terminal nucleotide linked to a cholesteryl derivative or dodecanoic acid bisdecylamide group, a 2′-amino-modified nucleotide, ‘a phosphoramidate, or a non-natural base comprising nucleotide. In some embodiments, an AGT dsRNA compound includes an E-vinylphosphonate nucleotide at the 5′ end of the antisense strand, also referred to herein as the guide strand.

Certain embodiments of AGT dsRNA compounds, 3′ and 5′ end of sense polynucleotides, and/or 3′ end of antisense polynucleotides of the invention, include at least one modified nucleotide, wherein the at least one modified nucleotide comprises: abasic nucleotide, ribitol, inverted nucleotide, inverted abasic nucleotide, inverted 2′-OMe nucleotide, inverted 2′-deoxy nucleotide. It is known to skilled in art, including an abasic or inverted abasic nucleotide at the end of oligonucleotide enhances stability (Czauderna et al. Structural variations and stabilizing modifications of synthetic siRNAs in mammalian cells. Nucleic Acids Res. 2003; 31(11):2705-2716. doi:10.1093/nar/gkg393).

Certain embodiments of AGT dsRNA compounds, antisense polynucleotides of the invention, include at least one modified nucleotide, wherein the at least one modified nucleotide comprises unlocked nucleic acid nucleotide (UNA) or/and glycol nucleic acid nucleotide (GNA). It is known to skilled in art, UNA and GNA are thermally destabilizing chemical modifications, can significantly improves the off-target profile of a siRNA compound (Janas, et al., Selection of GalNAc-conjugated siRNAs with limited off-target-driven rat hepatotoxicity. Nat Commun. 2018; 9(1):723. doi:10.1038/s41467-018-02989-4; Laursen et al., Utilization of unlocked nucleic acid (UNA) to enhance siRNA performance in vitro and in vivo. Mol BioSyst. 2010; 6:862-70).

Another modification that may be included in the RNA of certain embodiments of AGT dsRNA agents, AGT antisense polynucleotides, and/or AGT sense polynucleotides of the invention, comprises chemically linking to the RNA one or more ligands, moieties or conjugates that enhance one or more characteristics of the AGT dsRNA agent, AGT antisense polynucleotide, and/or AGT sense polynucleotide, respectively. Non-limiting examples of characteristics that may be enhanced are: AGT dsRNA agent, AGT antisense polynucleotide, and/or AGT sense polynucleotide activity, cellular distribution, delivery of an AGT dsRNA agent, pharmacokinetic properties of an AGT dsRNA agent, and cellular uptake of the AGT dsRNA agent. In some embodiments of the invention, an AGT dsRNA agent comprises one or more targeting groups or linking groups, which in certain embodiments of AGT dsRNA agents of the invention are conjugated to the sense strand. A non-limiting example of a targeting group is a compound comprising N-acetyl-galactosamine (GalNAc). The terms “targeting group”, “targeting agent”, “linking agent”, “targeting compound”, and “targeting ligand” may be used interchangeably herein. In certain embodiments of the invention an AGT dsRNA agent comprises a targeting compound that is conjugated to the 5′-terminal end of the sense strand. In certain embodiments of the invention an AGT dsRNA agent comprises a targeting compound that is conjugated to the 3′-terminal end of the sense strand. In some embodiments of the invention, an AGT dsRNA agent comprises a targeting group that comprises GalNAc. In certain embodiments of the invention an AGT dsRNA agent does not include a targeting compound conjugated to one or both of the 3′-terminal end and the 5′-terminal end of the sense strand. In certain embodiments of the invention an AGT dsRNA agent does not include a GalNAc containing targeting compound conjugated to one or both of the 5′-terminal end and the 3′-terminal end of the sense strand.

Additional targeting and linking agents are well known in the art, for example, targeting and linking agents that may be used in certain embodiments of the invention include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

Certain embodiments of a composition comprising an AGT dsRNA agent, AGT antisense polynucleotide, and/or AGT sense polynucleotide may comprise a ligand that alters distribution, targeting, or etc. of the AGT dsRNA agent. In some embodiments of a composition comprising an AGT dsRNA agent of the invention, the ligand increases affinity for a selected target, e.g., molecule, cell or cell type, compartment, e.g., a cellular or organ compartment, tissue, organ or region of the body, as, e.g., compared to a species absent such a ligand. A ligand useful in a composition and/or method of the invention may be a naturally occurring substance, such as a protein (e.g., human serum albumin (HSA), low-density lipoprotein (LDL), or globulin); a carbohydrate (e.g., a dextran, pullulan, chitin, chitosan, inulin, cyclodextrin or hyaluronic acid);

or a lipid. A ligand may also be a recombinant or synthetic molecule, such as a synthetic polymer, e.g., a synthetic polyamino acid or polyamine. Examples of polyamino acids are a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

A ligand included in a composition and/or method of the invention may comprise a targeting group, non-limiting examples of which are a cell or tissue targeting agent, e.g., a lectin, glycoprotein, lipid or protein, e.g., an antibody that binds to a specified cell type such as a kidney cell or a liver cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

A ligand included in a composition and/or method of the invention may be a protein, e.g., glycoprotein, or peptide, for example a molecule with a specific affinity for a co-ligand, or an antibody, for example an antibody, that binds to a specified cell type such as a cancer cell, endothelial cell, cardiac cell, or bone cell. A ligand useful in an embodiment of a composition and/or method of the invention can be a hormone or hormone receptor. A ligand useful in an embodiment of a composition and/or method of the invention can be a lipid, lectin, carbohydrates, vitamin, cofactos, multivalent lactose, multivalent galactose, N-acetyl-galactosamine, N-acetyl-gulucosamine multivalent mannose, or multivalent fucose. A ligand useful in an embodiment of a composition and/or method of the invention can be a substance that can increase uptake of the AGT dsRNA agent into the cell, for example, by disrupting the cell's cytoskeleton, e.g., by disrupting the cell's microtubules, microfilaments, and/or intermediate filaments. Non-limiting examples of this type of agent are: taxon, vincristine, vinblastine, cytochalasin, nocodazole, japlakinolide, latrunculin A, phalloidin, swinholide A, indanocine, and myoservin.

In some embodiments, a ligand attached to an AGT dsRNA agent of the invention functions as a pharmacokinetic (PK) modulator. An example of a PK modulator that may be used in compositions and methods of the invention includes but is not limited to: a lipophiles, a bile acid, a steroid, a phospholipid analogue, a peptide, a protein binding agent, PEG, a vitamin, cholesterol, a fatty acid, cholic acid, lithocholic acid, dialkylglycerides, diacylglyceride, a phospholipid, a sphingolipid, naproxen, ibuprofen, vitamin E, biotin, an aptamer that binds a serum protein, etc. Oligonucleotides comprising a number of phosphorothioate linkages are also known to bind to serum protein, thus short oligonucleotides, e.g., oligonucleotides of about bases, 10 bases, 15 bases or 20 bases, comprising multiple of phosphorothioate linkages in the backbone may also be used in compositions and/or methods of the invention as ligands.

AGT dsRNA Agent Compositions

In some embodiments of the invention, an AGT dsRNA agent is in a composition. A composition of the invention may include one or more AGT dsRNA agent and optionally one or more of a pharmaceutically acceptable carrier, a delivery agent, a targeting agent, detectable label, etc. A non-limiting example of a targeting agent that may be useful according to some embodiments of methods of the invention is an agent that directs an AGT dsRNA agent of the invention to and/or into a cell to be treated. A targeting agent of choice will depend upon such elements as: the nature of the AGT-associated disease or condition, and on the cell type being targeted. In a non-limiting example, in some embodiments of the invention it may be desirable to target an AGT dsRNA agent to and/or into a liver cell. It will be understood that in some embodiments of methods of the invention, a therapeutic agent comprises a AGT dsRNA agent with only a delivery agent, such as a delivery agent comprising N-Acetylgalactosamine (GalNAc), without any additional attached elements. For example, in some aspects of the invention an AGT dsRNA agent may be attached to a delivery compound comprising GalNAc and included in a composition comprising a pharmaceutically acceptable carrier and administered to a cell or subject without any detectable labels, or targeting agents, etc. attached to the AGT dsRNA agent.

In cases where an AGT dsRNA agent of the invention is administered with and/or attached to one or more delivery agents, targeting agents, labeling agents, etc. a skilled artisan will be aware of and able to select and use suitable agents for use in methods of the invention. Labeling agents may be used in certain methods of the invention to determine the location of an AGT dsRNA agent in cells and tissues and may be used to determine a cell, tissue, or organ location of a treatment composition comprising an AGT dsRNA agent that has been administered in methods of the invention. Procedures for attaching and utilizing labeling agents such as enzymatic labels, dyes, radiolabels, etc. are well known in the art. It will be understood that in some embodiments of compositions and methods of the invention, a labeling agent is attached to one or both of a sense polynucleotide and an antisense polynucleotide included in an AGT dsRNA agent.

Delivery of AGT dsRNA Agents and AGT Antisense Polynucleotide Agents

Certain embodiments of methods of the invention, includes delivery of an AGT dsRNA agent into a cell. As used herein the term, “delivery” means facilitating or effecting uptake or absorption into the cell. Absorption or uptake of an AGT dsRNA agent can occur through unaided diffusive or active cellular processes, or by use of delivery agents, targeting agents, etc. that may be associated with an AGT dsRNA agent of the invention. Delivery means that are suitable for use in methods of the invention include, but are not limited to: in vivo delivery, in which an AGT dsRNA agent is in injected into a tissue site or administered systemically. In some embodiments of the invention, an AGT dsRNA agent is attached to a delivery agent.

Non-limiting examples of methods that can be used to deliver AGT dsRNA agents to cells, tissues and/or subjects include: AGT dsRNA-GalNAc conjugates, SAMiRNA technology, LNP-based delivery methods, and naked RNA delivery. These and other delivery methods have been used successfully in the art to deliver therapeutic RNAi agents for treatment of various diseases and conditions, such as but not limited to: liver diseases, acute intermittent porphyria (AIP), hemophilia, pulmonary fibrosis, etc. Details of various delivery means are found in publications such as: Nikam, R. R. & K. R. Gore (2018) Nucleic Acid Ther, 28 (4), 209-224 August 2018; Springer A. D. & S. F. Dowdy (2018) Nucleic Acid Ther. June 1; 28(3): 109-118; Lee, K. et al., (2018) Arch Pharm Res, 41(9), 867-874; and Nair, J. K. et al., (2014) J. Am. Chem. Soc. 136:16958-16961, the content each of which is incorporated by reference herein.

Some embodiments of the invention comprise use of lipid nanoparticles (LNPs) to deliver an AGT dsRNA agent of the invention to a cell, tissue, and/or subject. LNPs are routinely used for in vivo delivery of AGT dsRNA agents, including therapeutic AGT dsRNA agents. One benefit of using an LNP or other delivery agent is an increased stability of the AGT RNA agent when it is delivered to a subject using the LNP or other delivery agent. In some embodiments of the invention an LNP comprises a cationic LNP that is loaded with one or more AGT RNAi molecules of the invention. The LNP comprising the AGT RNAi molecule(s) is administered to a subject, the LNPs and their attached AGT RNAi molecules are taken up by cells via endocytosis, their presence results in release of RNAi trigger molecules, which mediate RNAi.

Another non-limiting example of a delivery agent that may be used in embodiments of the invention to delivery an AGT dsRNA agent of the invention to a cell, tissue and/or subject is an agent comprising GalNAc that is attached to an AGT dsRNA agent of the invention and delivers the AGT dsRNA agent to a cell, tissue, and/or subject. Examples of certain additional delivery agents comprising GalNAc that can be used in certain embodiments of methods and composition of the invention are disclosed in PCT Application: WO2020191183A1. A non-limiting example of a GalNAc targeting ligand that can be used in compositions and methods of the invention to deliver an AGT dsRNA agent to a cell is a targeting ligand cluster. Examples of targeting ligand clusters that are presented herein are referred to as: GalNAc Ligand with phosphodiester link (GLO) and GalNAc Ligand with phosphorothioate link (GLS). The term “GLX-n” may be used herein to indicate the attached GalNAC-containing compound is any one of compounds GLS-1, GLS-2, GLS-3, GLS-4, GLS-5, GLS-6, GLS-7, GLS-8, GLS-9, GLS-10, GLS-11, GLS-12, GLS-13, GLS-14, GLS-15, GLS-16, GLO-1, GLO-2, GLO-3, GLO-4, GLO-5, GLO-6, GLO-7, GLO-8, GLO-9, GLO-10, GLO-11, GLO-12, GLO-13, GLO-14, GLO-15, and GLO-16, the structure of each of which is shown below, with the below with location of attachment of the GalNAc-targeting ligand to an RNAi agent of the invention at far right of each. It will be understood that any RNAi and dsRNA molecule of the invention can be attached to the GLS-1, GLS-2, GLS-3, GLS-4, GLS-5, GLS-6, GLS-7, GLS-8, GLS-9, GLS-10, GLS-11, GLS-12, GLS-13, GLS-14, GLS-15, GLS-16, GLO-1, GLO-2, GLO-3, GLO-4, GLO-5, GLO-6, GLO-7, GLO-8, GLO-9, GLO-10, GLO-11, GLO-12, GLO-13, GLO-14, GLO-15, and GLO-16, GLO-1 through GLO-16 and GLS-1 through GLS-16 structures.

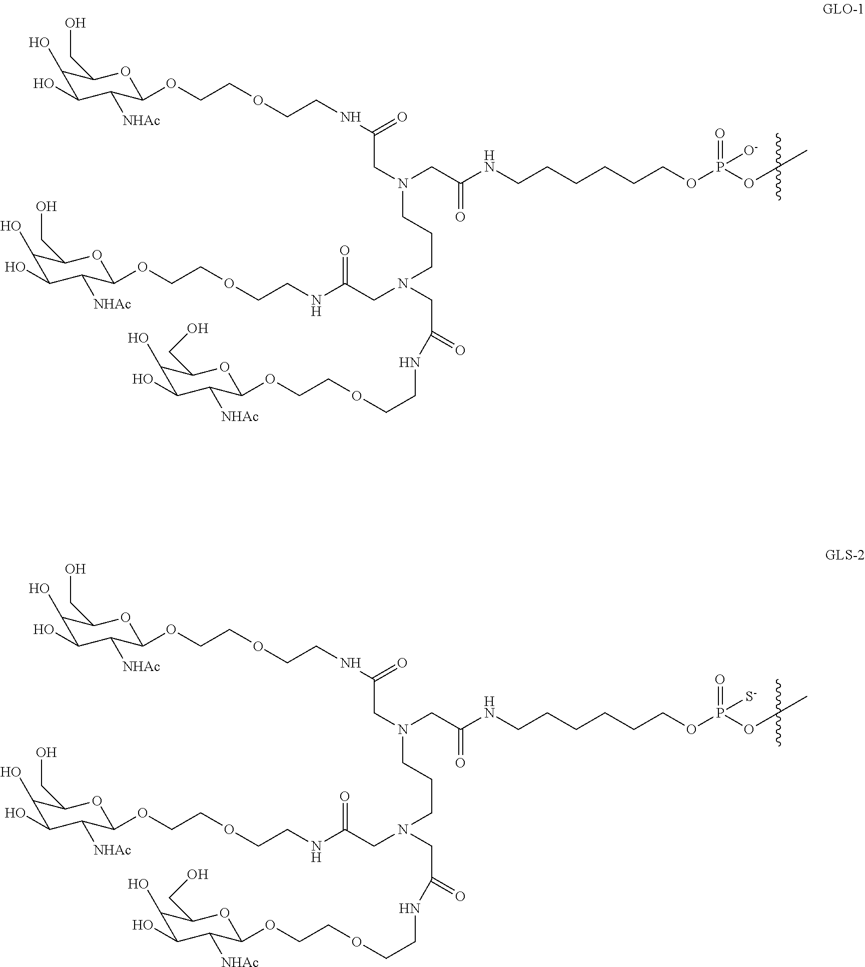 [SMILES: *OP(=O)([O-])OCCCCCCNC(=O)CN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCNC(=O)CN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

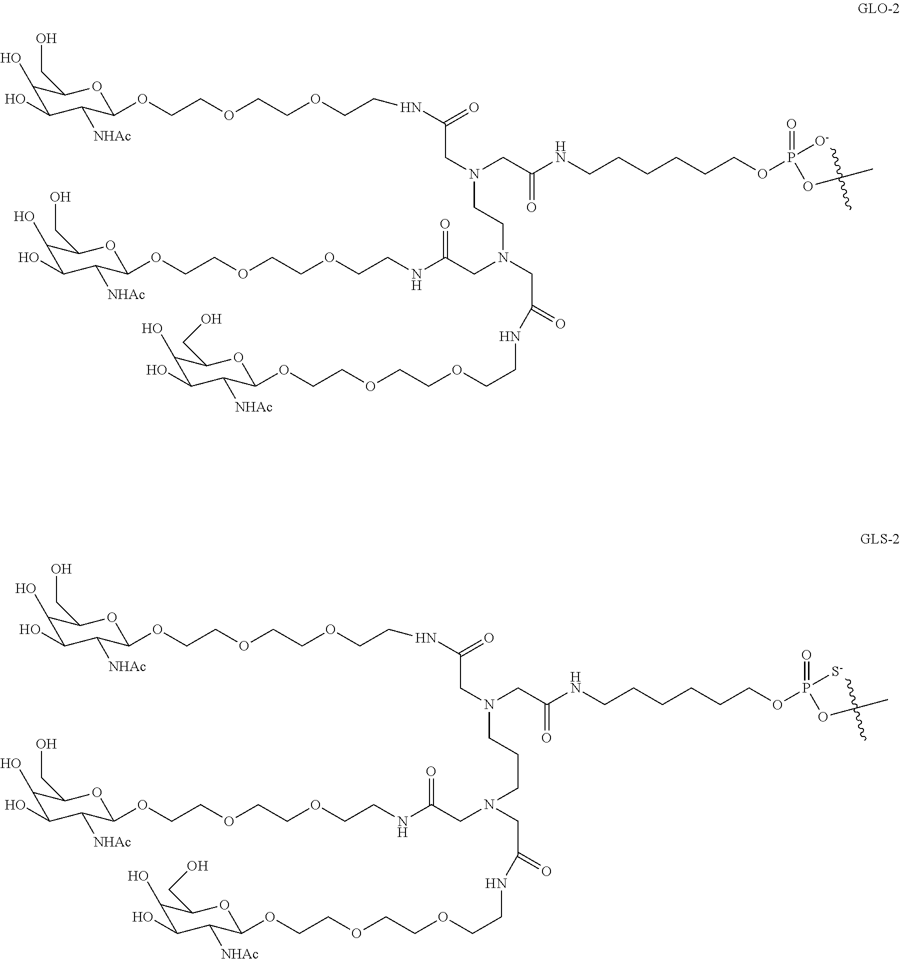 [SMILES: *OP(=O)([O-])OCCCCCCNC(=O)CN(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCCCCCNC(=O)CN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

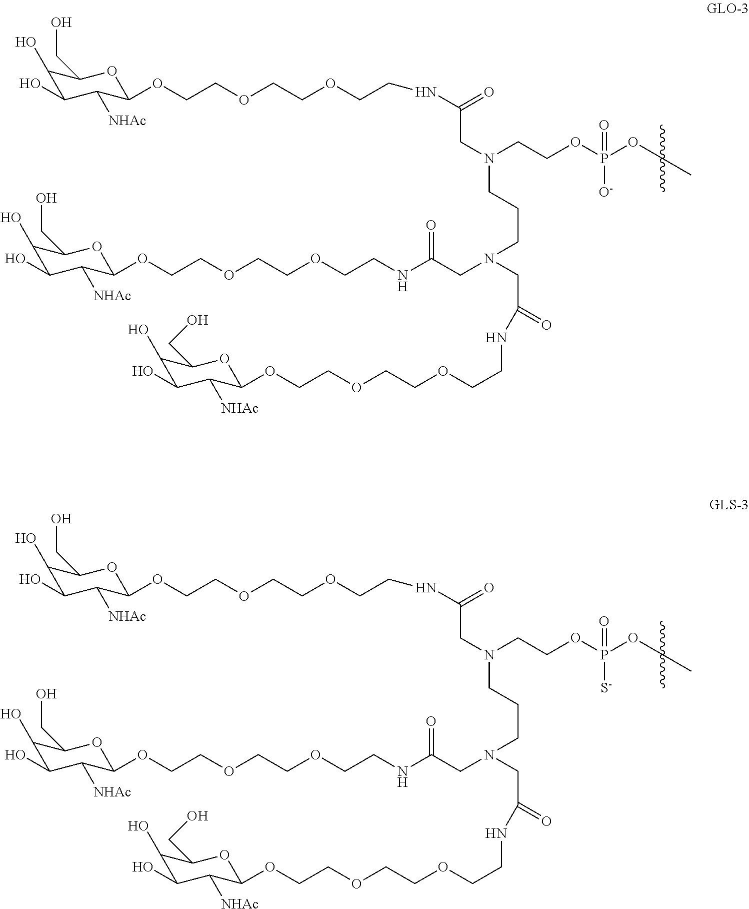 [SMILES: *OP(=O)([O-])OCCN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

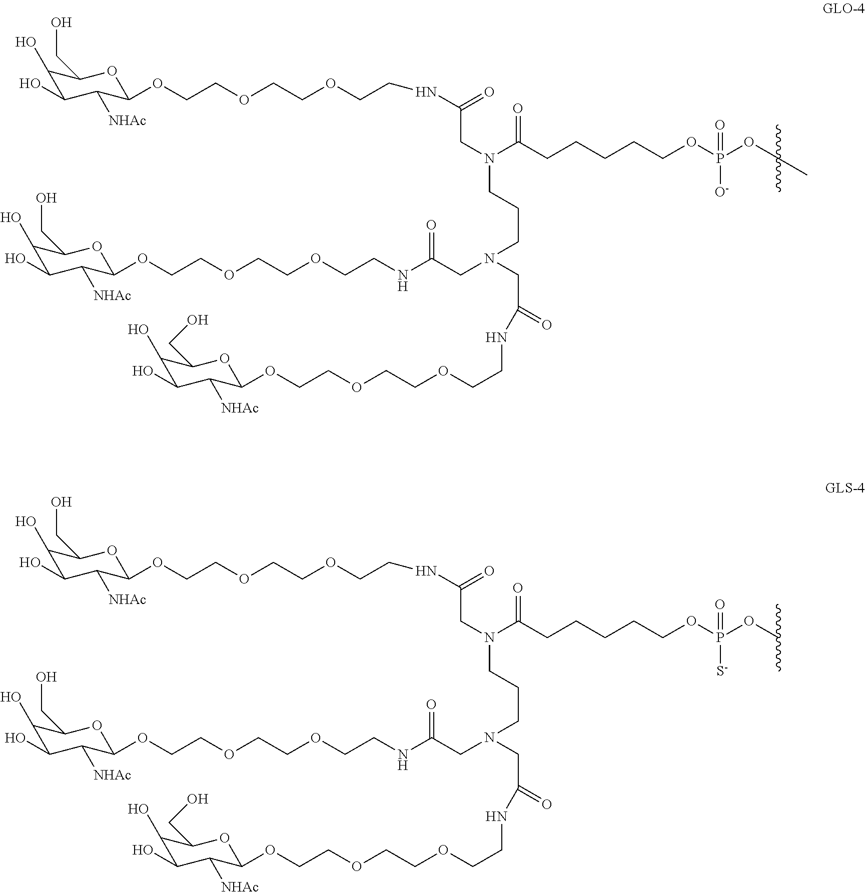 [SMILES: *OP(=O)([O-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

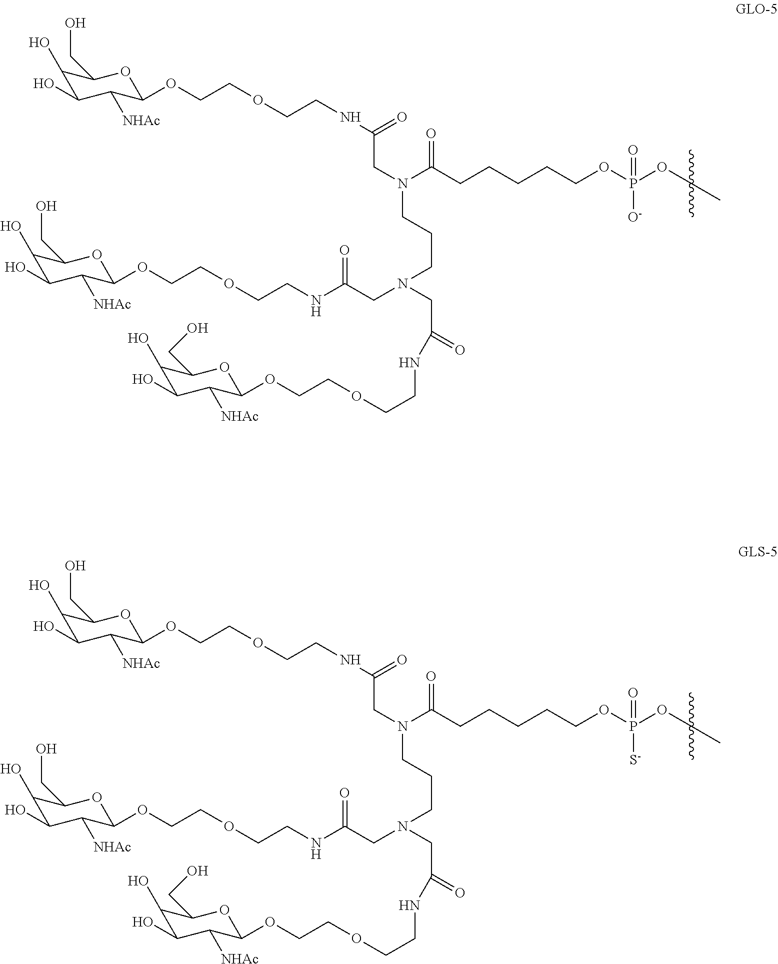 [SMILES: *OP(=O)([O-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

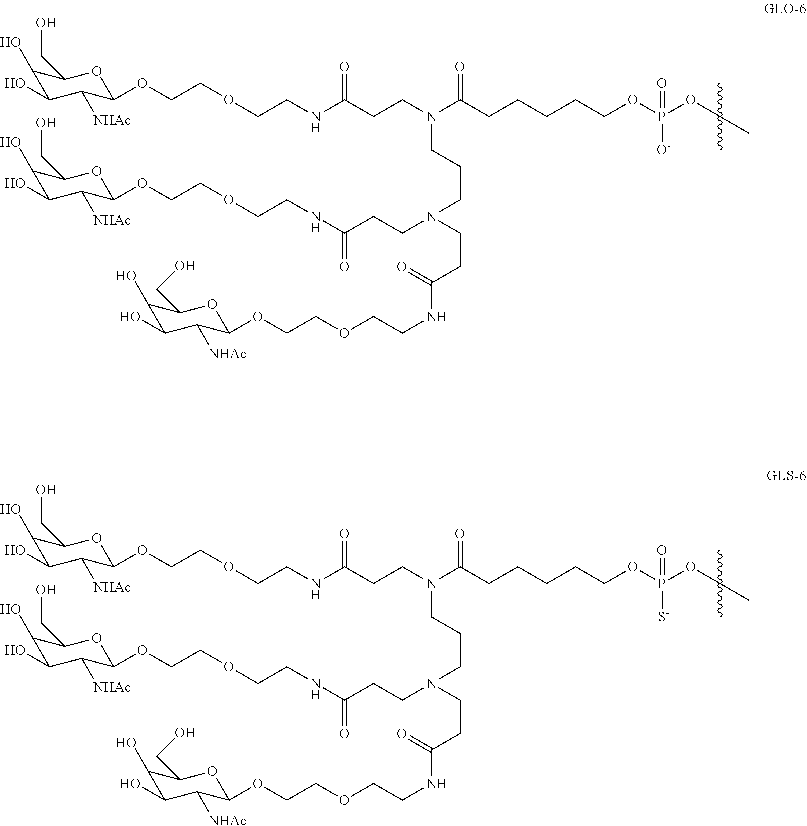 [SMILES: *OP(=O)([O-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O ; *OP(=O)([S-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O]

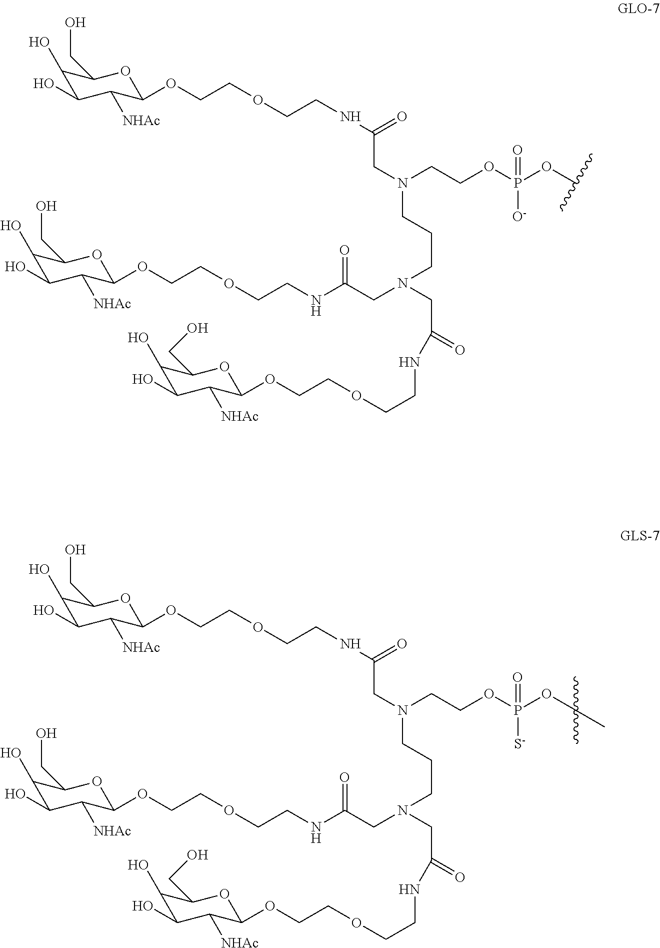 [SMILES: *OP(=O)([O-])OCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

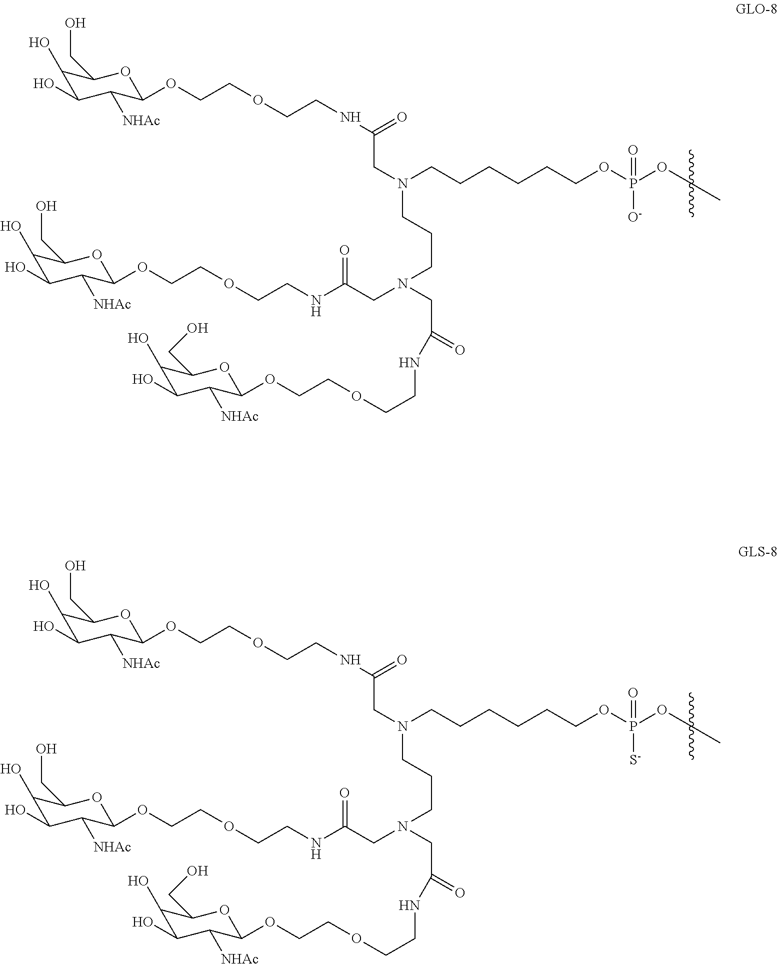 [SMILES: *OP(=O)([O-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

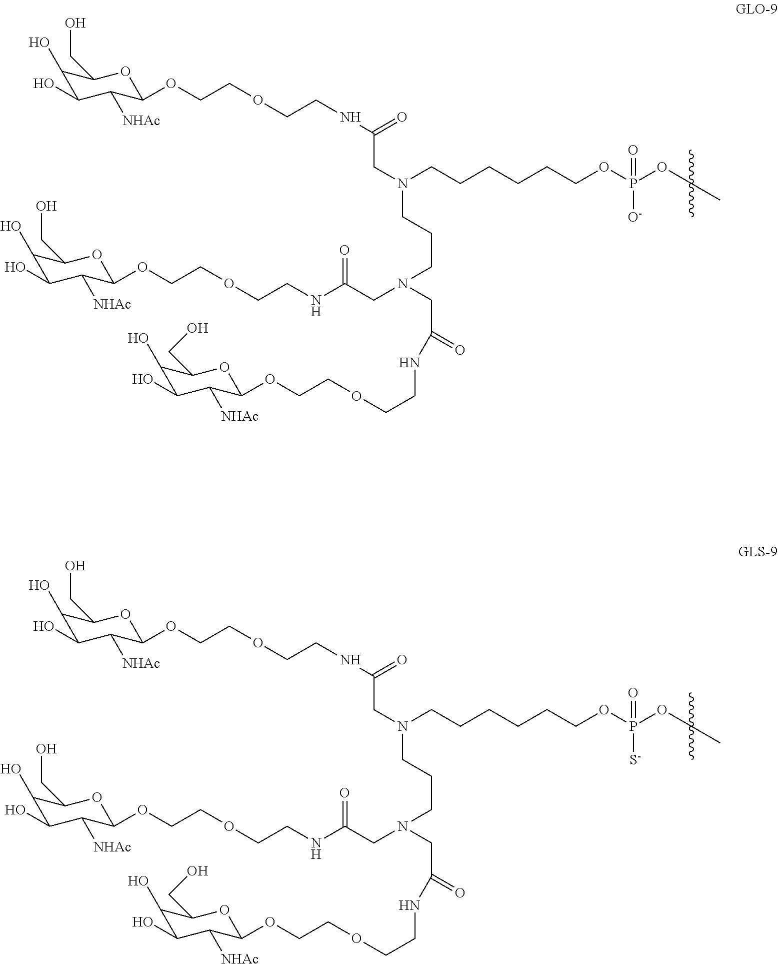 [SMILES: *OP(=O)([O-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

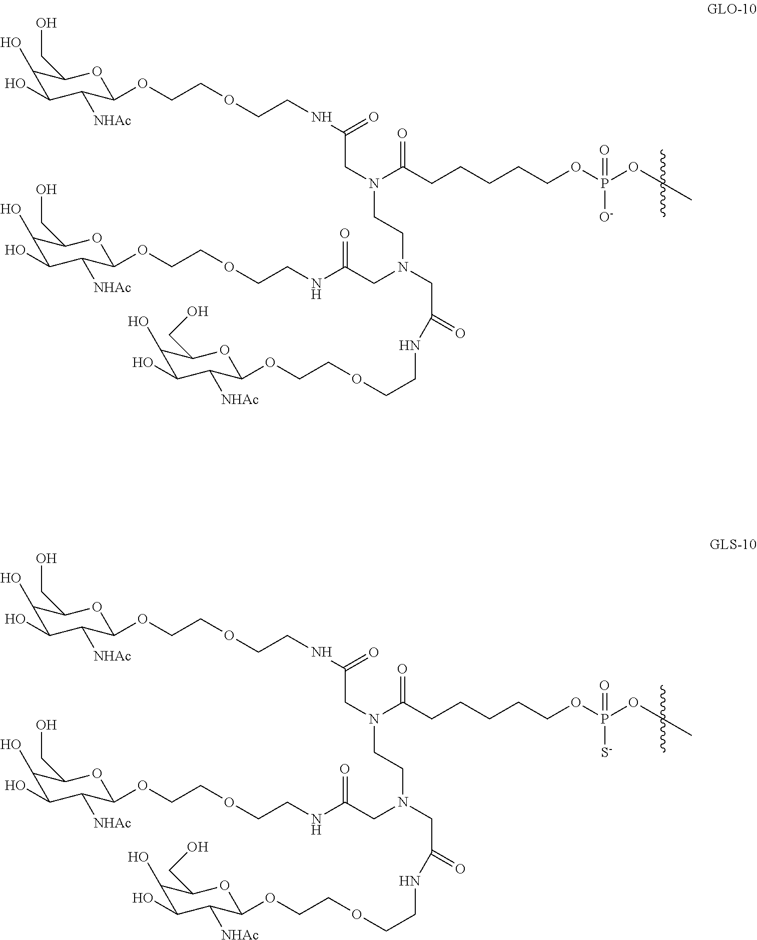 [SMILES: *OP(=O)([O-])OCCCCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

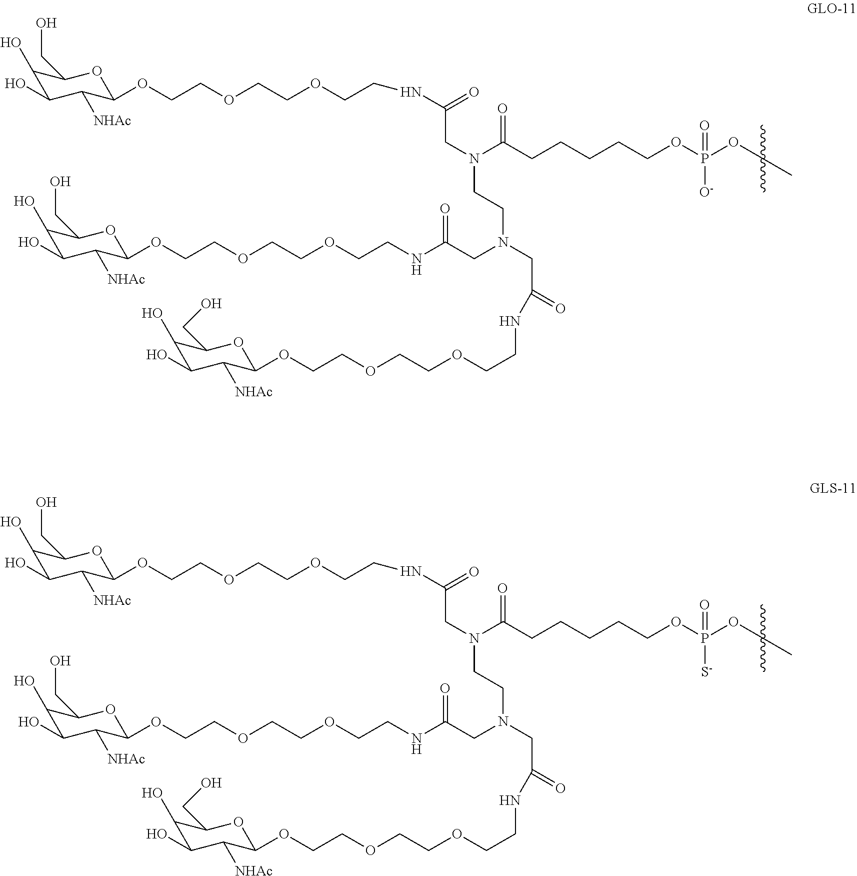 [SMILES: *OP(=O)([O-])OCCCCCC(=O)N(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCCCCC(=O)N(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

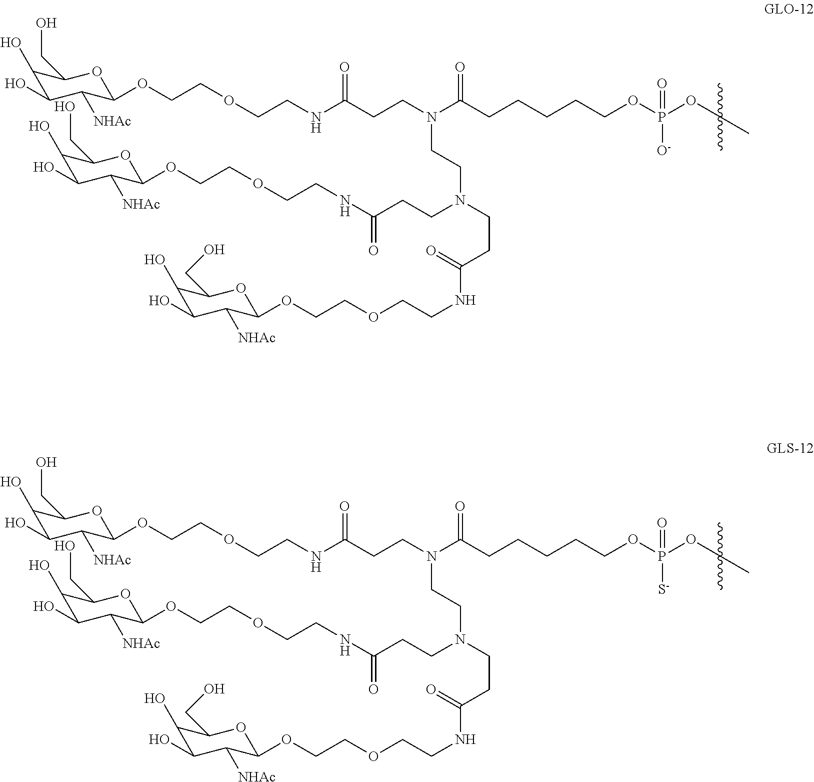 [SMILES: *OP(=O)([O-])OCCCCCC(=O)N(CCC(=O)NCCOCCOC1OC(CO)C(O)C(O)C1NC(C)=O)CCN(CCC(=O)NCCOCCOC1OC(CO)C(O)C(O)C1NC(C)=O)CCC(=O)NCCOCCOC1OC(CO)C(O)C(O)C1NC(C)=O ; *OP(=O)([S-])OCCCCCC(=O)N(CCC(=O)NCCOCCOC1OC(CO)C(O)C(O)C1NC(C)=O)CCN(CCC(=O)NCCOCCOC1OC(CO)C(O)C(O)C1NC(C)=O)CCC(=O)NCCOCCOC1OC(CO)C(O)C(O)C1NC(C)=O]

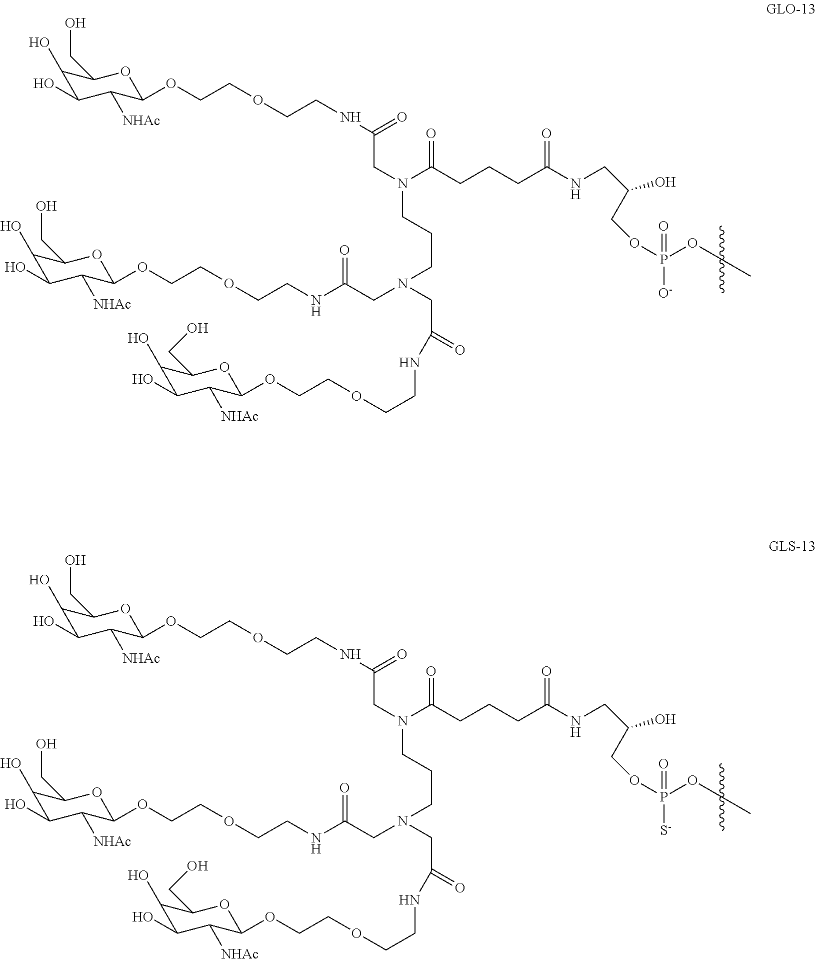 [SMILES: *OP(=O)([O-])OC[C@@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OC[C@@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

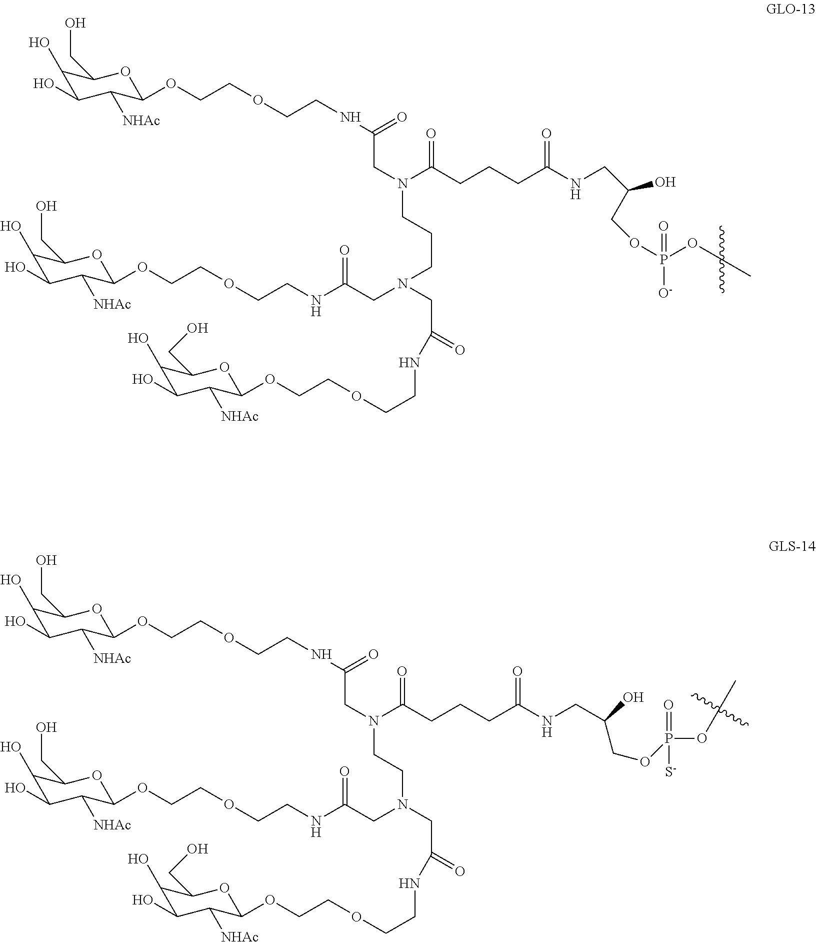 [SMILES: *OP(=O)([O-])OC[C@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OC[C@H](O)CNC(=O)CCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

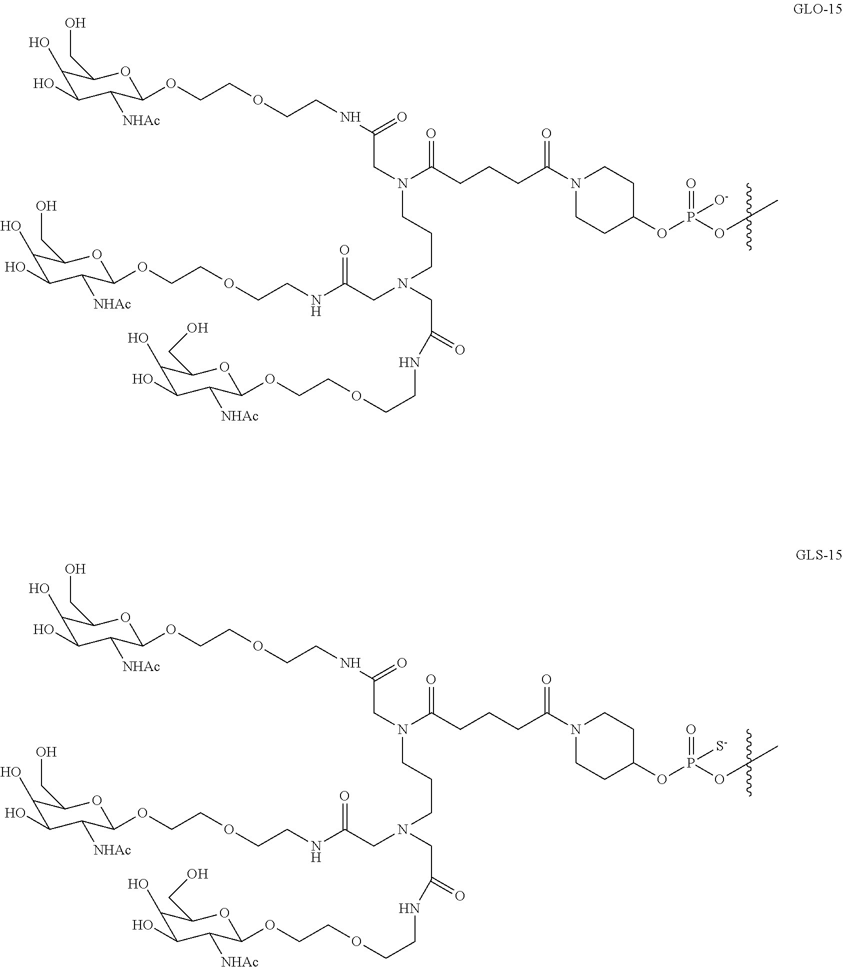 [SMILES: *OP(=O)([O-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1 ; *OP(=O)([S-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1]

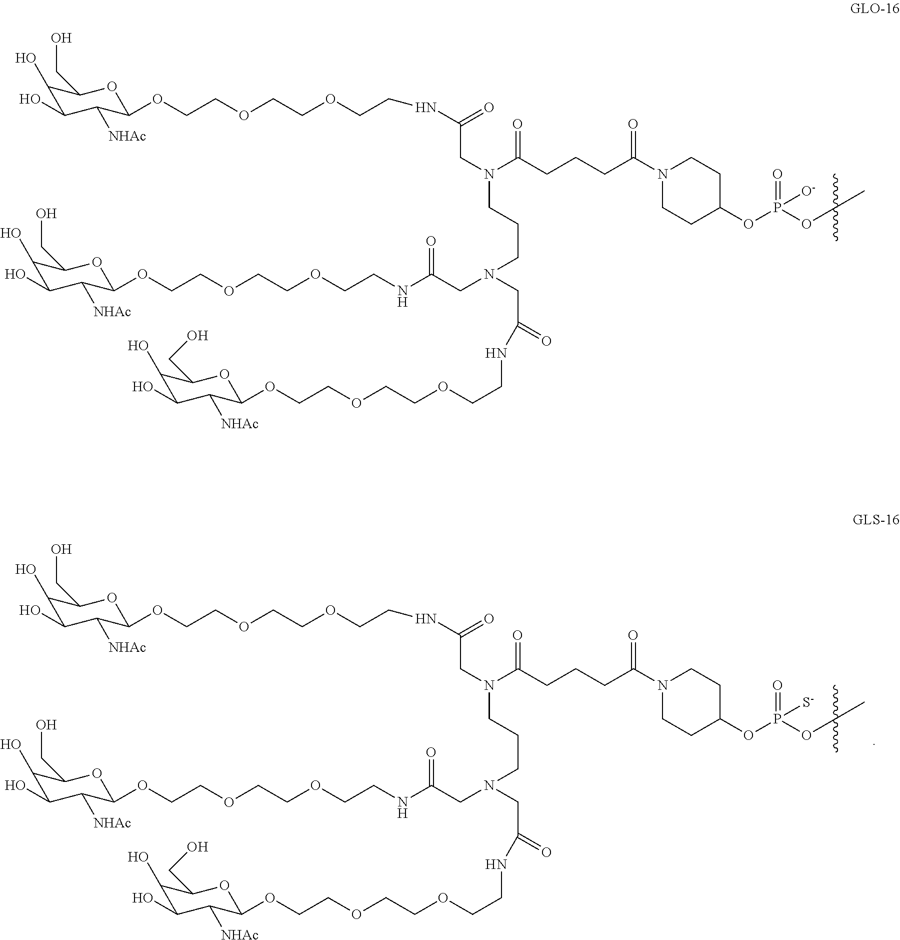 [SMILES: *OP(=O)([O-])OC1CCN(C(=O)CCCC(=O)N(CCCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOCCOC2OC(CO)C(O)C(O)C2NC(C)=O)CC1 ; *OP(=O)([S-])OC1CCN(C(=O)CCCC(=O)N(CCCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOCCOC2OC(CO)C(O)C(O)C2NC(C)=O)CC1]

In some embodiments of the invention, in vivo delivery can also be by a beta-glucan delivery system, such as those described in U.S. Pat. Nos. 5,032,401 and 5,607,677, and U.S. Publication No. 2005/0281781, which are hereby incorporated by reference in their entirety. In vitro introduction of an AGT RNAi agent into a cell may also be done using art-known methods such as electroporation and lipofection. In certain embodiments of methods of the invention, an AGT dsRNA is delivered without a targeting agent. These RNAs may be delivered as “naked” RNA molecules. As a non-limiting example, an AGT dsRNA of the invention may be administered to a subject to treat an AGT-associated disease or condition in the subject, such as a hypertensive disease, in a pharmaceutical composition comprising the RNAi agent, but not including a targeting agent such as a GalNAc targeting compound.

In addition to certain delivery means described herein, it will be understood that RNAi delivery means, such as but not limited to those described herein and those used in the art, can be used in conjunction with embodiments of AGT RNAi agents and treatment methods described herein.

AGT dsRNA agents of the invention may be administered to a subject in an amount and manner effective to reduce a level and activity of AGT polypeptide in a cell and/or subject. In some embodiments of methods of the invention one or more AGT dsRNA agents are administered to a cell and/or subject to treat a disease or condition associated with AGT expression and activity. Methods of the invention, in some embodiments, include administering one or more AGT dsRNA agents to a subject in need of such treatment to reduce a disease or condition associated with AGT expression in the subject. AGT dsRNA agents or AGT antisense polynucleotide agents of the invention can be administered to reduce AGT expression and/or activity in one more of in vitro, ex vivo, and in vivo cells.

In some embodiments of the invention, a level, and thus an activity, of AGT polypeptide in a cell is reduced by delivering (e.g. introducing) an AGT dsRNA agent or AGT antisense polynucleotide agent into a cell. Targeting agents and methods may be used to aid in delivery of an AGT dsRNA agent or AGT antisense polynucleotide agent to a specific cell type, cell subtype, organ, spatial region within a subject, and/or to a sub-cellular region within a cell. An AGT dsRNA agent can be administered in certain methods of the invention singly or in combination with one or more additional AGT dsRNA agents. In some embodiments 2, 3, 4, or more independently selected AGT dsRNA agents are administered to a subject.

In certain embodiments of the invention, an AGT dsRNA agent is administered to a subject to treat an AGT-associated disease or condition in conjunction with one or more additional therapeutic regimens for treating the AGT-associate disease or condition. Non-limiting examples of additional therapeutic regimens are: administering one or more AGT antisense polynucleotides of the invention, administering a non-AGT dsRNA therapeutic agent, and a behavioral modification. An additional therapeutic regimen may be administered at a time that is one or more of: prior to, simultaneous with, and following administration of an AGT dsRNA agent of the invention. It will be understood that simultaneous with as used herein, within five minutes of time zero, within 10 minutes of time zero, within 30 minutes of time zero, within 45 minutes of time zero, and within 60 minutes of time zero, with “time zero” the time of administration of the AGT dsRNA agent of the invention to the subject. Non-limiting examples of non-AGT dsRNA therapeutic agents are: additional therapeutic agents such as diuretics, angiotensin converting enzyme (ACE) inhibitors, angiotensin II receptor antagonists, beta-blockers, vasodilators, calcium channel blockers, aldosterone antagonists, α2-agonists, renin inhibitors, α-blockers, peripherally acting adrenergic agents, selective D1 receptor partial agonists, nonselective α-adrenergic Antagonists, synthetic, steroidal antimineralocorticoids, or combinations of any of the above, and therapeutic agents for hypertension formulated as pharmaceutical combinations. Non-limiting examples of behavior modification are: dietary regimens, counseling and exercise regimens. These and other therapeutic agents and behavioral modifications are known in the art and can be used to treat an AGT disease or condition in a subject, and can also be administered to a subject in combination with one or more AGT dsRNA agents of the invention to treat AGT disease or condition. An AGT dsRNA agent of the invention administered to a cell or a subject to treat an AGT-associated disease or condition may act in a synergistic manner with one or more other therapeutic agents or active ingredients, thereby increasing one or more therapeutic agents or the effectiveness of the active ingredient and/or increasing the effectiveness of the AGT dsRNA agent in treating an AGT-associated disease or condition.

The treatment method of the present invention comprises administration of an AGT dsRNA agent that may be used before the onset of an AGT-associated disease or condition and/or when an AGT-associated disease or condition is present, including the early, middle, late stages of the disease or condition and all times before and after any of these phases. The methods of the invention may also treat subjects who have previously been treated for an AGT-associated disease or condition with one or more other therapeutic agents and/or therapeutically active ingredients, wherein one or more other therapeutic agents and/or therapeutic active ingredients The active ingredient was unsuccessful, minimally successful, and/or no longer successful in treating the subject's AGT-associated disease or condition.

Vector-Encoded dsRNA

In certain embodiments of the invention, an AGT dsRNA agent can be delivered into a cell using a vector. AGT dsRNA agent transcription units can be included in a DNA or RNA vector. Prepare and use of such vectors encoding transgenes for delivering sequences into a cell and or subject are well known in the art. Vectors can be used in methods of the invention that result in transient expression of AGT dsRNA, for example for at least 1, 2, 3, 4, 5, 6, 7, 8, 9, 10 or more hours, 1, 2, 3, 4, 5, 6, 7, 8, 9, 10 or more weeks. The length of the transient expression can be determined using routine methods based on elements such as, but not limited to the specific vector construct selected and the target cell and/or tissue. Such transgenes can be introduced as a linear construct, a circular plasmid, or a viral vector, which can be an integrating or non-integrating vector. The transgene can also be constructed to permit it to be inherited as an extrachromosomal plasmid (Gassmann, et al., Proc. Natl. Acad. Sci. USA (1995) 92:1292).

An individual strand or strands of an AGT dsRNA agent can be transcribed from a promoter on an expression vector. Where two separate strands are to be expressed to generate, for example, a dsRNA, two separate expression vectors can be co-introduced to a cell using means such as transfection or infection. In certain embodiments, each individual strand of an AGT dsRNA agent of the invention can be transcribed by promoters that are both included on the same expression vector. In certain embodiments of the invention an AGT dsRNA agent is expressed as inverted repeat polynucleotides joined by a linker polynucleotide sequence such that the AGT dsRNA agent has a stem and loop structure.

Non-limiting examples of RNA expression vectors are DNA plasmids or viral vectors. Expression vectors useful in embodiments of the invention can be compatible with eukaryotic cells. Eukaryotic cell expression vectors are routinely used in the art and are available from a number of commercial sources. Delivery of AGT dsRNA expressing vectors can be systemic, such as by intravenous or intramuscular administration, by administration to target cells ex-planted from a subject followed by reintroduction into the subject, or by any other means that allows for introduction into a desired target cell.

Viral vector systems that may be included in an embodiment of a method of the include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Constructs for the recombinant expression of an AGT dsRNA agent may include regulatory elements, such as promoters, enhancers, etc., which may be selected to provide constitutive or regulated/inducible expression. Viral vector systems, and the use of promoters and enhancers, etc. are routine in the art and can be used in conjunction with methods and compositions described herein.

Certain embodiments of the invention include use of viral vectors for delivery of AGT dsRNA agents into cells. Numerous adenovirus-based delivery systems are routinely used in the art for deliver to, for example, lung, liver, the central nervous system, endothelial cells, and muscle. Non-limiting examples of viral vectors that may be used in methods of the invention are: AAV vectors, a pox virus such as a vaccinia virus, a Modified Virus Ankara (MVA), NYVAC, an avipox such as fowl pox or canary pox.

Certain embodiments of the invention include methods of delivering AGT dsRNA agents into cells using a vector and such vectors may be in a pharmaceutically acceptable carrier that may, but need not, include a slow release matrix in which the gene delivery vehicle is imbedded. In some embodiments, a vector for delivering an AGT dsRNA can be produced from a recombinant cell, and a pharmaceutical composition of the invention may include one or more cells that produced the AGT dsRNA delivery system.

Pharmaceutical Compositions Containing AGT dsRNA or ssRNA Agents

Certain embodiments of the invention include use of pharmaceutical compositions containing an AGT dsRNA agent or AGT antisense polynucleotide agent and a pharmaceutically acceptable carrier. The pharmaceutical composition containing the AGT dsRNA agent or AGT antisense polynucleotide agent can be used in methods of the invention to reduce AGT gene expression and AGT activity in a cell and is useful to treat an AGT-associated disease or condition. Such pharmaceutical compositions can be formulated based on the mode of delivery. Non-limiting examples of formulations for modes of delivery are: a composition formulated for subcutaneous delivery, a composition formulated for systemic administration via parenteral delivery, a composition formulated for intravenous (IV) delivery, a composition formulated for intrathecal delivery, a composition formulated for direct delivery into brain, etc. Administration of a pharmaceutic composition of the invention to deliver an AGT dsRNA agent or AGT antisense polynucleotide agent into a cell may be done using one or more means such as: topical (e.g., by a transdermal patch), pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; subdermal, e.g., via an implanted device; or intracranial, e.g., by intraparenchymal, intrathecal or intraventricular, administration. An AGT dsRNA agent or AGT antisense polynucleotide agent can also be delivered directly to a target tissue, for example directly into the liver, directly into a kidney, etc. It will be understood that “delivering an AGT dsRNA agent” or “delivering an AGT antisense polynucleotide agent” into a cell encompasses delivering an AGT dsRNA agent or AGT antisense polynucleotide agent, respectively, directly as well as expressing an AGT dsRNA agent in a cell from an encoding vector that is delivered into a cell, or by any suitable means with which the AGT dsRNA or AGT antisense polynucleotide agent becomes present in a cell. Preparation and use of formulations and means for delivering inhibitory RNAs are well known and routinely used in the art.

As used herein, a “pharmaceutical composition” comprises a pharmacologically effective amount of an AGT dsRNA agent or AGT antisense polynucleotide agent of the invention and a pharmaceutically acceptable carrier. The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract. Agents included in drug formulations are described further herein below.

As used herein terms such as: “pharmacologically effective amount,” “therapeutically effective amount” and “effective amount” refers to that amount of an AGT dsRNA agent or AGT antisense polynucleotide agent of the invention to produce the intended pharmacological, therapeutic or preventive result. For example, if a given clinical treatment is considered effective when there is at least a 10% reduction in a measurable parameter associated with a disease or disorder, a therapeutically effective amount of a drug for the treatment of that disease or disorder is the amount necessary to effect at least a 10% reduction in that parameter. For example, a therapeutically effective amount of an AGT dsRNA agent or AGT antisense polynucleotide agent can reduce AGT polypeptide levels by at least 10%. The pharmaceutical composition may comprise dsRNAi agents including duplexes such as AD00051 to AD00122-19-2, AD00163-3, AV01227 to AV01257, AV01711 shown in Table 1. In some embodiments, preferred dsRNAi agents include, for example, duplexes AD00158, AD00163, AD00159, AD00290, AD00300, or AD00122. In other embodiments, preferred dsRNAi agents include, for example, AD00158-1, AD00158-2, AD00163-1, AD00159-1, or AD00300-1. In other embodiments, such dsRNAi agents include duplex variants, eg, variants of duplex AD00158, AD00163, AD00163-3, AD00159, AD00290, AD00300, or AD00122.

### Effective Amounts

Methods of the invention, in some aspects comprise contacting a cell with an AGT dsRNA agent or AGT antisense polynucleotide agent in an effective amount to reduce AGT gene expression in the contacted cell. Certain embodiments of methods of the invention comprise administering an AGT dsRNA agent or an AGT antisense polynucleotide agent to a subject in an amount effective to reduce AGT gene expression and treat an AGT-associated disease or condition in the subject. An “effective amount” used in terms of reducing expression of AGT and/or for treating an AGT-associated disease or condition, is an amount necessary or sufficient to realize a desired biologic effect. For example, an effective amount of an AGT dsRNA agent or AGT antisense polynucleotide agent to treat an AGT-associated disease or condition could be that amount necessary to (i) slow or halt progression of the disease or condition; or (ii) reverse, reduce, or eliminate one or more symptoms of the disease or condition. In some aspects of the invention, an effective amount is that amount of an AGT dsRNA agent or AGT antisense polynucleotide agent that when administered to a subject in need of a treatment of an AGT-associated disease or condition, results in a therapeutic response that prevents and/or treats the disease or condition. According to some aspects of the invention, an effective amount is that amount of an AGT dsRNA agent or AGT antisense polynucleotide agent of the invention that when combined or co-administered with another therapeutic treatment for an AGT-associated disease or condition, results in a therapeutic response that prevents and/or treats the disease or condition. In some embodiments of the invention, a biologic effect of treating a subject with an AGT dsRNA agent or AGT antisense polynucleotide agent of the invention may be the amelioration and or absolute elimination of symptoms resulting from the AGT-associated disease or condition. In some embodiments of the invention, a biologic effect is the complete abrogation of the AGT-associated disease or condition, as evidenced for example, by a diagnostic test that indicates the subject is free of the AGT-associated disease or condition. A non-limiting example of a physiological symptom that may be detected includes a reduction in lipid accumulation in liver of a subject following administration of an agent of the invention. Additional art-known means of assessing the status of an AGT-associated disease or condition can be used to determine an effect of an agent and/or methods of the invention on an AGT-associated disease or condition.

Typically, an effective amount of an AGT dsRNA agent or AGT antisense polynucleotide agent to decrease AGT polypeptide activity to a level to treat an AGT-associated disease or condition will be determined in clinical trials, establishing an effective dose for a test population versus a control population in a blind study. In some embodiments, an effective amount will be that results in a desired response, e.g., an amount that diminishes an AGT-associated disease or condition in cells, tissues, and/or subjects with the disease or condition. Thus, an effective amount of an AGT dsRNA agent or AGT antisense polynucleotide agent to treat an AGT-associated disease or condition that can be treated by reducing AGT polypeptide activity may be the amount that when administered decreases the amount of AGT polypeptide activity in the subject to an amount that is less than the amount that would be present in the cell, tissue, and/or subject without the administration of the AGT dsRNA agent or AGT antisense polynucleotide agent. In certain aspects of the invention the level of AGT polypeptide activity, and/or AGT gene expression present in a cell, tissue, and/or subject that has not been contacted with or administered an AGT dsRNA agent or AGT antisense polynucleotide agent of the invention is referred to as a “control” amount. In some embodiments of methods of the invention a control amount for a subject is a pre-treatment amount for the subject, in other words, a level in a subject before administration of an AGT agent can be a control level for that subject and compared to a level of AGT polypeptide activity and/or AGT gene expression in the subject following siRNA administered to the subject. In the case of treating an AGT-associated disease or condition the desired response may be reducing or eliminating one or more symptoms of the disease or condition in the cell, tissue, and/or subject. The reduction or elimination may be temporary or may be permanent. It will be understood that the status of an AGT-associated disease or condition can be monitored using methods of determining AGT polypeptide activity, AGT gene expression, symptom evaluation, clinical testing, etc. In some aspects of the invention, a desired response to treatment of an AGT-associated disease or condition is delaying the onset or even preventing the onset of the disease or condition.

An effective amount of a compound that reduces the activity of an AGT polypeptide can also be determined by assessing the physiological effects of administration of an AGT dsRNA agent or AGT antisense polynucleotide agent on a cell or subject, such as reduction in AGT-associated disease or condition following administration. Assays and/or symptom monitoring in subjects can be used to determine the efficacy of the AGT dsRNA agents or AGT antisense polynucleotide agents of the invention, which can be administered in the pharmaceutical compounds of the invention, and to determine response to treatment. A non-limiting example is one or more blood pressure tests known in the art. As another non-limiting example, one or more blood pressure tests known in the art can be used to determine the status of an AGT-associated disorder in a subject before and after treatment of the subject with an AGT dsRNA agent of the invention. In another non-limiting example, the status of an AGT-associated disease in a subject is determined using one or more tests known in the art to lower blood pressure levels. In this example, the disease includes hypertension, and the test is used to determine the level of reduced blood pressure in a subject before and after treatment of the subject with an AGT dsRNA agent of the invention.

Some embodiments of the invention include methods of determining the efficacy of a dsRNA agent or AGT antisense polynucleotide agent of the invention administered to a subject to treat an AGT-associated disease or condition by assessing and/or monitoring one or more “physiological characteristics” of the AGT-associated disease or condition in the subject. Non-limiting examples of physiological characteristics of an AGT-associated disease or condition are serum AGT levels, mean blood pressure, diastolic blood pressure in a subject. Standard methods for determining such physiological characteristics are known in the art and include, but are not limited to, blood tests, imaging studies, physical examination, and so on.

It will be understood that the amount of an AGT dsRNA agent or AGT antisense polynucleotide agent administered to a subject can be modified based, at least in part, on such determinations of disease and/or condition status and/or physiological characteristics determined for a subject. The amount of treatment may be varied for example by increasing or decreasing the amount of an AGT-dsRNA agent or AGT antisense polynucleotide agent, by changing the composition in which the AGT dsRNA agent or AGT antisense polynucleotide agent, respectively, is administered, by changing the route of administration, by changing the dosage timing and so on. The effective amount of an AGT dsRNA agent or AGT antisense polynucleotide agent will vary with the particular condition being treated, the age and physical condition of the subject being treated; the severity of the condition, the duration of the treatment, the nature of the concurrent therapy (if any), the specific route of administration, and additional factors within the knowledge and expertise of the health practitioner. For example, an effective amount may depend upon the desired level of AGT polypeptide activity and or AGT gene expression that is effective to treat the AGT-associated disease or condition. A skilled artisan can empirically determine an effective amount of a particular AGT dsRNA agent or AGT antisense polynucleotide agent of the invention for use in methods of the invention without necessitating undue experimentation. Combined with the teachings provided herein, by selecting from among various AGT dsRNA agents or AGT antisense polynucleotide agents of the invention, and weighing factors such as potency, relative bioavailability, patient body weight, severity of adverse side-effects and preferred mode of administration, an effective prophylactic or therapeutic treatment regimen can be planned that is effective to treat the particular subject. As used in embodiments of the invention, an effective amount of an AGT dsRNA agent or AGT antisense polynucleotide agent of the invention can be that amount that when contacted with a cell results in a desired biological effect in the cell.

It will be recognized that AGT gene silencing may be determined in any cell expressing AGT, either constitutively or by genomic engineering, and by any appropriate assay. In some embodiments of the invention, AGT gene expression is reduced by at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% by administration of an AGT dsRNA agent of the invention. In some embodiments of the invention, AGT gene expression is reduced by at between 5% and 10%, 5% and 25%, 10% and 50%, 10% and 75%, 25% and 75%, 25% and 100%, or 50% and 100% by administration of an AGT dsRNA agent of the invention.

### Dosing

AGT dsRNA agents and AGT antisense polynucleotide agents are delivered in pharmaceutical compositions in dosages sufficient to inhibit expression of AGT genes. In certain embodiments of the invention, a dose of AGT dsRNA agent or AGT antisense polynucleotide agent is in a range of 0.01 to 200.0 milligrams per kilogram body weight of the recipient per day, generally in the range of 1 to 50 mg per kilogram body weight, 5 to 40 mg/kg body weight, 10 to 30 mg/kg body weight, 1 to 20 mg/kg body weight, 1 to 10 mg/kg body weight, 4 to 15 mg/kg body weight per day, inclusive. For example, the AGT dsRNA agent or AGT antisense polynucleotide agent can be administered in an amount that is from about 0.01 mg/kg, 0.05 mg/kg, 0.1 mg/kg, 0.2 mg/kg, 0.3 mg/kg, 0.4 mg/kg, 0.5 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, through 50 mg/kg body per single dose.

Various factors may be considered in the determination of dosage and timing of delivery of an AGT dsRNA agent of the invention. The absolute amount of an AGT dsRNA agent or AGT antisense polynucleotide agent delivered will depend upon a variety of factors including a concurrent treatment, the number of doses and the individual subject parameters including age, physical condition, size and weight. These are factors well known to those of ordinary skill in the art and can be addressed with no more than routine experimentation. In some embodiments, a maximum dose can be used, that is, the highest safe dose according to sound medical judgment.

Methods of the invention may in some embodiments include administering to a subject 1, 2, 3, 4, 5, 6, 7, 8, 9, 10, or more doses of an AGT dsRNA agent or AGT anti sense polynucleotide agent. In some instances, a pharmaceutical compound, (e.g., comprising an AGT dsRNA agent or comprising an AGT antisense polynucleotide agent) can be administered to a subject at least daily, every other day, weekly, every other week, monthly, etc. Doses may be administered once per day or more than once per day, for example, 2, 3, 4, 5, or more times in one 24 hour period. A pharmaceutical composition of the invention may be administered once daily, or the AGT dsRNA agent or AGT antisense polynucleotide agent may be administered as two, three, or more sub-doses at appropriate intervals throughout the day or even using continuous infusion or delivery through a controlled release formulation. In some embodiments of methods of the invention, a pharmaceutical composition of the invention is administered to a subject one or more times per day, one or more times per week, one or more times per month, or one or more times per year.

Methods of the invention, in some aspects, include administration of a pharmaceutical compound alone, in combination with one or more other AGT dsRNA agents or AGT antisense polynucleotide agents, and/or in combination with other drug therapies or treatment activities or regimens that are administered to subjects with an AGT-associated disease or condition. Pharmaceutical compounds may be administered in pharmaceutical compositions. Pharmaceutical compositions used in methods of the invention may be sterile and contain an amount of an AGT dsRNA agent or AGT antisense polynucleotide agent that will reduce activity of an AGT polypeptide to a level sufficient to produce the desired response in a unit of weight or volume suitable for administration to a subject. A dose administered to a subject of a pharmaceutical composition that includes an AGT dsRNA agent or AGT antisense polynucleotide agent to reduce AGT protein activity can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

### Treatment

As used herein, the term “prevent” or “preventing”, when used in reference to a disease, disorder or condition that would benefit from reduced expression of the AGT gene, When used to refer to a disease, disorder or condition thereof that will benefit from a decrease in the expression of the AGT gene, it means that the subject is less likely to develop symptoms associated with such disease, disorder or condition, and the symptoms associated with such disease, disorder or condition is caused by or associated to the activation of the renin-angiotensin-aldosterone system (RAAS), such as hypertension. The likelihood of developing high blood pressure is reduced in situations where, for example, an individual has one or more risk factors for high blood pressure but does not develop high blood pressure or develops high blood pressure that is less severe Failure to develop an associated disease, disorder, or condition, or a reduction in the development of symptoms associated with such a disease, disorder, or condition (e.g., clinically Prophylaxis is considered to be effective by reducing at least about 10% on a scale for having the disease or condition), or by delaying the manifestation of symptoms (for example, by days, weeks, months, or years).

Based on the average of correctly taken sitting blood pressure readings during two or more visits, a normotensive subject has a systolic blood pressure about 90-119 mmHg (about 12-15.9 kPa (kN/m²)) and a diastolic blood pressure about 60-79 mmHg (about 8.0-10.5 kPa (kN/m²)); systolic blood pressure about 120-139 mmHg (about 16.1-18.5 kPa (kN/m²)) in subjects with prehypertension, diastolic systolic blood pressure about 60-79 mmHg (about 8.0-10.5 kPa (kN/m²)); subjects with high blood pressure (eg, stage I hypertension) about 140-159 mmHg (about 18.7-21.2 kPa (kN/m²)), a diastolic blood pressure about 90-99 mmHg (about 12.0-13.2 kPa (kN/m²)); a systolic blood pressure about ≥160 mmHg (approximately ≥21.3 kPa (kN/m²)), diastolic blood pressure is about ≥100 mmHg (approximately ≥13.3 kPa (kN/m²)).

In certain embodiments, the angiotensinogen-related disease is essential hypertension. “Essential hypertension” is the result of environmental or genetic factors (eg, the result of no apparent underlying medical cause).

In certain embodiments, the angiotensinogen-related disorder is secondary hypertension. “Secondary hypertension” has an identifiable underlying condition that can have a variety of etiologies, including renal, vascular, and endocrine causes, for example, renal parenchymal disease (e.g., polycystic kidney, glomerular, or interstitial disease), Renal vascular disease (eg, renal artery stenosis, fibromuscular dysplasia), endocrine disorders (eg, adrenocorticoid or mineralocorticoid excess, pheochromocytoma, hyperthyroidism or hypothyroidism, growth hormone excess, parathyroid hyperthyroidism), coarctation of the aorta, or use of oral contraceptives.

In certain embodiments, the angiotensinogen-related disease is a hypertensive emergency, such as malignant hypertension and accelerated hypertension. “Accelerated hypertension” refers to a severe increase in blood pressure (ie equal to or greater than 180 mmHg systolic or 110 mmHg diastolic) with immediate damage to one or more end organs. Blood pressure must be lowered immediately to prevent further organ damage. “Malignant hypertension” refers to severe elevation of blood pressure (ie equal to or greater than 180 mmHg systolic or 110 mmHg diastolic) associated with direct injury to one or more end organs and papilledema. Blood pressure must be lowered immediately to prevent further organ damage. Nerve end-organ damage due to uncontrolled blood pressure can include hypertensive encephalopathy, cerebrovascular accident/infarction; subarachnoid hemorrhage and/or intracranial hemorrhage. Cardiovascular end-organ injury may include myocardial ischemia/infarction, acute left ventricular dysfunction, acute pulmonary edema, and/or aortic dissection. Other organ systems may also be affected by uncontrolled hypertension, which can lead to acute renal failure/insufficiency, retinopathy, eclampsia, or microangiopathic hemolytic anemia.

In certain embodiments, the angiotensinogen-related disorder is acute hypertension. “Acute hypertension” means a severe increase in blood pressure (ie equal to or greater than 180 mmHg systolic or 110 mmHg diastolic) without direct damage to one or more organs. Blood pressure can be safely lowered within hours.

In certain embodiments, the angiotensinogen-associated disorder is pregnancy-associated hypertension, e.g., chronic hypertension in pregnancy, gestational hypertension, preeclampsia, eclampsia, preeclampsia superimposed on chronic hypertension, HELLP syndrome, and gestational hypertension (also called gestational transient hypertension, chronic hypertension found in the second half of pregnancy, and pregnancy-induced hypertension (PIH)). A subject with “chronic hypertension of pregnancy” is a subject whose blood pressure exceeds 140/90 mmHg before pregnancy or before 20 weeks of pregnancy. “Gestational hypertension” or “pregnancy-induced hypertension” refers to hypertension with onset late in pregnancy (>20 weeks' gestation), without any other features of preeclampsia, and postpartum normalization of blood pressure. “Mild preeclampsia” was defined as two episodes of hypertension (blood pressure ≥140/90 mmHg) separated by at least six hours in a normotensive woman before 20 weeks of gestation, but without end-organ damage evidence of. In subjects with prior essential hypertension, a diagnosis of preeclampsia was made if systolic blood pressure increased by 30 mm Hg or diastolic blood pressure increased by 15 mm Hg. “Severe preeclampsia” is defined as the presence of one of the following symptoms or signs in preeclampsia: two episodes of systolic blood pressure of 160 mm Hg or higher or diastolic blood pressure of 110 mm Hg or higher at least 6 hours apart; proteinuria greater than 5 g in 24 hours or greater than 3+ in two random urine samples collected at least 4 hours apart, pulmonary edema or cyanosis, oliguria (<400 mL in 24 hours), persistent headache, epigastric pain, and and/or impaired liver function, thrombocytopenia, oligohydramnios, slowed fetal growth, or placental abruption. “Eclampsia” was defined as seizures not attributable to other causes in women with preeclampsia. “HELLP syndrome” (also known as edema-proteinuria-hypertensive pregnancy toxicity type B) refers to hemolysis, elevated levels of liver enzymes, and decreased levels of platelets in a pregnant subject.

In certain embodiments, the angiotensinogen-related disease is Refractory hypertension. “Refractory hypertension” refers to blood pressure above target (eg, 140/90 mmHg) despite concurrent use of three different classes of antihypertensive drugs, one of which is a thiazide diuretic. Subjects whose blood pressure was controlled with four or more medications were also considered to have Refractory hypertension.

For AGT-associated diseases and conditions, where reduction of the level and/or activity of AGT polypeptide is effective in treating the disease or condition, the methods of the invention and AGT dsRNA agents can be used for treatment to inhibit AGT expression. Examples of diseases and conditions that can be treated with the AGT dsRNA agents or AGT antisense polynucleotide agents of the invention and the methods of treatment of the invention include, but are not limited to: hypertension diseases, hypertension, borderline hypertension, essential hypertension Blood pressure, secondary hypertension, isolated systolic or diastolic hypertension, pregnancy-associated hypertension, diabetic hypertension, Refractory hypertension, refractory hypertension, paroxysmal hypertension, renovascular hypertension, Goldblatt hypertension, ocular hypertension, glaucoma, pulmonary hypertension, portal hypertension, systemic venous hypertension, systolic hypertension, unstable hypertension; hypertensive heart disease, hypertensive nephropathy, atherosclerosis, arteriosclerosis, vascular disease, diabetic nephropathy, diabetic retinopathy, chronic heart failure, cardiomyopathy, diabetic cardiomyopathy, glomerulosclerosis, aortic stenosis, Aortic aneurysm, ventricular fibrosis, heart failure, myocardial infarction, angina, stroke, renal disease, renal failure, systemic sclerosis, intrauterine growth retardation (IUGR), fetal growth restriction. Such diseases and conditions may be referred to herein as “AGT-associated diseases and conditions” and “Diseases and conditions caused and/or modulated by AGT”.

In some aspects of the invention, an AGT dsRNA agent or an AGT antisense polynucleotide agent of the invention can be administered to a subject at one or more times before or after diagnosis of an AGT-associated disease or condition. In some aspects of the invention, the subject is at risk of having or developing an AGT-associated disease or condition. A subject at risk of developing an AGT-associated disease or condition is one who has an increased likelihood of developing an AGT-associated disease or condition compared to a control risk of developing an AGT-associated disease or condition. In some embodiments of the invention, the level of risk is statistically significant compared to a control level of risk. A subject at risk can include, for example: a subject who is or will be with a pre-existing disease and/or genetic abnormality that makes the subject more susceptible to an AGT-associated disease or condition than a control subject without a pre-existing disease or genetic abnormality; Subjects with a family and/or personal history of an AGT-associated disease or condition; and subjects who have previously been treated for an AGT-associated disease or condition. It should be understood that pre-existing diseases and/or genetic abnormalities that make a subject more susceptible to an AGT-associated disease or condition can be diseases or genetic abnormalities that, when present, have previously been identified as being associated with the development of an AGT-associated disease or condition. A higher likelihood has a correlation.

It should be understood that an AGT dsRNA agent or an AGT antisense polynucleotide agent may be administered to a subject based on the medical condition of the individual subject. For example, a healthcare provider to a subject can evaluate the level of AGT measured in a sample obtained from the subject and determine that it is desirable to reduce the level of AGT in the subject by administering an AGT dsRNA agent or an AGT antisense polynucleotide agent of the invention. In one non-limiting example, a biological sample, such as a blood or serum sample, can be obtained from a subject and the subject's AGT level determined in the sample. administering the AGT dsRNA agent or the AGT antisense polynucleotide agent to the subject, and obtaining a blood or serum sample from the subject after administration, and using the sample to determine AGT levels and comparing the results to the subject's pre-dose (previous) sample Determined results for comparison. A subsequent decrease in the subject's AGT level in the sample compared to the pre-dose level indicates the efficacy of the administered AGT dsRNA agent or AGT antisense polynucleotide agent in reducing the subject's AGT level. In one non-limiting example, blood pressure can be considered a physiological feature of an AGT-associated disorder, even if the subject has not been diagnosed with an AGT-associated disorder, such as the diseases disclosed herein. A healthcare provider can monitor changes in a subject's blood pressure as a measure of the efficacy of an administered AGT dsRNA agent or AGT antisense polynucleotide agent of the invention.

Certain embodiments of the methods of the invention include adjusting a treatment comprising administering the present invention to a subject based at least in part on an assessment of a change in one or more physiological characteristics of an AGT-associated disease or condition in the subject as a result of the treatment. Invented dsRNA agent or AGT antisense polynucleotide agent. For example, in some embodiments of the invention, the effect of a dsRNA agent of the invention or an AGT antisense polynucleotide agent of the invention administered to a subject can be determined and used to help regulate subsequent administration of a dsRNA agent of the invention or an AGT antisense polynucleotide agent of the invention. The amount of the sense polynucleotide agent. In one non-limiting example, a subject is administered a dsRNA agent or an AGT antisense polynucleotide agent of the invention, and following administration, the subject's blood pressure is measured; and based at least in part on the determined levels, it is determined whether a higher amount of dsRNA is required The agent or the AGT antisense polynucleotide agent to enhance the physiological effect of the administered agent, such as lowering or further lowering a subject's blood pressure. In another non-limiting example, a dsRNA agent or an AGT antisense polynucleotide agent of the invention is administered to a subject, and the subject's blood pressure is determined following administration, and based at least in part on the determined levels, the desired effect on the subject is Lower amounts of dsRNA agents or AGT antisense polynucleotide agents are administered.

Accordingly, some embodiments of the invention include assessing changes in one or more physiological characteristics resulting from previous treatment of a subject to adjust the amount of a dsRNA agent or AGT antisense polynucleotide agent of the invention subsequently administered to the subject. Some embodiments of the methods of the invention comprise 1, 2, 3, 4, 5, 6 or more determinations of physiological characteristics of an AGT-associated disease or condition; assessing and/or monitoring administration of an AGT dsRNA agent or AGT of the invention the efficacy of the antisense polynucleotide agent; and optionally using the determined results to adjust one or more of the following: the dsRNA agent or AGT antisense polynucleotide agent of the invention treats the efficacy of an AGT-associated disease or condition in a subject Dosage, dosing regimen, and/or frequency of dosing. In some embodiments of the methods of the invention, the desired result of administering to a subject an effective amount of a dsRNA agent or an AGT antisense polynucleotide agent of the invention is: a decrease in the subject's blood pressure compared to a previous blood pressure determined for the subject; blood pressure is within the normal blood pressure range.

As used herein, the terms “treat”, “treated”, or “treating” when used with respect to an AGT-associated disease or condition may refer to a prophylactic treatment that decreases the likelihood of a subject developing the AGT-associated disease or condition, and also may refer to a treatment after the subject has developed an AGT-associated disease or condition in order to eliminate or reduce the level of the AGT-associated disease or condition, prevent the AGT-associated disease or condition from becoming more advanced (e.g., more severe), and/or slow the progression of the AGT-associated disease or condition in a subject compared to the subject in the absence of the therapy to reduce activity in the subject of AGT polypeptide.

Certain embodiments of agents, compositions, and methods of the invention can be used to inhibit AGT gene expression. As used herein in reference to expression of an AGT gene, the terms “inhibit,” “silence,” “reduce,” “down-regulate,” and “knockdown” mean the expression of the AGT gene, as measured by one or more of: a level of RNA transcribed from the gene, a level of activity of AGT expressed, and a level of AGT polypeptide, protein or protein subunit translated from the mRNA in a cell, group of cells, tissue, organ, or subject in which the AGT gene is transcribed, is reduced when the cell, group of cells, tissue, organ, or subject is contacted with (e.g., treated with) an AGT dsRNA agent of the invention, compared to a control level of RNA transcribed from the AGT gene, a level of activity of expressed AGT, or a level of AGT translated from the mRNA, respectively. In some embodiments, a control level is a level in a cell, tissue, organ or subject that has not been contacted with (e.g. treated with) the AGT dsRNA agent or AGT antisense polynucleotide agent.

### Administration Methods

A variety of administration routes for an AGT dsRNA agent or AGT antisense polynucleotide agent are available for use in methods of the invention. The particular delivery mode selected will depend at least in part, upon the particular condition being treated and the dosage required for therapeutic efficacy. Methods of this invention, generally speaking, may be practiced using any mode of administration that is medically acceptable, meaning any mode that produces effective levels of treatment of an AGT-associated disease or condition without causing clinically unacceptable adverse effects. In some embodiments of the invention, an AGT dsRNA agent or AGT antisense polynucleotide agent may be administered via an oral, enteral, mucosal, subcutaneous, and/or parenteral route. The term “parenteral” includes subcutaneous, intravenous, intrathecal, intramuscular, intraperitoneal, and intrasternal injection, or infusion techniques. Other routes include but are not limited to nasal (e.g., via a gastro-nasal tube), dermal, vaginal, rectal, sublingual, and inhalation. Delivery routes of the invention may include intrathecal, intraventricular, or intracranial. In some embodiments of the invention, an AGT dsRNA agent or AGT antisense polynucleotide agent may be placed within a slow release matrix and administered by placement of the matrix in the subject. In some aspects of the invention, an AGT dsRNA agent or AGT antisense polynucleotide agent may be delivered to a subject cell using nanoparticles coated with a delivery agent that targets a specific cell or organelle. Various delivery means, methods, agents are known in the art. Non-limiting examples of delivery methods and delivery agents are additionally provided elsewhere herein. In some aspects of the invention, the term “delivering” in reference to an AGT dsRNA agent or AGT antisense polynucleotide agent may mean administration to a cell or subject of one or more “naked” AGT dsRNA agent or AGT antisense polynucleotide agent sequences and in certain aspects of the invention “delivering” means administration to a cell or subject via transfection means, delivering a cell comprising an AGT dsRNA agent or AGT antisense polynucleotide agent to a subject, delivering a vector encoding an AGT dsRNA agent or AGT antisense polynucleotide agent into a cell and/or subject, etc. Delivery of an AGT dsRNA agent or AGT antisense polynucleotide agent using a transfection means may include administration of a vector to a cell and/or subject.

In some methods of the invention one or more AGT dsRNA agents or AGT antisense polynucleotide agents may be administered in formulations, which may be administered in pharmaceutically acceptable solutions, which may routinely contain pharmaceutically acceptable concentrations of salt, buffering agents, preservatives, compatible carriers, adjuvants, and optionally other therapeutic ingredients. In some embodiments of the invention an AGT dsRNA agent or AGT antisense polynucleotide agent may be formulated with another therapeutic agent for simultaneous administration. According to methods of the invention, an AGT dsRNA agent or AGT antisense polynucleotide agent may be administered in a pharmaceutical composition. In general, a pharmaceutical composition comprises an AGT dsRNA agent or AGT antisense polynucleotide agent and optionally, a pharmaceutically-acceptable carrier. Pharmaceutically-acceptable carriers are well-known to those of ordinary skill in the art. As used herein, a pharmaceutically-acceptable carrier means a non-toxic material that does not interfere with the effectiveness of the biological activity of the active ingredients, e.g., the ability of the AGT dsRNA agent or AGT antisense polynucleotide agent to inhibit AGT gene expression in a cell or subject. Numerous methods to administer and deliver dsRNA agents or AGT antisense polynucleotide agents for therapeutic use are known in the art and may be utilized in methods of the invention.

Pharmaceutically acceptable carriers include diluents, fillers, salts, buffers, stabilizers, solubilizers and other materials that are well-known in the art. Exemplary pharmaceutically acceptable carriers are described in U.S. Pat. No. 5,211,657 and others are known by those skilled in the art. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Some embodiments of methods of the invention include administering one or more AGT dsRNA agents or AGT antisense polynucleotide agents directly to a tissue. In some embodiments, the tissue to which the compound is administered is a tissue in which the AGT-associated disease or condition is present or is likely to arise, non-limiting examples of which are the liver or kidney. Direct tissue administration may be achieved by direct injection or other means. Many orally delivered compounds naturally travel to and through the liver and kidneys and some embodiments of treatment methods of the invention include oral administration of one or more AGT dsRNA agents to a subject. AGT dsRNA agents or AGT antisense polynucleotide agents, either alone or in conjunction with other therapeutic agents, may be administered once, or alternatively they may be administered in a plurality of administrations. If administered multiple times, the AGT dsRNA agent or AGT antisense polynucleotide agent may be administered via different routes. For example, though not intended to be limiting, a first (or first several) administrations may be made via subcutaneous means and one or more additional administrations may be oral and/or systemic administrations.

For embodiments of the invention in which it is desirable to administer an AGT dsRNA agent or AGT antisense polynucleotide agent systemically, the AGT dsRNA agent or AGT antisense polynucleotide agent may be formulated for parenteral administration by injection, e.g., by bolus injection or continuous infusion. Formulations for injection may be presented in unit dosage form, e.g., in ampoules or in multi-dose containers, with or without an added preservative. AGT dsRNA agent formulations (also referred to as pharmaceutical compositions) may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day may be used as needed to achieve appropriate systemic or local levels of one or more AGT dsRNA agents or AGT antisense polynucleotide agents and to achieve appropriate reduction in AGT protein activity.

In yet other embodiments, methods of the invention include use of a delivery vehicle such as biocompatible microparticle, nanoparticle, or implant suitable for implantation into a recipient, e.g., a subject. Exemplary bioerodible implants that may be useful in accordance with this method are described in PCT Publication No. WO 95/24929 (incorporated by reference herein), which describes a biocompatible, biodegradable polymeric matrix for containing a biological macromolecule.

Both non-biodegradable and biodegradable polymeric matrices can be used in methods of the invention to deliver one or more AGT dsRNA agents or AGT antisense polynucleotide agents to a subject. In some embodiments, a matrix may be biodegradable. Matrix polymers may be natural or synthetic polymers. A polymer can be selected based on the period of time over which release is desired, generally in the order of a few hours to a year or longer. Typically, release over a period ranging from between a few hours and three to twelve months can be used. The polymer optionally is in the form of a hydrogel that can absorb up to about 90% of its weight in water and further, optionally is cross-linked with multivalent ions or other polymers.

In general, AGT dsRNA agents or AGT antisense polynucleotide agents may be delivered in some embodiments of the invention using the bioerodible implant by way of diffusion, or by degradation of the polymeric matrix. Exemplary synthetic polymers for such use are well known in the art. Biodegradable polymers and non-biodegradable polymers can be used for delivery of AGT dsRNA agents or AGT antisense polynucleotide agents using art-known methods. Bioadhesive polymers such as bioerodible hydrogels (see H. S. Sawhney, C. P. Pathak and J. A. Hubell in Macromolecules, 1993, 26, 581-587, the teachings of which are incorporated by reference herein) may also be used to deliver AGT dsRNA agents or AGT antisense polynucleotide agents for treatment of an AGT-associated disease or condition. Additional suitable delivery systems can include time-release, delayed release or sustained release delivery systems. Such systems can avoid repeated administrations of an AGT dsRNA agent or AGT antisense polynucleotide agent, increasing convenience to the subject and the medical care professional. Many types of release delivery systems are available and known to those of ordinary skill in the art. (See for example: U.S. Pat. Nos. 5,075,109; 4,452,775; 4,675,189; 5,736,152; 3,854,480; 5,133,974; and 5,407,686 (the teaching of each of which is incorporated herein by reference). In addition, pump-based hardware delivery systems can be used, some of which are adapted for implantation.

The use of a long-term sustained release implant may be suitable for prophylactic treatment of subjects and for subjects at risk of developing a recurrent AGT-associated disease or condition. Long-term release, as used herein, means that the implant is constructed and arranged to deliver a therapeutic level of an AGT dsRNA agent or AGT antisense polynucleotide agent for at least up to 10 days, 20 days, 30 days, 60 days, 90 days, six months, a year, or longer. Long-term sustained release implants are well-known to those of ordinary skill in the art and include some of the release systems described above.

Therapeutic formulations of AGT dsRNA agents or AGT antisense polynucleotide agents may be prepared for storage by mixing the molecule or compound having the desired degree of purity with optional pharmaceutically acceptable carriers, excipients or stabilizers [Remington's Pharmaceutical Sciences 21^{st} edition, (2006)], in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

### Cells, Subjects, and Controls

Methods of the invention may be used in conjunction with cells, tissues, organs and/or subjects. In some aspects of the invention a subject is a human or vertebrate mammal including but not limited to a dog, cat, horse, cow, goat, mouse, rat, and primate, e.g., monkey. Thus, the invention can be used to treat AGT-associated diseases or conditions in human and non-human subjects. In some aspects of the invention a subject may be a farm animal, a zoo animal, a domesticated animal or non-domesticated animal and methods of the invention can be used in veterinary prevention and treatment regimens. In some embodiments of the invention, the subject is a human and methods of the invention can be used in human prevention and treatment regimens.

Non-limiting examples of subjects to which the present invention can be applied are subjects who are diagnosed with, suspected of having, or at risk of having a disease or condition associated with a higher than desirable AGT expression and/or activity, also referred to as “elevated levels of AGT expression”. Non-limiting examples of diseases and conditions associated with a higher than desirable levels of AGT expression and/or activity are described elsewhere herein. Methods of the invention may be applied to a subject who, at the time of treatment, has been diagnosed as having the disease or condition associated with a higher than desirable AGT expression and/or activity, or a subject who is considered to be at risk for having or developing a disease or condition associated with a higher than desirable AGT expression and/or activity. In some aspects of the invention a disease or condition associated with a higher than desirable AGT level of expression and/or activity is an acute disease or condition, and in certain aspects of the invention a disease or condition associated with a higher than desirable AGT level of expression and/or activity is a chronic disease or condition.

In one non-limiting example, an AGT dsRNA agent of the invention is administered to a patient diagnosed with hypertension, including essential hypertension, secondary hypertension, hypertensive emergencies such as malignant hypertension and accelerated hypertensive blood pressure, acute hypertension, pregnancy-related hypertension, and Refractory hypertension. The methods of the invention are applicable to subjects who have been diagnosed with, or considered to be at risk of having or developing, the disease or condition at the time of treatment.

In another non-limiting example, administration of an AGT dsRNA agent of the invention to treat a disease or condition caused by or associated with activation of the renin-angiotensin-aldosterone system (RAAS), or a symptom or progression thereof in response to A disease or condition in which the RAAS is inactivated. The term “angiotensinogen-related disease” includes diseases, disorders or conditions that benefit from reduced expression of AGT. These disorders are often associated with high blood pressure. Non-limiting examples of angiotensinogen-related diseases include hypertension, e.g., borderline hypertension (also known as prehypertension), essential hypertension (also known as native hypertension or essential hypertension), secondary hypertension (also known as non-native hypertension), isolated systolic or diastolic hypertension, pregnancy-associated hypertension (eg, preeclampsia, eclampsia, and postpartum preeclampsia), diabetic Hypertension, Intractable hypertension, Refractory hypertension, paroxysmal hypertension, renovascular hypertension (also known as renal hypertension), Goldblatt's hypertension, ocular hypertension, glaucoma, pulmonary hypertension Blood pressure, portal hypertension, systemic venous hypertension, systolic hypertension, unstable hypertension; hypertensive heart disease, hypertensive nephropathy, atherosclerosis, arteriosclerosis, vascular disease (including peripheral vascular disease), diabetic nephropathy, diabetic retinopathy, chronic heart failure, cardiomyopathy, diabetic cardiomyopathy, glomerulosclerosis, coarctation of the aorta, aortic aneurysm, ventricular fibrosis, sleep apnea, heart failure (eg, left ventricular systolic dysfunction), myocardial infarction, angina pectoris, stroke, renal disease (eg, chronic kidney disease or diabetic nephropathy, optionally in the case of pregnancy), renal failure (eg, chronic renal failure), cognitive disorders (eg, Alzheimer's disease) and systemic sclerosis (eg, scleroderma renal crisis). In certain embodiments, the AGT-associated disorder comprises intrauterine growth retardation (IUGR) or fetal growth restriction.

A cell to which methods of the invention may be applied include cells that are in vitro, in vivo, ex vivo cells. Cells may be in a subject, in culture and/or in suspension, or in any other suitable state or condition. The cells to which the method of the present invention can be applied may be: liver cells, hepatocytes, cardiac cells, pancreatic cells, cardiovascular cells, kidney cells or other types of vertebrate cells, including human and non-human mammals animal cells. In certain aspects of the invention, the cells to which the methods of the invention are applicable are healthy normal cells that are not known to be diseased cells. In certain embodiments of the invention, the methods and compositions of the invention are applied to cells of the liver, hepatocytes, heart cells, pancreas cells, cardiovascular cells, and/or kidney cells. In certain aspects of the invention, the control cells are normal cells, but it is understood that cells with a disease or condition can also be used as control cells in certain circumstances, for example when comparing treated cells with a disease or condition to cells with a disease or condition. In the case of disorders such as the result of untreated cells.

According to the methods of the invention, the level of AGT polypeptide activity can be determined and compared to a control level of AGT polypeptide activity. A control can be a predetermined value, which can take a variety of forms. It can be a single cutoff such as median or mean. It can be established based on comparing groups, eg, in a group with normal levels of AGT polypeptide and/or AGT polypeptide activity and a group with increased levels of AGT polypeptide and/or AGT polypeptide activity. Another non-limiting example of a comparison group may be a population with one or more symptoms or diagnosis of an AGT-associated disease or condition versus a population without one or more symptoms or diagnosis of a disease or condition; A subject group administered with the siRNA treatment of the present invention and a subject group not administered with the siRNA treatment of the present invention. Typically, controls can be based on apparently healthy normal individuals or apparently healthy cells in an appropriate age group. It should be understood that, in addition to predetermined values, a control according to the invention may be a sample of material tested in parallel with the experimental material. Examples include samples from control populations or control samples produced by manufacturing for testing in parallel with experimental samples. In some embodiments of the invention, controls may include cells or subjects that have not been contacted or treated with the AGT dsRNA agents of the invention, in which case the AGT polypeptide and/or control levels of AGT polypeptide activity may be compared to those of the present invention. The level of AGT polypeptide and/or AGT polypeptide activity in a cell or subject contacted with the inventive AGT dsRNA agent or AGT antisense polynucleotide agent.

In some embodiments of the invention, the control level may be a level of AGT polypeptide determined for a subject, wherein the level of AGT polypeptide determined for the same subject at different times is compared to the control level. In one non-limiting example, the level of AGT is determined in a biological sample obtained from a subject who has not received AGT treatment of the present invention. In some embodiments, the biological sample is a serum sample. AGT polypeptide levels determined in a sample obtained from a subject can serve as a baseline or control value for the subject. After one or more administrations of an AGT dsRNA agent to a subject in the treatment methods of the invention, one or more additional serum samples can be obtained from the subject, and the AGT polypeptide levels in the subsequent one or more samples can be compared to comparison to the subject's control/baseline level. Such comparisons can be used to assess the onset, progression or regression of an AGT-associated disease or condition in a subject. For example, a level of AGT polypeptide in a baseline sample obtained from a subject that is higher than the level obtained from the same subject after administration of an AGT dsRNA agent or an AGT antisense polynucleotide agent of the invention to the subject indicates regression of the AGT-associated disease or condition and Indicates the efficacy of the administered AGT dsRNA agent of the present invention in treating an AGT-associated disease or condition.

In some aspects of the invention, values of one or more of a level of AGT polypeptide and/or AGT polypeptide activity determined for a subject may serve as control values for later comparison of levels of AGT polypeptide and/or AGT activity, in that same subject, thus permitting assessment of changes from a “baseline” AGT polypeptide activity in a subject. Thus, an initial AGT polypeptide level and/or initial AGT polypeptide activity level may be present and/or determined in a subject and methods and compounds of the invention may be used to decrease the level of AGT polypeptide and/or AGT polypeptide activity in the subject, with the initial level serving as a control level for that subject.

Using the methods of the invention, an AGT dsRNA agent and/or an AGT antisense polynucleotide agent of the invention can be administered to a subject. Such dsRNAi agents include, for example, the duplexes AD00051 to AD00122-19-2, AD00163-3, AV01227 to AV01257, AV01711 shown in Table 1. In some embodiments, preferred dsRNAi agents include, for example, duplexes AD00158, AD00163, AD00163-3, AD00159, AD00290, AD00300, or AD00122. In other embodiments, preferred dsRNAi agents include, for example, AD00158-1, AD00158-2, AD00163-1, AD00159-1, or AD00300-1. In other embodiments, such dsRNAi agents include duplex variants, eg, variants of duplex AD00158, AD00163, AD00163-3, AD00159, AD00290, AD00300, or AD00122. The efficacy of administration and treatment of the invention can be assessed as compared to the pre-dose level of AGT polypeptide in a serum sample obtained from a subject at a previous time point, or to the level of a non-contact control (e.g., the level of AGT polypeptide in a control serum sample) In contrast, when administered and treated, the level of the AGT polypeptide in a serum sample obtained from the subject is reduced by at least 0.5%, 1%, 5%, 10%, 20%, 30%, 40%, 50%, 60%, 70% %, 80%, 90%, 95% or more. It is understood that both the level of AGT polypeptide and the level of AGT polypeptide activity correlate with the level of AGT gene expression. Certain embodiments of the methods of the invention comprise administering to a subject an AGT dsRNA and/or an AGT antisense agent of the invention in an amount effective to inhibit expression of the AGT gene, thereby reducing the level of AGT polypeptide and reducing the level of AGT polypeptide activity in the subject.

Some embodiments of the invention include determining the presence, absence and/or amount (also referred to herein as level) of an AGT polypeptide in one or more biological samples obtained from one or more subjects. This assay can be used to assess the efficacy of the therapeutic methods of the invention. For example, the methods and compositions of the invention can be used to determine the level of an AGT polypeptide in a biological sample obtained from a subject previously treated with administration of an AGT dsRNA agent and/or an AGT antisense agent of the invention. After administration and treatment, obtained from a subject is compared to the pre-administration level of AGT polypeptide in a serum sample obtained from the subject at a previous time point, or compared to the level of a non-contact control (eg, the level of AGT polypeptide in a control serum sample). The level of AGT polypeptide in the serum sample is reduced by at least 0.5%, 1%, 5%, 10%, 20%, 30%, 40%, 50%, 60%, 70%, 80%, 90%, 95% or more More, indicating the level of efficacy of the treatment administered to the subject.

In some embodiments of the invention, the physiological characteristics of an AGT-associated disease or condition determined for a subject can be used as a control result, and the determination of the physiological characteristics of the same subject at different times can be compared with the control results. In one non-limiting example, blood pressure (and/or other physiological characteristics of an AGT disease or condition) is measured from a subject who has never been administered an AGT treatment of the invention, which can be used as a baseline or control value for the subject. Following administration of one or more AGT dsRNA agents to a subject in the treatment methods of the invention, blood pressure is measured and compared to the subject's control/baseline levels, respectively. Such comparisons can be used to assess the onset, progression or regression of an AGT-associated disease or condition in a subject. For example, a baseline blood pressure obtained from a subject that is higher than the blood pressure measured from the same subject after administration of an AGT dsRNA agent or an AGT antisense polynucleotide agent of the invention to the subject indicates regression of the AGT-associated disease or condition and indicates the end of the administration. Efficacy of Invented AGT dsRNA Agents in Treating AGT-associated Diseases or Conditions.

In some aspects of the invention, the value determined for a subject for one or more physiological characteristics of an AGT-associated disease or condition may serve as a control value for later comparison of the same subject's physiological characteristics, thereby allowing the assessment of a subject's “Baseline” changes in physiological characteristics. Thus, it is possible to obtain an initial physiological profile in an individual, measure the initial physiological profile as a control for the subject, and show and/or determine that the methods and compounds of the present invention are useful for reducing the level of AGT polypeptide and/or the activity of an AGT polypeptide in an individual. Effect. Using the methods of the invention, the AGT dsRNA agents and/or AGT antisense polynucleotide agents of the invention can be administered to a subject in an amount effective to treat an AGT disease or condition. Efficacy of administrations and treatments of the invention can be assessed by determining changes in one or more physiological characteristics of an AGT disease or condition. In one non-limiting example, the subject's blood pressure is reduced by at least 0.5 mmHg, 1 mmHg, 2 mmHg, 3 mmHg, 4 mmHg, 5 mmHg, 6 mmHg, 7 mmHg, 8 mmHg, 9 mmHg, 10 mmHg, 11 mmHg, 12 mmHg, 13 mmHg, 14 mmHg, 15 mmHg, 16 mmHg, 17 mmHg, 18 mmHg, 19 mmHg, mmHg or more until the subject blood pressure is within the normal range.

Some embodiments of the invention include determining the presence, absence and/or changes in physiological characteristics of an AGT-associated disease or condition using methods such as, but not limited to: (1) measuring a subject's blood pressure; (2) Physiological characteristics of one or more biological samples obtained from multiple subjects; (3) or physical examination of subjects. This assay can be used to assess the efficacy of the therapeutic methods of the invention.

### Kits

Also within the scope of the invention are kits that comprise one or more AGT dsRNA agents and/or AGT antisense polynucleotide agents and instructions for its use in methods of the invention. Kits of the invention may include one or more of an AGT dsRNA agent, AGT sense polynucleotide, and AGT antisense polynucleotide agent that may be used to treat an AGT-associated disease or condition. Kits containing one or more AGT dsRNA agents, AGT sense polynucleotides, and AGT antisense polynucleotide agents can be prepared for use in treatment methods of the invention. Components of kits of the invention may be packaged either in aqueous medium or in lyophilized form. A kit of the invention may comprise a carrier being compartmentalized to receive in close confinement therein one or more container means or series of container means such as test tubes, vials, flasks, bottles, syringes, or the like. A first container means or series of container means may contain one or more compounds such as an AGT dsRNA agent and/or AGT sense or antisense polynucleotide agent. A second container means or series of container means may contain a targeting agent, a labelling agent, a delivery agent, etc. that may be included as a portion of an AGT dsRNA agent and/or AGT antisense polynucleotide to be administered in an embodiment of a treatment method of the invention.

A kit of the invention may also include instructions. Instructions typically will be in written form and will provide guidance for carrying-out a treatment embodied by the kit and for making a determination based upon that treatment.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Example 1. The Synthesis of RNAi Agent

The AGT RNAi agent duplexes shown in Tables 2-4 above were synthesized according to the following general procedure:

Sense and antisense strand sequences of siRNA were synthesized on oligonucleotide synthesizers using a well-established solid phase synthesis method based on phosphoramidite chemistry. Oligonucleotide chain propagation is achieved through 4-step cycles: a deprotection, a condensation, a capping and an oxidation or a sulfurization step for addition of each nucleotide. Syntheses were performed on a solid support made of controlled pore glass (CPG, 1000 Å). Monomer phosphoramidites were purchased from commercial sources. Phosphoramidites with GalNAc ligand cluster (GLPA1 and GLPA2 as non-limiting examples) were synthesized according to the procedures of Examples 2-3 herein. For siRNAs used for in vitro screening (Table 2.), syntheses were carried out at 2 μmol scale, and for siRNAs used for in vivo testing (Table 3, 4), syntheses were carried out at scale of 5 μmol or larger. In the case where the GalNAc ligand (GLO-0 as a non-limiting example) is attached at 3′-end of sense strand, GalNAc ligand attached CPG solid support was used. In the case where the GalNAc ligand (GLS-1 or GLS-2 as non-limiting example) is attached at 5′-end of sense strand, a GalNAc phosphoramidite (GLPA1 or GLPA2 as a non-limiting example) was used for the last coupling reaction. Trichloroacetic acid (TCA) 3% in dichloromethane was used for deprotection of 4,4′-dimethoxytrityl protecting group (DMT). 5-Ethylthio-1H-tetrazole was used as an activator. 12 in THF/Py/H₂O and phenylacetyl disulfide (PADS) in pyridine/MeCN was used for oxidation and sulfurization reactions, respectively. After the final solid phase synthesis step, solid support bound oligomer was cleaved and protecting groups were removed by treating with a 1:1 volume solution of 40 wt. % methylamine in water and 28% ammonium hydroxide solution. For the synthesis of siRNAs used for in vitro screening, crude mixture was concentrated. The remaining solid was dissolved in 1.0 M NaOAc, and ice cold EtOH was added to precipitate out the single strand product as the sodium salt, which was used for annealing without further purification. For the synthesis of siRNAs used for in vivo testing, crude single strand product was further purified by ion pairing reversed phase HPLC (IP-RP-HPLC). Purified single strand oligonucleotide product from IP-RP-HPLC was converted to sodium salt by dissolving in 1.0 M NaOAc and precipitation by addition of ice cold EtOH. Annealing of equimolar complementary sense stand and antisense strand oligonucleotide in water was performed to form the double strand siRNA product, which was lyophilized to afford a fluffy white solid.

### Example 2. Preparation of Intermediate-A and Intermediate-B

As shown in Scheme 1 below, Intermediate-A was synthesized by treating commercially available galactosamine pentaacetate with trimethylsilyl trifluoromethanesulfonate (TMSOTf) in dichloromethane (DCM). This was followed by glycosylation with Cbz protected 2-(2-aminoethoxy)ethan-1-ol to give Compound II. The Cbz protecting group was removed by hydrogenation to afford Intermediate-A as a trifluoroacetate (TFA) salt. Intermediate B was synthesized based on the same scheme except Cbz protected 2-(2-(2-aminoethoxy)ethoxy)ethan-1-ol was used as the starting material.

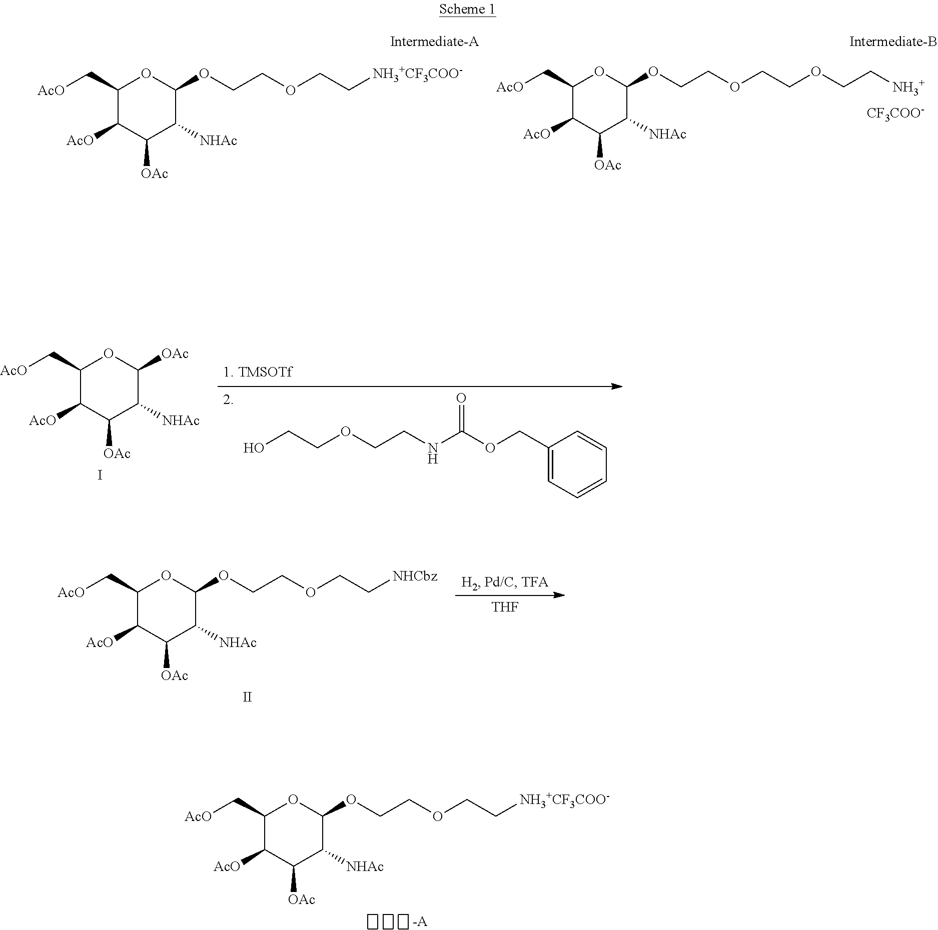

To a solution of Compound I (20.0 g, 51.4 mmol) in 100 mL 1,2-dichloroethane (DCE) was added TMSOTf (17.1 g, 77.2 mmol). The resulting reaction solution was stirred at 60° C. for 2 hrs, and then at 25° C. for 1 hr. Cbz protected 2-(2-aminoethoxy)ethan-1-ol (13.5 g, 56.5 mmol) in DCE (100 mL) dried over 4 Å powder molecular sieves (10 g) was added dropwise to the above mentioned reaction solution at 0° C. under N₂ atmosphere. The resulting reaction mixture was stirred at 25° C. for 16 hrs under N₂ atmosphere. The reaction mixture was filtered and washed with sat. NaHCO₃ (200 mL), water (200 mL) and sat. brine (200 mL). The organic layer was dried over anhydrous Na₂SO₄, filtered and concentrated under reduced pressure to give a crude product, which was triturated with 2-Methyltetrahydrofuran/heptane (5/3, v/v, 1.80 L) for 2 hrs. Resulting mixture was filtered and dried to give Compound II (15.0 g, 50.3% yield) as a white solid.

To a dried and argon purged hydrogenation bottle was carefully added 10% Pd/C (1.50 g), followed by 10 mL tetrahydrofuran (THF) and then a solution of Compound II (15.0 g, 26.4 mmol) in THF (300 mL) and TFA (trifluoroacetic acid, 3.00 g, 26.4 mmol). The resulting mixture was degassed and purged with H₂ three times and stirred at 25° C. for 3 hrs under H₂ (45 psi) atmosphere. Thin-layer chromatography (TLC, solvent: DCM:MeOH=10:1) indicated Compound II was consumed completely. The reaction mixture was filtered and concentrated under reduced pressure. Residue was dissolved in anhydrous DCM (500 mL) and concentrated. This process was repeated 3 times to give Intermediate-A (14.0 g, 96.5% yield) as a foamy white solid. ¹H NMR (400 MHz DMSO-d₆): δ ppm 7.90 (d, J=9.29 Hz, 1H), 7.78 (br s, 3H), 5.23 (d, J=3.26 Hz, 1H), 4.98 (dd, J=11.29, 3.26 Hz, 1H), 4.56 (d, J=8.53 Hz, 1H), 3.98-4.07 (m, 3H), 3.79-3.93 (m, 2H), 3.55-3.66 (m, 5H), 2.98 (br d, J=4.77 Hz, 2H), 2.11 (s, 3H), 2.00 (s, 3H), 1.90 (s, 3H), 1.76 (s, 3H).

Intermediate-B was synthesized using similar procedures for synthesis of Intermediate-A. ¹H NMR (400 MHz DMSO-d₆): δ ppm 7.90 (br d, J=9.03 Hz, 4H), 5.21 (d, J=3.51 Hz, 1H), 4.97 (dd, J=11.1 Hz, 1H), 4.54 (d, J=8.53 Hz, 1H), 3.98-4.06 (m, 3H), 3.88 (dt, J=10.9 Hz, 1H), 3.76-3.83 (m, 1H), 3.49-3.61 (m, 9H), 2.97 (br s, 2H), 2.10 (s, 3H), 1.99 (s, 3H), 1.88 (s, 3H), 1.78 (s, 3H). Mass calc. for C₂₀H₃₄N₂O₁₁: 478.22; found: 479.3 (M+H⁺).

### Example 3. Synthesis of GalNAc Ligand Cluster Phosphoramidite GLPA1, GLPA2 and GLPA15

Scheme 2 below was followed to prepare GLPA1 and GLPA2. Starting from benzyl protected propane-1,3-diamine, it was alkylated with tert-butyl 2-bromoacetate to afford triester Compound I. The benzyl protecting group was removed by hydrogenation to afford secondary amine Compound II. Amide coupling with 6-hydroxyhexanoic acid afforded Compound III. tert-Butyl protecting groups were then removed upon treatment of HCl in dioxane to generate triacid Compound IV. Amide coupling between triacid compound IV and Intermediate-A or Intermediate-B was performed to afford Compound Va or Vb. Phosphoramidite GLPA1 or GLPA2 was synthesized by phosphitylation of Compound Va or Vb with 2-Cyanoethyl N,N-diisopropylchlorophosphoramidite and a catalytic amount of 1H-tetrazole.

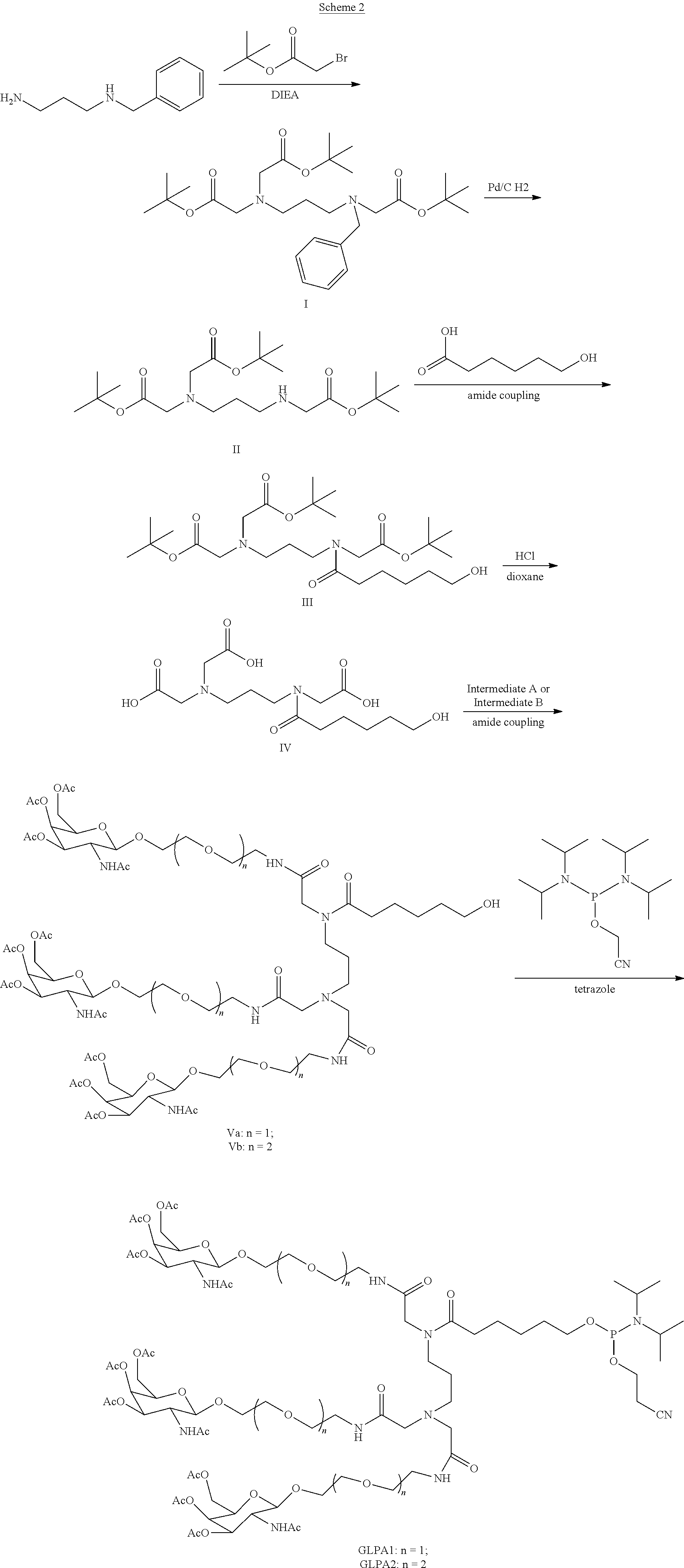

To a solution of N-Benzyl-1,3-propanediamine (5.00 g, 30.4 mmol) in dimethylformamide (DMF, 100 mL) was added tert-butyl 2-bromoacetate (23.7 g, 121 mmol), followed by addition of diisopropylethylamine (DIEA, 23.61 g, 182 mmol) dropwise. The resulting reaction mixture was stirred at 25-30° C. for 16 hrs. LCMS showed N-Benzyl-1,3-propanediamine was consumed completely. Reaction mixture was diluted with H₂O (500 mL) and extracted with EtOAc (500 mL×2). The combined organics were washed with sat. brine (1 L), dried over anhydrous Na₂SO₄, filtered, and concentrated under reduced pressure to give crude product, which was purified by silica gel column chromatography (gradient: petroleum ether:ethyl acetate from 20:1 to 5:1). Compound I (12.1 g, 78.4% yield) was obtained as a colorless oil. ¹H NMR (400 MHz, CDCl₃): δ ppm 7.26-7.40 (m, 5H), 3.79 (s, 2H), 3.43 (s, 4H), 3.21 (s, 2H), 2.72 (dt, J=16.9, 7.34 Hz, 4H), 1.70 (quin, J=7.2 Hz, 2H), 1.44-1.50 (m, 27H).

A dried hydrogenation bottle was purged with Argon three times. Pd/C (200 mg, 10%) was added, followed by MeOH (5 mL) and then a solution of Compound I (1.00 g, 1.97 mmol) in MeOH (5 mL). The reaction mixture was degassed under vacuum and refilled with H₂. This process was repeated three times. The mixture was stirred at 25° C. for 12 hrs under H₂ (15 psi) atmosphere. LCMS showed Compound I was consumed completely. The reaction mixture was filtered under reduced pressure under N₂ atmosphere. Filtrate was concentrated under reduced pressure to give Compound II (655 mg, 79.7% yield) as yellow oil, which was used for the next step without further purification. ¹H NMR (400 MHz, CDCl₃): δ ppm 3.44 (s, 4H), 3.31 (s, 2H), 2.78 (t, J=7.1 Hz, 2H), 2.68 (t, J=6.9 Hz, 2H), 1.88 (br s, 1H), 1.69 (quin, J=7.03 Hz, 2H), 1.44-1.50 (s, 27H).

A mixture of Compound II (655 mg, 1.57 mmol), 6-hydroxyhexanoic acid (249 mg, 1.89 mmol), DIEA (1.02 g, 7.86 mmol), 1-ethyl-3-(3-dimethylaminopropyl)carbodiimide hydrochloride (EDCI, 904 mg, 4.72 mmol), and 1-Hydroxybenzotriazole (HOBt, 637 mg, 4.72 mmol) in DMF (6 mL) was degassed and purged with N₂ three times, and then was stirred at 25° C. for 3 hrs under N₂ atmosphere. LCMS indicated desired product. The reaction mixture was diluted with H₂O (10 mL) and extracted with EtOAc 20 mL (10 mL×2). Organics were combined and washed with sat. brine (20 mL), dried over anhydrous Na₂SO₄, filtered, and concentrated to give crude product, which was purified by silica gel column chromatography (gradient: petroleum ether:ethyl acetate from 5:1 to 1:1) to afford Compound III (650 mg, 77.8% yield) as a yellow oil. ¹H NMR (400 MHz, CDCl₃): δ ppm 3.90-3.95 (s, 2H), 3.63 (t, J=6.40 Hz, 2H), 3.38-3.45 (m, 6H), 2.72 (t, J=6.65 Hz, 2H), 2.40 (t, J=7.28 Hz, 2H), 1.55-1.75 (m, 8H), 1.44 (s, 27H). Mass calc. for C₂₇H₅₀N₂O₈: 530.36; found: 531.3 (M+H⁺).

A mixture of Compound III (5.5 g, 10.3 mmol) in HCl/dioxane (2M, 55 mL) was stirred at 25° C. for 3 hrs. LCMS showed complete consumption of Compound III. Reaction mixture was filtered, washed with EtOAc (50 mL), and dried under reduced pressure to give crude product. It was dissolved in CH₃CN (50 mL), volatiles were removed under vacuum. This process was repeated three times to give Compound IV (2.05 g, 54.5% yield) as a white solid. ¹H NMR (400 MHz, D₂O): δ ppm 4.21 (s, 1H), 4.07 (d, J=4.5 Hz, 4H), 3.99 (s, 1H), 3.45-3.52 (m, 3H), 3.42 (t, J=6.5 Hz, 1H), 3.32-3.38 (m, 1H), 3.24-3.31 (m, 1H), 2.37 (t, J=7.4 Hz, 1H), 2.24 (t, J=7.4 Hz, 1H), 1.99 (dt, J=15.5, 7.53 Hz, 1H), 1.85-1.94 (m, 1H), 1.85-1.94 (m, 1H), 1.39-1.56 (m, 4H), 1.19-1.31 (m, 2H).

A mixture of Compound IV (500 mg, 1.05 mmol), Intermediate-A (2.02 g, 3.67 mmol), DIEA (813 mg, 6.30 mmol), EDCI (704 mg, 3.67 mmol) and HOBt (496 mg, 3.67 mmol) in DME (10 mL) was degassed and purged with N₂ for 3 times, and then the mixture was stirred at 25° C. for 3 hrs under N₂ atmosphere. LCMS indicated desired product. The reaction mixture was quenched by addition of H₂O (10 mL), extracted with DCM (10 mL×2). The combined organics were extracted with 10% citric acid (20 mL). The aqueous phase was neutralized with saturated NaHCO₃ solution and re-extracted with DCM (10 mL×2). Organics were dried over sodium sulfate, filtered and concentrated under reduced pressure to give Compound Va (570 mg, 0.281 mmol, 26.8% yield) as a white solid. ¹H NMR: (400 MHz, CDCl₃) ppm δ 7.84-8.12 (m, 3H), 6.85-7.15 (m, 2H), 6.66-6.81 (m, 1H), 5.36 (br d, J=2.7 Hz, 3H), 5.11-5.27 (m, 3H), 4.63-4.85 (m, 3H), 3.90-4.25 (m, 18H), 3.37-3.75 (m, 28H), 3.15-3.28 (m, 4H), 2.64 (br d, J=6.53 Hz, 2H), 2.30-2.46 (m, 2H), 2.13-2.18 (m, 9H), 2.05 (s, 9H), 1.94-2.03 (m, 18H), 1.68 (br s, 2H), 1.45 (br s, 2H), 1.12 (br t, J=7.0 Hz, 2H).

To a solution of Compound Va (260 mg, 0.161 mmol) in anhydrous DCM (5 mL) was added diisopropylammonium tetrazolide (30.3 mg, 0.177 mmol), followed by dropwise addition of 3-bis(diisopropylamino)phosphanyloxypropanenitrile (194 mg, 0.645 mmol) at ambient temperature under N₂. The reaction mixture was stirred at 20˜25° C. for 2 hrs. LCMS indicated Compound Va was consumed completely. After cooling to −20° C., the reaction mixture was added to a stirred solution of brine/saturated aq. NaHCO₃(1:1, 5 mL) at 0° C. After stirring for 1 min, DCM (5 mL) was added. Layers were separated. Organics were washed with brine/saturated aq. NaHCO₃ solution (1:1, 5 mL), dried over Na₂SO₄, filtered, and concentrated to˜1 mL of volume. The residue solution was added dropwise to 20 mL methyl tert-butyl ether (MTBE) with stirring. This resulted in precipitation of white solid. The mixture was centrifuged, and solid was collected. The solid was redissolved in 1 mL of DCM and precipitated by addition of MTBE (20 mL). Solid was again isolated by centrifuge. The solid collected was dissolved in anhydrous CH₃CN. Volatiles were removed. This process was repeated two more times to afford GalNAc ligand phosphoramidite compound GLPA1 (153 mg, 84.4 μmol) as a white solid. ¹H NMR (400 MHz, CDCl₃): ppm δ 7.71-8.06 (m, 2H), 6.60-7.06 (m, 3H), 5.37 (br d, J=3.0 Hz, 3H), 5.18-5.32 (m, 3H), 4.70-4.86 (m, 3H), 3.92-4.25 (m, 18H), 3.42-3.85 (m, 30H), 3.25 (m, 4H), 2.59-2.75 (m, 4H), 2.27-2.44 (m, 2H), 2.15-2.20 (s, 9H) 2.07 (s, 9H), 1.96-2.03 (m, 18H), 1.65 (br s, 4H), 1.44 (br d, J=7.28 Hz, 2H), 1.14-1.24 (m, 12H). ³¹P NMR (CDCl₃): ppm δ 147.15.

GalNAc ligand phosphoramidite compound GLPA2 was synthesized using the same procedure except Intermediate-B was used. ¹H NMR (400 MHz, CDCl₃): ppm δ 7.94-8.18 (m, 1H), 7.69 (br s, 1H), 6.66-7.10 (m, 3H), 5.35 (d, J=3.5 Hz, 3H), 5.07-5.25 (m, 3H), 4.76-4.86 (m, 3H), 4.01-4.31 (m, 10H), 3.91-4.01 (m, 8H), 3.74-3.86 (m, 4H), 3.52-3.71 (m, 30H), 3.42-3.50 (m, 6H), 3.15-3.25 (m, 4H), 2.52-2.70 (m, 4H), 2.22-2.45 (m, 2H), 2.15-2.22 (s, 9H), 2.06 (s, 9H), 1.95-2.03 (m, 18H), 1.77 (br s, 2H), 1.58-1.66 (m, 4H), 1.40 (m, 2H), 1.08-1.24 (m, 12H). ³¹P NMR (CDCl₃): ppm δ 147.12.

Scheme 3 below was followed to prepare GLPA15.

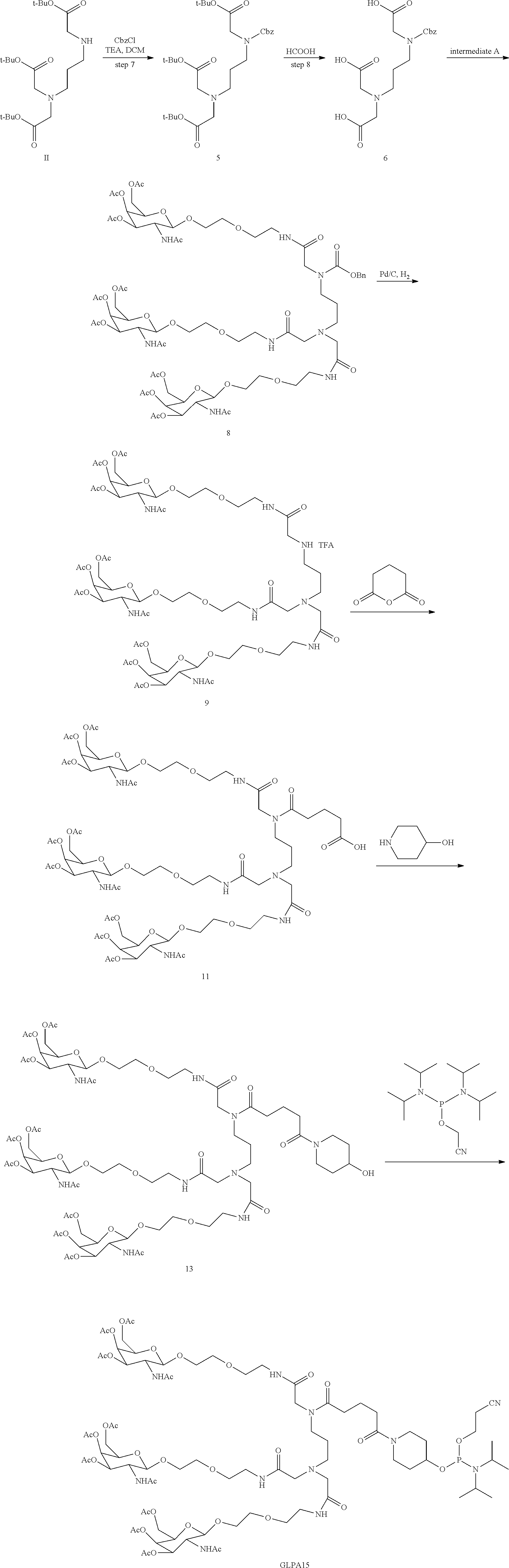

To a solution of intermediate compound II (275 g, 660 mmol, 1.00 eq.) in dichloromethane (2.75 L) was added triethylamine (133 g, 1.32 mol, 2.00 eq.), followed by dropwise addition of Cbz-Cl (169 g, 990 mmol, 1.50 eq.). The reaction solution was stirred at 25° C. for 2 hours, and LCMS showed that compound II was completely converted. The reaction solution was washed successively with NaHCO₃(800 mL) saturated solution and saturated brine (500 mL), and the organic phase was dried with anhydrous Na₂SO₄. After removing the desiccant by filtration, the filtrate was concentrated to dryness. The crude product was subjected to column chromatography (SiO₂, petroleum ether (PE)/ethyl acetate (EA)=100/1 to 5/1, v/v) to obtain a colorless oily compound 5(290 g, 527 mmol, yield 75.7%). ¹H NMR (400 MHz in DMSO-d₆): δ ppm 7.23-7.40 (m, 5H), 5.00-5.12 (m, 2H), 3.86-3.95 (m, 2H), 3.23-3.39 (m, 6H), 2.55-2.67 (m, 2H), 1.56-1.64 (m, 2H), 1.31-1.46 (m, 27H). MS (ESI) [M+H]⁺ m/z: 551.6.

HCOOH (2.9 L) was added to compound 5(145 g, 263 mmol, 1.00 eq), and the solution was stirred at 60° C. for 12 hours. LCMS showed that the conversion of compound 5 was complete. Add 1.5 L toluene and 1.5 L acetonitrile to the reaction solution, concentrate under reduced pressure to about 500 mL, then add toluene/acetonitrile (1:1, v/v, about 750 mL) and concentrate to about 500 mL, then add acetonitrile (about 1000 mL) and concentrated to dryness, the crude product was pulverized at 60° C. for 2 hours with 700 mL of acetonitrile, and filtered. The solid was collected and dried to obtain white solid compound 6(105 g, quantitative). ¹H NMR (400 MHz in DMSO-d₆): δ ppm 7.26-7.40 (m, 5H), 5.02-5.10 (m, 2H), 3.89-4.00 (m, 2H), 3.36-3.45 (m, 4H), 3.24-3.34 (m, 2H), 2.59-2.72 (m, 2H), 1.40 (s, 2H). MS (ESI) [M+H]⁺ m/z: 383.0.

To a DMF (1.0 L) solution of compound 6(100 g, 261 mmol) and Intermediate-A (502 g, 915. mmol, 3.50 eq.) was added O-benzotriazole-N,N,N′,N′-Tetramethyluronium tetrafluoroboric acid (TBTU) (327 g, 1.02 mol, 3.90 eq.), triethylamine (212 g, 2.09 mol, 8.00 eq.), the reaction was carried out at 25° C. for 1 hour, LCMS showed that compound 6 was converted finish. The reaction solution was added to 4000 mL of water, and extracted with methyl tert-butyl ether (2000 mL twice) to remove impurities, and the remaining aqueous phase was extracted with dichloromethane (3000 mL twice). The dichloromethane phase was washed successively with 10% citric acid aqueous solution (2000 mL divided into two times), saturated NaHCO₃ (2.0 L divided into two times), saturated brine (2.0 L), and dried over anhydrous Na₂SO₄. The filtrate was filtered and concentrated under reduced pressure to obtain white solid compound 8(260 g, 159 mmol, yield 60.9%). ¹H NMR (400 MHz in DMSO-d₆): δ ppm 7.99-8.08 (m, 2H), 7.93 (br d, J=5.50 Hz, 1H), 7.79-7.86 (m, 3H), 7.26-7.39 (m, 5H), 5.22 (d, J=3.13 Hz, 3H), 4.95-5.08 (m, 5H), 4.54 (br d, J=8.38 Hz, 3H), 4.03 (s, 9H), 3.81-3.93 (m, 5H), 3.76 (br d, J=4.88 Hz, 3H), 3.44-3.62 (m, 10H), 3.34-3.43 (m, 6H), 3.24 (br d, J=6.13 Hz, 7H), 3.02-3.09 (m, 4H), 2.40-2.47 (m, 2H), 2.10 (s, 9H), 1.99 (s, 9H), 1.89 (s, 9H), 1.77 (s, 9H), 1.57-1.68 (m, 2H). MS (ESI) [M+H]⁺ m/z: 816.4.

2 L hydrogenation kettle with argon and carefully add dry Pd/C (9 g), add MeOH (50 mL) to wet Pd/C, then slowly add compound 8(90 g, 55.1 mmol, 1.00 eq.) under argon atmosphere and trifluoroacetic acid (6.29 g, 55.1 mmol, 1.00 eq.) in MeOH (850 mL). The mixture was degassed/replaced by adding H₂ three times to a hydrogen atmosphere and stirred at 25° C. for 10 h. LCMS showed that the conversion of compound 8 was complete, Pd/C was removed by filtration, and the filtrate was concentrated under reduced pressure to obtain compound 9(80 g, yield 90.2%). ¹H NMR (400 MHz in DMSO-d₆): δ ppm 9.12 (br s, 2H), 8.50 (br t, J=5.19 Hz, 1H), 8.10 (br t, J=5.50 Hz, 2H), 7.85-7.91 (m, 3H), 5.22 (d, J=3.25 Hz, 3H), 4.95-5.01 (m, 3H), 4.52-4.58 (m, 3H), 4.03 (s, 9H), 3.84-3.93 (m, 3H), 3.75-3.83 (m, 3H), 3.39-3.61 (m, 16H), 3.23-3.32 (m, 6H), 3.15-3.18 (m, 3H), 2.97-3.05 (m, 2H), 2.54-2.61 (m, 2H), 2.10 (s, 9H), 2.00 (s, 9H), 1.89 (s, 9H), 1.77-1.80 (m, 9H), 1.70-1.76 (m, 2H). MS (ESI) [M+H]⁺ m/z: 749.3.

To dichloromethane (2.7 L) solution of compound 9(270 g, 168 mmol, 1.00 eq.) and glutaric anhydride (28.6 g, 252 mmol, 1.50 eq) was added triethylamine (67.8 g, 672 mmol, 4.00 eq.), the solution was stirred at 25° C. for 1 hour, and LCMS showed that compound 9 was completely converted to compound 11. 4-Hydroxypiperidine (42.4 g, 420 mmol, 2.50 eq.) and TBTU (107 g, 335 mmol, 2.00 eq.) were added to the reaction solution, and stirring was continued at 25° C. for 1 hour. LCMS showed complete conversion of compound 11. The reaction was quenched by slowly adding saturated NH₄Cl (3.0 L), the layers were separated, the aqueous phase was extracted with dichloromethane (2×1000 mL) and combined with the previous organic phase. The combined organic phases were washed with a 1:1 (v/v) mixture (3.0 L) of saturated NaHCO₃(aq) and saturated brine, dried over anhydrous Na₂SO₄, filtered and concentrated under reduced pressure. The crude product was dissolved in 1.5 L of dichloromethane and added dropwise to methyl tert-butyl ether (7.5 L), A translucent white precipitate gradually formed during the dropwise addition. The precipitate was filtered in vacuo, and the solid was collected and dried in vacuo to obtain compound 13(207 g, yield 72.8%) as a white solid. ¹H NMR (400 MHz in DMSO-d₆): δ ppm 8.05 (br d, J=2.00 Hz, 2H), 7.82 (br d, J=7.38 Hz, 3H), 5.21 (br s, 3H), 4.98 (br d, J=10.26 Hz, 3H), 4.72 (br s, 1H), 4.54 (br d, J=7.88 Hz, 3H), 4.03 (br s, 9H), 3.74-3.94 (m, 9H), 3.45-3.71 (m, 12H), 3.40 (br s, 6H), 3.24 (br s, 7H), 3.07 (br d, J=14.13 Hz, 5H), 2.91-3.01 (m, 1H), 2.24-2.44 (m, 5H), 2.20 (br s, 1H), 2.10 (s, 9H), 1.96-2.04 (m, 9H), 1.89 (br s, 9H), 1.74-1.81 (m, 9H), 1.51-1.73 (m, 6H), 1.07-1.36 (m, 3H). MS (ESI) [M+H]⁺ m/z: 848.0.

To the dichloromethane (2.0 L) solution of compound 13(200 g, 118 mmol, 1.00 eq.) and tetrazole diisopropylammonium (8.08 g, 47.2 mmol, 0.40 eq.) was added 3-double (diisopropyl amino) phosphonyl oxypropionitrile (53.3 g, 177 mmol, 1.50 eq.), the reaction solution was stirred at 40° C. for 2 hours, and LCMS showed that the conversion of compound 13 was complete. The reaction solution was washed with a 1:1 mixture (2.0 L) of saturated NaHCO₃ and saturated brine, dried over anhydrous Na₂SO₄, and the crude product obtained after the filtrate was concentrated was dissolved in dichloromethane (1.2 L), added dropwise to the stirred Methyl tert-butyl ether (6.0 L), filter the suspension, rinse the filter cake with tert-butyl ether, collect the solid and dry it in vacuum, dissolve the product in dichloromethane (1.0 L) and concentrate to dryness, repeated the operation 4 times to remove residual tert-butyl ether to obtain GLPA15(164 g, yield 73.3%). ¹H NMR (400 MHz in DMSO-d₆): δ ppm 8.05 (br d, J=6.50 Hz, 2H), 7.81 (br d, J=9.01 Hz, 3H), 5.22 (d, J=3.25 Hz, 3H), 4.98 (dd, J=11.26, 3.25 Hz, 3H), 4.55 (br d, J=8.50 Hz, 3H), 4.03 (s, 9H), 3.64-3.97 (m, 12H), 3.55-3.63 (m, 6H), 3.50 (br s, 5H), 3.40 (br d, J=6.13 Hz, 6H), 3.17-3.30 (m, 9H), 3.07 (br d, J=14.26 Hz, 4H), 2.76 (t, J=5.82 Hz, 2H), 2.18-2.47 (m, 6H), 2.10 (s, 9H), 1.99 (s, 9H), 1.89 (s, 9H), 1.78 (s, 9H), 1.52-1.74 (m, 6H), 1.12-1.19 (m, 12H). ³¹P NMR (DMSO-d₆): ppm δ 145.25. MS (ESI) [M+H]⁺ m/z: 1895.7.

In certain studies, methods are provided for attaching a targeting group comprising GalNAc (also referred to herein as a GalNAc delivery compound) to the 5′-end of the sense strand, which involves GalNAc phosphoramidite (GLPA1) is used in the coupling step, using a synthetic process such as that used in oligonucleotide chain elongation (i.e. addition of nucleotides at the 5′ end of the sense strand) to ligate them to the 5′-end of the sense strand.

In some studies, a method of attaching a targeting group comprising GalNAc to the 3′-end of a sense strand comprised the use of a solid support (CPG) that included a GLO-n. In some studies, the method of attaching a targeting group comprising GalNAc to the 3′-end of a sense strand included linking the GalNAc targeting group to a CPG solid support through an ester bond, and synthesizing the sense strand When using the resulting CPG with an attached GalNAc targeting group, this results in the GalNAc targeting group being attached at the 3′-end of the sense strand.

Other GalNAc phosphoramidite compounds (GLPAn) can also be obtained by using a reasonable corresponding intermediate, using a method similar to this article or well-known in the art, and connected to a suitable position of the siRNA duplex as a targeting group.

### Example 4. In Vitro Screening of AGT siRNA Duplexes

Hep3B cells were digested with trypsin and adjusted to a suitable density, and then seeded into 96-well plates. Simultaneously with seeding, cells were transfected with test siRNA or control siRNA using Lipofectamine RNAiMax (Invitrogen-13778-150) according to the manufacturer's recommendations. siRNAs were tested in triplicate at two concentrations (0.2 nM to 1.0 nM), while control siRNA was tested at eight concentrations of in sequential 3-fold dilutions from 4.6 pM to 10 nM in triplicate.

24 hours after transfection, the medium was removed and cells were harvested for RNA extraction. Total RNA was extracted using TRIzol™ agent (Invitrogen-15596018) according to the manual.

cDNA was synthesized using PrimeScript™ RT Kit and gDNA Eraser (Perfect Real Time) (TaKaRa-RR047A) according to the manual. AGT cDNA was detected by qPCR. GAPDH cDNA was tested in parallel as an internal control. PCR was performed as follows: 30 seconds at 95° C., followed by 40 cycles between 10 seconds at 95° C. and 30 seconds at 60° C.

Data Analysis

The expression of the AGT gene in each sample was determined by relative quantification (RQ) using the comparative Ct (ΔΔCt) method; this method measures the difference in Ct (ΔCt) between the target gene and the housekeeping gene (GAPDH).

The equations are listed below:

ΔCT=target gene mean Ct−GAPDH mean Ct

ΔΔCT=ΔCT(sample)−ΔCT(random control or Lipofectamine RNAiMax control);

Relative quantification of target gene mRNA=2\hat{ }(−ΔΔCT)

Inhibition %=(control RQ−sample RQ)/control RQ×100%.

Table 5 provides the experimental results of in vitro studies on the inhibition of AGT expression using various AGT RNAi agents; the double-stranded sequences used correspond to the compounds shown in Table 2.

| Average inhibition % | |
|---|---|
| 1 nM | 0.2 nM |

| Duplex AD# | average | SD | average | SD |
|---|---|---|---|---|
| AD00051 | 63.49 | 5.27 | 57.80 | 4.84 |
| AD00053 | 82.93 | 1.46 | 60.04 | 3.22 |
| AD00054 | 81.76 | 0.05 | 27.18 | 12.85 |
| AD00055 | 63.11 | 2.61 | 27.67 | 13.05 |
| AD00056 | 44.67 | 3.42 | −10.57 | 8.88 |
| AD00057 | 38.36 | 1.00 | 6.39 | 2.89 |
| AD00058 | −6.34 | 7.17 | −10.93 | 13.00 |
| AD00059 | 40.95 | 2.41 | −15.25 | 11.41 |
| AD00060 | 41.58 | 1.21 | 1.71 | 1.62 |
| AD00061 | 34.74 | 7.07 | −52.46 | 7.61 |
| AD00062 | 11.16 | 3.26 | −27.82 | 22.96 |
| AD00063 | 25.99 | 1.34 | −28.28 | 34.49 |
| AD00064 | 48.56 | 10.83 | −22.86 | 4.06 |
| AD00065 | 17.68 | 12.71 | −19.42 | 11.46 |
| AD00066 | 74.76 | 0.61 | 32.99 | 9.82 |
| AD00067 | −15.01 | 11.39 | −70.58 | 12.60 |
| AD00068 | 48.42 | 7.31 | 10.85 | 3.85 |
| AD00070 | 14.21 | 6.91 | −41.65 | 19.32 |
| AD00071 | 71.20 | 0.94 | 40.90 | 9.35 |
| AD00072 | 58.69 | 3.25 | −30.13 | 4.78 |
| AD00073 | 23.87 | 7.61 | −10.98 | 10.03 |
| AD00074 | 78.21 | 3.00 | −13.57 | 4.32 |
| AD00075 | 38.92 | 2.08 | 20.51 | 2.14 |
| AD00076 | 73.89 | 1.40 | 52.43 | 1.99 |
| AD00077 | 73.85 | 1.28 | 56.13 | 1.41 |
| AD00078 | 51.59 | 4.46 | −24.43 | 20.40 |
| AD00079 | 90.61 | 0.61 | 76.62 | 5.02 |
| AD00080 | 88.44 | 1.14 | 16.00 | 15.09 |
| AD00081 | 88.79 | 1.96 | 29.91 | 20.77 |
| AD00082 | 77.98 | 0.45 | 60.15 | 10.15 |
| AD00083 | −25.46 | 6.04 | −24.08 | 6.71 |
| AD00084 | 29.29 | 3.07 | −19.97 | 13.61 |
| AD00085 | 54.82 | 2.64 | 6.94 | 4.85 |
| AD00086 | 61.17 | 2.03 | 26.87 | 16.50 |
| AD00087 | 52.12 | 5.08 | −42.78 | 64.89 |
| AD00088 | 17.16 | 13.36 | −25.66 | 4.89 |
| AD00090 | 72.23 | 5.81 | 14.30 | 6.12 |
| AD00091 | 27.41 | 2.11 | −53.58 | 26.95 |
| AD00092 | 78.93 | 0.53 | 29.92 | 2.95 |
| AD00093 | 80.84 | 0.97 | 30.70 | 14.04 |
| AD00094 | 57.64 | 7.56 | 35.89 | 9.00 |
| AD00095 | 63.51 | 1.25 | 28.95 | 12.77 |
| AD00097 | 81.91 | 2.26 | 37.49 | 7.94 |
| AD00098 | 74.20 | 0.29 | 57.41 | 2.59 |
| AD00099 | 53.49 | 2.05 | 1.42 | 22.51 |
| AD00100 | 88.90 | 0.91 | 62.84 | 5.16 |
| AD00101 | 66.98 | 2.39 | 11.83 | 13.72 |

### Example 5. In Vivo Testing of AGT siRNA Duplexes

To evaluate the in vivo activity of AGT siRNA, mice infected with AAV encoding the human AGT gene were used (4 mice per group). Fourteen days before siRNA administration, female C57BL/6J mice were infected by intravenous injection of 1×10\hat{ }11 viral particle of adeno-associated virus 8 (AAV8) vector encoding human AGT gene. On day 0, mice were subcutaneously injected with a single dose of 2.5 mg/kg or 3 mg/kg of AGT siRNA agent or PBS. Blood samples were collected on day 0, before siRNA administration and at the end of day 7. Human AGT protein concentration was measured by ELISA assay according to the manufacturer's recommended protocol (IBL America, Human Angiotensinogen ELISA Kit). Percent knockdown was calculated by comparing human AGT mRNA levels in mouse livers (determined by qPCR) or human AGT protein levels in plasma samples on day 7 between siRNA-treated and PBS-treated groups. The results are shown in Table 6-9.

TABLE 6
AGT single 3 mpk dose screening in AAV-human AGT transduced
mice; Use GalNAc corresponding to the sequence, chemical modification
and delivery shown in Table 3, the GLO-0 in said compounds
in Table 3 refers to the compound GalNAc3 in Jayaprakash, et
al., (2014) J. Am. Chem. Soc., 136, 16958-16961.

| | Percent knockdown of human | Percent knockdown of |
|---|---|---|
| | AGT mRNA in mouse liver | human AGT in mouse |
| ID# | measured by qPCR | plasma measured by ELISA |
| AD00052 | 86% | 67% |
| AD00113 | 39% | 20% |
| AD00114 | 78% | NA |
| AD00115 | 81% | NA |
| AD00116 | 88% | NA |
| AD00122 | 95% | 83% |
| AD00123 | 89% | 76% |
| AD00124 | 68% | NA |
| AD00125 | 86% | NA |
| AD00126 | 92% | 62% |

NA means not measured.

TABLE 7
AGT single 3 mpk dose screening in AAV-human AGT transduced
mice; using compounds corresponding to the sequence, chemical
modification and delivery shown in Table 3, wherein the
GLO-0 refers to the compound GalNAc3 in Jayaprakash, et
al., (2014) J. Am. Chem. Soc., 136, 16958-16961.

| | Percent knockdown of human | Percent knockdown of human |
|---|---|---|
| | AGT mRNA in mouse liver | AGT in mouse plasma |
| ID# | measured by qPCR | measured by ELISA |
| AD00052 | 72% | 65% |
| AD00154 | 37% | NA |
| AD00155 | 51% | NA |
| AD00156 | 48% | NA |
| AD00157 | 71% | NA |
| AD00158 | 96% | 87% |
| AD00159 | 94% | 80% |
| AD00160 | 68% | NA |
| AD00161 | 77% | NA |
| AD00162 | 85% | NA |
| AD00163 | 94% | 81% |

NA means not measured.

TABLE 8
AGT single 2.5 mpk dose screening in AAV-human AGT transduced
mice; using compounds corresponding to the sequence, chemical
modification and delivery shown in Table 3, wherein the
GLO-0 refers to the compound GalNAc3 in Jayaprakash, et
al., (2014) J. Am. Chem. Soc., 136, 16958-16961. .

| | Percent knockdown of human |
|---|---|
| | AGT mRNA in mouse liver |
| AD# | measured by qPCR |
| AD00052 | 73.2% |
| AD00252 | 25.9% |
| AD00257 | 39.1% |
| AD00260 | 42.2% |
| AD00123 | 74.2% |
| AD00284 | 79.6% |
| AD00158 | 92.7% |
| AD00288 | 86.9% |
| AD00163 | 88.9% |
| AD00289 | 66.3% |
| AD00159 | 85.7% |
| AD00290 | 85.2% |
| AD00285 | 26.4% |
| AD00286 | 54.1% |
| AD00287 | 0.5% |
| AD00256 | 72.3% |
| AD00282 | 81.5% |
| AD00283 | 59.8% |
| AD00291 | −29.9% |
| AD00292 | 78.1% |
| AD00293 | 17.9% |
| AD00294 | 60.8% |
| AD00298 | −14.0% |
| AD00299 | 53.2% |
| AD00300 | 89.4% |
| AD00301 | −47.3% |
| AD00302 | 62.1% |

TABLE 9
AGT single 2.5 mpk dose screening in AAV-human AGT transduced
mice; using compounds corresponding to the sequence, chemical
modification and delivery shown in Table 3 or Table 4, wherein
the GLO-O refers to the compound GalNAc3 in Jayaprakash,
et al., (2014) J. Am. Chem. Soc., 136, 16958-16961.

| | Percent knockdown | Percent knockdown |
|---|---|---|
| | of human AGT in | of human AGT mRNA |
| | mouse plasma | in mouse liver |
| ID# | measured by ELISA | measured by qPCR |
| | | NA |
| AD00158 | 86% | NA |
| AD00158-1 | 85% | NA |
| AD00158-2 | 68% | NA |
| AD00122 | 83% | 84% |
| AD00159 | 77% | NA |
| AD00159-1 | 77% | 90% |
| AD00163 | 81% | 76% |
| AD00163-1 | 89% | 90% |
| AD00300 | 66% | NA |
| AD00300-1 | 69% | 69% |

NA means not measured.

### Example 6. In Vivo Testing of AGT siRNA Duplexes

In order to evaluate the in vivo activity of AGT siRNA, a total of 15 male cynomolgus monkeys (13-22 years old, weighing 7-9 kg) were recruited in this study. The animals will be randomly divided into 5 groups, 3 in each group, and each animal will be given subcutaneous injection 2 mg/kg test article, the test article used corresponds to the compounds shown in Table 4 (AD00158-1, AD00158-2, AD00163-1, AD00159-1, AD00300-1).

After an overnight fast, on days-14 (pre-dose), -7 (pre-dose), 1 (pre-dose) and post-dose days 8, 15, 22, 29, 43, 57, 64, 71, 78, 85 and 92 days for blood collection. The collected blood samples were left at room temperature for at least 30 minutes to clot, and then centrifuged at 350 rpm for 10 minutes at 4° C. Transfer the collected serum (approximately 1.0 mL) into two pre-labeled polypropylene screw cap vials (0.5 ml/vial, one for ELISA assay and the other for spare) and store in a −80° C. freezer until testing.

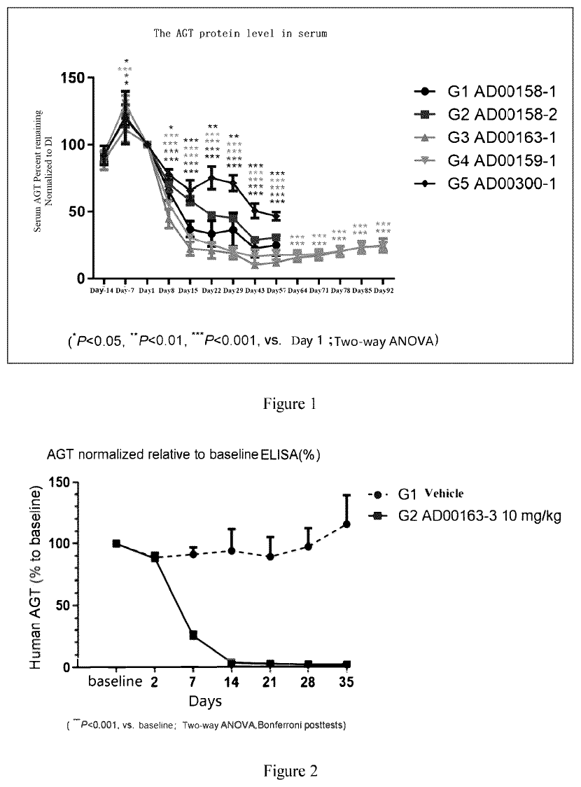

The AGT protein level in serum was determined by Elisa method. The results of the percentage remaining compared to the AGT level in the plasma of the monkeys on the first day are shown in FIG. 1.

### Example 7. In Vitro Screening of AGT siRNA Duplexes

The in vitro research was carried out according to the method of Example 4, and the experimental results are shown in Table 10.

Table 10 provides the experimental results of in vitro studies of inhibition of AGT expression using various AGT RNAi agents; the double-stranded sequences used correspond to the compounds shown in Table 2.

| Average inhibition % | |
|---|---|
| 1 nM | 0.2 nM |

| Duplex AD# | average | SD | average | SD |
|---|---|---|---|---|
| AV01227 | 93.57 | 0.03 | 85.93 | 0.60 |
| AV01228 | 91.34 | 3.40 | 82.13 | 0.53 |
| AV01229 | 80.82 | 3.46 | 56.60 | 1.35 |
| AV01230 | 78.49 | 1.26 | 39.11 | 0.04 |
| AV01231 | 49.54 | 6.13 | 0.54 | 6.09 |
| AV01232 | 80.88 | 1.05 | 53.41 | 1.59 |
| AV01233 | 89.59 | 1.75 | 75.97 | 2.40 |
| AV01234 | 89.20 | 1.86 | 73.90 | 1.12 |
| AV01235 | 86.81 | 0.00 | 70.59 | 3.93 |
| AV01236 | 90.40 | 0.66 | 79.11 | 2.11 |
| AV01237 | 82.94 | 0.71 | 67.31 | 2.28 |
| AV01238 | 84.52 | 0.63 | 71.77 | 1.19 |
| AV01239 | 81.62 | 2.26 | 65.69 | 2.63 |
| AV01240 | 87.57 | 1.05 | 73.71 | 1.72 |
| AV01241 | 88.57 | 0.37 | 75.29 | 1.65 |
| AV01242 | 88.03 | 1.08 | 76.62 | 1.46 |
| AV01243 | 90.29 | 0.30 | 82.39 | 1.85 |
| AV01244 | 86.57 | 0.96 | 75.39 | 2.98 |
| AV01245 | 81.30 | 3.09 | 68.25 | 4.27 |
| AV01246 | 74.37 | 3.11 | 55.34 | 6.77 |
| AV01247 | 63.44 | 8.48 | 39.83 | 6.91 |
| AV01248 | 73.97 | 1.99 | 48.68 | 2.50 |
| AV01249 | 64.97 | 6.78 | 12.91 | 15.95 |
| AV01250 | 88.45 | 0.15 | 70.96 | 0.83 |
| AV01251 | 89.94 | 0.35 | 72.82 | 2.77 |
| AV01252 | 87.26 | 1.72 | 65.79 | 2.21 |
| AV01253 | 86.71 | 2.13 | 66.42 | 0.25 |
| AV01254 | 90.45 | 0.53 | 81.11 | 1.59 |
| AV01255 | 89.78 | 0.42 | 83.48 | 2.51 |
| AV01256 | 89.36 | 0.69 | 78.84 | 0.72 |
| AV01257 | 92.88 | 0.13 | 85.27 | 0.49 |

### Example 8. In Vivo Testing of AD00163-3 in an AAV Mouse Model

After the adaptation period, 12 female C₅₇BL/6J mice were randomly divided into two groups according to body weight: model (vehicle) group and AD00163-3 (1 mg/kg) group. Each mouse was injected with 1×10\hat{ }11 vg of AAV-AGT virus through the tail vein on the first day to establish the animal model, and the injection volume was 100 μL/mouse. On the 15th day, the mice in each group were given PBS or AD00163-3 in Table 4 by subcutaneous injection, and the administration volume was 5 mL/kg. Before administration on day 15, blood was collected from each mouse through the submandibular vein, and serum samples were collected after centrifugation. On day 22, all mice were sacrificed by CO₂, whole blood was collected by cardiac puncture, and serum samples were collected after centrifugation. Human AGT protein concentration was measured by ELISA assay according to the manufacturer's recommended protocol (IBL America, Human Angiotensinogen ELISA Kit). The percent knockdown was calculated by comparing the human-derived AGT protein levels in mouse plasma samples on day 7 (after administration) of the siRNA-treated group and the PBS-treated group. The data showed that AD00163-3 (1 mg/kg) treatment could significantly reduce the expression of human AGT protein in mouse serum by 91%.

### Example 9 Test of AD00163-3 in Spontaneous Hypertension Model of Cynomolgus Monkeys

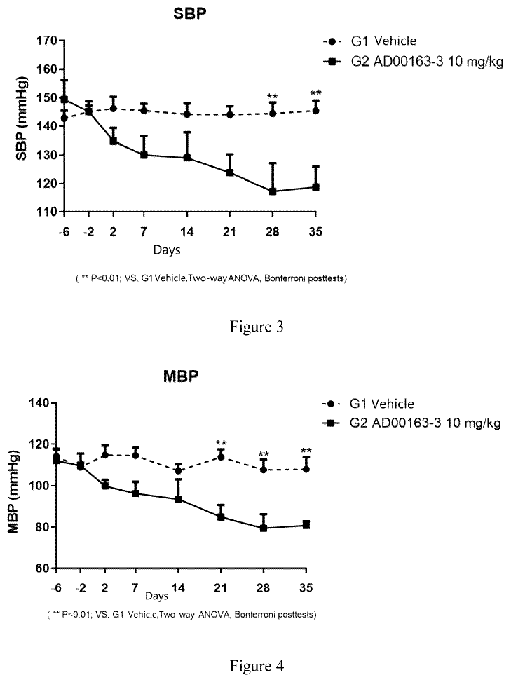

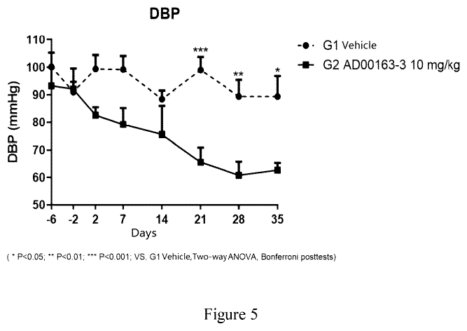

Ten cynomolgus monkeys with elevated blood pressure were randomly divided into two groups (5 monkeys each) to receive either saline or AD00163-3 in Table 4 at 10 mg/kg. Blood samples were collected on days −6 and −2 (pre-dose) and days 2, 7, 14, 21, 28 and 35 (post-dose). Serum AGT concentrations were measured by ELISA according to the manufacturer's recommended protocol, and blood pressure was measured using a tail-cuff device. As shown in FIGS. 2 and 3, respectively, while serum AGT decreases (98% reduction on day 35 after administration), a single subcutaneous dose of 10 mg/kg of AD00163-3 resulted in a significant decrease in SBP by 28 mmHg (SBP from 147 mmHg baseline to 119 mmHg) on day 35 after administration, while control group SBP did not change significantly during the same period (SBP from 144 mmHg baseline to 145 mmHg). Significant reductions in mean and diastolic (MBP and DBP) pressures were also observed as shown in FIGS. 4 and 5, respectively.

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated herein in their entirety herein by reference.

1. A double-stranded ribonucleic acid (dsRNA) agent for inhibiting expression of Angiotensinogen (AGT), wherein the dsRNA agent comprises a sense strand and an antisense strand, nucleotide positions 2 to 18 in the antisense strand comprising a region of complementarity to an AGT RNA transcript, wherein the region of complementarity comprises at least 15 contiguous nucleotides that differ by 0, 1, 2, or 3 nucleotides from one of the antisense sequences listed in one of Tables 1-4, and optionally includes a targeting ligand;
preferably, the region complementary to the AGT RNA transcript comprises at least 15, 16, 17, 18 or 19 contiguous nucleotides that differed by no more than 3 nucleotides from one of the antisense sequences listed in one of Tables 1-4.

2. (canceled)

3. The dsRNA agent of claim 1, wherein the antisense strand of the dsRNA is at least substantially complementary to any one target region in SEQ ID NO: 519, and is provided in any one of Tables 1-4; or
the antisense strand of the dsRNA is fully complementary to any one of the target regions in SEQ ID NO: 519 and is provided in any one of Tables 1-4.

4. (canceled)

5. The dsRNA agent of claim 1, wherein the dsRNA agent comprises the sense strand sequence listed in any one of Tables 1-4, wherein the sense strand sequence is at least substantially complementary to the antisense strand sequence in the dsRNA agent; or
the sense strand sequence is fully complementary to the antisense strand sequence in the dsRNA agent.

1. A double-stranded ribonucleic acid (dsRNA) agent for inhibiting expression of Angiotensinogen (AGT), wherein the dsRNA agent comprises a sense strand and an antisense strand, nucleotide positions 2 to 18 in the antisense strand comprising a region of complementarity to an AGT RNA transcript, wherein the region of complementarity comprises at least 15 contiguous nucleotides that differ by 0, 1, 2, or 3 nucleotides from one of the antisense sequences listed in one of Tables 1-4, and optionally includes a targeting ligand;
preferably, the region complementary to the AGT RNA transcript comprises at least 15, 16, 17, 18 or 19 contiguous nucleotides that differed by no more than 3 nucleotides from one of the antisense sequences listed in one of Tables 1-4.

2. (canceled)

3. The dsRNA agent of claim 1, wherein the antisense strand of the dsRNA is at least substantially complementary to any one target region in SEQ ID NO: 519, and is provided in any one of Tables 1-4, or
the antisense strand of the dsRNA is fully complementary to any one of the target regions in SEQ ID NO: 519 and is provided in any one of Tables 1-4.

4. (canceled)

5. The dsRNA agent of claim 1, wherein the dsRNA agent comprises the sense strand sequence listed in any one of Tables 1-4, wherein the sense strand sequence is at least substantially complementary to the antisense strand sequence in the dsRNA agent; or
the sense strand sequence is fully complementary to the antisense strand sequence in the dsRNA agent.

6. (canceled)

7. The dsRNA agent of claim 1, wherein the dsRNA agent comprises the antisense strand sequence listed in any one of Tables 1-4.

8. The dsRNA agent of claim 1, wherein the dsRNA agent comprises a sequence listed as a duplex sequence in any one of Tables 1-4.

9. The dsRNA agent of claim 1, wherein the dsRNA agent comprises at least one modified nucleotide;
optionally, said at least one modified nucleotide comprises: 2′-O-methyl nucleotide, 2′-Fluoro nucleotide, 2′-deoxy nucleotide, 2′3′-seco nucleotide mimic, locked nucleotide, unlocked nucleic acid nucleotide (UNA), glycol nucleic acid nucleotide (GNA), 2′-F-Arabino nucleotide, 2′-methoyxyethyl nucleotide, abasic nucleotide, ribitol, inverted nucleotide, inverted abasic nucleotide, inverted 2′-Ome nucleotide, inverted 2′-deoxy nucleotide, 2′-amino-modified nucleotide, 2′-alkyl-modified nucleotide, mopholino nucleotide, and 3′-OMe nucleotide, a nucleotide including a 5′-phosphorothioate group, or a terminal nucleotide linked to a cholesteryl derivative or dodecanoic acid bisdecylamide group, a 2′-amino-modified nucleotide, a phosphoramidite, or a non-natural base including nucleotide;
preferably, all or substantially all nucleotides in the antisense strand are modified nucleotides;
preferably, all or substantially all nucleotides of the sense and antisense strands are modified nucleotides;
optionally, the sense strand is a modified sense strand sequence set forth in one of Tables 2-4; or
the antisense strand is a modified antisense strand sequence set forth in one of Tables 2-4.

10-11. (canceled)

12. The dsRNA agent of claim 9, wherein the dsRNA agent comprises an E-vinylphosphonate nucleotide at the 5′ end of the antisense strand.

13. The dsRNA agent of claim 1, wherein the dsRNA agent comprises at least one phosphorothioate internucleoside linkage, or
the sense strand comprises at least one phosphorothioate internucleoside linkage; or
the antisense strand comprises at least one phosphorothioate internucleoside linkage; or
the sense strand comprises 1, 2, 3, 4, 5, or 6 phosphorothioate internucleoside linkages; or
the antisense strand comprises 1, 2, 3, 4, 5, or 6 phosphorothioate internucleoside linkages.

14-20. (canceled)

21. The dsRNA agent of claim 1, wherein the sense strand is complementary or substantially complementary to the antisense strand, and the region of complementarity is 16 to 23 nucleotides in length; or
the complementary region is 19 to 21 nucleotides in length.

22. (canceled)

23. The dsRNA agent of claim 1, wherein each strand is no more than 30 nucleotides in length; or
each strand is no more than 25 nucleotides in length; or
each strand is no more than 23 nucleotides in length.

24-25. (canceled)

26. The dsRNA agent of claim 1, wherein the dsRNA agent comprises at least one modified nucleotide and further comprises one or more targeting or linking groups:
optionally, the one or more targeting groups or linking groups are conjugated to the sense strand;
optionally, the targeting group or linking group comprises N-acetyl-galactosamine (GalNAc); or the targeting group has the following structure:

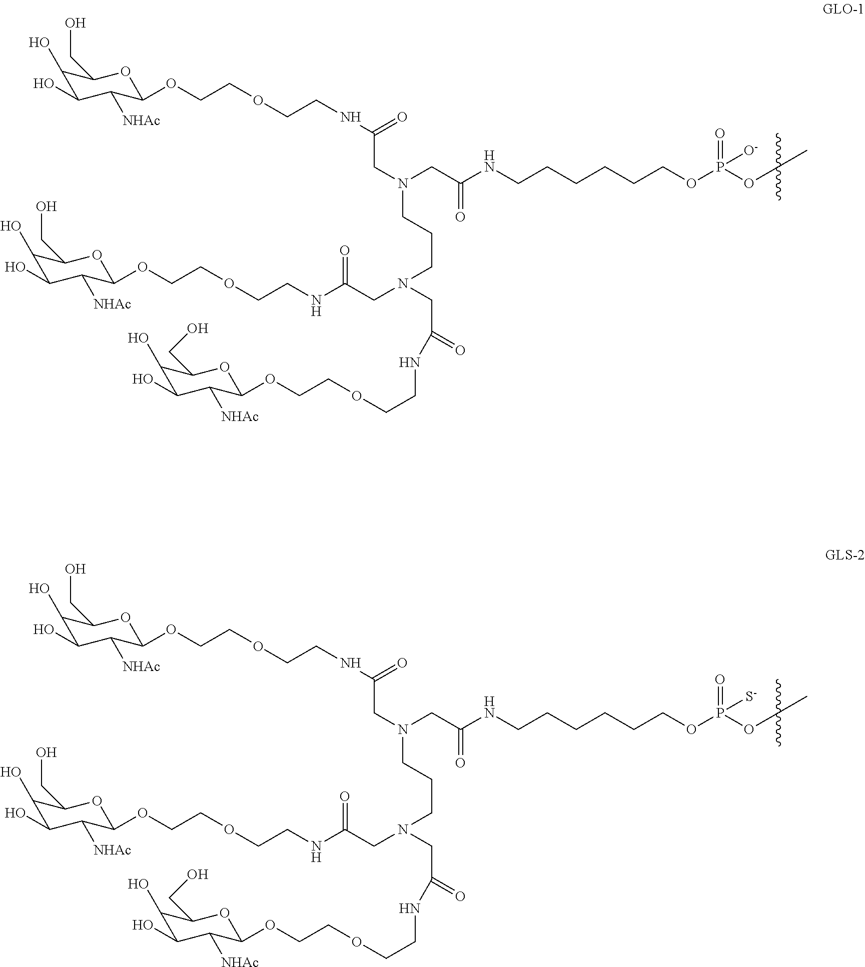 [SMILES: *OP(=O)([O-])OCCCCCCNC(=O)CN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCNC(=O)CN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

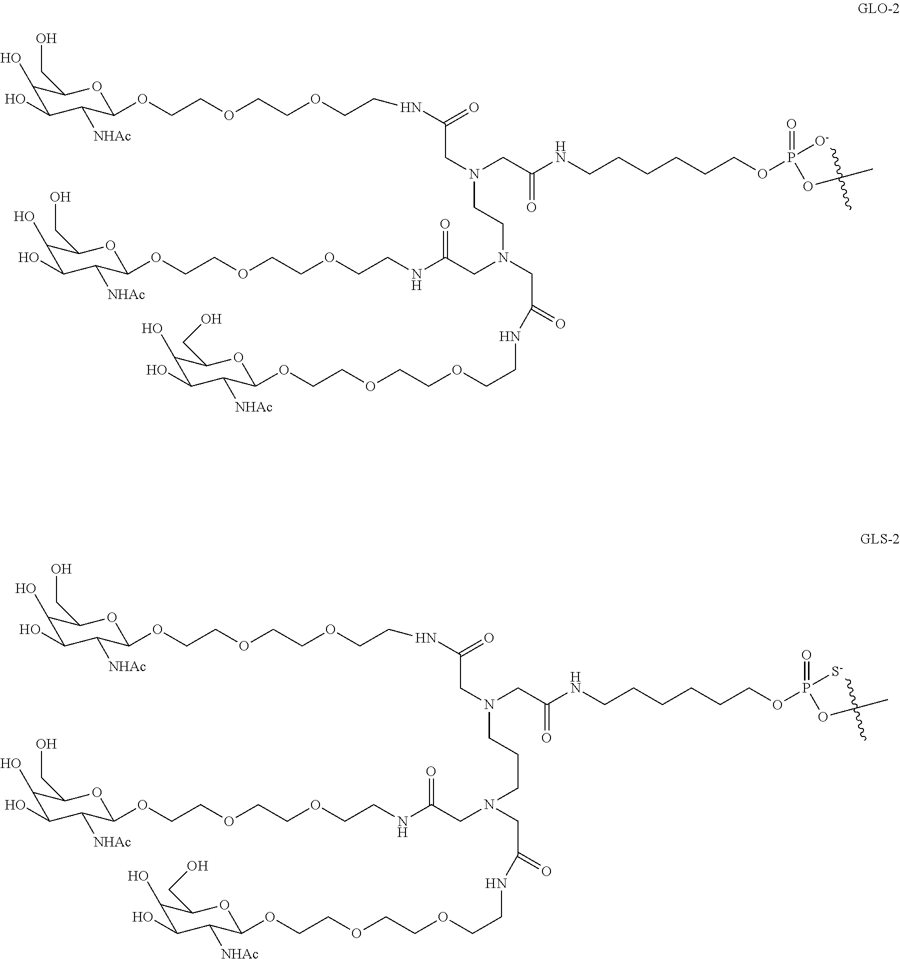 [SMILES: *OP(=O)([O-])OCCCCCCNC(=O)CN(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCCCCCNC(=O)CN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

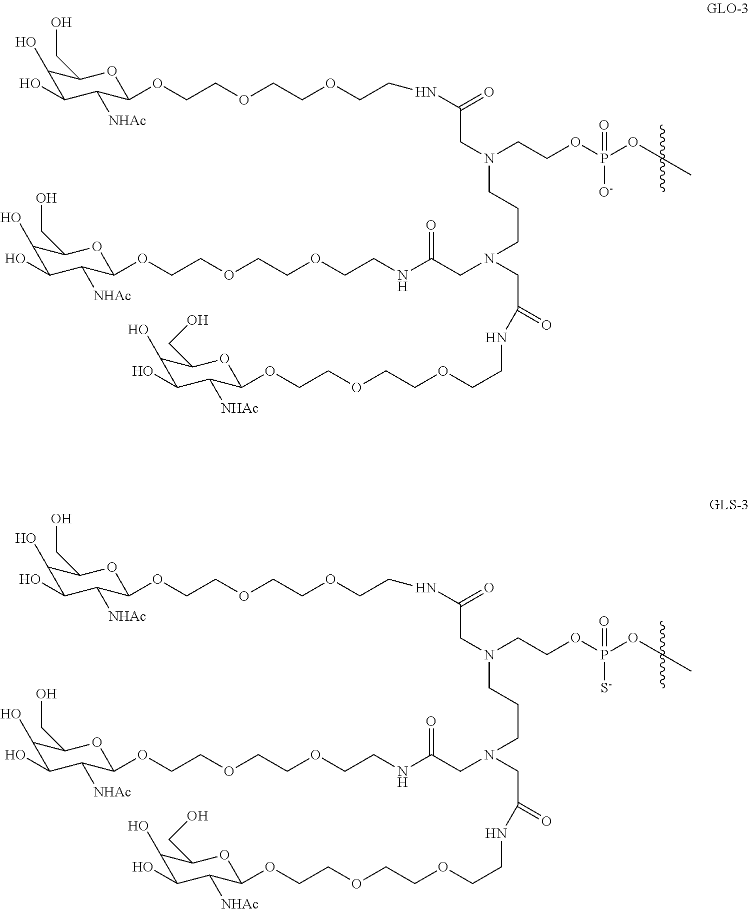 [SMILES: *OP(=O)([O-])OCCN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

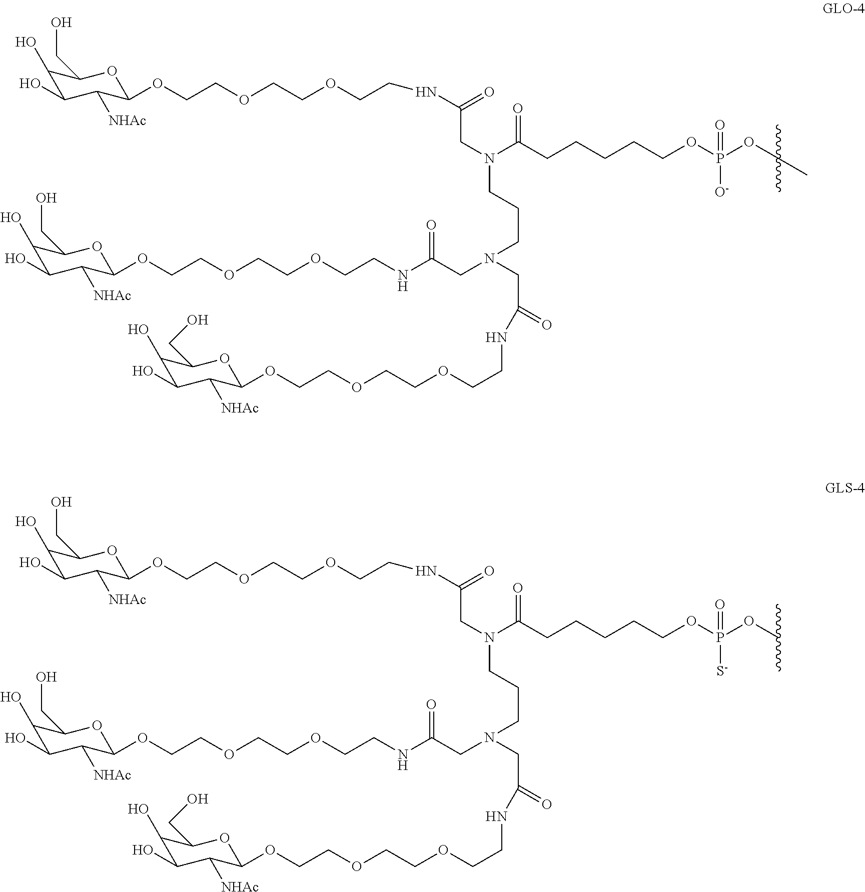 [SMILES: *OP(=O)([O-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

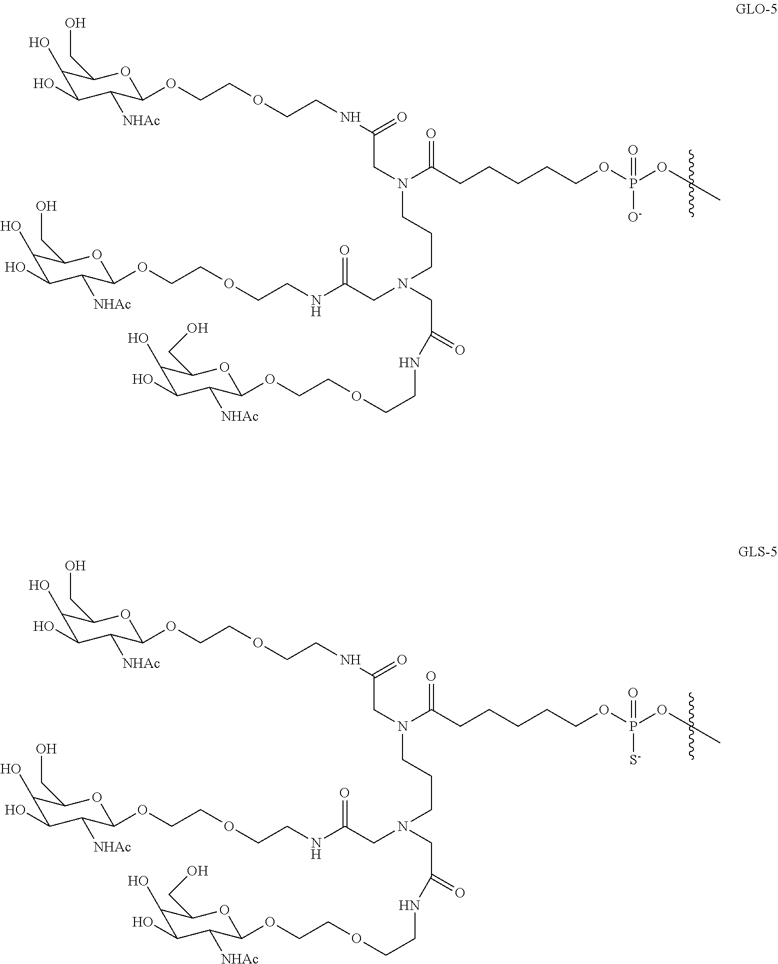 [SMILES: *OP(=O)([O-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

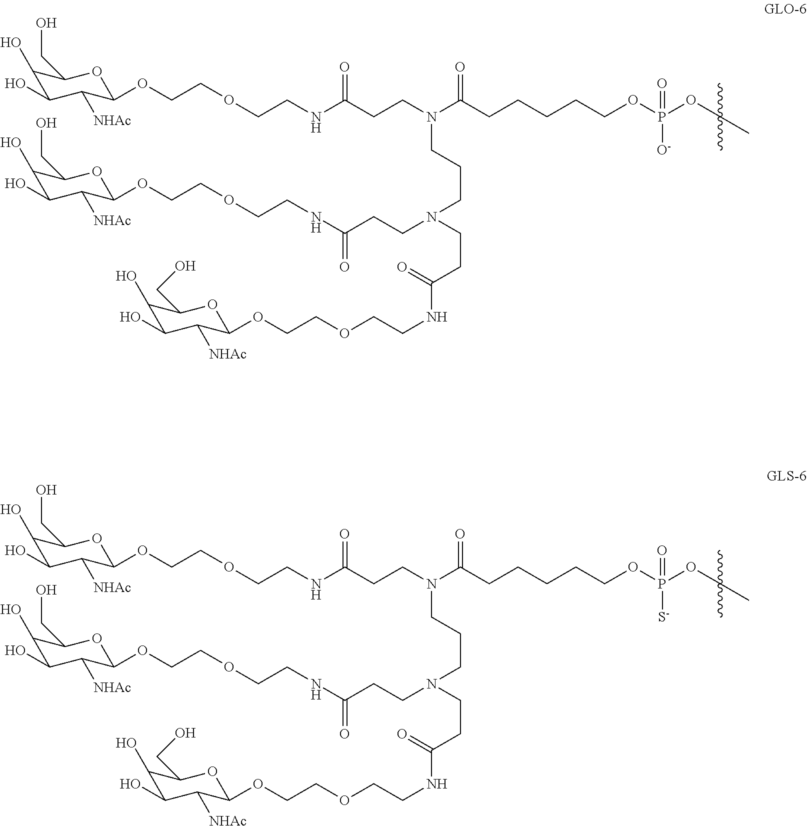 [SMILES: *OP(=O)([O-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O ; *OP(=O)([S-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O]

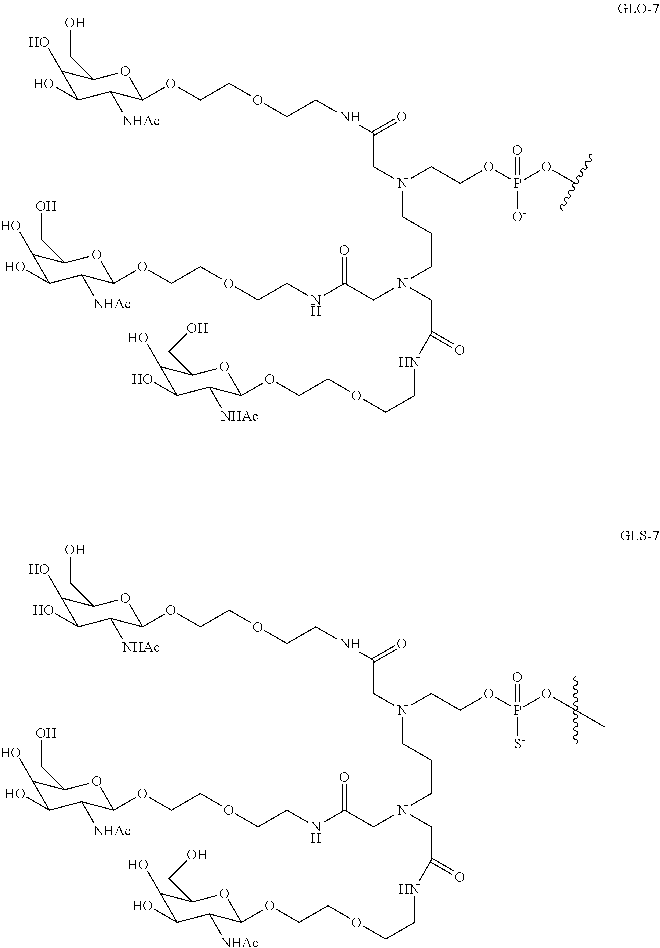 [SMILES: *OP(=O)([O-])OCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

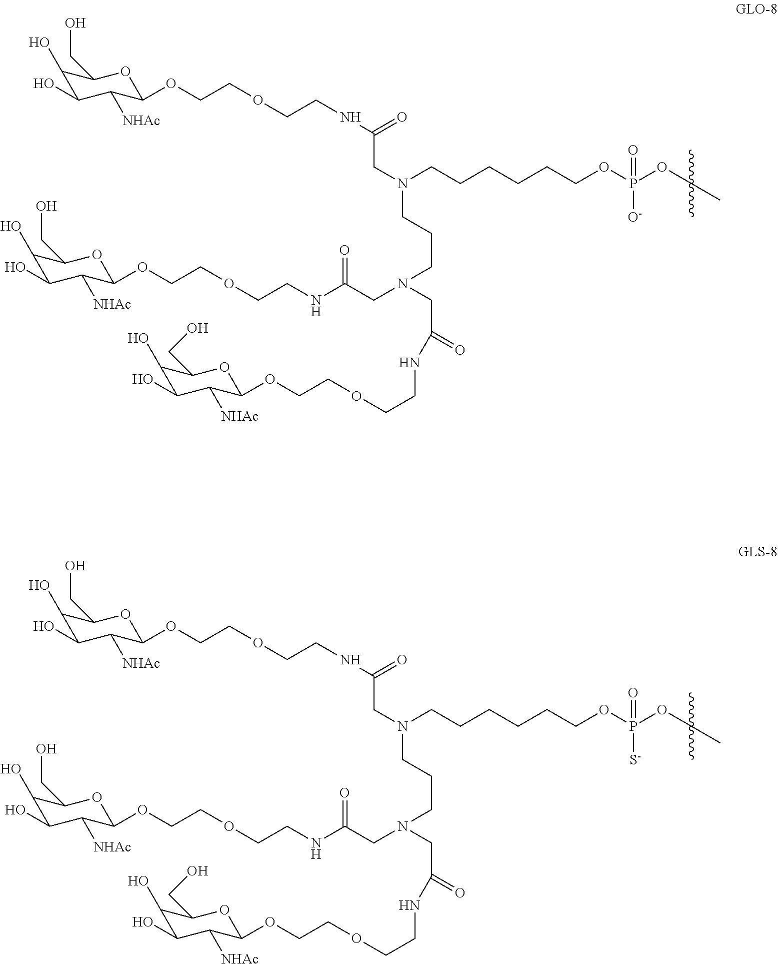 [SMILES: *OP(=O)([O-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

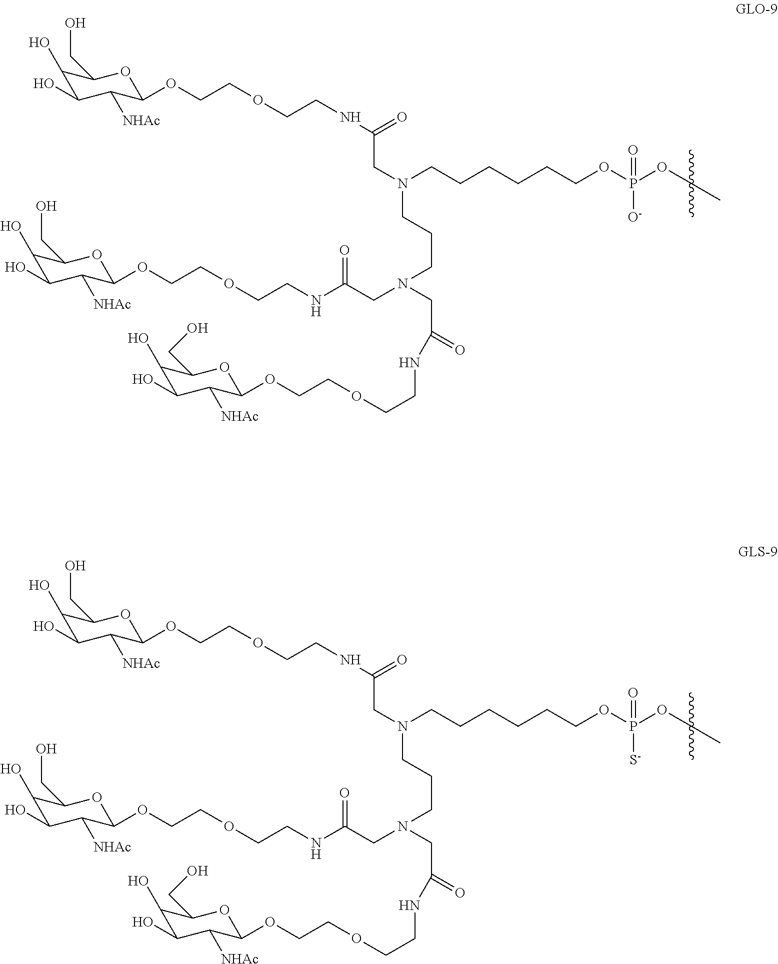 [SMILES: *OP(=O)([O-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

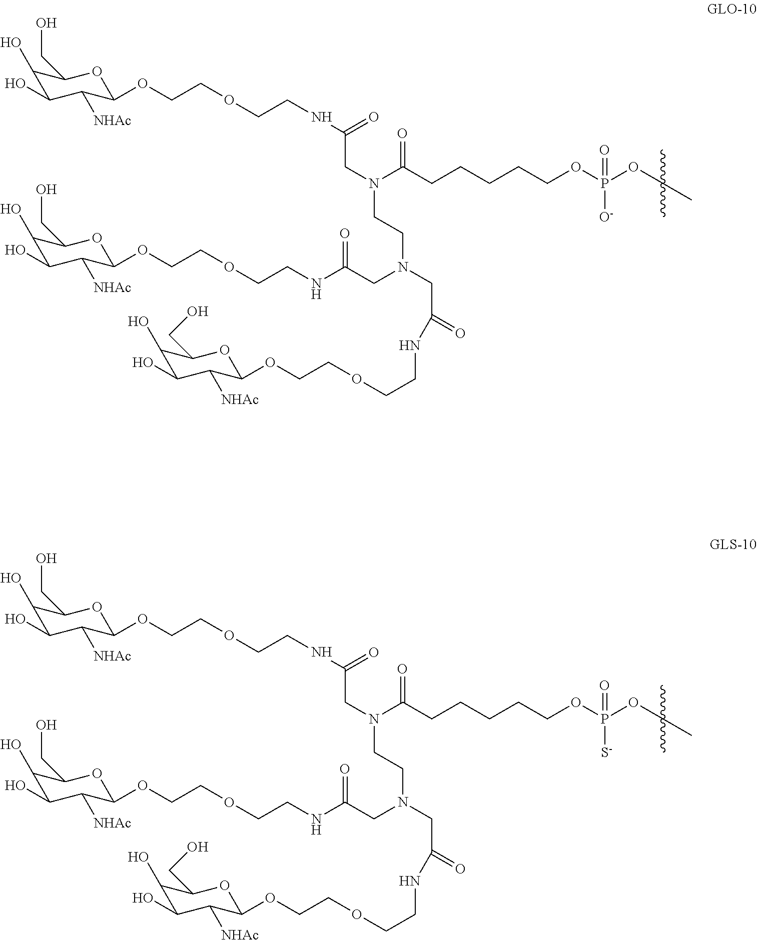 [SMILES: *OP(=O)([O-])OCCCCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

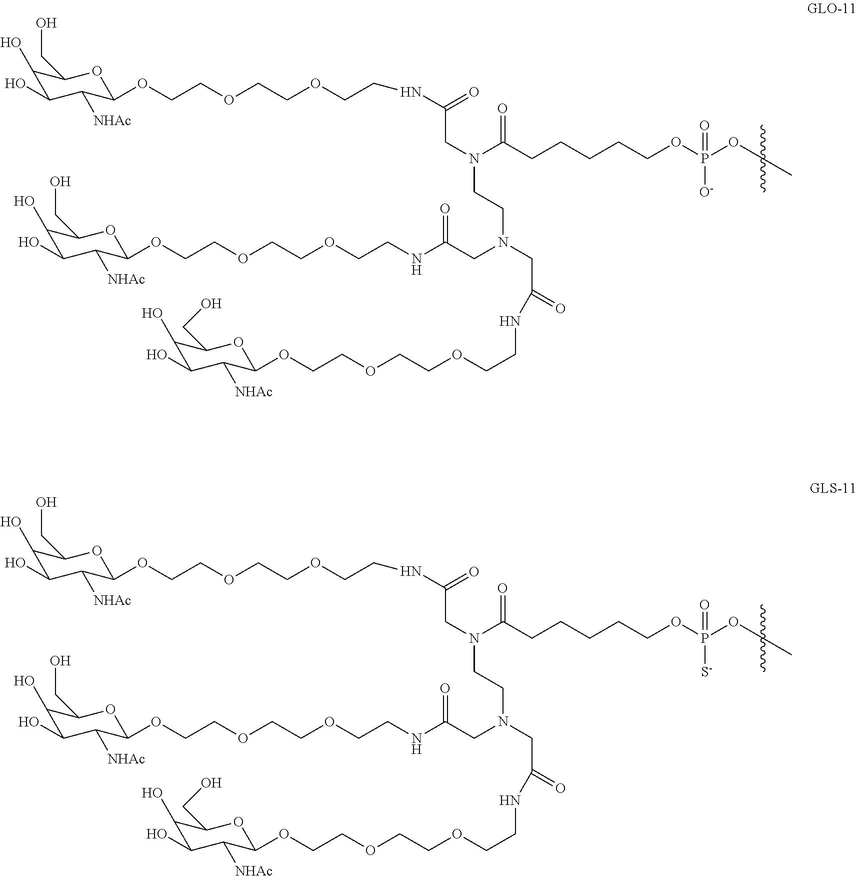 [SMILES: *OP(=O)([O-])OCCCCCC(=O)N(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCCCCC(=O)N(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

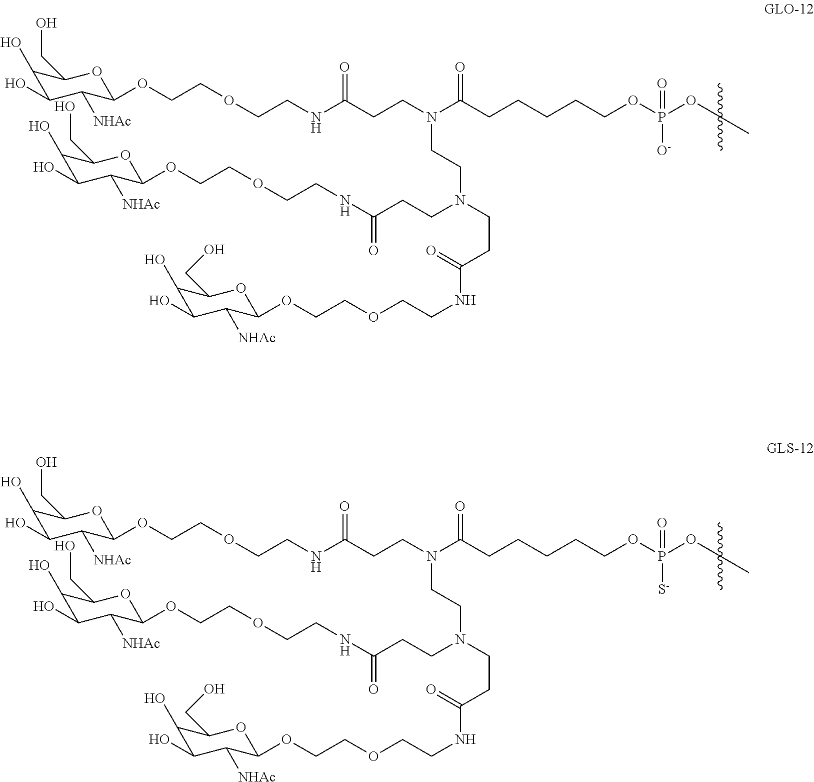 [SMILES: *OP(=O)([O-])OCCCCCC(=O)N(CCC(=O)NCCOCCOC1OC(CO)C(O)C(O)C1NC(C)=O)CCN(CCC(=O)NCCOCCOC1OC(CO)C(O)C(O)C1NC(C)=O)CCC(=O)NCCOCCOC1OC(CO)C(O)C(O)C1NC(C)=O ; *OP(=O)([S-])OCCCCCC(=O)N(CCC(=O)NCCOCCOC1OC(CO)C(O)C(O)C1NC(C)=O)CCN(CCC(=O)NCCOCCOC1OC(CO)C(O)C(O)C1NC(C)=O)CCC(=O)NCCOCCOC1OC(CO)C(O)C(O)C1NC(C)=O]

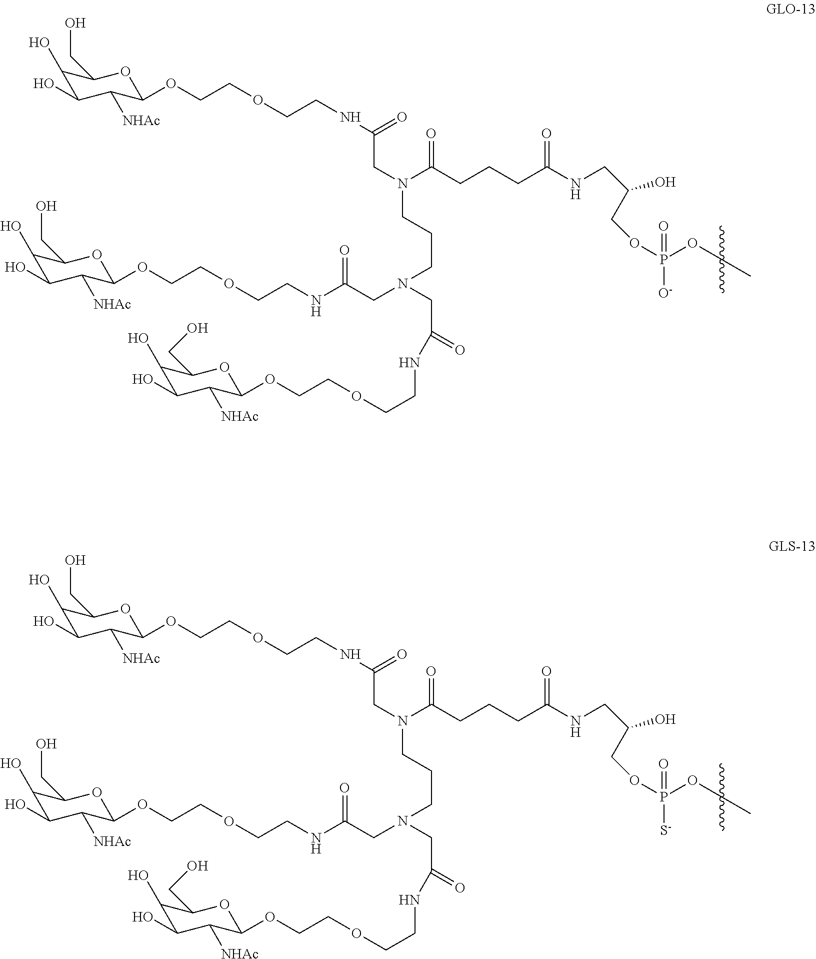 [SMILES: *OP(=O)([O-])OC[C@@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OC[C@@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

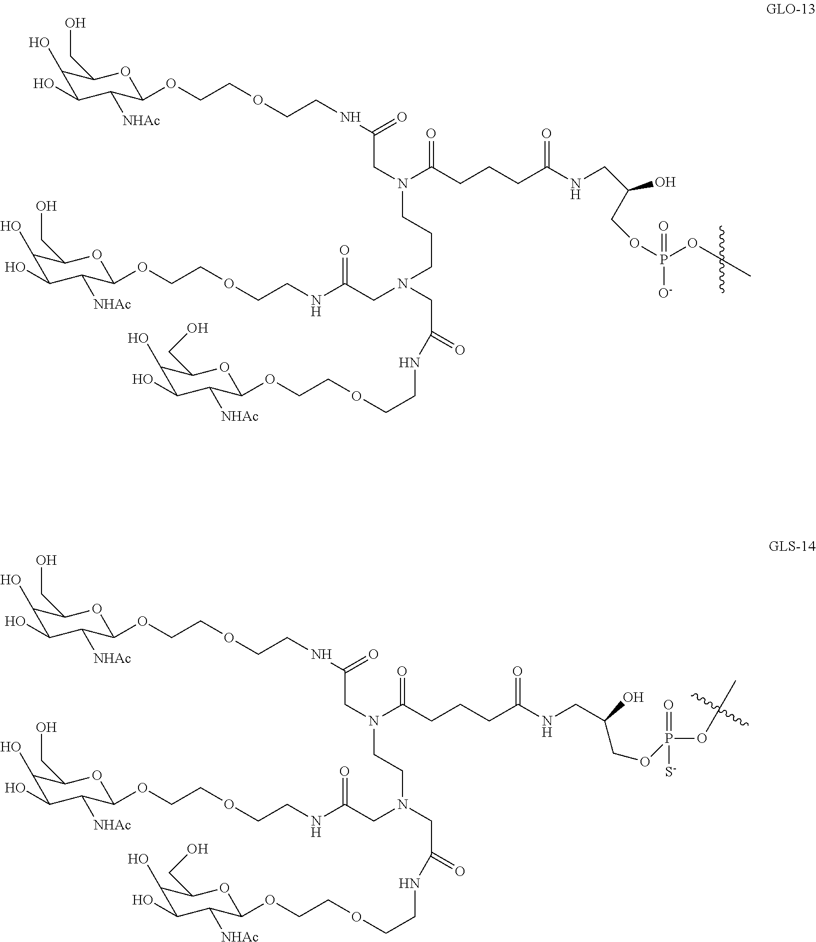 [SMILES: *OP(=O)([O-])OC[C@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OC[C@H](O)CNC(=O)CCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

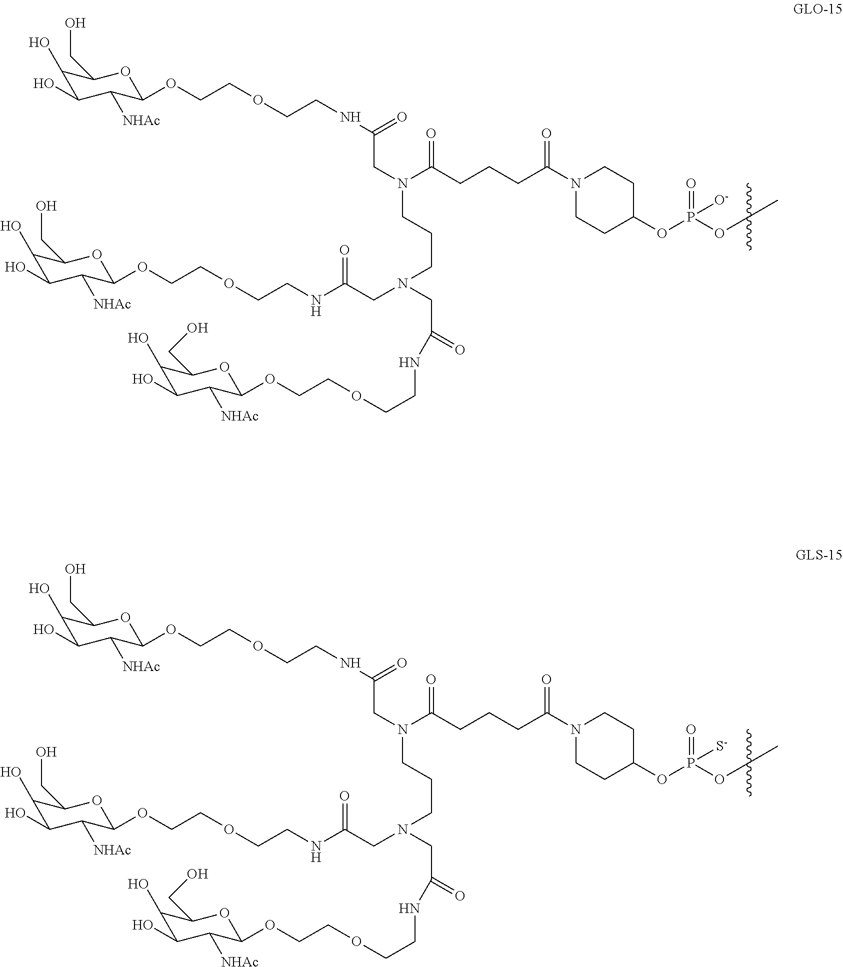 [SMILES: *OP(=O)([O-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1 ; *OP(=O)([S-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1]

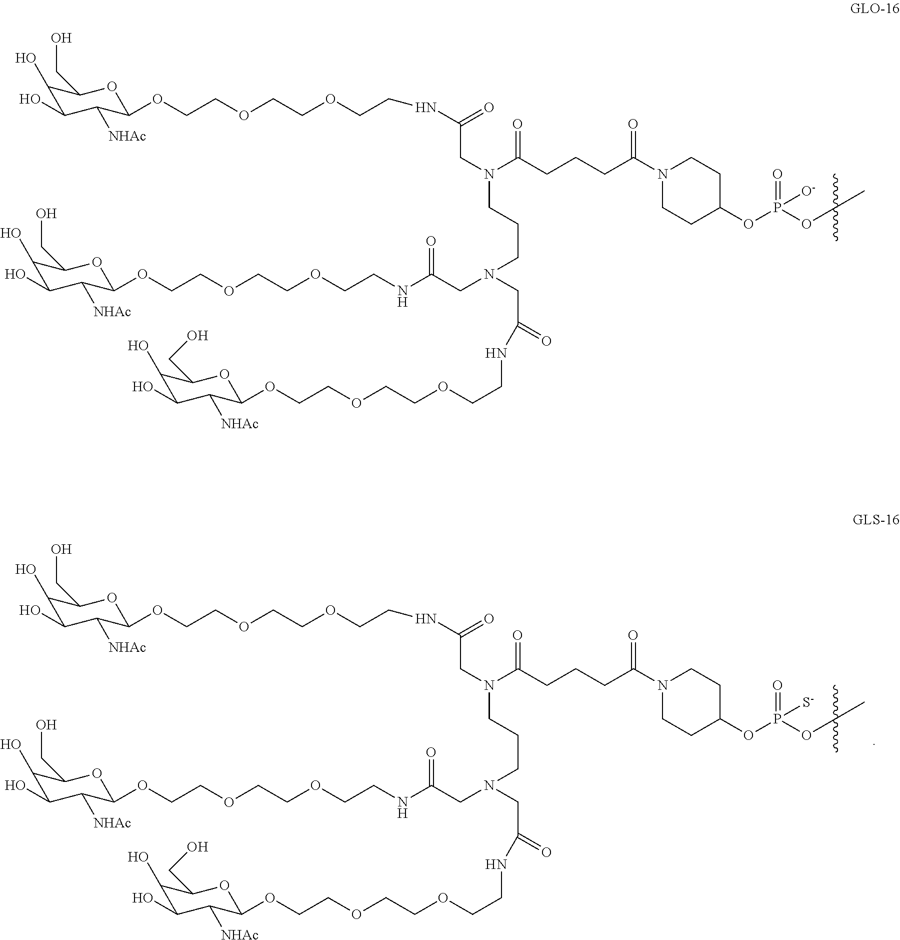 [SMILES: *OP(=O)([O-])OC1CCN(C(=O)CCCC(=O)N(CCCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOCCOC2OC(CO)C(O)C(O)C2NC(C)=O)CC1 ; *OP(=O)([S-])OC1CCN(C(=O)CCCC(=O)N(CCCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOCCOC2OC(CO)C(O)C(O)C2NC(C)=O)CC1]

27-29. (canceled)

30. The dsRNA agent of claim 1, wherein the dsRNA agent comprises a targeting group conjugated to the 5′-terminal or 3′-terminal end of the sense strand.

31. (canceled)

32. The dsRNA agent of claim 1, wherein the antisense strand comprises an inverted abasic residue at the 3′-terminal end; or
the sense strand comprises one or two inverted abasic residues at the 3′ and/or 5′ ends.

33. (canceled)

34. The dsRNA agent of claim 1, wherein the dsRNA agent has two blunt ends; or
at least one strand comprises a 3′ overhang of at least 1 or 2 nucleotide.

35-36. (canceled)

37. A composition comprising the dsRNA agent of claim 1,
optionally, the composition further comprises a pharmaceutically acceptable carrier;
optionally, the composition further comprises one or more additional therapeutic agents.

38-39. (canceled)

40. The composition of claim 37, wherein the composition is packaged in a kit, container, pack, dispenser, pre-filled syringe, or vial.

41. The composition of claim 37, wherein the composition is formulated for subcutaneous administration or is formulated for intravenous (IV) administration.

42. A cell comprising the dsRNA agent of claim 1, optionally, the cell is a mammalian cell, optionally a human cell.

43. (canceled)

44. A method of inhibiting AGT gene expression in a cell, comprising:
(i) preparing cells comprising an effective amount of the double-stranded ribonucleic acid (dsRNA) agent claim 1;
optionally, the method further comprises (ii) maintaining the cells for sufficient time to obtain degradation of the mRNA transcript of the AGT gene, thereby inhibiting the expression of the AGT gene in the cells.

45-50. (canceled)

51. A method of inhibiting AGT gene expression in a subject, the method comprising administering to the subject an effective amount of the double-stranded ribonucleic acid (dsRNA) agent claim 1;
optionally, the dsRNA agent is administered to the subject subcutaneously or by IV administration.

52-53. (canceled)

54. The method of claim 51, further comprising assessing inhibition of the AGT gene after administering the dsRNA agent to the subject, wherein the means for assessing include:
(i) determine one or more physiological characteristics of an AGT-associated disease or condition in the subject, and
(ii) comparing the determined physiological characteristics with the baseline pre-treatment physiological characteristics of the AGT-associated disease or condition and/or the control physiological characteristics of the AGT-associated disease or condition,
wherein the comparison indicates one or more of the presence or absence of inhibition of expression of the AGT gene in the subject;
optionally, the determined physiological characteristic is hypertension, which includes essential hypertension, secondary hypertension, hypertensive emergencies (such as malignant hypertension and accelerated hypertension), acute Hypertension, pregnancy-related hypertension, Refractory hypertension;
preferably, a reduction in the subject's blood pressure indicates a reduction of AGT gene expression in the subject.

55-56. (canceled)

57. A method for treating a disease or condition associated with an AGT protein, the method comprising administering an effective amount of the double-stranded ribonucleic acid (dsRNA) agent of claim 1, to inhibit AGT gene expression;
optionally, the disease or condition is caused by or associated with: activation of the renin-angiotensin-aldosterone system (RAAS) or a symptom or progression thereof in response to activation of the RAAS, The disease or condition is usually associated with hypertension, including but not limited to one or more of the following: Hypertensive disease, hypertension, borderline hypertension, essential hypertension, secondary hypertension, isolated Systolic or diastolic hypertension, pregnancy-associated hypertension, diabetic hypertension, Refractory hypertension, refractory hypertension, Paroxysmal hypertension, renovascular hypertension, Goldblatt's hypertension, ocular hypertension, glaucoma, pulmonary hypertension, portal hypertension, systemic venous hypertension, systolic hypertension, unstable hypertension; hypertensive heart disease, hypertensive nephropathy, atherosclerosis sclerosis, arteriosclerosis, vascular disease, diabetic nephropathy, diabetic retinopathy, chronic heart failure, cardiomyopathy, diabetic cardiomyopathy, glomerulosclerosis, aortic stenosis, aortic aneurysm, ventricular fibrosis, heart failure, myocardial infarction, angina, stroke, kidney disease, renal failure, systemic sclerosis, intrauterine growth retardation (IUGR), fetal growth restriction.

58. (canceled)

59. The method of claim 57, further comprising administering to the subject an additional treatment regime;
optionally, the additional treatment regimen comprises: administering to the subject one or more AGT antisense polynucleotides of the invention, administering to the subject a non-AGT dsRNA therapeutic agent, and a behavioral modification in the subject,
optionally, the non-AGT dsRNA therapeutic agent is one or more of the following: additional therapeutic agents such as diuretics, angiotensin converting enzyme (ACE) inhibitors, vascular Tensin II receptor antagonists, beta-blockers, vasodilators, calcium channel blockers, aldosterone antagonists, alpha2-agonists, renin inhibitors, alpha-blockers, peripherally acting adrenergic agents, selective D1 receptor partial agonists, non-selective alpha-adrenergic antagonists, synthetic, steroidal antimineralocorticoids, or combinations of any of the foregoing, and therapeutic agents for hypertension formulated as combinations of agents.

60-61. (canceled)

62. The method of claim 57, wherein the dsRNA agent is administered to the subject subcutaneously or by IV administration.

63. (canceled)

64. The method of claim 57, further comprising determining the efficacy of the administered double-stranded ribonucleic acid (dsRNA) agent in the subject preferably, the means for determining the efficacy of treatment in the subject comprises:
(i) determining one or more physiological characteristics of an AGT-associated disease or condition in a subject, and
(ii) comparing the identified physical characteristics with the baseline pre-treatment physical characteristics of the AGT-associated disease or condition,
wherein the comparison indicates one or more of the presence, absence, and level of efficacy of administering the double-stranded ribonucleic acid (dsRNA) agent to the subject;
optionally, the determined physiological characteristic is: hypertension, which includes essential hypertension, secondary hypertension, hypertensive emergencies (such as malignant hypertension and accelerated hypertension), Acute hypertension, pregnancy-related hypertension, Refractory hypertension;
preferably, a reduction in the subject's blood pressure indicates the presence of efficacy of the administration of the double-stranded ribonucleic acid (dsRNA) agent to the subject.

65-67. (canceled)

68. A method for decreasing a level of AGT protein in a subject compared to a baseline pre-treatment level of AGT protein in the subject, said method comprising administering to the subject an effective amount of the double-stranded ribonucleic acid (dsRNA) of claim 1, to reduce the level of AGT gene expression;
optionally, the dsRNA agent is administered to the subject subcutaneously or IV.

69. (canceled)

70. A method for altering the physiological characteristics of an AGT-associated disease or condition in a subject compared to the baseline pre-treatment physiological characteristics of the AGT-associated disease or condition in the subject, said method comprising administering to a subject an effective amount of a double-stranded ribonucleic acid (dsRNA) agent of claim 1, to change the physiological characteristics of an AGT-associated disease or condition in a subject;
optionally, the dsRNA agent is administered to the subject subcutaneously or IV,
optionally, the physiological characteristic is hypertension, which includes essential hypertension, secondary hypertension, hypertensive emergencies (such as malignant hypertension and accelerated hypertension), acute hypertensive Blood pressure, pregnancy-related hypertension, Refractory hypertension.

71-72. (canceled)

73. The dsRNA agent of claim 1, comprising a sense strand that differs by 0, 1, 2, or 3 nucleotides from formula (A): 5′-Z₁AGCUUGUUUGUGAAACZ₂-3′ formula (A) (SEQ ID NO: 656), wherein Z₁ is a nucleotide sequence comprising 0-15 nucleotides motifs, Z₂ is selected from one of A, U, C, G or absent, or
comprising an antisense strand differing by 0, 1, 2, or 3 nucleotides from formula (B): 5′-Z₃GUUUCACAAACAAGCUZ₄-3′ formula (B) (SEQ ID NO: 657), wherein Z₃ is selected from one of A, U, C, G or absent, and Z₄ is a nucleotide sequence comprising 0-nucleotides motifs.

74. (canceled)


SEQUENCE LISTING (673 sequences)

SEQ ID NO: 1 - RNA, 21 nt, synthetic construct
  source 1..21: mol_type = other RNA
gcgtcatccacaatgagagta

SEQ ID NO: 2 - RNA, 21 nt, synthetic construct
  source 1..21: mol_type = other RNA
gtcatccacaatgagagtaca

SEQ ID NO: 3 - RNA, 21 nt, synthetic construct
  source 1..21: mol_type = other RNA
gatccacaatgagagtaccta

SEQ ID NO: 4 - RNA, 21 nt, synthetic construct
  source 1..21: mol_type = other RNA
gtccacaatgagagtacctga

SEQ ID NO: 5 - RNA, 21 nt, synthetic construct
  source 1..21: mol_type = other RNA
gttcttgggcttccgtatata

SEQ ID NO: 6 - RNA, 21 nt, synthetic construct
  source 1..21: mol_type = other RNA
gttgggcttccgtatatatga

SEQ ID NO: 7 - RNA, 21 nt, synthetic construct
  source 1..21: mol_type = other RNA
gtgggcttccgtatatatgga

SEQ ID NO: 8 - RNA, 21 nt, synthetic construct
  source 1..21: mol_type = other RNA
ggcttccgtatatatggcata

SEQ ID NO: 9 - RNA, 21 nt, synthetic construct
  source 1..21: mol_type = other RNA
gcttccgtatatatggcatga

SEQ ID NO: 10 - RNA, 21 nt, synthetic construct
  source 1..21: mol_type = other RNA
gcgtatatatggcatgcacaa

SEQ ID NO: 11 - RNA, 21 nt, synthetic construct
  source 1..21: mol_type = other RNA
ggttccttggaaggacaagaa

SEQ ID NO: 12 - RNA, 21 nt, synthetic construct
  source 1..21: mol_type = other RNA
gagaagattgacaggttcata

SEQ ID NO: 13 - RNA, 21 nt, synthetic construct
  source 1..21: mol_type = other RNA
gatgcaggctgtgacaggata

SEQ ID NO: 14 - RNA, 21 nt, synthetic construct
  source 1..21: mol_type = other RNA
ggagttctgggtggacaacaa

SEQ ID NO: 15 - RNA, 21 nt, synthetic construct
  source 1..21: mol_type = other RNA
gcaacagcacctcagtgtcta

SEQ ID NO: 16 - RNA, 21 nt, synthetic construct
  source 1..21: mol_type = other RNA
ggggtctcactttccagcaaa

SEQ ID NO: 17 - RNA, 21 nt, synthetic construct
  source 1..21: mol_type = other RNA
gtcactttccagcaaaactca

SEQ ID NO: 18 - RNA, 21 nt, synthetic construct
  source 1..21: mol_type = other RNA
gccagcaaaactccctcaaca

SEQ ID NO: 19 - RNA, 21 nt, synthetic construct
  source 1..21: mol_type = other RNA
gagcaaaactccctcaactga

SEQ ID NO: 20 - RNA, 21 nt, synthetic construct
  source 1..21: mol_type = other RNA
ggagctgaacctgcaaaaata

SEQ ID NO: 21 - RNA, 21 nt, synthetic construct
  source 1..21: mol_type = other RNA
gctgaacctgcaaaaattgaa

SEQ ID NO: 22 - RNA, 21 nt, synthetic construct
  source 1..21: mol_type = other RNA
ggaaccgcccattcctgttta

SEQ ID NO: 23 - RNA, 21 nt, synthetic construct
  source 1..21: mol_type = other RNA
gaaccgcccattcctgtttga

SEQ ID NO: 24 - RNA, 21 nt, synthetic construct
  source 1..21: mol_type = other RNA
gttcctgtttgctgtgtatga

SEQ ID NO: 25 - RNA, 21 nt, synthetic construct
  source 1..21: mol_type = other RNA
gctgtttgctgtgtatgatca

SEQ ID NO: 26 - RNA, 21 nt, synthetic construct
  source 1..21: mol_type = other RNA
gtgtttgctgtgtatgatcaa

SEQ ID NO: 27 - RNA, 21 nt, synthetic construct
  source 1..21: mol_type = other RNA
gttgctgtgtatgatcaaaga

SEQ ID NO: 28 - RNA, 21 nt, synthetic construct
  source 1..21: mol_type = other RNA
gtcccaccttttcttctaata

SEQ ID NO: 29 - RNA, 21 nt, synthetic construct
  source 1..21: mol_type = other RNA
gaccttttcttctaatgagta

SEQ ID NO: 30 - RNA, 21 nt, synthetic construct
  source 1..21: mol_type = other RNA
gccttttcttctaatgagtca

SEQ ID NO: 31 - RNA, 21 nt, synthetic construct
  source 1..21: mol_type = other RNA
gcgtttctccttggtctaaga

SEQ ID NO: 32 - RNA, 21 nt, synthetic construct
  source 1..21: mol_type = other RNA
gtttctccttggtctaagtga

SEQ ID NO: 33 - RNA, 21 nt, synthetic construct
  source 1..21: mol_type = other RNA
ggtttgctgggtttattttaa

SEQ ID NO: 34 - RNA, 21 nt, synthetic construct
  source 1..21: mol_type = other RNA
gtttgctgggtttattttaga

SEQ ID NO: 35 - RNA, 21 nt, synthetic construct
  source 1..21: mol_type = other RNA
gttgctgggtttattttagaa

SEQ ID NO: 36 - RNA, 21 nt, synthetic construct
  source 1..21: mol_type = other RNA
ggggtttattttagagaatga

SEQ ID NO: 37 - RNA, 21 nt, synthetic construct
  source 1..21: mol_type = other RNA
gggtttattttagagaatgga

SEQ ID NO: 38 - RNA, 21 nt, synthetic construct
  source 1..21: mol_type = other RNA
gggcaagaaccagtgtttaga

SEQ ID NO: 39 - RNA, 21 nt, synthetic construct
  source 1..21: mol_type = other RNA
ggcaagaaccagtgtttagca

SEQ ID NO: 40 - RNA, 21 nt, synthetic construct
  source 1..21: mol_type = other RNA
gcaagaaccagtgtttagcga

SEQ ID NO: 41 - RNA, 21 nt, synthetic construct
  source 1..21: mol_type = other RNA
gctgttccaaaaagaattcca

SEQ ID NO: 42 - RNA, 21 nt, synthetic construct
  source 1..21: mol_type = other RNA
ggttccaaaaagaattccaaa

SEQ ID NO: 43 - RNA, 21 nt, synthetic construct
  source 1..21: mol_type = other RNA
gttccaaaaagaattccaaca

SEQ ID NO: 44 - RNA, 21 nt, synthetic construct
  source 1..21: mol_type = other RNA
gcaaaaagaattccaaccgaa

SEQ ID NO: 45 - RNA, 21 nt, synthetic construct
  source 1..21: mol_type = other RNA
gaaaaagaattccaaccgaca

SEQ ID NO: 46 - RNA, 21 nt, synthetic construct
  source 1..21: mol_type = other RNA
gcaaccgaccagcttgtttga

SEQ ID NO: 47 - RNA, 21 nt, synthetic construct
  source 1..21: mol_type = other RNA
gaaccgaccagcttgtttgta

SEQ ID NO: 48 - RNA, 21 nt, synthetic construct
  source 1..21: mol_type = other RNA
ggaccagcttgtttgtgaaaa

SEQ ID NO: 49 - RNA, 21 nt, synthetic construct
  source 1..21: mol_type = other RNA
gaccagcttgtttgtgaaaca

SEQ ID NO: 50 - RNA, 21 nt, synthetic construct
  source 1..21: mol_type = other RNA
gccagcttgtttgtgaaacaa

SEQ ID NO: 51 - RNA, 21 nt, synthetic construct
  source 1..21: mol_type = other RNA
gtcatccacaatgagagtaca

SEQ ID NO: 52 - RNA, 21 nt, synthetic construct
  source 1..21: mol_type = other RNA
gtgggcttccgtatatatgga

SEQ ID NO: 53 - RNA, 21 nt, synthetic construct
  source 1..21: mol_type = other RNA
gctgtttgctgtgtatgatca

SEQ ID NO: 54 - RNA, 21 nt, synthetic construct
  source 1..21: mol_type = other RNA
ggtttgctgggtttattttaa

SEQ ID NO: 55 - RNA, 21 nt, synthetic construct
  source 1..21: mol_type = other RNA
gccagcttgtttgtgaaacaa

SEQ ID NO: 56 - RNA, 21 nt, synthetic construct
  source 1..21: mol_type = other RNA
gtcccaccttttcttctaata

SEQ ID NO: 57 - RNA, 21 nt, synthetic construct
  source 1..21: mol_type = other RNA
gccttttcttctaatgagtca

SEQ ID NO: 58 - RNA, 21 nt, synthetic construct
  source 1..21: mol_type = other RNA
ggggtttattttagagaatga

SEQ ID NO: 59 - RNA, 21 nt, synthetic construct
  source 1..21: mol_type = other RNA
ggttccaaaaagaattccaaa

SEQ ID NO: 60 - RNA, 21 nt, synthetic construct
  source 1..21: mol_type = other RNA
gttccaaaaagaattccaaca

SEQ ID NO: 61 - RNA, 21 nt, synthetic construct
  source 1..21: mol_type = other RNA
catccacaatgagagtaccta

SEQ ID NO: 62 - RNA, 21 nt, synthetic construct
  source 1..21: mol_type = other RNA
cttcttgggcttccgtatata

SEQ ID NO: 63 - RNA, 21 nt, synthetic construct
  source 1..21: mol_type = other RNA
catgcaggctgtgacaggata

SEQ ID NO: 64 - RNA, 21 nt, synthetic construct
  source 1..21: mol_type = other RNA
gctgaacctgcaaaaattgaa

SEQ ID NO: 65 - RNA, 21 nt, synthetic construct
  source 1..21: mol_type = other RNA
caccttttcttctaatgagta

SEQ ID NO: 66 - RNA, 21 nt, synthetic construct
  source 1..21: mol_type = other RNA
ccgtttctccttggtctaaga

SEQ ID NO: 67 - RNA, 21 nt, synthetic construct
  source 1..21: mol_type = other RNA
actgttccaaaaagaattcca

SEQ ID NO: 68 - RNA, 21 nt, synthetic construct
  source 1..21: mol_type = other RNA
caaaaagaattccaaccgaca

SEQ ID NO: 69 - RNA, 21 nt, synthetic construct
  source 1..21: mol_type = other RNA
cgaccagcttgtttgtgaaaa

SEQ ID NO: 70 - RNA, 21 nt, synthetic construct
  source 1..21: mol_type = other RNA
gaccagcttgtttgtgaaaca

SEQ ID NO: 71 - RNA, 21 nt, synthetic construct
  source 1..21: mol_type = other RNA
tcgtcatccacaatgagagta

SEQ ID NO: 72 - RNA, 21 nt, synthetic construct
  source 1..21: mol_type = other RNA
gtccacaatgagagtacctga

SEQ ID NO: 73 - RNA, 21 nt, synthetic construct
  source 1..21: mol_type = other RNA
agggtctcactttccagcaaa

SEQ ID NO: 74 - RNA, 21 nt, synthetic construct
  source 1..21: mol_type = other RNA
ctgtttgctgtgtatgatcaa

SEQ ID NO: 75 - RNA, 21 nt, synthetic construct
  source 1..21: mol_type = other RNA
tttgctgtgtatgatcaaaga

SEQ ID NO: 76 - RNA, 21 nt, synthetic construct
  source 1..21: mol_type = other RNA
gtttctccttggtctaagtga

SEQ ID NO: 77 - RNA, 21 nt, synthetic construct
  source 1..21: mol_type = other RNA
gcaagaaccagtgtttagcga

SEQ ID NO: 78 - RNA, 21 nt, synthetic construct
  source 1..21: mol_type = other RNA
ccaaaaagaattccaaccgaa

SEQ ID NO: 79 - RNA, 21 nt, synthetic construct
  source 1..21: mol_type = other RNA
caaccgaccagcttgtttgta

SEQ ID NO: 80 - RNA, 21 nt, synthetic construct
  source 1..21: mol_type = other RNA
gaccttttcttctaatgagta

SEQ ID NO: 81 - RNA, 21 nt, synthetic construct
  source 1..21: mol_type = other RNA
gacctttctttctagcgagta

SEQ ID NO: 82 - RNA, 21 nt, synthetic construct
  source 1..21: mol_type = other RNA
gaccttttcttctaatgagta

SEQ ID NO: 83 - RNA, 21 nt, synthetic construct
  source 1..21: mol_type = other RNA
caccagcttgtttgtgaaaca

SEQ ID NO: 84 - RNA, 21 nt, synthetic construct
  source 1..21: mol_type = other RNA
caccagcttgtttgtgaaaca

SEQ ID NO: 85 - RNA, 21 nt, synthetic construct
  source 1..21: mol_type = other RNA
gcgtttctccttggtctaaga

SEQ ID NO: 86 - RNA, 21 nt, synthetic construct
  source 1..21: mol_type = other RNA
gcgtttctccttggtctaaga

SEQ ID NO: 87 - RNA, 21 nt, synthetic construct
  source 1..21: mol_type = other RNA
gcaaaaagaattccaaccgaa

SEQ ID NO: 88 - RNA, 19 nt, synthetic construct
  source 1..19: mol_type = other RNA
ccttttcttctaatgagta

SEQ ID NO: 89 - RNA, 19 nt, synthetic construct
  source 1..19: mol_type = other RNA
ccagcttgtttgtgaaaca

SEQ ID NO: 90 - RNA, 19 nt, synthetic construct
  source 1..19: mol_type = other RNA
gtttctccttggtctaaga

SEQ ID NO: 91 - RNA, 19 nt, synthetic construct
  source 1..19: mol_type = other RNA
aaaaagaattccaaccgaa

SEQ ID NO: 92 - RNA, 19 nt, synthetic construct
  source 1..19: mol_type = other RNA
cccaccttttcttctaata

SEQ ID NO: 93 - RNA, 19 nt, synthetic construct
  source 1..19: mol_type = other RNA
ccttttcttctaatgagtt

SEQ ID NO: 94 - RNA, 19 nt, synthetic construct
  source 1..19: mol_type = other RNA
ccagcttgtttgtgaaact

SEQ ID NO: 95 - RNA, 19 nt, synthetic construct
  source 1..19: mol_type = other RNA
gtttctccttggtctaagt

SEQ ID NO: 96 - RNA, 19 nt, synthetic construct
  source 1..19: mol_type = other RNA
aaaaagaattccaaccgat

SEQ ID NO: 97 - RNA, 19 nt, synthetic construct
  source 1..19: mol_type = other RNA
cccaccttttcttctaatt

SEQ ID NO: 98 - RNA, 21 nt, synthetic construct
  source 1..21: mol_type = other RNA
tactctcattgtggatgacgc

SEQ ID NO: 99 - RNA, 21 nt, synthetic construct
  source 1..21: mol_type = other RNA
tgtactctcattgtggatgac

SEQ ID NO: 100 - RNA, 21 nt, synthetic construct
  source 1..21: mol_type = other RNA
taggtactctcattgtggatc

SEQ ID NO: 101 - RNA, 21 nt, synthetic construct
  source 1..21: mol_type = other RNA
tcaggtactctcattgtggac

SEQ ID NO: 102 - RNA, 21 nt, synthetic construct
  source 1..21: mol_type = other RNA
tatatacggaagcccaagaac

SEQ ID NO: 103 - RNA, 21 nt, synthetic construct
  source 1..21: mol_type = other RNA
tcatatatacggaagcccaac

SEQ ID NO: 104 - RNA, 21 nt, synthetic construct
  source 1..21: mol_type = other RNA
tccatatatacggaagcccac

SEQ ID NO: 105 - RNA, 21 nt, synthetic construct
  source 1..21: mol_type = other RNA
tatgccatatatacggaagcc

SEQ ID NO: 106 - RNA, 21 nt, synthetic construct
  source 1..21: mol_type = other RNA
tcatgccatatatacggaagc

SEQ ID NO: 107 - RNA, 21 nt, synthetic construct
  source 1..21: mol_type = other RNA
ttgtgcatgccatatatacgc

SEQ ID NO: 108 - RNA, 21 nt, synthetic construct
  source 1..21: mol_type = other RNA
ttcttgtccttccaaggaacc

SEQ ID NO: 109 - RNA, 21 nt, synthetic construct
  source 1..21: mol_type = other RNA
tatgaacctgtcaatcttctc

SEQ ID NO: 110 - RNA, 21 nt, synthetic construct
  source 1..21: mol_type = other RNA
tatcctgtcacagcctgcatc

SEQ ID NO: 111 - RNA, 21 nt, synthetic construct
  source 1..21: mol_type = other RNA
ttgttgtccacccagaactcc

SEQ ID NO: 112 - RNA, 21 nt, synthetic construct
  source 1..21: mol_type = other RNA
tagacactgaggtgctgttgc

SEQ ID NO: 113 - RNA, 21 nt, synthetic construct
  source 1..21: mol_type = other RNA
tttgctggaaagtgagacccc

SEQ ID NO: 114 - RNA, 21 nt, synthetic construct
  source 1..21: mol_type = other RNA
tgagttttgctggaaagtgac

SEQ ID NO: 115 - RNA, 21 nt, synthetic construct
  source 1..21: mol_type = other RNA
tgttgagggagttttgctggc

SEQ ID NO: 116 - RNA, 21 nt, synthetic construct
  source 1..21: mol_type = other RNA
tcagttgagggagttttgctc

SEQ ID NO: 117 - RNA, 21 nt, synthetic construct
  source 1..21: mol_type = other RNA
tatttttgcaggttcagctcc

SEQ ID NO: 118 - RNA, 21 nt, synthetic construct
  source 1..21: mol_type = other RNA
ttcaatttttgcaggttcagc

SEQ ID NO: 119 - RNA, 21 nt, synthetic construct
  source 1..21: mol_type = other RNA
taaacaggaatgggcggttcc

SEQ ID NO: 120 - RNA, 21 nt, synthetic construct
  source 1..21: mol_type = other RNA
tcaaacaggaatgggcggttc

SEQ ID NO: 121 - RNA, 21 nt, synthetic construct
  source 1..21: mol_type = other RNA
tcatacacagcaaacaggaac

SEQ ID NO: 122 - RNA, 21 nt, synthetic construct
  source 1..21: mol_type = other RNA
tgatcatacacagcaaacagc

SEQ ID NO: 123 - RNA, 21 nt, synthetic construct
  source 1..21: mol_type = other RNA
ttgatcatacacagcaaacac

SEQ ID NO: 124 - RNA, 21 nt, synthetic construct
  source 1..21: mol_type = other RNA
tctttgatcatacacagcaac

SEQ ID NO: 125 - RNA, 21 nt, synthetic construct
  source 1..21: mol_type = other RNA
tattagaagaaaaggtgggac

SEQ ID NO: 126 - RNA, 21 nt, synthetic construct
  source 1..21: mol_type = other RNA
tactcattagaagaaaaggtc

SEQ ID NO: 127 - RNA, 21 nt, synthetic construct
  source 1..21: mol_type = other RNA
tgactcattagaagaaaaggc

SEQ ID NO: 128 - RNA, 21 nt, synthetic construct
  source 1..21: mol_type = other RNA
tcttagaccaaggagaaacgc

SEQ ID NO: 129 - RNA, 21 nt, synthetic construct
  source 1..21: mol_type = other RNA
tcacttagaccaaggagaaac

SEQ ID NO: 130 - RNA, 21 nt, synthetic construct
  source 1..21: mol_type = other RNA
ttaaaataaacccagcaaacc

SEQ ID NO: 131 - RNA, 21 nt, synthetic construct
  source 1..21: mol_type = other RNA
tctaaaataaacccagcaaac

SEQ ID NO: 132 - RNA, 21 nt, synthetic construct
  source 1..21: mol_type = other RNA
ttctaaaataaacccagcaac

SEQ ID NO: 133 - RNA, 21 nt, synthetic construct
  source 1..21: mol_type = other RNA
tcattctctaaaataaacccc

SEQ ID NO: 134 - RNA, 21 nt, synthetic construct
  source 1..21: mol_type = other RNA
tccattctctaaaataaaccc

SEQ ID NO: 135 - RNA, 21 nt, synthetic construct
  source 1..21: mol_type = other RNA
tctaaacactggttcttgccc

SEQ ID NO: 136 - RNA, 21 nt, synthetic construct
  source 1..21: mol_type = other RNA
tgctaaacactggttcttgcc

SEQ ID NO: 137 - RNA, 21 nt, synthetic construct
  source 1..21: mol_type = other RNA
tcgctaaacactggttcttgc

SEQ ID NO: 138 - RNA, 21 nt, synthetic construct
  source 1..21: mol_type = other RNA
tggaattctttttggaacagc

SEQ ID NO: 139 - RNA, 21 nt, synthetic construct
  source 1..21: mol_type = other RNA
tttggaattctttttggaacc

SEQ ID NO: 140 - RNA, 21 nt, synthetic construct
  source 1..21: mol_type = other RNA
tgttggaattctttttggaac

SEQ ID NO: 141 - RNA, 21 nt, synthetic construct
  source 1..21: mol_type = other RNA
ttcggttggaattctttttgc

SEQ ID NO: 142 - RNA, 21 nt, synthetic construct
  source 1..21: mol_type = other RNA
tgtcggttggaattctttttc

SEQ ID NO: 143 - RNA, 21 nt, synthetic construct
  source 1..21: mol_type = other RNA
tcaaacaagctggtcggttgc

SEQ ID NO: 144 - RNA, 21 nt, synthetic construct
  source 1..21: mol_type = other RNA
tacaaacaagctggtcggttc

SEQ ID NO: 145 - RNA, 21 nt, synthetic construct
  source 1..21: mol_type = other RNA
ttttcacaaacaagctggtcc

SEQ ID NO: 146 - RNA, 21 nt, synthetic construct
  source 1..21: mol_type = other RNA
tgtttcacaaacaagctggtc

SEQ ID NO: 147 - RNA, 21 nt, synthetic construct
  source 1..21: mol_type = other RNA
ttgtttcacaaacaagctggc

SEQ ID NO: 148 - RNA, 21 nt, synthetic construct
  source 1..21: mol_type = other RNA
tgtactctcattgtggatgac

SEQ ID NO: 149 - RNA, 21 nt, synthetic construct
  source 1..21: mol_type = other RNA
tccatatatacggaagcccac

SEQ ID NO: 150 - RNA, 21 nt, synthetic construct
  source 1..21: mol_type = other RNA
tgatcatacacagcaaacagc

SEQ ID NO: 151 - RNA, 21 nt, synthetic construct
  source 1..21: mol_type = other RNA
ttaaaataaacccagcaaacc

SEQ ID NO: 152 - RNA, 21 nt, synthetic construct
  source 1..21: mol_type = other RNA
ttgtttcacaaacaagctggc

SEQ ID NO: 153 - RNA, 21 nt, synthetic construct
  source 1..21: mol_type = other RNA
tattagaagaaaaggtgggac

SEQ ID NO: 154 - RNA, 21 nt, synthetic construct
  source 1..21: mol_type = other RNA
tgactcattagaagaaaaggc

SEQ ID NO: 155 - RNA, 21 nt, synthetic construct
  source 1..21: mol_type = other RNA
tcattctctaaaataaacccc

SEQ ID NO: 156 - RNA, 21 nt, synthetic construct
  source 1..21: mol_type = other RNA
tttggaattctttttggaacc

SEQ ID NO: 157 - RNA, 21 nt, synthetic construct
  source 1..21: mol_type = other RNA
tgttggaattctttttggaac

SEQ ID NO: 158 - RNA, 21 nt, synthetic construct
  source 1..21: mol_type = other RNA
taggtactctcattgtggatg

SEQ ID NO: 159 - RNA, 21 nt, synthetic construct
  source 1..21: mol_type = other RNA
tatatacggaagcccaagaag

SEQ ID NO: 160 - RNA, 21 nt, synthetic construct
  source 1..21: mol_type = other RNA
tatcctgtcacagcctgcatg

SEQ ID NO: 161 - RNA, 21 nt, synthetic construct
  source 1..21: mol_type = other RNA
ttcaatttttgcaggttcagc

SEQ ID NO: 162 - RNA, 21 nt, synthetic construct
  source 1..21: mol_type = other RNA
tactcattagaagaaaaggtg

SEQ ID NO: 163 - RNA, 21 nt, synthetic construct
  source 1..21: mol_type = other RNA
tcttagaccaaggagaaacgg

SEQ ID NO: 164 - RNA, 21 nt, synthetic construct
  source 1..21: mol_type = other RNA
tggaattctttttggaacagt

SEQ ID NO: 165 - RNA, 21 nt, synthetic construct
  source 1..21: mol_type = other RNA
tgtcggttggaattctttttg

SEQ ID NO: 166 - RNA, 21 nt, synthetic construct
  source 1..21: mol_type = other RNA
ttttcacaaacaagctggtcg

SEQ ID NO: 167 - RNA, 21 nt, synthetic construct
  source 1..21: mol_type = other RNA
tgtttcacaaacaagctggtc

SEQ ID NO: 168 - RNA, 21 nt, synthetic construct
  source 1..21: mol_type = other RNA
tactctcattgtggatgacga

SEQ ID NO: 169 - RNA, 21 nt, synthetic construct
  source 1..21: mol_type = other RNA
tcaggtactctcattgtggac

SEQ ID NO: 170 - RNA, 21 nt, synthetic construct
  source 1..21: mol_type = other RNA
tttgctggaaagtgagaccct

SEQ ID NO: 171 - RNA, 21 nt, synthetic construct
  source 1..21: mol_type = other RNA
ttgatcatacacagcaaacag

SEQ ID NO: 172 - RNA, 21 nt, synthetic construct
  source 1..21: mol_type = other RNA
tctttgatcatacacagcaaa

SEQ ID NO: 173 - RNA, 21 nt, synthetic construct
  source 1..21: mol_type = other RNA
tcacttagaccaaggagaaac

SEQ ID NO: 174 - RNA, 21 nt, synthetic construct
  source 1..21: mol_type = other RNA
tcgctaaacactggttcttgc

SEQ ID NO: 175 - RNA, 21 nt, synthetic construct
  source 1..21: mol_type = other RNA
ttcggttggaattctttttgg

SEQ ID NO: 176 - RNA, 21 nt, synthetic construct
  source 1..21: mol_type = other RNA
tacaaacaagctggtcggttg

SEQ ID NO: 177 - RNA, 21 nt, synthetic construct
  source 1..21: mol_type = other RNA
tactcattagaagaaaaggtc

SEQ ID NO: 178 - RNA, 21 nt, synthetic construct
  source 1..21: mol_type = other RNA
tactcattagaagaaaaggtc

SEQ ID NO: 179 - RNA, 21 nt, synthetic construct
  source 1..21: mol_type = other RNA
tactcattagaagaaaaggtc

SEQ ID NO: 180 - RNA, 21 nt, synthetic construct
  source 1..21: mol_type = other RNA
tgtttcacaaacaagctggtg

SEQ ID NO: 181 - RNA, 21 nt, synthetic construct
  source 1..21: mol_type = other RNA
tgtttcacaaacaagctggtg

SEQ ID NO: 182 - RNA, 21 nt, synthetic construct
  source 1..21: mol_type = other RNA
tcttagaccaaggagaaacgc

SEQ ID NO: 183 - RNA, 21 nt, synthetic construct
  source 1..21: mol_type = other RNA
tcttagaccaaggagaaacgc

SEQ ID NO: 184 - RNA, 21 nt, synthetic construct
  source 1..21: mol_type = other RNA
ttcggttggaattctttttgc

SEQ ID NO: 185 - RNA, 19 nt, synthetic construct
  source 1..19: mol_type = other RNA
tactcattagaagaaaagg

SEQ ID NO: 186 - RNA, 19 nt, synthetic construct
  source 1..19: mol_type = other RNA
tgtttcacaaacaagctgg

SEQ ID NO: 187 - RNA, 19 nt, synthetic construct
  source 1..19: mol_type = other RNA
tcttagaccaaggagaaac

SEQ ID NO: 188 - RNA, 19 nt, synthetic construct
  source 1..19: mol_type = other RNA
ttcggttggaattcttttt

SEQ ID NO: 189 - RNA, 19 nt, synthetic construct
  source 1..19: mol_type = other RNA
tattagaagaaaaggtggg

SEQ ID NO: 190 - RNA, 19 nt, synthetic construct
  source 1..19: mol_type = other RNA
aactcattagaagaaaagg

SEQ ID NO: 191 - RNA, 19 nt, synthetic construct
  source 1..19: mol_type = other RNA
agtttcacaaacaagctgg

SEQ ID NO: 192 - RNA, 19 nt, synthetic construct
  source 1..19: mol_type = other RNA
acttagaccaaggagaaac

SEQ ID NO: 193 - RNA, 19 nt, synthetic construct
  source 1..19: mol_type = other RNA
atcggttggaattcttttt

SEQ ID NO: 194 - RNA, 19 nt, synthetic construct
  source 1..19: mol_type = other RNA
aattagaagaaaaggtggg

SEQ ID NO: 195 - RNA, 21 nt, synthetic construct
  source 1..21: mol_type = other RNA
gcgtcatccacaatgagagta

SEQ ID NO: 196 - RNA, 21 nt, synthetic construct
  source 1..21: mol_type = other RNA
gatccacaatgagagtaccta

SEQ ID NO: 197 - RNA, 21 nt, synthetic construct
  source 1..21: mol_type = other RNA
gtccacaatgagagtacctga

SEQ ID NO: 198 - RNA, 21 nt, synthetic construct
  source 1..21: mol_type = other RNA
gttcttgggcttccgtatata

SEQ ID NO: 199 - RNA, 21 nt, synthetic construct
  source 1..21: mol_type = other RNA
gttgggcttccgtatatatga

SEQ ID NO: 200 - RNA, 21 nt, synthetic construct
  source 1..21: mol_type = other RNA
gtgggcttccgtatatatgga

SEQ ID NO: 201 - RNA, 21 nt, synthetic construct
  source 1..21: mol_type = other RNA
ggcttccgtatatatggcata

SEQ ID NO: 202 - RNA, 21 nt, synthetic construct
  source 1..21: mol_type = other RNA
gcttccgtatatatggcatga

SEQ ID NO: 203 - RNA, 21 nt, synthetic construct
  source 1..21: mol_type = other RNA
gcgtatatatggcatgcacaa

SEQ ID NO: 204 - RNA, 21 nt, synthetic construct
  source 1..21: mol_type = other RNA
ggttccttggaaggacaagaa

SEQ ID NO: 205 - RNA, 21 nt, synthetic construct
  source 1..21: mol_type = other RNA
gagaagattgacaggttcata

SEQ ID NO: 206 - RNA, 21 nt, synthetic construct
  source 1..21: mol_type = other RNA
gatgcaggctgtgacaggata

SEQ ID NO: 207 - RNA, 21 nt, synthetic construct
  source 1..21: mol_type = other RNA
ggagttctgggtggacaacaa

SEQ ID NO: 208 - RNA, 21 nt, synthetic construct
  source 1..21: mol_type = other RNA
gcaacagcacctcagtgtcta

SEQ ID NO: 209 - RNA, 21 nt, synthetic construct
  source 1..21: mol_type = other RNA
ggggtctcactttccagcaaa

SEQ ID NO: 210 - RNA, 21 nt, synthetic construct
  source 1..21: mol_type = other RNA
gtcactttccagcaaaactca

SEQ ID NO: 211 - RNA, 21 nt, synthetic construct
  source 1..21: mol_type = other RNA
gccagcaaaactccctcaaca

SEQ ID NO: 212 - RNA, 21 nt, synthetic construct
  source 1..21: mol_type = other RNA
gagcaaaactccctcaactga

SEQ ID NO: 213 - RNA, 21 nt, synthetic construct
  source 1..21: mol_type = other RNA
ggagctgaacctgcaaaaata

SEQ ID NO: 214 - RNA, 21 nt, synthetic construct
  source 1..21: mol_type = other RNA
gctgaacctgcaaaaattgaa

SEQ ID NO: 215 - RNA, 21 nt, synthetic construct
  source 1..21: mol_type = other RNA
ggaaccgcccattcctgttta

SEQ ID NO: 216 - RNA, 21 nt, synthetic construct
  source 1..21: mol_type = other RNA
gaaccgcccattcctgtttga

SEQ ID NO: 217 - RNA, 21 nt, synthetic construct
  source 1..21: mol_type = other RNA
gttcctgtttgctgtgtatga

SEQ ID NO: 218 - RNA, 21 nt, synthetic construct
  source 1..21: mol_type = other RNA
gctgtttgctgtgtatgatca

SEQ ID NO: 219 - RNA, 21 nt, synthetic construct
  source 1..21: mol_type = other RNA
gtgtttgctgtgtatgatcaa

SEQ ID NO: 220 - RNA, 21 nt, synthetic construct
  source 1..21: mol_type = other RNA
gttgctgtgtatgatcaaaga

SEQ ID NO: 221 - RNA, 21 nt, synthetic construct
  source 1..21: mol_type = other RNA
gtcccaccttttcttctaata

SEQ ID NO: 222 - RNA, 21 nt, synthetic construct
  source 1..21: mol_type = other RNA
gaccttttcttctaatgagta

SEQ ID NO: 223 - RNA, 21 nt, synthetic construct
  source 1..21: mol_type = other RNA
gccttttcttctaatgagtca

SEQ ID NO: 224 - RNA, 21 nt, synthetic construct
  source 1..21: mol_type = other RNA
gcgtttctccttggtctaaga

SEQ ID NO: 225 - RNA, 21 nt, synthetic construct
  source 1..21: mol_type = other RNA
gtttctccttggtctaagtga

SEQ ID NO: 226 - RNA, 21 nt, synthetic construct
  source 1..21: mol_type = other RNA
ggtttgctgggtttattttaa

SEQ ID NO: 227 - RNA, 21 nt, synthetic construct
  source 1..21: mol_type = other RNA
gtttgctgggtttattttaga

SEQ ID NO: 228 - RNA, 21 nt, synthetic construct
  source 1..21: mol_type = other RNA
gttgctgggtttattttagaa

SEQ ID NO: 229 - RNA, 21 nt, synthetic construct
  source 1..21: mol_type = other RNA
ggggtttattttagagaatga

SEQ ID NO: 230 - RNA, 21 nt, synthetic construct
  source 1..21: mol_type = other RNA
gggtttattttagagaatgga

SEQ ID NO: 231 - RNA, 21 nt, synthetic construct
  source 1..21: mol_type = other RNA
gggcaagaaccagtgtttaga

SEQ ID NO: 232 - RNA, 21 nt, synthetic construct
  source 1..21: mol_type = other RNA
ggcaagaaccagtgtttagca

SEQ ID NO: 233 - RNA, 21 nt, synthetic construct
  source 1..21: mol_type = other RNA
gcaagaaccagtgtttagcga

SEQ ID NO: 234 - RNA, 21 nt, synthetic construct
  source 1..21: mol_type = other RNA
gctgttccaaaaagaattcca

SEQ ID NO: 235 - RNA, 21 nt, synthetic construct
  source 1..21: mol_type = other RNA
ggttccaaaaagaattccaaa

SEQ ID NO: 236 - RNA, 21 nt, synthetic construct
  source 1..21: mol_type = other RNA
gttccaaaaagaattccaaca

SEQ ID NO: 237 - RNA, 21 nt, synthetic construct
  source 1..21: mol_type = other RNA
gcaaaaagaattccaaccgaa

SEQ ID NO: 238 - RNA, 21 nt, synthetic construct
  source 1..21: mol_type = other RNA
gaaaaagaattccaaccgaca

SEQ ID NO: 239 - RNA, 21 nt, synthetic construct
  source 1..21: mol_type = other RNA
gcaaccgaccagcttgtttga

SEQ ID NO: 240 - RNA, 21 nt, synthetic construct
  source 1..21: mol_type = other RNA
gaaccgaccagcttgtttgta

SEQ ID NO: 241 - RNA, 21 nt, synthetic construct
  source 1..21: mol_type = other RNA
ggaccagcttgtttgtgaaaa

SEQ ID NO: 242 - RNA, 21 nt, synthetic construct
  source 1..21: mol_type = other RNA
gaccagcttgtttgtgaaaca

SEQ ID NO: 243 - RNA, 21 nt, synthetic construct
  source 1..21: mol_type = other RNA
gccagcttgtttgtgaaacaa

SEQ ID NO: 244 - RNA, 21 nt, synthetic construct
  source 1..21: mol_type = other RNA
gtcatccacaatgagagtaca

SEQ ID NO: 245 - RNA, 21 nt, synthetic construct
  source 1..21: mol_type = other RNA
tactctcattgtggatgacgc

SEQ ID NO: 246 - RNA, 21 nt, synthetic construct
  source 1..21: mol_type = other RNA
taggtactctcattgtggatc

SEQ ID NO: 247 - RNA, 21 nt, synthetic construct
  source 1..21: mol_type = other RNA
tcaggtactctcattgtggac

SEQ ID NO: 248 - RNA, 21 nt, synthetic construct
  source 1..21: mol_type = other RNA
tatatacggaagcccaagaac

SEQ ID NO: 249 - RNA, 21 nt, synthetic construct
  source 1..21: mol_type = other RNA
tcatatatacggaagcccaac

SEQ ID NO: 250 - RNA, 21 nt, synthetic construct
  source 1..21: mol_type = other RNA
tccatatatacggaagcccac

SEQ ID NO: 251 - RNA, 21 nt, synthetic construct
  source 1..21: mol_type = other RNA
tatgccatatatacggaagcc

SEQ ID NO: 252 - RNA, 21 nt, synthetic construct
  source 1..21: mol_type = other RNA
tcatgccatatatacggaagc

SEQ ID NO: 253 - RNA, 21 nt, synthetic construct
  source 1..21: mol_type = other RNA
ttgtgcatgccatatatacgc

SEQ ID NO: 254 - RNA, 21 nt, synthetic construct
  source 1..21: mol_type = other RNA
ttcttgtccttccaaggaacc

SEQ ID NO: 255 - RNA, 21 nt, synthetic construct
  source 1..21: mol_type = other RNA
tatgaacctgtcaatcttctc

SEQ ID NO: 256 - RNA, 21 nt, synthetic construct
  source 1..21: mol_type = other RNA
tatcctgtcacagcctgcatc

SEQ ID NO: 257 - RNA, 21 nt, synthetic construct
  source 1..21: mol_type = other RNA
ttgttgtccacccagaactcc

SEQ ID NO: 258 - RNA, 21 nt, synthetic construct
  source 1..21: mol_type = other RNA
tagacactgaggtgctgttgc

SEQ ID NO: 259 - RNA, 21 nt, synthetic construct
  source 1..21: mol_type = other RNA
tttgctggaaagtgagacccc

SEQ ID NO: 260 - RNA, 21 nt, synthetic construct
  source 1..21: mol_type = other RNA
tgagttttgctggaaagtgac

SEQ ID NO: 261 - RNA, 21 nt, synthetic construct
  source 1..21: mol_type = other RNA
tgttgagggagttttgctggc

SEQ ID NO: 262 - RNA, 21 nt, synthetic construct
  source 1..21: mol_type = other RNA
tcagttgagggagttttgctc

SEQ ID NO: 263 - RNA, 21 nt, synthetic construct
  source 1..21: mol_type = other RNA
tatttttgcaggttcagctcc

SEQ ID NO: 264 - RNA, 21 nt, synthetic construct
  source 1..21: mol_type = other RNA
ttcaatttttgcaggttcagc

SEQ ID NO: 265 - RNA, 21 nt, synthetic construct
  source 1..21: mol_type = other RNA
taaacaggaatgggcggttcc

SEQ ID NO: 266 - RNA, 21 nt, synthetic construct
  source 1..21: mol_type = other RNA
tcaaacaggaatgggcggttc

SEQ ID NO: 267 - RNA, 21 nt, synthetic construct
  source 1..21: mol_type = other RNA
tcatacacagcaaacaggaac

SEQ ID NO: 268 - RNA, 21 nt, synthetic construct
  source 1..21: mol_type = other RNA
tgatcatacacagcaaacagc

SEQ ID NO: 269 - RNA, 21 nt, synthetic construct
  source 1..21: mol_type = other RNA
ttgatcatacacagcaaacac

SEQ ID NO: 270 - RNA, 21 nt, synthetic construct
  source 1..21: mol_type = other RNA
tctttgatcatacacagcaac

SEQ ID NO: 271 - RNA, 21 nt, synthetic construct
  source 1..21: mol_type = other RNA
tattagaagaaaaggtgggac

SEQ ID NO: 272 - RNA, 21 nt, synthetic construct
  source 1..21: mol_type = other RNA
tactcattagaagaaaaggtc

SEQ ID NO: 273 - RNA, 21 nt, synthetic construct
  source 1..21: mol_type = other RNA
tgactcattagaagaaaaggc

SEQ ID NO: 274 - RNA, 21 nt, synthetic construct
  source 1..21: mol_type = other RNA
tcttagaccaaggagaaacgc

SEQ ID NO: 275 - RNA, 21 nt, synthetic construct
  source 1..21: mol_type = other RNA
tcacttagaccaaggagaaac

SEQ ID NO: 276 - RNA, 21 nt, synthetic construct
  source 1..21: mol_type = other RNA
ttaaaataaacccagcaaacc

SEQ ID NO: 277 - RNA, 21 nt, synthetic construct
  source 1..21: mol_type = other RNA
tctaaaataaacccagcaaac

SEQ ID NO: 278 - RNA, 21 nt, synthetic construct
  source 1..21: mol_type = other RNA
ttctaaaataaacccagcaac

SEQ ID NO: 279 - RNA, 21 nt, synthetic construct
  source 1..21: mol_type = other RNA
tcattctctaaaataaacccc

SEQ ID NO: 280 - RNA, 21 nt, synthetic construct
  source 1..21: mol_type = other RNA
tccattctctaaaataaaccc

SEQ ID NO: 281 - RNA, 21 nt, synthetic construct
  source 1..21: mol_type = other RNA
tctaaacactggttcttgccc

SEQ ID NO: 282 - RNA, 21 nt, synthetic construct
  source 1..21: mol_type = other RNA
tgctaaacactggttcttgcc

SEQ ID NO: 283 - RNA, 21 nt, synthetic construct
  source 1..21: mol_type = other RNA
tcgctaaacactggttcttgc

SEQ ID NO: 284 - RNA, 21 nt, synthetic construct
  source 1..21: mol_type = other RNA
tggaattctttttggaacagc

SEQ ID NO: 285 - RNA, 21 nt, synthetic construct
  source 1..21: mol_type = other RNA
tttggaattctttttggaacc

SEQ ID NO: 286 - RNA, 21 nt, synthetic construct
  source 1..21: mol_type = other RNA
tgttggaattctttttggaac

SEQ ID NO: 287 - RNA, 21 nt, synthetic construct
  source 1..21: mol_type = other RNA
ttcggttggaattctttttgc

SEQ ID NO: 288 - RNA, 21 nt, synthetic construct
  source 1..21: mol_type = other RNA
tgtcggttggaattctttttc

SEQ ID NO: 289 - RNA, 21 nt, synthetic construct
  source 1..21: mol_type = other RNA
tcaaacaagctggtcggttgc

SEQ ID NO: 290 - RNA, 21 nt, synthetic construct
  source 1..21: mol_type = other RNA
tacaaacaagctggtcggttc

SEQ ID NO: 291 - RNA, 21 nt, synthetic construct
  source 1..21: mol_type = other RNA
ttttcacaaacaagctggtcc

SEQ ID NO: 292 - RNA, 21 nt, synthetic construct
  source 1..21: mol_type = other RNA
tgtttcacaaacaagctggtc

SEQ ID NO: 293 - RNA, 21 nt, synthetic construct
  source 1..21: mol_type = other RNA
ttgtttcacaaacaagctggc

SEQ ID NO: 294 - RNA, 21 nt, synthetic construct
  source 1..21: mol_type = other RNA
tgtactctcattgtggatgac

SEQ ID NO: 295 - RNA, 21 nt, synthetic construct
  source 1..21: mol_type = other RNA
gtcatccacaatgagagtaca

SEQ ID NO: 296 - RNA, 21 nt, synthetic construct
  source 1..21: mol_type = other RNA
gtgggcttccgtatatatgga

SEQ ID NO: 297 - RNA, 21 nt, synthetic construct
  source 1..21: mol_type = other RNA
gctgtttgctgtgtatgatca

SEQ ID NO: 298 - RNA, 21 nt, synthetic construct
  source 1..21: mol_type = other RNA
ggtttgctgggtttattttaa

SEQ ID NO: 299 - RNA, 21 nt, synthetic construct
  source 1..21: mol_type = other RNA
gccagcttgtttgtgaaacaa

SEQ ID NO: 300 - RNA, 21 nt, synthetic construct
  source 1..21: mol_type = other RNA
gtcccaccttttcttctaata

SEQ ID NO: 301 - RNA, 21 nt, synthetic construct
  source 1..21: mol_type = other RNA
gccttttcttctaatgagtca

SEQ ID NO: 302 - RNA, 21 nt, synthetic construct
  source 1..21: mol_type = other RNA
ggggtttattttagagaatga

SEQ ID NO: 303 - RNA, 21 nt, synthetic construct
  source 1..21: mol_type = other RNA
ggttccaaaaagaattccaaa

SEQ ID NO: 304 - RNA, 21 nt, synthetic construct
  source 1..21: mol_type = other RNA
gttccaaaaagaattccaaca

SEQ ID NO: 305 - RNA, 21 nt, synthetic construct
  source 1..21: mol_type = other RNA
catccacaatgagagtaccta

SEQ ID NO: 306 - RNA, 21 nt, synthetic construct
  source 1..21: mol_type = other RNA
cttcttgggcttccgtatata

SEQ ID NO: 307 - RNA, 21 nt, synthetic construct
  source 1..21: mol_type = other RNA
catgcaggctgtgacaggata

SEQ ID NO: 308 - RNA, 21 nt, synthetic construct
  source 1..21: mol_type = other RNA
gctgaacctgcaaaaattgaa

SEQ ID NO: 309 - RNA, 21 nt, synthetic construct
  source 1..21: mol_type = other RNA
caccttttcttctaatgagta

SEQ ID NO: 310 - RNA, 21 nt, synthetic construct
  source 1..21: mol_type = other RNA
ccgtttctccttggtctaaga

SEQ ID NO: 311 - RNA, 21 nt, synthetic construct
  source 1..21: mol_type = other RNA
actgttccaaaaagaattcca

SEQ ID NO: 312 - RNA, 21 nt, synthetic construct
  source 1..21: mol_type = other RNA
caaaaagaattccaaccgaca

SEQ ID NO: 313 - RNA, 21 nt, synthetic construct
  source 1..21: mol_type = other RNA
cgaccagcttgtttgtgaaaa

SEQ ID NO: 314 - RNA, 21 nt, synthetic construct
  source 1..21: mol_type = other RNA
gaccagcttgtttgtgaaaca

SEQ ID NO: 315 - RNA, 21 nt, synthetic construct
  source 1..21: mol_type = other RNA
tcgtcatccacaatgagagta

SEQ ID NO: 316 - RNA, 21 nt, synthetic construct
  source 1..21: mol_type = other RNA
gtccacaatgagagtacctga

SEQ ID NO: 317 - RNA, 21 nt, synthetic construct
  source 1..21: mol_type = other RNA
agggtctcactttccagcaaa

SEQ ID NO: 318 - RNA, 21 nt, synthetic construct
  source 1..21: mol_type = other RNA
ctgtttgctgtgtatgatcaa

SEQ ID NO: 319 - RNA, 21 nt, synthetic construct
  source 1..21: mol_type = other RNA
tttgctgtgtatgatcaaaga

SEQ ID NO: 320 - RNA, 21 nt, synthetic construct
  source 1..21: mol_type = other RNA
gtttctccttggtctaagtga

SEQ ID NO: 321 - RNA, 21 nt, synthetic construct
  source 1..21: mol_type = other RNA
gcaagaaccagtgtttagcga

SEQ ID NO: 322 - RNA, 21 nt, synthetic construct
  source 1..21: mol_type = other RNA
ccaaaaagaattccaaccgaa

SEQ ID NO: 323 - RNA, 21 nt, synthetic construct
  source 1..21: mol_type = other RNA
caaccgaccagcttgtttgta

SEQ ID NO: 324 - RNA, 21 nt, synthetic construct
  source 1..21: mol_type = other RNA
gtcccaccttttcttctaata

SEQ ID NO: 325 - RNA, 21 nt, synthetic construct
  source 1..21: mol_type = other RNA
gttccaaaaagaattccaaca

SEQ ID NO: 326 - RNA, 21 nt, synthetic construct
  source 1..21: mol_type = other RNA
gccttttcttctaatgagtca

SEQ ID NO: 327 - RNA, 21 nt, synthetic construct
  source 1..21: mol_type = other RNA
gccagcttgtttgtgaaacaa

SEQ ID NO: 328 - RNA, 21 nt, synthetic construct
  source 1..21: mol_type = other RNA
ggttccaaaaagaattccaaa

SEQ ID NO: 329 - RNA, 21 nt, synthetic construct
  source 1..21: mol_type = other RNA
ggtttgctgggtttattttaa

SEQ ID NO: 330 - RNA, 21 nt, synthetic construct
  source 1..21: mol_type = other RNA
caccttttcttctaatgagta

SEQ ID NO: 331 - RNA, 21 nt, synthetic construct
  source 1..21: mol_type = other RNA
gaccagcttgtttgtgaaaca

SEQ ID NO: 332 - RNA, 21 nt, synthetic construct
  source 1..21: mol_type = other RNA
ccgtttctccttggtctaaga

SEQ ID NO: 333 - RNA, 21 nt, synthetic construct
  source 1..21: mol_type = other RNA
cgaccagcttgtttgtgaaaa

SEQ ID NO: 334 - RNA, 21 nt, synthetic construct
  source 1..21: mol_type = other RNA
caaaaagaattccaaccgaca

SEQ ID NO: 335 - RNA, 21 nt, synthetic construct
  source 1..21: mol_type = other RNA
tcgtcatccacaatgagagta

SEQ ID NO: 336 - RNA, 21 nt, synthetic construct
  source 1..21: mol_type = other RNA
gtccacaatgagagtacctga

SEQ ID NO: 337 - RNA, 21 nt, synthetic construct
  source 1..21: mol_type = other RNA
agggtctcactttccagcaaa

SEQ ID NO: 338 - RNA, 21 nt, synthetic construct
  source 1..21: mol_type = other RNA
ctgtttgctgtgtatgatcaa

SEQ ID NO: 339 - RNA, 21 nt, synthetic construct
  source 1..21: mol_type = other RNA
tttgctgtgtatgatcaaaga

SEQ ID NO: 340 - RNA, 21 nt, synthetic construct
  source 1..21: mol_type = other RNA
gtttctccttggtctaagtga

SEQ ID NO: 341 - RNA, 21 nt, synthetic construct
  source 1..21: mol_type = other RNA
gcaagaaccagtgtttagcga

SEQ ID NO: 342 - RNA, 21 nt, synthetic construct
  source 1..21: mol_type = other RNA
ccaaaaagaattccaaccgaa

SEQ ID NO: 343 - RNA, 21 nt, synthetic construct
  source 1..21: mol_type = other RNA
caaccgaccagcttgtttgta

SEQ ID NO: 344 - RNA, 21 nt, synthetic construct
  source 1..21: mol_type = other RNA
tgtactctcattgtggatgac

SEQ ID NO: 345 - RNA, 21 nt, synthetic construct
  source 1..21: mol_type = other RNA
tccatatatacggaagcccac

SEQ ID NO: 346 - RNA, 21 nt, synthetic construct
  source 1..21: mol_type = other RNA
tgatcatacacagcaaacagc

SEQ ID NO: 347 - RNA, 21 nt, synthetic construct
  source 1..21: mol_type = other RNA
ttaaaataaacccagcaaacc

SEQ ID NO: 348 - RNA, 21 nt, synthetic construct
  source 1..21: mol_type = other RNA
ttgtttcacaaacaagctggc

SEQ ID NO: 349 - RNA, 21 nt, synthetic construct
  source 1..21: mol_type = other RNA
tattagaagaaaaggtgggac

SEQ ID NO: 350 - RNA, 21 nt, synthetic construct
  source 1..21: mol_type = other RNA
tgactcattagaagaaaaggc

SEQ ID NO: 351 - RNA, 21 nt, synthetic construct
  source 1..21: mol_type = other RNA
tcattctctaaaataaacccc

SEQ ID NO: 352 - RNA, 21 nt, synthetic construct
  source 1..21: mol_type = other RNA
tttggaattctttttggaacc

SEQ ID NO: 353 - RNA, 21 nt, synthetic construct
  source 1..21: mol_type = other RNA
tgttggaattctttttggaac

SEQ ID NO: 354 - RNA, 21 nt, synthetic construct
  source 1..21: mol_type = other RNA
taggtactctcattgtggatg

SEQ ID NO: 355 - RNA, 21 nt, synthetic construct
  source 1..21: mol_type = other RNA
tatatacggaagcccaagaag

SEQ ID NO: 356 - RNA, 21 nt, synthetic construct
  source 1..21: mol_type = other RNA
tatcctgtcacagcctgcatg

SEQ ID NO: 357 - RNA, 21 nt, synthetic construct
  source 1..21: mol_type = other RNA
ttcaatttttgcaggttcagc

SEQ ID NO: 358 - RNA, 21 nt, synthetic construct
  source 1..21: mol_type = other RNA
tactcattagaagaaaaggtg

SEQ ID NO: 359 - RNA, 21 nt, synthetic construct
  source 1..21: mol_type = other RNA
tcttagaccaaggagaaacgg

SEQ ID NO: 360 - RNA, 21 nt, synthetic construct
  source 1..21: mol_type = other RNA
tggaattctttttggaacagt

SEQ ID NO: 361 - RNA, 21 nt, synthetic construct
  source 1..21: mol_type = other RNA
tgtcggttggaattctttttg

SEQ ID NO: 362 - RNA, 21 nt, synthetic construct
  source 1..21: mol_type = other RNA
ttttcacaaacaagctggtcg

SEQ ID NO: 363 - RNA, 21 nt, synthetic construct
  source 1..21: mol_type = other RNA
tgtttcacaaacaagctggtc

SEQ ID NO: 364 - RNA, 21 nt, synthetic construct
  source 1..21: mol_type = other RNA
tactctcattgtggatgacga

SEQ ID NO: 365 - RNA, 21 nt, synthetic construct
  source 1..21: mol_type = other RNA
tcaggtactctcattgtggac

SEQ ID NO: 366 - RNA, 21 nt, synthetic construct
  source 1..21: mol_type = other RNA
tttgctggaaagtgagaccct

SEQ ID NO: 367 - RNA, 21 nt, synthetic construct
  source 1..21: mol_type = other RNA
ttgatcatacacagcaaacag

SEQ ID NO: 368 - RNA, 21 nt, synthetic construct
  source 1..21: mol_type = other RNA
tctttgatcatacacagcaaa

SEQ ID NO: 369 - RNA, 21 nt, synthetic construct
  source 1..21: mol_type = other RNA
tcacttagaccaaggagaaac

SEQ ID NO: 370 - RNA, 21 nt, synthetic construct
  source 1..21: mol_type = other RNA
tcgctaaacactggttcttgc

SEQ ID NO: 371 - RNA, 21 nt, synthetic construct
  source 1..21: mol_type = other RNA
ttcggttggaattctttttgg

SEQ ID NO: 372 - RNA, 21 nt, synthetic construct
  source 1..21: mol_type = other RNA
tacaaacaagctggtcggttg

SEQ ID NO: 373 - RNA, 21 nt, synthetic construct
  source 1..21: mol_type = other RNA
tattagaagaaaaggtgggac

SEQ ID NO: 374 - RNA, 21 nt, synthetic construct
  source 1..21: mol_type = other RNA
tgttggaattctttttggaac

SEQ ID NO: 375 - RNA, 21 nt, synthetic construct
  source 1..21: mol_type = other RNA
tgactcattagaagaaaaggc

SEQ ID NO: 376 - RNA, 21 nt, synthetic construct
  source 1..21: mol_type = other RNA
ttgtttcacaaacaagctggc

SEQ ID NO: 377 - RNA, 21 nt, synthetic construct
  source 1..21: mol_type = other RNA
tttggaattctttttggaacc

SEQ ID NO: 378 - RNA, 21 nt, synthetic construct
  source 1..21: mol_type = other RNA
ttaaaataaacccagcaaacc

SEQ ID NO: 379 - RNA, 21 nt, synthetic construct
  source 1..21: mol_type = other RNA
tactcattagaagaaaaggtg

SEQ ID NO: 380 - RNA, 21 nt, synthetic construct
  source 1..21: mol_type = other RNA
tgtttcacaaacaagctggtc

SEQ ID NO: 381 - RNA, 21 nt, synthetic construct
  source 1..21: mol_type = other RNA
tcttagaccaaggagaaacgg

SEQ ID NO: 382 - RNA, 21 nt, synthetic construct
  source 1..21: mol_type = other RNA
ttttcacaaacaagctggtcg

SEQ ID NO: 383 - RNA, 21 nt, synthetic construct
  source 1..21: mol_type = other RNA
tgtcggttggaattctttttg

SEQ ID NO: 384 - RNA, 21 nt, synthetic construct
  source 1..21: mol_type = other RNA
tactctcattgtggatgacga

SEQ ID NO: 385 - RNA, 21 nt, synthetic construct
  source 1..21: mol_type = other RNA
tcaggtactctcattgtggac

SEQ ID NO: 386 - RNA, 21 nt, synthetic construct
  source 1..21: mol_type = other RNA
tttgctggaaagtgagaccct

SEQ ID NO: 387 - RNA, 21 nt, synthetic construct
  source 1..21: mol_type = other RNA
ttgatcatacacagcaaacag

SEQ ID NO: 388 - RNA, 21 nt, synthetic construct
  source 1..21: mol_type = other RNA
tctttgatcatacacagcaaa

SEQ ID NO: 389 - RNA, 21 nt, synthetic construct
  source 1..21: mol_type = other RNA
tcacttagaccaaggagaaac

SEQ ID NO: 390 - RNA, 21 nt, synthetic construct
  source 1..21: mol_type = other RNA
tcgctaaacactggttcttgc

SEQ ID NO: 391 - RNA, 21 nt, synthetic construct
  source 1..21: mol_type = other RNA
ttcggttggaattctttttgg

SEQ ID NO: 392 - RNA, 21 nt, synthetic construct
  source 1..21: mol_type = other RNA
tacaaacaagctggtcggttg

SEQ ID NO: 393 - RNA, 21 nt, synthetic construct
  source 1..21: mol_type = other RNA
gtgggcttccgtatatatgga

SEQ ID NO: 394 - RNA, 21 nt, synthetic construct
  source 1..21: mol_type = other RNA
gctgtttgctgtgtatgatca

SEQ ID NO: 395 - RNA, 21 nt, synthetic construct
  source 1..21: mol_type = other RNA
ggtttgctgggtttattttaa

SEQ ID NO: 396 - RNA, 21 nt, synthetic construct
  source 1..21: mol_type = other RNA
gccagcttgtttgtgaaacaa

SEQ ID NO: 397 - RNA, 21 nt, synthetic construct
  source 1..21: mol_type = other RNA
gtcccaccttttcttctaata

SEQ ID NO: 398 - RNA, 21 nt, synthetic construct
  source 1..21: mol_type = other RNA
gccttttcttctaatgagtca

SEQ ID NO: 399 - RNA, 21 nt, synthetic construct
  source 1..21: mol_type = other RNA
ggggtttattttagagaatga

SEQ ID NO: 400 - RNA, 21 nt, synthetic construct
  source 1..21: mol_type = other RNA
ggttccaaaaagaattccaaa

SEQ ID NO: 401 - RNA, 21 nt, synthetic construct
  source 1..21: mol_type = other RNA
gttccaaaaagaattccaaca

SEQ ID NO: 402 - RNA, 21 nt, synthetic construct
  source 1..21: mol_type = other RNA
catccacaatgagagtaccta

SEQ ID NO: 403 - RNA, 21 nt, synthetic construct
  source 1..21: mol_type = other RNA
cttcttgggcttccgtatata

SEQ ID NO: 404 - RNA, 21 nt, synthetic construct
  source 1..21: mol_type = other RNA
catgcaggctgtgacaggata

SEQ ID NO: 405 - RNA, 21 nt, synthetic construct
  source 1..21: mol_type = other RNA
gctgaacctgcaaaaattgaa

SEQ ID NO: 406 - RNA, 21 nt, synthetic construct
  source 1..21: mol_type = other RNA
actgttccaaaaagaattcca

SEQ ID NO: 407 - RNA, 21 nt, synthetic construct
  source 1..21: mol_type = other RNA
caaaaagaattccaaccgaca

SEQ ID NO: 408 - RNA, 21 nt, synthetic construct
  source 1..21: mol_type = other RNA
cgaccagcttgtttgtgaaaa

SEQ ID NO: 409 - RNA, 21 nt, synthetic construct
  source 1..21: mol_type = other RNA
tcgtcatccacaatgagagta

SEQ ID NO: 410 - RNA, 21 nt, synthetic construct
  source 1..21: mol_type = other RNA
gtccacaatgagagtacctga

SEQ ID NO: 411 - RNA, 21 nt, synthetic construct
  source 1..21: mol_type = other RNA
agggtctcactttccagcaaa

SEQ ID NO: 412 - RNA, 21 nt, synthetic construct
  source 1..21: mol_type = other RNA
ctgtttgctgtgtatgatcaa

SEQ ID NO: 413 - RNA, 21 nt, synthetic construct
  source 1..21: mol_type = other RNA
tttgctgtgtatgatcaaaga

SEQ ID NO: 414 - RNA, 21 nt, synthetic construct
  source 1..21: mol_type = other RNA
gtttctccttggtctaagtga

SEQ ID NO: 415 - RNA, 21 nt, synthetic construct
  source 1..21: mol_type = other RNA
gcaagaaccagtgtttagcga

SEQ ID NO: 416 - RNA, 21 nt, synthetic construct
  source 1..21: mol_type = other RNA
ccaaaaagaattccaaccgaa

SEQ ID NO: 417 - RNA, 21 nt, synthetic construct
  source 1..21: mol_type = other RNA
caaccgaccagcttgtttgta

SEQ ID NO: 418 - RNA, 21 nt, synthetic construct
  source 1..21: mol_type = other RNA
gtcccaccttttcttctaata

SEQ ID NO: 419 - RNA, 21 nt, synthetic construct
  source 1..21: mol_type = other RNA
gttccaaaaagaattccaaca

SEQ ID NO: 420 - RNA, 21 nt, synthetic construct
  source 1..21: mol_type = other RNA
gccttttcttctaatgagtca

SEQ ID NO: 421 - RNA, 21 nt, synthetic construct
  source 1..21: mol_type = other RNA
gccagcttgtttgtgaaacaa

SEQ ID NO: 422 - RNA, 21 nt, synthetic construct
  source 1..21: mol_type = other RNA
ggttccaaaaagaattccaaa

SEQ ID NO: 423 - RNA, 21 nt, synthetic construct
  source 1..21: mol_type = other RNA
ggtttgctgggtttattttaa

SEQ ID NO: 424 - RNA, 21 nt, synthetic construct
  source 1..21: mol_type = other RNA
caccttttcttctaatgagta

SEQ ID NO: 425 - RNA, 21 nt, synthetic construct
  source 1..21: mol_type = other RNA
gaccagcttgtttgtgaaaca

SEQ ID NO: 426 - RNA, 21 nt, synthetic construct
  source 1..21: mol_type = other RNA
ccgtttctccttggtctaaga

SEQ ID NO: 427 - RNA, 21 nt, synthetic construct
  source 1..21: mol_type = other RNA
cgaccagcttgtttgtgaaaa

SEQ ID NO: 428 - RNA, 21 nt, synthetic construct
  source 1..21: mol_type = other RNA
caaaaagaattccaaccgaca

SEQ ID NO: 429 - RNA, 21 nt, synthetic construct
  source 1..21: mol_type = other RNA
tcgtcatccacaatgagagta

SEQ ID NO: 430 - RNA, 21 nt, synthetic construct
  source 1..21: mol_type = other RNA
gtccacaatgagagtacctga

SEQ ID NO: 431 - RNA, 21 nt, synthetic construct
  source 1..21: mol_type = other RNA
agggtctcactttccagcaaa

SEQ ID NO: 432 - RNA, 21 nt, synthetic construct
  source 1..21: mol_type = other RNA
ctgtttgctgtgtatgatcaa

SEQ ID NO: 433 - RNA, 21 nt, synthetic construct
  source 1..21: mol_type = other RNA
tttgctgtgtatgatcaaaga

SEQ ID NO: 434 - RNA, 21 nt, synthetic construct
  source 1..21: mol_type = other RNA
gtttctccttggtctaagtga

SEQ ID NO: 435 - RNA, 21 nt, synthetic construct
  source 1..21: mol_type = other RNA
gcaagaaccagtgtttagcga

SEQ ID NO: 436 - RNA, 21 nt, synthetic construct
  source 1..21: mol_type = other RNA
caaccgaccagcttgtttgta

SEQ ID NO: 437 - RNA, 21 nt, synthetic construct
  source 1..21: mol_type = other RNA
ccaaccgaccagcttgtttga

SEQ ID NO: 438 - RNA, 21 nt, synthetic construct
  source 1..21: mol_type = other RNA
gaccttttcttctaatgagta

SEQ ID NO: 439 - RNA, 21 nt, synthetic construct
  source 1..21: mol_type = other RNA
gacctttctttctagcgagta

SEQ ID NO: 440 - RNA, 21 nt, synthetic construct
  source 1..21: mol_type = other RNA
gaccttttcttctaatgagta

SEQ ID NO: 441 - RNA, 21 nt, synthetic construct
  source 1..21: mol_type = other RNA
caccagcttgtttgtgaaaca

SEQ ID NO: 442 - RNA, 21 nt, synthetic construct
  source 1..21: mol_type = other RNA
caccagcttgtttgtgaaaca

SEQ ID NO: 443 - RNA, 21 nt, synthetic construct
  source 1..21: mol_type = other RNA
gcgtttctccttggtctaaga

SEQ ID NO: 444 - RNA, 21 nt, synthetic construct
  source 1..21: mol_type = other RNA
gcgtttctccttggtctaaga

SEQ ID NO: 445 - RNA, 21 nt, synthetic construct
  source 1..21: mol_type = other RNA
gcaaaaagaattccaaccgaa

SEQ ID NO: 446 - RNA, 19 nt, synthetic construct
  source 1..19: mol_type = other RNA
ccttttcttctaatgagta

SEQ ID NO: 447 - RNA, 19 nt, synthetic construct
  source 1..19: mol_type = other RNA
ccagcttgtttgtgaaaca

SEQ ID NO: 448 - RNA, 19 nt, synthetic construct
  source 1..19: mol_type = other RNA
gtttctccttggtctaaga

SEQ ID NO: 449 - RNA, 19 nt, synthetic construct
  source 1..19: mol_type = other RNA
aaaaagaattccaaccgaa

SEQ ID NO: 450 - RNA, 19 nt, synthetic construct
  source 1..19: mol_type = other RNA
cccaccttttcttctaata

SEQ ID NO: 451 - RNA, 19 nt, synthetic construct
  source 1..19: mol_type = other RNA
ccttttcttctaatgagtt

SEQ ID NO: 452 - RNA, 19 nt, synthetic construct
  source 1..19: mol_type = other RNA
ccagcttgtttgtgaaact

SEQ ID NO: 453 - RNA, 19 nt, synthetic construct
  source 1..19: mol_type = other RNA
gtttctccttggtctaagt

SEQ ID NO: 454 - RNA, 19 nt, synthetic construct
  source 1..19: mol_type = other RNA
aaaaagaattccaaccgat

SEQ ID NO: 455 - RNA, 19 nt, synthetic construct
  source 1..19: mol_type = other RNA
cccaccttttcttctaatt

SEQ ID NO: 456 - RNA, 21 nt, synthetic construct
  source 1..21: mol_type = other RNA
tccatatatacggaagcccac

SEQ ID NO: 457 - RNA, 21 nt, synthetic construct
  source 1..21: mol_type = other RNA
tgatcatacacagcaaacagc

SEQ ID NO: 458 - RNA, 21 nt, synthetic construct
  source 1..21: mol_type = other RNA
ttaaaataaacccagcaaacc

SEQ ID NO: 459 - RNA, 21 nt, synthetic construct
  source 1..21: mol_type = other RNA
ttgtttcacaaacaagctggc

SEQ ID NO: 460 - RNA, 21 nt, synthetic construct
  source 1..21: mol_type = other RNA
tattagaagaaaaggtgggac

SEQ ID NO: 461 - RNA, 21 nt, synthetic construct
  source 1..21: mol_type = other RNA
tgactcattagaagaaaaggc

SEQ ID NO: 462 - RNA, 21 nt, synthetic construct
  source 1..21: mol_type = other RNA
tcattctctaaaataaacccc

SEQ ID NO: 463 - RNA, 21 nt, synthetic construct
  source 1..21: mol_type = other RNA
tttggaattctttttggaacc

SEQ ID NO: 464 - RNA, 21 nt, synthetic construct
  source 1..21: mol_type = other RNA
tgttggaattctttttggaac

SEQ ID NO: 465 - RNA, 21 nt, synthetic construct
  source 1..21: mol_type = other RNA
taggtactctcattgtggatg

SEQ ID NO: 466 - RNA, 21 nt, synthetic construct
  source 1..21: mol_type = other RNA
tatatacggaagcccaagaag

SEQ ID NO: 467 - RNA, 21 nt, synthetic construct
  source 1..21: mol_type = other RNA
tatcctgtcacagcctgcatg

SEQ ID NO: 468 - RNA, 21 nt, synthetic construct
  source 1..21: mol_type = other RNA
ttcaatttttgcaggttcagc

SEQ ID NO: 469 - RNA, 21 nt, synthetic construct
  source 1..21: mol_type = other RNA
tggaattctttttggaacagt

SEQ ID NO: 470 - RNA, 21 nt, synthetic construct
  source 1..21: mol_type = other RNA
tgtcggttggaattctttttg

SEQ ID NO: 471 - RNA, 21 nt, synthetic construct
  source 1..21: mol_type = other RNA
ttttcacaaacaagctggtcg

SEQ ID NO: 472 - RNA, 21 nt, synthetic construct
  source 1..21: mol_type = other RNA
tactctcattgtggatgacga

SEQ ID NO: 473 - RNA, 21 nt, synthetic construct
  source 1..21: mol_type = other RNA
tcaggtactctcattgtggac

SEQ ID NO: 474 - RNA, 21 nt, synthetic construct
  source 1..21: mol_type = other RNA
tttgctggaaagtgagaccct

SEQ ID NO: 475 - RNA, 21 nt, synthetic construct
  source 1..21: mol_type = other RNA
ttgatcatacacagcaaacag

SEQ ID NO: 476 - RNA, 21 nt, synthetic construct
  source 1..21: mol_type = other RNA
tctttgatcatacacagcaaa

SEQ ID NO: 477 - RNA, 21 nt, synthetic construct
  source 1..21: mol_type = other RNA
tcacttagaccaaggagaaac

SEQ ID NO: 478 - RNA, 21 nt, synthetic construct
  source 1..21: mol_type = other RNA
tcgctaaacactggttcttgc

SEQ ID NO: 479 - RNA, 21 nt, synthetic construct
  source 1..21: mol_type = other RNA
ttcggttggaattctttttgg

SEQ ID NO: 480 - RNA, 21 nt, synthetic construct
  source 1..21: mol_type = other RNA
tacaaacaagctggtcggttg

SEQ ID NO: 481 - RNA, 21 nt, synthetic construct
  source 1..21: mol_type = other RNA
tattagaagaaaaggtgggac

SEQ ID NO: 482 - RNA, 21 nt, synthetic construct
  source 1..21: mol_type = other RNA
tgttggaattctttttggaac

SEQ ID NO: 483 - RNA, 21 nt, synthetic construct
  source 1..21: mol_type = other RNA
tgactcattagaagaaaaggc

SEQ ID NO: 484 - RNA, 21 nt, synthetic construct
  source 1..21: mol_type = other RNA
ttgtttcacaaacaagctggc

SEQ ID NO: 485 - RNA, 21 nt, synthetic construct
  source 1..21: mol_type = other RNA
tttggaattctttttggaacc

SEQ ID NO: 486 - RNA, 21 nt, synthetic construct
  source 1..21: mol_type = other RNA
ttaaaataaacccagcaaacc

SEQ ID NO: 487 - RNA, 21 nt, synthetic construct
  source 1..21: mol_type = other RNA
tactcattagaagaaaaggtg

SEQ ID NO: 488 - RNA, 21 nt, synthetic construct
  source 1..21: mol_type = other RNA
tgtttcacaaacaagctggtc

SEQ ID NO: 489 - RNA, 21 nt, synthetic construct
  source 1..21: mol_type = other RNA
tcttagaccaaggagaaacgg

SEQ ID NO: 490 - RNA, 21 nt, synthetic construct
  source 1..21: mol_type = other RNA
ttttcacaaacaagctggtcg

SEQ ID NO: 491 - RNA, 21 nt, synthetic construct
  source 1..21: mol_type = other RNA
tgtcggttggaattctttttg

SEQ ID NO: 492 - RNA, 21 nt, synthetic construct
  source 1..21: mol_type = other RNA
tactctcattgtggatgacga

SEQ ID NO: 493 - RNA, 21 nt, synthetic construct
  source 1..21: mol_type = other RNA
tcaggtactctcattgtggac

SEQ ID NO: 494 - RNA, 21 nt, synthetic construct
  source 1..21: mol_type = other RNA
tttgctggaaagtgagaccct

SEQ ID NO: 495 - RNA, 21 nt, synthetic construct
  source 1..21: mol_type = other RNA
ttgatcatacacagcaaacag

SEQ ID NO: 496 - RNA, 21 nt, synthetic construct
  source 1..21: mol_type = other RNA
tctttgatcatacacagcaaa

SEQ ID NO: 497 - RNA, 21 nt, synthetic construct
  source 1..21: mol_type = other RNA
tcacttagaccaaggagaaac

SEQ ID NO: 498 - RNA, 21 nt, synthetic construct
  source 1..21: mol_type = other RNA
tcgctaaacactggttcttgc

SEQ ID NO: 499 - RNA, 21 nt, synthetic construct
  source 1..21: mol_type = other RNA
tacaaacaagctggtcggttg

SEQ ID NO: 500 - RNA, 21 nt, synthetic construct
  source 1..21: mol_type = other RNA
tcaaacaagctggtcggttgg

SEQ ID NO: 501 - RNA, 21 nt, synthetic construct
  source 1..21: mol_type = other RNA
tactcattagaagaaaaggtc

SEQ ID NO: 502 - RNA, 21 nt, synthetic construct
  source 1..21: mol_type = other RNA
tactcattagaagaaaaggtc

SEQ ID NO: 503 - RNA, 21 nt, synthetic construct
  source 1..21: mol_type = other RNA
tactcattagaagaaaaggtc

SEQ ID NO: 504 - RNA, 21 nt, synthetic construct
  source 1..21: mol_type = other RNA
tgtttcacaaacaagctggtg

SEQ ID NO: 505 - RNA, 21 nt, synthetic construct
  source 1..21: mol_type = other RNA
tgtttcacaaacaagctggtg

SEQ ID NO: 506 - RNA, 21 nt, synthetic construct
  source 1..21: mol_type = other RNA
tcttagaccaaggagaaacgc

SEQ ID NO: 507 - RNA, 21 nt, synthetic construct
  source 1..21: mol_type = other RNA
tcttagaccaaggagaaacgc

SEQ ID NO: 508 - RNA, 21 nt, synthetic construct
  source 1..21: mol_type = other RNA
ttcggttggaattctttttgc

SEQ ID NO: 509 - RNA, 19 nt, synthetic construct
  source 1..19: mol_type = other RNA
tactcattagaagaaaagg

SEQ ID NO: 510 - RNA, 19 nt, synthetic construct
  source 1..19: mol_type = other RNA
tgtttcacaaacaagctgg

SEQ ID NO: 511 - RNA, 19 nt, synthetic construct
  source 1..19: mol_type = other RNA
tcttagaccaaggagaaac

SEQ ID NO: 512 - RNA, 19 nt, synthetic construct
  source 1..19: mol_type = other RNA
ttcggttggaattcttttt

SEQ ID NO: 513 - RNA, 19 nt, synthetic construct
  source 1..19: mol_type = other RNA
tattagaagaaaaggtggg

SEQ ID NO: 514 - RNA, 19 nt, synthetic construct
  source 1..19: mol_type = other RNA
aactcattagaagaaaagg

SEQ ID NO: 515 - RNA, 19 nt, synthetic construct
  source 1..19: mol_type = other RNA
agtttcacaaacaagctgg

SEQ ID NO: 516 - RNA, 19 nt, synthetic construct
  source 1..19: mol_type = other RNA
acttagaccaaggagaaac

SEQ ID NO: 517 - RNA, 19 nt, synthetic construct
  source 1..19: mol_type = other RNA
atcggttggaattcttttt

SEQ ID NO: 518 - RNA, 19 nt, synthetic construct
  source 1..19: mol_type = other RNA
aattagaagaaaaggtggg

SEQ ID NO: 519 - RNA, 2116 nt, Homo sapiens
  source 1..2116: mol_type = mRNA
gaagaagctgccgttgttctgggtactacagcagaagggtatgcggaagcgagcaccccagtctgagatggctcctgccggtgtgagcctgagggccaccatcctctgcctcctggcctgggctggcctggctgcaggtgaccgggtgtacatacaccccttccacctcgtcatccacaatgagagtacctgtgagcagctggcaaaggccaatgccgggaagcccaaagaccccaccttcatacctgctccaattcaggccaagacatcccctgtggatgaaaaggccctacaggaccagctggtgctagtcgctgcaaaacttgacaccgaagacaagttgagggccgcaatggtcgggatgctggccaacttcttgggcttccgtatatatggcatgcacagtgagctatggggcgtggtccatggggccaccgtcctctccccaacggctgtctttggcaccctggcctctctctatctgggagccttggaccacacagctgacaggctacaggcaatcctgggtgttccttggaaggacaagaactgcacctcccggctggatgcgcacaaggtcctgtctgccctgcaggctgtacagggcctgctagtggcccagggcagggctgatagccaggcccagctgctgctgtccacggtggtgggcgtgttcacagccccaggcctgcacctgaagcagccgtttgtgcagggcctggctctctatacccctgtggtcctcccacgctctctggacttcacagaactggatgttgctgctgagaagattgacaggttcatgcaggctgtgacaggatggaagactggctgctccctgatgggagccagtgtggacagcaccctggctttcaacacctacgtccacttccaagggaagatgaagggcttctccctgctggccgagccccaggagttctgggtggacaacagcacctcagtgtctgttcccatgctctctggcatgggcaccttccagcactggagtgacatccaggacaacttctcggtgactcaagtgcccttcactgagagcgcctgcctgctgctgatccagcctcactatgcctctgacctggacaaggtggagggtctcactttccagcaaaactccctcaactggatgaagaaactatctccccggaccatccacctgaccatgccccaactggtgctgcaaggatcttatgacctgcaggacctgctcgcccaggctgagctgcccgccattctgcacaccgagctgaacctgcaaaaattgagcaatgaccgcatcagggtgggggaggtgctgaacagcattttttttgagcttgaagcggatgagagagagcccacagagtctacccaacagcttaacaagcctgaggtcttggaggtgaccctgaaccgcccattcctgtttgctgtgtatgatcaaagcgccactgccctgcacttcctgggccgcgtggccaacccgctgagcacagcatgaggccagggccccagaacacagtgcctggcaaggcctctgcccctggcctttgaggcaaaggccagcagcagataacaaccccggacaaatcagcgatgtgtcacccccagtctcccaccttttcttctaatgagtcgactttgagctggaaagcagccgtttctccttggtctaagtgtgctgcatggagtgagcagtagaagcctgcagcggcacaaatgcacctcccagtttgctgggtttattttagagaatgggggtggggaggcaagaaccagtgtttagcgcgggactactgttccaaaaagaattccaaccgaccagcttgtttgtgaaacaaaaaagtgttcccttttcaagttgagaacaaaaattgggttttaaaattaaagtatacatttttgcattgccttcggtttgtatttagtgtcttgaatgtaagaacatgacctccgtgtagtgtctgtaataccttagttttttccacagatgcttgtgatttttgaacaatacgtgaaagatgcaagcacctgaatttctgtttgaatgcggaaccatagctggttatttctcccttgtgttagtaataaacgtcttgccacaataagcctccaaaaa

SEQ ID NO: 520 - RNA, 1434 nt, Mus musculus
  source 1..1434: mol_type = mRNA
atgactcccacgggggcaggcctgaaggccaccatcttctgcatcttgacctgggtcagcctgacggctggggaccgcgtatacatccaccccttccatctcctttaccacaacaagagcacctgcgcccagctggagaaccccagtgtggagacactcccagagtcaacgttcgagcctgtgcccattcaggccaagacctcccctgtgaatgagaagaccctgcatgatcagctcgtgctggccgccgagaagctagaggatgaggaccggaagcgggctgcccaggtcgcaatgatcgccaacttcgtgggcttccgcatgtacaagatgctgaatgaggcaggaagtggggccagtggggccatcctctcaccaccagctctctttggcaccctggtctctttctaccttggatccttagatcccacggccagccagctgcagacgctgctggatgtccctgtgaaggagggagactgcacctcccgactagatggacacaaggtcctcgctgccctgcgggccattcagggcttgctggtcacccagggtgggagcagcagccagacacccctgctacagtccattgtggtggggctcttcactgctccaggctttcgtctaaagcactcatttgttcagagcctggctctctttacccctgccctcttcccacgctctctggatttatccactgacccagttcttgccactgagaaaatcaacaggttcataaaggctgtgacagggtggaagatgaacttgccactggagggggtcagtacagacagcaccctacttttcaacacctacgttcacttccaaggaacgatgagaggtttctctcagctgcctggagtccatgaattctgggtggacaacagcatctcggtgtctgtgcccatgatctccggcactggcaacttccagcactggagtgacacccagaacaacttctccgtgacgtgcgtgcccctaggtgagagagccaccctgctgctcatccagccccactgcacctcagatctcgacagggtggaggccctcatcttccggaacgacctcctgacttggatagagaacccgcctcctcgggccatccgcctgactctgccccagctggaaatccgaggatcctacaatctgcaggacctgctggctgaggacaagctgcccacccttttgggtgcggaggcaaatctgaacaacattggtgacaccaacccccgagtgggagaggttctcaatagcatcctcctcgaactcaaagcaggagaggaggaacagccgaccacgtctgtccagcagcctggctcaccggaggcactggatgtgaccctgagcagccccttcctgttcgccatctacgagcaggactcaggcacgctgcactttctgggcagagtgaataacccccagagtgtggtgtga

SEQ ID NO: 521 - RNA, 1458 nt, Macaca fascicularis
  source 1..1458: mol_type = mRNA
atgcagaagcgagcaccccagtccgagatggctcctgccagcgtgagcctgagggccaccatcctctgcctcctggcctgggctggcctggccacaggtgaccgggtgtacatacaccccttccacctcgtcatccacaatgagagtacctgtgagcagctggcaaaggccgatgctgggaagcccaaagatcccaccttcacacctgttccgatacaggccaagacgtctcctgtggatgaaaaggccctgcaggaccagctagtgctggttgccgcaaaactcgacaccgaggacaagttgagagccgcgatggtcgggatgctggccaacttcttgggcttccgtatatatggcatgcacagtgagctatggggcgtggtccatggggccaccatcctctccccaacggctgtctttggcaccctggcctctctctacctgggagcgttggaccacacagccgacaggctacaggcaatcctgggcgtcccttggaaggacaagaactgcacctcccggctggatgcgcacaaggtcctctctgccctgcaggctgtacagggcctgctggtggcccagggcagggctgacggccagtcccagctgctgttgtccacagtggtgggtctcttcacagccccagatctgcacctgaagcagccgtttgtgcagggcctggctctctatgcccctgtggtcctcccacgctctctggacttcacagacctggaagtcgctgctgagaagattgacaggttcatgcaggctgtgacaggatggaagattagcagccccctgacgggagccagtgcggacagcaccctggttttcaacacctacgtccatttccaagggaagatgagggacttcttcctgctggctgagccccaggagttctgggtggacaacagcacctcagtgtctgtccccatgctgtctggcgtgggcaccttccagcactggagcgacgcccaggacaacttctcagtgactcaagtgccctttactgagagcgcctgcttgctgctgattcagcctcactacgcctctgacctggacaaggtggagggtctcactttccagcaaaactccctcaactggatgaagaaactgtctccccgggccatccacctgaccatgccccgactggtgctgcgaggatcttatgacctgcaggacctgcttgcccaggctgagctgcccgccattctgggcaccgagctgaacctgcaaaaattgagcaatgacaacctcagggtggggaaggtgctgaacagcattctttttgaactcgaagcggatgagagagagcccacagagtctacccgacagctgaacaggcctgagttcttggaggtgaccctggaccgcccattcctgtttgctgtgtatgatcaaagtgccactgccctgcacttcctgggccgtgtggccaacccgctgagcccagcatga

SEQ ID NO: 522 - RNA, 21 nt, synthetic construct
  source 1..21: mol_type = other RNA
caccagcttgtttgtgaaaca

SEQ ID NO: 523 - RNA, 21 nt, synthetic construct
  source 1..21: mol_type = other RNA
tgtttcacaaacaagctggtg

SEQ ID NO: 524 - RNA, 21 nt, synthetic construct
  source 1..21: mol_type = other RNA
ccaaccgaccagcttgtttga

SEQ ID NO: 525 - RNA, 21 nt, synthetic construct
  source 1..21: mol_type = other RNA
tcaaacaagctggtcggttgg

SEQ ID NO: 526 - RNA, 21 nt, synthetic construct
  source 1..21: mol_type = other RNA
caccagcttgtttgtgaaaca

SEQ ID NO: 527 - RNA, 21 nt, synthetic construct
  source 1..21: mol_type = other RNA
tgtttcacaaacaagctggtg

SEQ ID NO: 528 - RNA, 21 nt, synthetic construct
  source 1..21: mol_type = other RNA
caccagcttgtttgtgaaaca

SEQ ID NO: 529 - RNA, 21 nt, synthetic construct
  source 1..21: mol_type = other RNA
caccagcttgtttgtgaaact

SEQ ID NO: 530 - RNA, 21 nt, synthetic construct
  source 1..21: mol_type = other RNA
caccagcttgtttgtgaaacc

SEQ ID NO: 531 - RNA, 21 nt, synthetic construct
  source 1..21: mol_type = other RNA
caccagcttgtttgtgaaacg

SEQ ID NO: 532 - RNA, 17 nt, synthetic construct
  source 1..17: mol_type = other RNA
agcttgtttgtgaaaca

SEQ ID NO: 533 - RNA, 18 nt, synthetic construct
  source 1..18: mol_type = other RNA
cagcttgtttgtgaaaca

SEQ ID NO: 534 - RNA, 19 nt, synthetic construct
  source 1..19: mol_type = other RNA
acagcttgtttgtgaaaca

SEQ ID NO: 535 - RNA, 19 nt, synthetic construct
  source 1..19: mol_type = other RNA
tcagcttgtttgtgaaaca

SEQ ID NO: 536 - RNA, 19 nt, synthetic construct
  source 1..19: mol_type = other RNA
gcagcttgtttgtgaaaca

SEQ ID NO: 537 - RNA, 19 nt, synthetic construct
  source 1..19: mol_type = other RNA
ccagcttgtttgtgaaaca

SEQ ID NO: 538 - RNA, 20 nt, synthetic construct
  source 1..20: mol_type = other RNA
accagcttgtttgtgaaaca

SEQ ID NO: 539 - RNA, 20 nt, synthetic construct
  source 1..20: mol_type = other RNA
tccagcttgtttgtgaaaca

SEQ ID NO: 540 - RNA, 20 nt, synthetic construct
  source 1..20: mol_type = other RNA
gccagcttgtttgtgaaaca

SEQ ID NO: 541 - RNA, 20 nt, synthetic construct
  source 1..20: mol_type = other RNA
cccagcttgtttgtgaaaca

SEQ ID NO: 542 - RNA, 21 nt, synthetic construct
  source 1..21: mol_type = other RNA
gaccagcttgtttgtgaaaca

SEQ ID NO: 543 - RNA, 21 nt, synthetic construct
  source 1..21: mol_type = other RNA
aaccagcttgtttgtgaaaca

SEQ ID NO: 544 - RNA, 21 nt, synthetic construct
  source 1..21: mol_type = other RNA
taccagcttgtttgtgaaaca

SEQ ID NO: 545 - RNA, 22 nt, synthetic construct
  source 1..22: mol_type = other RNA
cgaccagcttgtttgtgaaaca

SEQ ID NO: 546 - RNA, 23 nt, synthetic construct
  source 1..23: mol_type = other RNA
ccgaccagcttgtttgtgaaaca

SEQ ID NO: 547 - RNA, 24 nt, synthetic construct
  source 1..24: mol_type = other RNA
accgaccagcttgtttgtgaaaca

SEQ ID NO: 548 - RNA, 25 nt, synthetic construct
  source 1..25: mol_type = other RNA
aaccgaccagcttgtttgtgaaaca

SEQ ID NO: 549 - RNA, 26 nt, synthetic construct
  source 1..26: mol_type = other RNA
caaccgaccagcttgtttgtgaaaca

SEQ ID NO: 550 - RNA, 32 nt, synthetic construct
  source 1..32: mol_type = other RNA
gaattccaaccgaccagcttgtttgtgaaaca

SEQ ID NO: 551 - RNA, 21 nt, synthetic construct
  source 1..21: mol_type = other RNA
caccagcttgtttgtgaaaca

SEQ ID NO: 552 - RNA, 21 nt, synthetic construct
  source 1..21: mol_type = other RNA
caccagcttgtttgtgaaaca

SEQ ID NO: 553 - RNA, 21 nt, synthetic construct
  source 1..21: mol_type = other RNA
caccagcttgtttgtgaaaca

SEQ ID NO: 554 - RNA, 21 nt, synthetic construct
  source 1..21: mol_type = other RNA
caccagcttgtttgtgaaaca

SEQ ID NO: 555 - RNA, 21 nt, synthetic construct
  source 1..21: mol_type = other RNA
caccagcttgtttgtgaaaca

SEQ ID NO: 556 - RNA, 21 nt, synthetic construct
  source 1..21: mol_type = other RNA
caccagcttgtttgtaaaaca

SEQ ID NO: 557 - RNA, 21 nt, synthetic construct
  source 1..21: mol_type = other RNA
caccagcttgtttgtgaaata

SEQ ID NO: 558 - RNA, 21 nt, synthetic construct
  source 1..21: mol_type = other RNA
caccagcttgtttgtgaaaca

SEQ ID NO: 559 - RNA, 21 nt, synthetic construct
  source 1..21: mol_type = other RNA
tgtttcacaaacaagctggtg

SEQ ID NO: 560 - RNA, 21 nt, synthetic construct
  source 1..21: mol_type = other RNA
agtttcacaaacaagctggtg

SEQ ID NO: 561 - RNA, 21 nt, synthetic construct
  source 1..21: mol_type = other RNA
ggtttcacaaacaagctggtg

SEQ ID NO: 562 - RNA, 21 nt, synthetic construct
  source 1..21: mol_type = other RNA
cgtttcacaaacaagctggtg

SEQ ID NO: 563 - RNA, 17 nt, synthetic construct
  source 1..17: mol_type = other RNA
tgtttcacaaacaagct

SEQ ID NO: 564 - RNA, 18 nt, synthetic construct
  source 1..18: mol_type = other RNA
tgtttcacaaacaagctg

SEQ ID NO: 565 - RNA, 19 nt, synthetic construct
  source 1..19: mol_type = other RNA
tgtttcacaaacaagctgt

SEQ ID NO: 566 - RNA, 19 nt, synthetic construct
  source 1..19: mol_type = other RNA
tgtttcacaaacaagctga

SEQ ID NO: 567 - RNA, 19 nt, synthetic construct
  source 1..19: mol_type = other RNA
tgtttcacaaacaagctgc

SEQ ID NO: 568 - RNA, 19 nt, synthetic construct
  source 1..19: mol_type = other RNA
tgtttcacaaacaagctgg

SEQ ID NO: 569 - RNA, 20 nt, synthetic construct
  source 1..20: mol_type = other RNA
tgtttcacaaacaagctggt

SEQ ID NO: 570 - RNA, 20 nt, synthetic construct
  source 1..20: mol_type = other RNA
tgtttcacaaacaagctgga

SEQ ID NO: 571 - RNA, 20 nt, synthetic construct
  source 1..20: mol_type = other RNA
tgtttcacaaacaagctggc

SEQ ID NO: 572 - RNA, 20 nt, synthetic construct
  source 1..20: mol_type = other RNA
tgtttcacaaacaagctggg

SEQ ID NO: 573 - RNA, 21 nt, synthetic construct
  source 1..21: mol_type = other RNA
tgtttcacaaacaagctggtc

SEQ ID NO: 574 - RNA, 21 nt, synthetic construct
  source 1..21: mol_type = other RNA
tgtttcacaaacaagctggtt

SEQ ID NO: 575 - RNA, 21 nt, synthetic construct
  source 1..21: mol_type = other RNA
tgtttcacaaacaagctggta

SEQ ID NO: 576 - RNA, 22 nt, synthetic construct
  source 1..22: mol_type = other RNA
tgtttcacaaacaagctggtcg

SEQ ID NO: 577 - RNA, 23 nt, synthetic construct
  source 1..23: mol_type = other RNA
tgtttcacaaacaagctggtcgg

SEQ ID NO: 578 - RNA, 24 nt, synthetic construct
  source 1..24: mol_type = other RNA
tgtttcacaaacaagctggtcggt

SEQ ID NO: 579 - RNA, 25 nt, synthetic construct
  source 1..25: mol_type = other RNA
tgtttcacaaacaagctggtcggtt

SEQ ID NO: 580 - RNA, 26 nt, synthetic construct
  source 1..26: mol_type = other RNA
tgtttcacaaacaagctggtcggttg

SEQ ID NO: 581 - RNA, 32 nt, synthetic construct
  source 1..32: mol_type = other RNA
tgtttcacaaacaagctggtcggttggaattc

SEQ ID NO: 582 - RNA, 23 nt, synthetic construct
  source 1..23: mol_type = other RNA
tgtttcacaaacaagctggtgtt

SEQ ID NO: 583 - RNA, 23 nt, synthetic construct
  source 1..23: mol_type = other RNA
tgtttcacaaacaagctggtgga

SEQ ID NO: 584 - RNA, 21 nt, synthetic construct
  source 1..21: mol_type = other RNA
tgtttcacaaacaagctggtg

SEQ ID NO: 585 - RNA, 21 nt, synthetic construct
  source 1..21: mol_type = other RNA
tgtttcacaaacaagctggtg

SEQ ID NO: 586 - RNA, 21 nt, synthetic construct
  source 1..21: mol_type = other RNA
tgtttcacaaacaagctggtg

SEQ ID NO: 587 - RNA, 21 nt, synthetic construct
  source 1..21: mol_type = other RNA
tgtttcacaaacaagctggtg

SEQ ID NO: 588 - RNA, 21 nt, synthetic construct
  source 1..21: mol_type = other RNA
tatttcacaaacaagctggtg

SEQ ID NO: 589 - RNA, 21 nt, synthetic construct
  source 1..21: mol_type = other RNA
tgtttcacaaacaagctggtg

SEQ ID NO: 590 - RNA, 21 nt, synthetic construct
  source 1..21: mol_type = other RNA
caccagcttgtttgtgaaaca

SEQ ID NO: 591 - RNA, 21 nt, synthetic construct
  source 1..21: mol_type = other RNA
caccagcttgtttgtgaaact

SEQ ID NO: 592 - RNA, 21 nt, synthetic construct
  source 1..21: mol_type = other RNA
caccagcttgtttgtgaaacc

SEQ ID NO: 593 - RNA, 21 nt, synthetic construct
  source 1..21: mol_type = other RNA
caccagcttgtttgtgaaacg

SEQ ID NO: 594 - RNA, 17 nt, synthetic construct
  source 1..17: mol_type = other RNA
agcttgtttgtgaaaca

SEQ ID NO: 595 - RNA, 18 nt, synthetic construct
  source 1..18: mol_type = other RNA
cagcttgtttgtgaaaca

SEQ ID NO: 596 - RNA, 19 nt, synthetic construct
  source 1..19: mol_type = other RNA
acagcttgtttgtgaaaca

SEQ ID NO: 597 - RNA, 19 nt, synthetic construct
  source 1..19: mol_type = other RNA
tcagcttgtttgtgaaaca

SEQ ID NO: 598 - RNA, 19 nt, synthetic construct
  source 1..19: mol_type = other RNA
gcagcttgtttgtgaaaca

SEQ ID NO: 599 - RNA, 19 nt, synthetic construct
  source 1..19: mol_type = other RNA
ccagcttgtttgtgaaaca

SEQ ID NO: 600 - RNA, 20 nt, synthetic construct
  source 1..20: mol_type = other RNA
accagcttgtttgtgaaaca

SEQ ID NO: 601 - RNA, 20 nt, synthetic construct
  source 1..20: mol_type = other RNA
tccagcttgtttgtgaaaca

SEQ ID NO: 602 - RNA, 20 nt, synthetic construct
  source 1..20: mol_type = other RNA
gccagcttgtttgtgaaaca

SEQ ID NO: 603 - RNA, 20 nt, synthetic construct
  source 1..20: mol_type = other RNA
cccagcttgtttgtgaaaca

SEQ ID NO: 604 - RNA, 21 nt, synthetic construct
  source 1..21: mol_type = other RNA
gaccagcttgtttgtgaaaca

SEQ ID NO: 605 - RNA, 21 nt, synthetic construct
  source 1..21: mol_type = other RNA
aaccagcttgtttgtgaaaca

SEQ ID NO: 606 - RNA, 21 nt, synthetic construct
  source 1..21: mol_type = other RNA
taccagcttgtttgtgaaaca

SEQ ID NO: 607 - RNA, 22 nt, synthetic construct
  source 1..22: mol_type = other RNA
cgaccagcttgtttgtgaaaca

SEQ ID NO: 608 - RNA, 23 nt, synthetic construct
  source 1..23: mol_type = other RNA
ccgaccagcttgtttgtgaaaca

SEQ ID NO: 609 - RNA, 24 nt, synthetic construct
  source 1..24: mol_type = other RNA
accgaccagcttgtttgtgaaaca

SEQ ID NO: 610 - RNA, 25 nt, synthetic construct
  source 1..25: mol_type = other RNA
aaccgaccagcttgtttgtgaaaca

SEQ ID NO: 611 - RNA, 26 nt, synthetic construct
  source 1..26: mol_type = other RNA
caaccgaccagcttgtttgtgaaaca

SEQ ID NO: 612 - RNA, 32 nt, synthetic construct; identical to SEQ ID NO: 550
  source 1..32: mol_type = other RNA

SEQ ID NO: 613 - RNA, 21 nt, synthetic construct
  source 1..21: mol_type = other RNA
caccagcttgtttgtgaaaca

SEQ ID NO: 614 - RNA, 21 nt, synthetic construct
  source 1..21: mol_type = other RNA
caccagcttgtttgtgaaaca

SEQ ID NO: 615 - RNA, 21 nt, synthetic construct
  source 1..21: mol_type = other RNA
caccagcttgtttgtgaaaca

SEQ ID NO: 616 - RNA, 21 nt, synthetic construct
  source 1..21: mol_type = other RNA
caccagcttgtttgtgaaaca

SEQ ID NO: 617 - RNA, 21 nt, synthetic construct
  source 1..21: mol_type = other RNA
caccagcttgtttgtgaaaca

SEQ ID NO: 618 - RNA, 21 nt, synthetic construct
  source 1..21: mol_type = other RNA
caccagcttgtttgtaaaaca

SEQ ID NO: 619 - RNA, 21 nt, synthetic construct
  source 1..21: mol_type = other RNA
caccagcttgtttgtgaaata

SEQ ID NO: 620 - RNA, 21 nt, synthetic construct
  source 1..21: mol_type = other RNA
caccagcttgtttgtgaaaca

SEQ ID NO: 621 - RNA, 21 nt, synthetic construct
  source 1..21: mol_type = other RNA
tgtttcacaaacaagctggtg

SEQ ID NO: 622 - RNA, 21 nt, synthetic construct
  source 1..21: mol_type = other RNA
agtttcacaaacaagctggtg

SEQ ID NO: 623 - RNA, 21 nt, synthetic construct
  source 1..21: mol_type = other RNA
ggtttcacaaacaagctggtg

SEQ ID NO: 624 - RNA, 21 nt, synthetic construct
  source 1..21: mol_type = other RNA
cgtttcacaaacaagctggtg

SEQ ID NO: 625 - RNA, 17 nt, synthetic construct
  source 1..17: mol_type = other RNA
tgtttcacaaacaagct

SEQ ID NO: 626 - RNA, 18 nt, synthetic construct
  source 1..18: mol_type = other RNA
tgtttcacaaacaagctg

SEQ ID NO: 627 - RNA, 19 nt, synthetic construct
  source 1..19: mol_type = other RNA
tgtttcacaaacaagctgt

SEQ ID NO: 628 - RNA, 19 nt, synthetic construct
  source 1..19: mol_type = other RNA
tgtttcacaaacaagctga

SEQ ID NO: 629 - RNA, 19 nt, synthetic construct
  source 1..19: mol_type = other RNA
tgtttcacaaacaagctgc

SEQ ID NO: 630 - RNA, 19 nt, synthetic construct
  source 1..19: mol_type = other RNA
tgtttcacaaacaagctgg

SEQ ID NO: 631 - RNA, 20 nt, synthetic construct
  source 1..20: mol_type = other RNA
tgtttcacaaacaagctggt

SEQ ID NO: 632 - RNA, 20 nt, synthetic construct
  source 1..20: mol_type = other RNA
tgtttcacaaacaagctgga

SEQ ID NO: 633 - RNA, 20 nt, synthetic construct
  source 1..20: mol_type = other RNA
tgtttcacaaacaagctggc

SEQ ID NO: 634 - RNA, 20 nt, synthetic construct
  source 1..20: mol_type = other RNA
tgtttcacaaacaagctggg

SEQ ID NO: 635 - RNA, 21 nt, synthetic construct
  source 1..21: mol_type = other RNA
tgtttcacaaacaagctggtc

SEQ ID NO: 636 - RNA, 21 nt, synthetic construct
  source 1..21: mol_type = other RNA
tgtttcacaaacaagctggtt

SEQ ID NO: 637 - RNA, 21 nt, synthetic construct
  source 1..21: mol_type = other RNA
tgtttcacaaacaagctggta

SEQ ID NO: 638 - RNA, 22 nt, synthetic construct
  source 1..22: mol_type = other RNA
tgtttcacaaacaagctggtcg

SEQ ID NO: 639 - RNA, 23 nt, synthetic construct
  source 1..23: mol_type = other RNA
tgtttcacaaacaagctggtcgg

SEQ ID NO: 640 - RNA, 24 nt, synthetic construct
  source 1..24: mol_type = other RNA
tgtttcacaaacaagctggtcggt

SEQ ID NO: 641 - RNA, 25 nt, synthetic construct
  source 1..25: mol_type = other RNA
tgtttcacaaacaagctggtcggtt

SEQ ID NO: 642 - RNA, 26 nt, synthetic construct
  source 1..26: mol_type = other RNA
tgtttcacaaacaagctggtcggttg

SEQ ID NO: 643 - RNA, 32 nt, synthetic construct; identical to SEQ ID NO: 581
  source 1..32: mol_type = other RNA

SEQ ID NO: 644 - RNA, 23 nt, synthetic construct
  source 1..23: mol_type = other RNA
tgtttcacaaacaagctggtgtt

SEQ ID NO: 645 - RNA, 23 nt, synthetic construct
  source 1..23: mol_type = other RNA
tgtttcacaaacaagctggtgga

SEQ ID NO: 646 - RNA, 21 nt, synthetic construct
  source 1..21: mol_type = other RNA
tgtttcacaaacaagctggtg

SEQ ID NO: 647 - RNA, 21 nt, synthetic construct
  source 1..21: mol_type = other RNA
tgtttcacaaacaagctggtg

SEQ ID NO: 648 - RNA, 21 nt, synthetic construct
  source 1..21: mol_type = other RNA
tgtttcacaaacaagctggtg

SEQ ID NO: 649 - RNA, 21 nt, synthetic construct
  source 1..21: mol_type = other RNA
tgtttcacaaacaagctggtg

SEQ ID NO: 650 - RNA, 21 nt, synthetic construct
  source 1..21: mol_type = other RNA
tatttcacaaacaagctggtg

SEQ ID NO: 651 - RNA, 21 nt, synthetic construct
  source 1..21: mol_type = other RNA
tgtttcacaaacaagctggtg

SEQ ID NO: 652 - RNA, 18 nt, synthetic construct
  source 1..18: mol_type = other RNA
ccagcttgtttgtgaaac

SEQ ID NO: 653 - RNA, 18 nt, synthetic construct
  source 1..18: mol_type = other RNA
gtttcacaaacaagctgg

SEQ ID NO: 654 - RNA, 21 nt, synthetic construct
  source 1..21: mol_type = other RNA
gcgtttctccttggtctaaga

SEQ ID NO: 655 - RNA, 21 nt, synthetic construct
  source 1..21: mol_type = other RNA
tcttagaccaaggagaaacgc

SEQ ID NO: 656 - RNA, 32 nt, synthetic construct
  source 1..32: mol_type = other RNA
  variation 1..15: note = n can be A, T, C, G or absent
  variation 32: note = n can be A, T, C, G or absent
nnnnnnnnnnnnnnnagcttgtttgtgaaacn

SEQ ID NO: 657 - RNA, 32 nt, synthetic construct
  source 1..32: mol_type = other RNA
  variation 1: note = n can be A, T, C, G or absent
  variation 18..32: note = n can be A, T, C, G or absent
ngtttcacaaacaagctnnnnnnnnnnnnnnn

SEQ ID NO: 658 - RNA, 31 nt, synthetic construct
  source 1..31: mol_type = other RNA
  variation 1..13: note = n can be A, T, C, G or absent
nnnnnnnnnnnnncagcttgtttgtgaaaca

SEQ ID NO: 659 - RNA, 31 nt, synthetic construct
  source 1..31: mol_type = other RNA
  variation 19..31: note = n can be A, T, C, G or absent
tgtttcacaaacaagctgnnnnnnnnnnnnn

SEQ ID NO: 660 - RNA, 10 nt, synthetic construct
  source 1..10: mol_type = other RNA
ccaaccgacc

SEQ ID NO: 661 - RNA, 11 nt, synthetic construct
  source 1..11: mol_type = other RNA
tccaaccgacc

SEQ ID NO: 662 - RNA, 12 nt, synthetic construct
  source 1..12: mol_type = other RNA
ttccaaccgacc

SEQ ID NO: 663 - RNA, 13 nt, synthetic construct
  source 1..13: mol_type = other RNA
attccaaccgacc

SEQ ID NO: 664 - RNA, 14 nt, synthetic construct
  source 1..14: mol_type = other RNA
aattccaaccgacc

SEQ ID NO: 665 - RNA, 15 nt, synthetic construct
  source 1..15: mol_type = other RNA
gaattccaaccgacc

SEQ ID NO: 666 - RNA, 10 nt, synthetic construct
  source 1..10: mol_type = other RNA
ggtcggttgg

SEQ ID NO: 667 - RNA, 11 nt, synthetic construct
  source 1..11: mol_type = other RNA
ggtcggttgga

SEQ ID NO: 668 - RNA, 12 nt, synthetic construct
  source 1..12: mol_type = other RNA
ggtcggttggaa

SEQ ID NO: 669 - RNA, 13 nt, synthetic construct
  source 1..13: mol_type = other RNA
ggtcggttggaat

SEQ ID NO: 670 - RNA, 14 nt, synthetic construct
  source 1..14: mol_type = other RNA
ggtcggttggaatt

SEQ ID NO: 671 - RNA, 15 nt, synthetic construct
  source 1..15: mol_type = other RNA
ggtcggttggaattc

SEQ ID NO: 672 - RNA, 14 nt, synthetic construct
  source 1..14: mol_type = other RNA
gaattccaaccgac

SEQ ID NO: 673 - RNA, 14 nt, synthetic construct
  source 1..14: mol_type = other RNA
gtcggttggaattc